SEQUENCE LISTING (75 sequences)

SEQ ID NO: 1 - protein, 404 aa, Homo sapiens
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - protein, 402 aa, Mus musculus
MPAGTAARAWVLVLALWGAVAGGQNITARIGEPLVLSCKGAPKKPPQQLEWKLNTGRTEAWKVLSPQGGPWDSVARILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHEDEGTYSCVATHPSHGPQESPPVSIRVTETGDEGPAEGSVGESGLGTLALALGILGGLGVVALLVGAILWRKRQPRREERKAPESQEDEEERAELNQSEEAEMPENGAGGP

SEQ ID NO: 3 - protein, 553 aa, synthetic construct
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLAQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 549 aa, synthetic construct
GQNITARIGEPLVLSCKGAPKKPPQQLEWKLNTGRTEAWKVLSPQGGPWDSVARILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHEDEGTYSCVATHPSHGPQESPPVSIRVTETGDEGPAEGSVGESGLGTQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 6 - protein, 2473 aa, Mus musculus
MPALRPAALRALLWLWLCGAGPAHALQCRGGQEPCVNEGTCVTYHNGTGFCRCPEGFLGEYCQHRDPCEKNRCQNGGTCVPQGMLGKATCRCAPGFTGEDCQYSTSHPCFVSRPCQNGGTCHMLSRDTYECTCQVGFTGKQCQWTDACLSHPCENGSTCTSVASQFSCKCPAGLTGQKCEADINECDIPGRCQHGGTCLNLPGSYRCQCPQGFTGQHCDSPYVPCAPSPCVNGGTCRQTGDFTFECNCLPGFEGSTCERNIDDCPNHKCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGILCDENIDNCDPDPCHHGQCQDGIDSYTCICNPGYMGAICSDQIDECYSSPCLNDGRCIDLVNGYQCNCQPGTSGLNCEINFDDCASNPCMHGVCVDGINRYSCVCSPGFTGQRCNIDIDECASNPCRKGATCINDVNGFRCICPEGPHHPSCYSQVNECLSNPCIHGNCTGGLSGYKCLCDAGWVGVNCEVDKNECLSNPCQNGGTCNNLVNGYRCTCKKGFKGYNCQVNIDECASNPCLNQGTCFDDVSGYTCHCMLPYTGKNCQTVLAPCSPNPCENAAVCKEAPNFESFSCLCAPGWQGKRCTVDVDECISKPCMNNGVCHNTQGSYVCECPPGFSGMDCEEDINDCLANPCQNGGSCVDHVNTFSCQCHPGFIGDKCQTDMNECLSEPCKNGGTCSDYVNSYTCTCPAGFHGVHCENNIDECTESSCFNGGTCVDGINSFSCLCPVGFTGPFCLHDINECSSNPCLNAGTCVDGLGTYRCICPLGYTGKNCQTLVNLCSRSPCKNKGTCVQEKARPHCLCPPGWDGAYCDVLNVSCKAAALQKGVPVEHLCQHSGICINAGNTHHCQCPLGYTGSYCEEQLDECASNPCQHGATCNDFIGGYRCECVPGYQGVNCEYEVDECQNQPCQNGGTCIDLVNHFKCSCPPGTRGLLCEENIDECAGGPHCLNGGQCVDRIGGYTCRCLPGFAGERCEGDINECLSNPCSSEGSLDCVQLKNNYNCICRSAFTGRHCETFLDVCPQKPCLNGGTCAVASNMPDGFICRCPPGFSGARCQSSCGQVKCRRGEQCIHTDSGPRCFCLNPKDCESGCASNPCQHGGTCYPQRQPPHYSCRCPPSFGGSHCELYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSELESPRNAQLLYLLAVAVVIILFFILLGVIMAKRKRKHGFLWLPEGFTLRRDSSNHKRREPVGQDAVGLKNLSVQVSEANLIGSGTSEHWVDDEGPQPKKAKAEDEALLSEDDPIDRRPWTQQHLEAADIRHTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVLCGPNRSFLSLKHTPMGKKARRPNTKSTMPTSLPNLAKEAKDAKGSRRKKCLNEKVQLSESSVTLSPVDSLESPHTYVSDATSSPMITSPGILQASPTPLLAAAAPAAPVHTQHALSFSNLHDMQPLAPGASTVLPSVSQLLSHHHIAPPGSSSAGSLGRLHPVPVPADWMNRVEMNETQYSEMFGMVLAPAEGAHPGIAAPQSRPPEGKHMSTQREPLPPIVTFQLIPKGSIAQAAGAPQTQSSCPPAVAGPLPSMYQIPEMPRLPSVAFPPTMMPQQEGQVAQTIVPTYHPFPASVGKYPTPPSQHSYASSNAAERTPSHGGHLQGEHPYLTPSPESPDQWSSSSPHSASDWSDVTTSPTPGGGGGGQRGPGTHMSEPPHSNMQVYA

SEQ ID NO: 7 - protein, 1885 aa, synthetic construct
LQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 1887 aa, synthetic construct
LQCRGGQEPCVNEGTCVTYHNGTGFCRCPEGFLGEYCQHRDPCEKNRCQNGGTCVPQGMLGKATCRCAPGFTGEDCQYSTSHPCFVSRPCQNGGTCHMLSRDTYECTCQVGFTGKQCQWTDACLSHPCENGSTCTSVASQFSCKCPAGLTGQKCEADINECDIPGRCQHGGTCLNLPGSYRCQCPQGFTGQHCDSPYVPCAPSPCVNGGTCRQTGDFTFECNCLPGFEGSTCERNIDDCPNHKCQNGGVCVDGVNTYNCRCPPQWTGQFCTEDVDECLLQPNACQNGGTCTNRNGGYGCVCVNGWSGDDCSENIDDCAYASCTPGSTCIDRVASFSCLCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEVNECQSNPCVNNGQCVDKVNRFQCLCPPGFTGPVCQIDIDDCSSTPCLNGAKCIDHPNGYECQCATGFTGILCDENIDNCDPDPCHHGQCQDGIDSYTCICNPGYMGAICSDQIDECYSSPCLNDGRCIDLVNGYQCNCQPGTSGLNCEINFDDCASNPCMHGVCVDGINRYSCVCSPGFTGQRCNIDIDECASNPCRKGATCINDVNGFRCICPEGPHHPSCYSQVNECLSNPCIHGNCTGGLSGYKCLCDAGWVGVNCEVDKNECLSNPCQNGGTCNNLVNGYRCTCKKGFKGYNCQVNIDECASNPCLNQGTCFDDVSGYTCHCMLPYTGKNCQTVLAPCSPNPCENAAVCKEAPNFESFSCLCAPGWQGKRCTVDVDECISKPCMNNGVCHNTQGSYVCECPPGFSGMDCEEDINDCLANPCQNGGSCVDHVNTFSCQCHPGFIGDKCQTDMNECLSEPCKNGGTCSDYVNSYTCTCPAGFHGVHCENNIDECTESSCFNGGTCVDGINSFSCLCPVGFTGPFCLHDINECSSNPCLNAGTCVDGLGTYRCICPLGYTGKNCQTLVNLCSRSPCKNKGTCVQEKARPHCLCPPGWDGAYCDVLNVSCKAAALQKGVPVEHLCQHSGICINAGNTHHCQCPLGYTGSYCEEQLDECASNPCQHGATCNDFIGGYRCECVPGYQGVNCEYEVDECQNQPCQNGGTCIDLVNHFKCSCPPGTRGLLCEENIDECAGGPHCLNGGQCVDRIGGYTCRCLPGFAGERCEGDINECLSNPCSSEGSLDCVQLKNNYNCICRSAFTGRHCETFLDVCPQKPCLNGGTCAVASNMPDGFICRCPPGFSGARCQSSCGQVKCRRGEQCIHTDSGPRCFCLNPKDCESGCASNPCQHGGTCYPQRQPPHYSCRCPPSFGGSHCELYTAPTSTPPATCQSQYCADKARDGICDEACNSHACQWDGGDCSLTMEDPWANCTSTLRCWEYINNQCDEQCNTAECLFDNFECQRNSKTCKYDKYCADHFKDNHCDQGCNSEECGWDGLDCASDQPENLAEGTLIIVVLLPPEQLLQDSRSFLRALGTLLHTNLRIKQDSQGALMVYPYFGEKSAAMKKQKMTRRSLPEEQEQEQEVIGSKIFLEIDNRQCVQDSDQCFKNTDAAAALLASHAIQGTLSYPLVSVFSELESPRNAQQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1290 aa, synthetic construct
ARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 640 aa, Homo sapiens
MGQPSLTWMLMVVVASWFITTAATDTSEARWCSECHSNATCTEDEAVTTCTCQEGFTGDGLTCVDLDECAIPGAHNCSANSSCVNTPGSFSCVCPEGFRLSPGLGCTDVDECAEPGLSHCHALATCVNVVGSYLCVCPAGYRGDGWHCECSPGSCGPGLDCVPEGDALVCADPCQAHRTLDEYWRSTEYGEGYACDTDLRGWYRFVGQGGARMAETCVPVLRCNTAAPMWLNGTHPSSDEGIVSRKACAHWSGHCCLWDASVQVKACAGGYYVYNLTAPPECHLAYCTDPSSVEGTCEECSIDEDCKSNNGRWHCQCKQDFNITDISLLEHRLECGANDMKVSLGKCQLKSLGFDKVFMYLSDSRCSGFNDRDNRDWVSVVTPARDGPCGTVLTRNETHATYSNTLYLADEIIIRDLNIKINFACSYPLDMKVSLKTALQPMVSALNIRVGGTGMFTVRMALFQTPSYTQPYQGSSVTLSTEAFLYVGTMLDGGDLSRFALLMTNCYATPSSNATDPLKYFIIQDRCPHTRDSTIQVVENGESSQGRFSVQMFRFAGNYDLVYLHCEVYLCDTMNEKCKPTCSGTRFRSGSVIDQSRVLNLGPITRKGVQATVSRAFSSLGLLKVWLPLLLSATLTLTFQ

SEQ ID NO: 11 - protein, 642 aa, Mus musculus
MGIPLTWMLLVMMVTSWFTLAEASNSTEARRCSECHNNATCTVDGVVTTCSCQTGFTGDGLVCEDMDECATPWTHNCSNSSCVNTPGSFKCSCQDGFRLTPELSCTDVDECSEQGLSNCHALATCVNTEGDYLCVCPEGFTGDGWYCECSPGSCEPGLDCLPQGPDGKLVCQDPCNTYETLTEYWRSTEYGVGYSCDAGLHGWYRFTGQGGVRMAETCVPVLRCNTAAPMWLNGSHPSSSEGIVSRTACAHWSDQCCRWSTEIQVKACPGGFYIYNLTAPPECNLAYCTDPSSVEGTCEECRVDEDCISDNGRWRCQCKQDSNITDVSQLEYRLECGANDIKMSLRKCQLQSLGFMNVFMYLNDRQCSGFSESDERDWMSIVTPARNGPCGTVLRRNETHATYSNTLYLANAIIIRDIIIRMNFECSYPLDMKVSLKTSLQPMVSALNISLGGTGKFTVRMALFQSPTYTQPHQGPSVMLSTEAFLYVGTMLDGGDLSRFVLLMTNCYATPSSNSTDPVKYFIIQDSCPRTEDTTIQVTENGESSQARFSVQMFRFAGNYDLVYLHCEVYLCDSTSEQCKPTCSGTRFRSGNFIDQTRVLNLGPITRQGVQASVSKAASSNLRLLSIWLLLFPSATLIFMVQ

SEQ ID NO: 12 - protein, 701 aa, Homo sapiens
MDLWNLSWFLFLDALLVISGLATPENFDVDGGMDQDIFDINEGLGLDLFEGDIRLDRAQIRNSIIGEKYRWPHTIPYVLEDSLEMNAKGVILNAFERYRLKTCIDFKPWAGETNYISVFKGSGCWSSVGNRRVGKQELSIGANCDRIATVQHEFLHALGFWHEQSRSDRDDYVRIMWDRILSGREHNFNTYSDDISDSLNVPYDYTSVMHYSKTAFQNGTEPTIVTRISDFEDVIGQRMDFSDSDLLKLNQLYNCSSSLSFMDSCSFELENVCGMIQSSGDNADWQRVSQVPRGPESDHSNMGQCQGSGFFMHFDSSSVNVGATAVLESRTLYPKRGFQCLQFYLYNSGSESDQLNIYIREYSADNVDGNLTLVEEIKEIPTGSWQLYHVTLKVTKKFRVVFEGRKGSGASLGGLSIDDINLSETRCPHHIWHIRNFTQFIGSPNGTLYSPPFYSSKGYAFQIYLNLAHVTNAGIYFHLISGANDDQLQWPCPWQQATMTLLDQNPDIRQRMSNQRSITTDPFMTTDNGNYFWDRPSKVGTVALFSNGTQFRRGGGYGTSAFITHERLKSRDFIKGDDVYILLTVEDISHLNSTQIQLTPAPSVQDLCSKTTCKNDGVCTVRDGKAECRCQSGEDWWYMGERCEKRGSTRDTIVIAVSSTVAVFALMLIITLVSVYCTRKKYRERMSSNRPNLTPQNQHAF

SEQ ID NO: 13 - protein, 704 aa, Mus musculus
MDARHQPWFLVFATFLLVSGLPAPEKFVKDIDGGIDQDIFDINQGLGLDLFEGDIKLEANGKNSIIGDHKRWPHTIPYVLEDSLEMNAKGVILNAFERYRLKTCIDFKPWSGEANYISVFKGSGCWSSVGNIHAGKQELSIGTNCDRIATVQHEFLHALGFWHEQSRADRDDYVIIVWDRIQPGKEHNFNIYNDSVSDSLNVPYDYTSVMHYSKTAFQNGTESTIVTRISEFEDVIGQRMDFSDYDLLKLNQLYNCTSSLSFMDSCDFELENICGMIQSSGDSADWQRVSQVLSGPESDHSKMGQCKDSGFFMHFNTSILNEGATAMLESRLLYPKRGFQCLEFYLYNSGSGNDQLNIYTREYTTGQQGGVLTLQRQIKEVPIGSWQLHYVTLQVTKKFRVVFEGLRGPGTSSGGLSIDDINLSETRCPHHIWHIQNFTQILGGQDTSVYSPPFYSSKGYAFQIYMDLRSSTNVGIYFHLISGANDDQLQWPCPWQQATMTLLDQNPDIRQRMFNQRSITTDPTMTSDNGSYFWDRPSKVGVTDVFPNGTQFSRGIGYGTTVFITRERLKSREFIKGDDIYILLTVEDISHLNSTSAVPDPVPTLAVHNACSEVVCQNGGICVVQDGRAECKCPAGEDWWYMGKRCEKRGSTRDTVIIAVSSTVTVFAVMLIITLVSVYCTRRKYRKKARANTAAMTLENQHAF

SEQ ID NO: 14 - protein, 422 aa, Homo sapiens
MSSSCSGLSRVLVAVATALVSASSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLASLGILSFLGLVAGALALGLWLRLRRGGKDGSPKPGFLASVIPVDRRPGAPNL

SEQ ID NO: 15 - protein, 432 aa, Mus musculus
MSSSCSGLTRVLVAVATALVSSSSPCPQAWGPPGVQYGQPGRPVMLCCPGVSAGTPVSWFRDGDSRLLQGPDSGLGHRLVLAQVDSPDEGTYVCQTLDGVSGGMVTLKLGFPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPSTGPLQDEIPDWSQGHGQQLEAVVAQEDSPAPARPSLQPDPRPLDHRDPLEQVAVLASLGIFSCLGLAVGALALGLWLRLRRSGKDGPQKPGLLAPMIPVEKLPGIPNLQRTPENFS

SEQ ID NO: 16 - protein, 348 aa, Homo sapiens
SSPCPQAWGPPGVQYGQPGRSVKLCCPGVTAGDPVSWFRDGEPKLLQGPDSGLGHELVLAQADSTDEGTYICQTLDGALGGTVTLQLGYPPARPVVSCQAADYENFSCTWSPSQISGLPTRYLTSYRKKTVLGADSQRRSPSTGPWPCPQDPLGAARCVVHGAEFWSQYRINVTEVNPLGASTRLLDVSLQSILRPDPPQGLRVESVPGYPRRLRASWTYPASWPCQPHFLLKFRLQYRPAQHPAWSTVEPAGLEEVITDAVAGLPHAVRVSARDFLDAGTWSTWSPEAWGTPSTGTIPKEIPAWGQLHTQPEVEPQVDSPAPPRPSLQPHPRLLDHRDSVEQVAVLA

SEQ ID NO: 17 - protein, 347 aa, Mus musculus
SPCPQAWGPPGVQYGQPGRPVMLCCPGVSAGTPVSWFRDGDSRLLQGPDSGLGHRLVLAQVDSPDEGTYVCQTLDGVSGGMVTLKLGFPPARPEVSCQAVDYENFSCTWSPGQVSGLPTRYLTSYRKKTLPGAESQRESPSTGPWPCPQDPLEASRCVVHGAEFWSEYRINVTEVNPLGASTCLLDVRLQSILRPDPPQGLRVESVPGYPRRLHASWTYPASWRRQPHFLLKFRLQYRPAQHPAWSTVEPIGLEEVITDAVAGLPHAVRVSARDFLDAGTWSAWSPEAWGTPSTGPLQDEIPDWSQGHGQQLEAVVAQEDSPAPARPSLQPDPRPLDHRDPLEQVAV

SEQ ID NO: 18 - protein, 199 aa, Homo sapiens
MNCVCRLVLVVLSLWPDTAVAPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL

SEQ ID NO: 19 - protein, 19 aa, synthetic construct
MGWSCIILFLVATATGVHS

SEQ ID NO: 20 - protein, 20 aa, synthetic construct
METDTLLLWVLLLWVPGSTG

SEQ ID NO: 21 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 107 aa, synthetic construct [sequence omitted]

SEQ ID NO: 23 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 123 aa, synthetic construct
EVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSS

SEQ ID NO: 25 - protein, 107 aa, synthetic construct
ELQMTQSPSSLSASVGDRVTITCQASQDIGNYLNWYQQKPGKAPKLLIYDASYLETGVPSRFSGSGSGTDFTFTISSLQPEDIATYYCQQYDNLPFTFGPGTKVDIK

SEQ ID NO: 26 - protein, 122 aa, synthetic construct
QVQLVQSGGGLVQPGGSLRVSCAASGFTSNIFWMSWVRQAPGKGLEWVANIDEDGSEKNYVDSVKGRFTISRDNAKNSLYLQMNSLRAEDTAVYYCARESFYYGSGTYFDFWGQGTLVTVSS

SEQ ID NO: 27 - protein, 110 aa, synthetic construct
ELVVTQPPSVSGAPGQRVTISCTGSSSNIGAGYDVHWYQQLPGTAPKLLIYGNKNRPSGVPDRFSGSKSGTSASLAITGLRAEDEADYYCQSFDSSLGWVFGGGTQLTVL

SEQ ID NO: 28 - protein, 471 aa, synthetic construct
MGWSCIILFLVATATGVHSEVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 234 aa, synthetic construct
METDTLLLWVLLLWVPGSTGELQMTQSPSSLSASVGDRVTITCQASQDIGNYLNWYQQKPGKAPKLLIYDASYLETGVPSRFSGSGSGTDFTFTISSLQPEDIATYYCQQYDNLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 470 aa, synthetic construct
MGWSCIILFLVATATGVHSQVQLVQSGGGLVQPGGSLRVSCAASGFTSNIFWMSWVRQAPGKGLEWVANIDEDGSEKNYVDSVKGRFTISRDNAKNSLYLQMNSLRAEDTAVYYCARESFYYGSGTYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 236 aa, synthetic construct
METDTLLLWVLLLWVPGSTGELVVTQPPSVSGAPGQRVTISCTGSSSNIGAGYDVHWYQQLPGTAPKLLIYGNKNRPSGVPDRFSGSKSGTSASLAITGLRAEDEADYYCQSFDSSLGWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 120 aa, synthetic construct
EIQLVQSGSELKKPGASVKVSCKASGYTFTNFGMNWVRQAPGQGLEWMGYINTNTGESIYSEEFKGRFVFSLDTSVSTAYLQICSLKAEDTAVYFCARSRMVTAYGMDYWGQGTTVTVSS

SEQ ID NO: 33 - protein, 107 aa, synthetic construct
EIVMTQSPATLSLSPGERATLSCKASQNVGTAVAWYQQKPGQSPRLLIFSASNRYTGVPARFSGSGSGTDFTLTISSLQSEDFAVYFCQQYSSYPLTFGQGTKLEIK

SEQ ID NO: 34 - protein, 119 aa, synthetic construct
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 35 - protein, 107 aa, synthetic construct
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIK

SEQ ID NO: 36 - protein, 468 aa, synthetic construct
MGWSCIILFLVATATGVHSEIQLVQSGSELKKPGASVKVSCKASGYTFTNFGMNWVRQAPGQGLEWMGYINTNTGESIYSEEFKGRFVFSLDTSVSTAYLQICSLKAEDTAVYFCARSRMVTAYGMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 37 - protein, 234 aa, synthetic construct
METDTLLLWVLLLWVPGSTGEIVMTQSPATLSLSPGERATLSCKASQNVGTAVAWYQQKPGQSPRLLIFSASNRYTGVPARFSGSGSGTDFTLTISSLQSEDFAVYFCQQYSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 467 aa, synthetic construct
MGWSCIILFLVATATGVHSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 39 - protein, 234 aa, synthetic construct
METDTLLLWVLLLWVPGSTGDIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 118 aa, synthetic construct
QVHLKESGPGLVAPSQSLSITCTVSGFSLTSYAVHWVRQPPGKGLEWLGVIWAGGNTNYNSVFMSRLTISKDNSKSQVFLKMNSLQTDDTAIYYCARLDDYDERFVYWGQGTLVTVSS

SEQ ID NO: 41 - protein, 113 aa, synthetic construct
DIVMSQSPSSLAVSVGEKVSMNCKSSQSLLYSSNHKNYLAWFQQKPGQSPKLLIYWASTRESGVPDRFTGSGSGTDFTLTISSVKAEDLAVYYCQQYYTYTWTFGGGTKLEIK

SEQ ID NO: 42 - protein, 466 aa, synthetic construct
MGWSCIILFLVATATGVHSQVHLKESGPGLVAPSQSLSITCTVSGFSLTSYAVHWVRQPPGKGLEWLGVIWAGGNTNYNSVFMSRLTISKDNSKSQVFLKMNSLQTDDTAIYYCARLDDYDERFVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 240 aa, synthetic construct
METDTLLLWVLLLWVPGSTGDIVMSQSPSSLAVSVGEKVSMNCKSSQSLLYSSNHKNYLAWFQQKPGQSPKLLIYWASTRESGVPDRFTGSGSGTDFTLTISSVKAEDLAVYYCQQYYTYTWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 118 aa, synthetic construct
QSLEESGGRLVTPGTPLTLTCTVSGFSLTSYDMNWVRQAPGKGLEWIGVVRGSGRTYYASWAKGRFTIARTSSTTVDLKMTSLTTGDTATYFCARGDANNNYYEFDIWGPGTLVTVSS

SEQ ID NO: 45 - protein, 111 aa, synthetic construct
QDMTQTPSPVSAAVGGTVTINCQSSQSVYGDAWLSWFQQKPGQPPKLLIYSASTLASGVPSRFKGSGSGTQFTLTISDLESDDAATYYCAGGYDGINDIRAFGGGTEVVVK

SEQ ID NO: 46 - protein, 466 aa, synthetic construct
MGWSCIILFLVATATGVHSQSLEESGGRLVTPGTPLTLTCTVSGFSLTSYDMNWVRQAPGKGLEWIGVVRGSGRTYYASWAKGRFTIARTSSTTVDLKMTSLTTGDTATYFCARGDANNNYYEFDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 47 - protein, 238 aa, synthetic construct
METDTLLLWVLLLWVPGSTGQDMTQTPSPVSAAVGGTVTINCQSSQSVYGDAWLSWFQQKPGQPPKLLIYSASTLASGVPSRFKGSGSGTQFTLTISDLESDDAATYYCAGGYDGINDIRAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 122 aa, synthetic construct
EVQLVESGGGLVQPGGSLRLSCAASGYSFTSYGMSWVRQAPGKGLEWVSYIYPYSGATYYADSVKGRFTISADTSKNTAYLQMNSLRAEDTAVYYCARHSGYYRISSAMDVWGQGTLVTVSS

SEQ ID NO: 49 - protein, 107 aa, synthetic construct
DIQMTQSPSSLSASVGDRVTITCRASQSISSYLAWYQQKPGKAPKLLIYGASSRASGVPSRFSGSGSGTDFTLTISSLQPEDFATYYCQQYYSSPLTFGQGTKVEIK

SEQ ID NO: 50 - protein, 122 aa, synthetic construct
EVQLVESGGGLVQPGGSLRLSCAASGYTFSSYGMSWVRQAPGKGLEWVSYIYPYSGATYYADSVKGRFTISADTSKNTAYLQMNSLRAEDTAVYYCARHSGYYRISSAMDVWGQGTLVTVSS

SEQ ID NO: 51 - protein, 107 aa, synthetic construct
DIQMTQSPSSLSASVGDRVTITCRASQSNRRFLAWYQQKPGKAPKLLIYGASSRASGVPSRFSGSGSGTDFTLTISSLQPEDFATYYCQQYYISPLTFGQGTKVEIK

SEQ ID NO: 52 - protein, 122 aa, synthetic construct; identical to SEQ ID NO: 50

SEQ ID NO: 53 - protein, 107 aa, synthetic construct
DIQMTQSPSSLSASVGDRVTITCRASQSVRSFLAWYQQKPGKAPKLLIYRASIRASGVPSRFSGSGSGTDFTLTISSLQPEDFATYYCQQYYISPWTFGQGTKVEIK

SEQ ID NO: 54 - protein, 122 aa, synthetic construct; identical to SEQ ID NO: 50

SEQ ID NO: 55 - protein, 107 aa, synthetic construct; identical to SEQ ID NO: 51

SEQ ID NO: 56 - protein, 470 aa, synthetic construct
MGWSCIILFLVATATGVHSEVQLVESGGGLVQPGGSLRLSCAASGYSFTSYGMSWVRQAPGKGLEWVSYIYPYSGATYYADSVKGRFTISADTSKNTAYLQMNSLRAEDTAVYYCARHSGYYRISSAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 234 aa, synthetic construct
METDTLLLWVLLLWVPGSTGDIQMTQSPSSLSASVGDRVTITCRASQSISSYLAWYQQKPGKAPKLLIYGASSRASGVPSRFSGSGSGTDFTLTISSLQPEDFATYYCQQYYSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 470 aa, synthetic construct
MGWSCIILFLVATATGVHSEVQLVESGGGLVQPGGSLRLSCAASGYTFSSYGMSWVRQAPGKGLEWVSYIYPYSGATYYADSVKGRFTISADTSKNTAYLQMNSLRAEDTAVYYCARHSGYYRISSAMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 59 - protein, 234 aa, synthetic construct
METDTLLLWVLLLWVPGSTGDIQMTQSPSSLSASVGDRVTITCRASQSNRRFLAWYQQKPGKAPKLLIYGASSRASGVPSRFSGSGSGTDFTLTISSLQPEDFATYYCQQYYISPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 470 aa, synthetic construct; identical to SEQ ID NO: 58

SEQ ID NO: 61 - protein, 234 aa, synthetic construct
METDTLLLWVLLLWVPGSTGDIQMTQSPSSLSASVGDRVTITCRASQSVRSFLAWYQQKPGKAPKLLIYRASIRASGVPSRFSGSGSGTDFTLTISSLQPEDFATYYCQQYYISPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 470 aa, synthetic construct; identical to SEQ ID NO: 58

SEQ ID NO: 63 - protein, 234 aa, synthetic construct; identical to SEQ ID NO: 59

SEQ ID NO: 64 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 65 - protein, 166 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 66 - protein, 327 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 67 - protein, 646 aa, synthetic construct
MGWSCIILFLVATATGVHSEVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 68 - protein, 652 aa, synthetic construct
MGWSCIILFLVATATGVHSEVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRN

SEQ ID NO: 69 - protein, 813 aa, synthetic construct
MGWSCIILFLVATATGVHSEVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 70 - protein, 471 aa, synthetic construct
MGWSCIILFLVATATGVHSEVQLVESGGGLVQPGRSLRLSCAASGFTFDDYAMHWVRQAPGKGLEWVSGISWNSGSIDYADSVKGRFTISRDNAKNSLYLQMNSLRVEDTALYYCAKDGSRVFGVGGGFDFWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVKLTCLVKGFYPSDIAVEWESNGQPENNYKTTPPVLDSDGSFFLYSSLTVDKSRWQQGNVFSCSVMHEALHNHYTQKSLSLSPG

SEQ ID NO: 71 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNHYTQKSLSLSPG

SEQ ID NO: 72 - protein, 402 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNHYTQKSLSLSPG

SEQ ID NO: 73 - protein, 563 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGSGGGSPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNHYTQKSLSLSPG

SEQ ID NO: 74 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNRFTQKSLSLSPG

SEQ ID NO: 75 - protein, 402 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENGGGSGGKSKAVEQVKNAFNKLQEKGIYKAMSEFDIFINYIEAYMTMKIRNGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVSGFYPSDIAVEWESNGQPENNYKTTPPVLDSDGSFKLYSKLTVDKSRWQQGNVFSCSVMHEALHNRFTQKSLSLSPG

(19) United States
(12) Patent Application Publication
(10) Pub. No.: US 2025/0353906 A1
(43) Pub. Date: Nov. 20, 2025

(54) COMPOSITIONS AND METHODS FOR TARGETED DELIVERY OF THERAPEUTIC AGENTS

(71) Applicant: Flagship Pioneering Innovations VII, LLC, Cambridge, MA (US)

(72) Inventors: Daniel BLOM, Brookline, MA (US); Adrienne Marie ROTHSCHILDS, Cambridge, MA (US); Nicholas McCartney PLUGIS, Duxbury, MA (US); Charlotte Marie NICOD, Cambridge, MA (US); Stephen MARSHALL, Kittery Point, ME (US); Avak KAHVEJIAN, Lexington, MA (US); Yann Paul Guy Régis ECHELARD, Jamaica Plain, MA (US); Noubar Boghos AFEYAN, Lexington, MA (US); Raffi AFEYAN, Lexington, MA (US); Scott Moore CARLSON, Boston, MA (US); Vivek KOHAR, Bar Harbor, ME (US); Tao FANG, Cambridge, MA (US); Nabil AZHAR, Ashburn, VA (US)

(21) Appl. No.: 19/120,373

(22) PCT Filed: Oct. 13, 2023
(86) PCT No.: PCT/US2023/076910

§ 371 Date: Apr. 11, 2025

Related U.S. Application Data
(60) Provisional application No. 63/416,233, filed on Oct. 14, 2022.

(51) Int. Cl. C07K 16/28; A61K 39/00

(52) U.S. Cl. CPC C07K 16/28; C07K 16/2803; C07K 16/2887; A61K 2039/505; C07K 2317/76; C07K 2317/92

(57) ABSTRACT

Macromolecule compositions and related methods that effect targeted delivery of therapeutic agents to effector targets in a desired cell, tissue and/or organ of interest while minimizing or avoiding undesirable delivery to other cells, tissues or organs are provided. Compositions and methods related to macromolecules, such as an ANDbody™, that include an effector target binding domain specific for an effector target, and an address binding domain specific for an address target are described. The macromolecules are linked to small molecules.

### SEQUENCE LISTING

The instant application contains a Sequence Listing which has been submitted electronically in XML format and is hereby incorporated by reference in its entirety. Said XML copy, created on Oct. 13, 2023, is named 51666-003WO2_Sequence_Listing_10_13_23 and is 96,536 bytes in size.

### BACKGROUND OF THE INVENTION

Undesirable off-target effects are a problem for otherwise desirable therapeutic targets that are present in healthy as well as diseased tissues.

### SUMMARY OF THE INVENTION

The present disclosure describes, in part, macromolecule compositions and related methods that effect targeted delivery of therapeutic agents to effector targets in a desired cell, tissue and/or organ of interest while minimizing or avoiding undesirable delivery to other cells, tissues or organs. Generally, compositions described herein comprise macromolecules, such as an ANDbody™, that include an effector target binding domain specific for an effector target, and an address binding domain specific for an address target. The address target is generally sufficiently restricted in the subject to target the macromolecule to the desired cell, tissue or organ. In some embodiments, the effector target binding domain does not influence an effector target in the absence of an address target binding domain. Moreover, the address target binding domain does not influence signaling upon binding the address target. However, localization of the effector target binding domain by the address target binding domain enables the effector target binding domain to bind the effector target sufficiently to elicit an influence on signaling by the effector target in the target cell or tissue. In addition, the macromolecules described herein are linked to one or more small molecules. The compositions described herein can be used, e.g., to specifically deliver a therapeutic agent (for example, the effector target binding domain, the small molecule, or both) to a desired location, e.g., a cell, tissue or organ, in a subject, while avoiding undesirable off-target effects.

In one aspect, the present disclosure provides a method of localizing a macromolecule at a target tissue or cell of a subject, the method comprising administering to the subject a macromolecule comprising a first binding site and a second binding site, wherein (a) the first binding site is specific for an effector target in the subject, and (b) the second binding site is specific for an address target expressed in the target tissue or cell in the subject; wherein: (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and allowing the macromolecule to localize at the target tissue or cell of the subject, wherein the macromolecule is linked to a small molecule.

In some embodiments, the macromolecule and the small molecule are connected by a linker (for example a cleavable linker or a non-cleavable linker).

One or more small molecules may be linked to the macromolecule.

In some embodiments, at least 25% of the macromolecule detectable in the subject is detected at the target tissue or cell at a time point between 1 and 7 days following administration of the macromolecule to the subject.

In some embodiments, the potency of the first binding site at the target tissue or cell is substantially increased relative to a reference macromolecule lacking the second binding site.

In some embodiments, the first binding site has a low affinity for the effector target.

In some embodiments, the first binding site has a low avidity for the effector target.

In some embodiments, the affinity of the first binding site for the effector target is lower than the affinity of the second binding site for the address target.

In some embodiments, the avidity of the first binding site for the effector target is lower than the avidity of the second binding site for the address target.

In some embodiments, effector target signaling by the macromolecule in a non-target tissue or cell of the subject is substantially decreased relative to a reference macromolecule lacking the second binding site.

In some embodiments, the address target is regionally expressed in the subject. In some embodiments, the address target is locally expressed in the subject. In some embodiments, the expression of the address target is restricted to a cell type in the subject.

In some embodiments, the address target is expressed only by a cell in the subject when in a specific cell state.

In some embodiments, the address target is expressed only by a cell in the subject in a disease state.

In some embodiments, the first binding site or the second binding site comprises a polypeptide.

In some embodiments, the polypeptide is an antibody or antigen-binding fragment thereof.

In some embodiments, the macromolecule is an antibody comprising a first binding site that is specific for the effector target in the subject and a second binding site that is specific for the address target.

In some embodiments, the polypeptide is a ligand of the effector target or a ligand of the address target.

In some embodiments, (a) the first binding site comprises an antibody or antigen-binding fragment thereof and the second binding site comprises a ligand of the address target; or (b) the first binding site comprises a ligand of the effector target and the second binding site comprises an antibody or antigen-binding fragment thereof.

In some embodiments, the target tissue is skin and the second binding site is specific for desmoglein-1 (DSG-1).

In some embodiments, the target tissue is lung tissue and the second binding site is specific for RAGE.

In some embodiments, the target tissue is kidney tissue and the second binding site is specific for cadherin 16 (CDH16).

In some embodiments, the target tissue is intestine tissue and the second binding site is specific for cadherin 17 (CDH17).

In another aspect, the present disclosure provides a macromolecule comprising a first binding site and a second binding site, wherein: (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein: (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein localization of the macromolecule to a non-target tissue or cell is substantially reduced relative to localization of a reference macromolecule lacking the second binding site, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein localization of the macromolecule to a target tissue or cell is substantially increased relative to localization of a reference macromolecule lacking the second binding site, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein at least 25% of the macromolecule administered to a subject is detected at the target tissue or cell at a time point between 1 and 7 days following administration, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein the affinity of the first binding site for the effector target is lower than the affinity of the second binding site for the address target, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein the avidity of the first binding site for the effector target is lower than the avidity of the second binding site for the address target, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and wherein the potency of the first binding site at the target tissue or cell is substantially increased relative to a reference macromolecule lacking the second binding site, wherein the macromolecule is linked to a small molecule.

In some embodiments of any of the above aspects, the macromolecule and the small molecule are connected by a linker (for example, a cleavable linker or a non-cleavable linker).

One or more small molecules may be linked to the macromolecule.

In some embodiments, the first binding site has a low affinity for the effector target.

In some embodiments, the first binding site has a low avidity for the effector target.

In some embodiments, the affinity of the first binding site for the effector target is lower than the affinity of the second binding site for the address target.

In some embodiments, the avidity of the first binding site for the effector target is lower than the avidity of the second binding site for the address target.

In some embodiments, (a) the Kd of the first binding site for the effector target is higher than the Kd of the second binding site for the address target; (b) the EC₅₀ of the first binding site for the effector target is higher than the EC₅₀ of the second binding site for the address target; or (c) the IC₅₀ of the first binding site for the effector target is higher than the IC₅₀ of the second binding site for the address target.

In some embodiments, the first binding site has an affinity to the effector target of at least about 2 times, at least about 5 times, or at least about 10 times less than the affinity of the second binding site to the address target.

In some embodiments, the affinity of the second binding site to the address target has a Kd of greater than about 1 nM, greater than about 2 nM, or greater than about 50 nm.

In some embodiments, the effector target is a protein, lipid, or sugar.

In some embodiments, the effector target is a cell membrane-associated target.

In some embodiments, the effector target is a protein. In some embodiments, the effector target is a secreted protein.

In some embodiments, the effector target is encoded by a gene selected from the group consisting of the genes recited in Table 1.

In some embodiments, the macromolecule agonizes the effector target.

In some embodiments, the macromolecule antagonizes the effector target.

In some embodiments, the address target is a protein, lipid, or sugar.

In some embodiments, the address target is a protein.

In some embodiments, expression of the effector target or the address target is expression of an RNA sequence encoding the effector target or the address target.

In some embodiments, the expression level of the effector target or the address target is assessed by using a RNA sequence dataset.

In some embodiments, the RNA sequence dataset is a Genotype-Tissue Expression (GTEx) dataset or a Human Protein Atlas (HPA) dataset.

In some embodiments, expression of the effector target or the address target is protein expression.

In some embodiments, the effector target is systemically expressed in the subject.

In some embodiments, the effector target is regionally expressed in the subject.

In some embodiments, the effector target is locally expressed in the subject.

In some embodiments, the address target is regionally expressed in the subject.

In some embodiments, the address target is locally expressed in the subject.

In some embodiments, the expression of the address target is restricted to a cell type in the subject.

In some embodiments, the address target is a soluble protein or an extracellular matrix (ECM)-associated protein and is not present in detectable amounts on the cell surface.

In some embodiments, the address target is expressed in the ECM and is not present in detectable amounts elsewhere in the subject.

In some embodiments, the address target is expressed only by a cell in the subject when in a specific cell state.

In some embodiments, the address target is expressed only by a cell in the subject when in a disease state.

In some embodiments, the address target is not expressed in a tissue in which binding of the second binding site to the effector target is deleterious to the subject.

In some embodiments, the binding site for the address target does not bind in detectable amounts to the binding site of a natural ligand of the address target.

In some embodiments, expression of the effector target or address target includes expression in one or more of minor salivary gland, thyroid, lung, breast, mammary tissue, pancreas, adrenal gland, liver, kidney, kidney cortex, kidney medulla, adipose-visceral tissue, omentum, small intestine, terminal ileum, fallopian tube, ovary, uterus, skin, skin not sun exposed, suprapubic skin, cervix, endocervix, ectocervix, vagina, skin sun exposed, lower leg skin, eneanterior cingulate cortex, Brodmann area 24 (BA24), basal ganglia, caudate nucleus, putamen, nucleus acumbens, hypothalamus, amygdala, hippocampus, cerebellum, cerebellar hemisphere, substantia nigra, pituitary gland, spinal cord, cervical spinal cord, artery, aorta, heart, atrial appendage, coronary artery, left ventricle, esophagus, esophagus mucosa, esophagus muscularis, gastroesophageal junction, spleen, stomach, colon, transverse colon, sigmoid colon, testis, whole blood cells, EBV-transformed lymphocytes, artery-tibial, or nerve-tibial tissues.

In some embodiments, expression of the effector target or address target includes expression in skin tissue, lung tissue, kidney tissue, or intestine tissue. In some embodiments, expression of the address target is substantially higher in skin tissue, lung tissue, kidney tissue, or intestine tissue than in any other tissue.

In some embodiments, the effector target and/or the address target is expressed on a structural tissue in the subject.

In some embodiments, the effector target and address target are on the same cell.

In some embodiments, the effector target and address target are on different cells.

In some embodiments, the effector target and address target are on different cells of the same cell type.

In some embodiments, the effector target and address target are on different cells of different cell types.

In some embodiments, the effector target and address target are on different cells in the same tissue.

In some embodiments, (a) the effector target is on a circulating cell and the address target is on a tissue-restricted cell; or (b) the effector target is on a tissue-restricted cell and the address target is on a circulating cell.

In some embodiments, the effector target and address target are on different cells located within 100 nm of each other in the subject.

In some embodiments, either the effector target or the address target is present on a cell surface.

In some embodiments, the macromolecule is a DNA polynucleotide.

In some embodiments, the macromolecule comprises an RNA or RNA-polypeptide conjugate.

In some embodiments, the macromolecule comprises a polypeptide. In some embodiments, the macromolecule is a polypeptide.

In some embodiments, the polypeptide is an antibody or antigen-binding fragment thereof.

In some embodiments, the first binding site and the second binding site each comprise a VH and/or a VL.

In some embodiments, the macromolecule is an antibody comprising a first binding site that is specific for the effector target in the subject and a second binding site that is specific for the address target.

In some embodiments, the macromolecule is an asymmetric antibody or a symmetric antibody.

In some embodiments, the antibody or antigen-binding fragment thereof comprises an scFv, BsIgG, a BsAb fragment, a BiTE, a dual-affinity re-targeting protein (DART), a tandem diabody (TandAb), a diabody, an Fab2, a di-scFv, chemically linked F(ab′)2, an Ig molecule with 2, 3 or 4 different antigen binding sites, a DVI-IgG four-in-one, an ImmTac, an HSAbody, an IgG-IgG, a Cov-X-Body, an scFv1-PEG-scFv2, an appended IgG, an DVD-IgG, an affibody, an affilin, an affimer, an affitin, an alphabody, an anticalin, an avimer, a DARPin, a Fynomer, a monobody, a nanoCLAMP, a bis-Fab, an Fv, a Fab, a Fab′-SH, a linear antibody, an scFv, an antibody with only a heavy chain (Humabody), an ScFab, an IgG antibody fragment, a single-chain variable region antibody, a single-domain heavy chain antibody. a bispecific triplebody, a BiKE, a CrossMAb, a dsDb, an scDb, tandem a dAb/VHH, a triple dAb VHH, a tetravalent dAb/VHH, a Fab-scFv, a Fab-Fv, or a DART-Fc, an adnectin, a Kunitz-type inhibitor, or a receptor decoy.

In some embodiments, the polypeptide is a ligand of the effector target or a ligand of the address target.

In some embodiments, the ligand is a natural ligand, a modified ligand, or a synthetic ligand.

In some embodiments, the effector target or address target is a receptor and the polypeptide is a ligand thereof.

In some embodiments, the first binding site comprises an antibody or antigen-binding fragment thereof and the second binding site comprises a ligand of the address target.

In some embodiments, the first binding site comprises a ligand of the effector target and the second binding site comprises an antibody or antigen-binding fragment thereof.

In some embodiments, the amino acid sequences of the first and second binding sites are at least about 10% identical, at least about 20% identical, at least about 30% identical, at least about 40% identical, at least about 50% identical, at least about 60% identical, or at least about 70% identical.

In some embodiments, the address target has a Gini coefficient higher than about 0.4, about 0.5, about 0.57, about 0.65, about 0.7, about 0.85, about 0.90, or about 0.95.

In some embodiments, the address target has a Tau coefficient higher than about 0.67, about 0.75, about 0.8, about 0.85, about 0.90, or about 0.95.

In some embodiments, the effector target has a Gini coefficient lower than about 0.25, about 0.20, or about 0.15.

In some embodiments, the effector target has a Tau coefficient lower than about 0.25, about 0.20, or about 0.15.

In some embodiments, the macromolecule further comprises a third binding site. In some embodiments, the third binding site is the same as the first binding site. In some embodiments, the third binding site is the same as the second binding site.

In some embodiments, the first binding site and second binding site are directly joined to each other in the macromolecule.

In some embodiments, the first binding site and the second binding site in the macromolecule are joined by a stable domain.

In some embodiments, the effector target is Notch2 and the address target is RAGE.

In some embodiments, RAGE signaling is not influenced by the second site binding the RAGE address target.

In some embodiments, the effector target is Notch2 and the address target is uromodulin (UMOD).

In some embodiments, UMOD signaling is not influenced by the second site binding the UMOD address target.

In some embodiments, the effector target is Notch2 and the address target is meprin A subunit beta (MEP1B).

In some embodiments, MEP1B signaling is not influenced by the second site binding the MEP1B address target.

In some embodiments, the effector target is IL11Ra and the address target is RAGE. In some embodiments, RAGE signaling is not influenced by the second site binding the RAGE address target.

In some embodiments, the effector target is IL 11Ra and the address target is UMOD. In some embodiments, UMOD signaling is not influenced by the second site binding the UMOD address target.

In some embodiments, the subject is a human.

In another aspect, the present disclosure provides a method of delivering a moiety to a target tissue or cell in a subject, comprising administering to the subject a macromolecule of any one of claims 1-86, wherein the target tissue comprises the address target.

In some embodiments, the moiety is a molecule.

In some embodiments, the moiety is not a toxin.

In some embodiments, the moiety is a cell.

In some embodiments, the moiety is not a T cell or an NK cell.

In some embodiments, the target tissue is not a tumor.

In another aspect, the present disclosure provides a method of modulating an effector target in a target tissue, comprising administering to the tissue a macromolecule of any one of claims 1-86, wherein the target tissue comprises the address target and the effector target.

In another aspect, the present disclosure provides a method of biasing a binding agent away from binding an effector target when the effector target is found in the heart or lungs, comprising administering the macromolecule of any one of claims 1-86, wherein the address target is not substantially expressed in the heart or lungs.

In another aspect, the present disclosure provides a method of modulating a target tissue in a subject, comprising administering to the subject a macromolecule of any one of claims 1-86, wherein the target tissue comprises the address target and the effector target.

In another aspect, the present disclosure provides a method of treating a subject having a disease or condition associated with an effector target, comprising administering to the subject a macromolecule of any one of claims 1-86, wherein the first binding site of the macromolecule binds the effector target.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein the second binding site localizes the first binding site to the address target such that the first binding site influences effector target signaling in the target tissue or cell, wherein the first binding site does not substantially influence effector target signaling in the absence of localization by the second binding site, and wherein the second binding site does not bind to the binding site of the natural ligand of the address target, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein the second binding site localizes the first binding site to the address target such that the first binding site influences effector target signaling in the target tissue or cell, wherein the first binding site does not substantially influence effector target signaling in the absence of localization by the second binding site, and wherein the first binding site and second binding site are directly joined to each other in the macromolecule, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein the second binding site localizes the first binding site to the address target such that the first binding site influences effector target signaling in the target tissue or cell, wherein the first binding site does not substantially influence effector target signaling in the absence of localization by the second binding site, and wherein the first binding site and second binding are joined to each other by a stable domain, wherein the macromolecule is linked to a small molecule.

In another aspect, the present disclosure provides a macromolecule comprising a first binding site and a second binding site, wherein (a) the first binding site is specific for an effector target in a subject, and (b) the second binding site is specific for an address target expressed in a target tissue or cell in the subject; wherein the second binding site localizes the first binding site to the address target such that the first binding site influences effector target signaling in the target tissue or cell, wherein the first binding site does not substantially influence effector target signaling in the absence of localization by the second binding site, and wherein the effector target and/or the address target is expressed on a structural tissue in a host, wherein the macromolecule is linked to a small molecule.

In another aspect, the present disclosure provides a pharmaceutical composition comprising the macromolecule of any one of the above embodiments.

In another aspect, the present disclosure provides a pharmaceutical composition comprising a macromolecule and one or more pharmaceutically acceptable excipients, wherein the macromolecule comprises a first binding site and a second binding site, wherein (a) the first binding site is specific for an effector target in a subject, and (b) the second binding site is specific for an address target expressed in a target tissue or cell in the subject; wherein the second binding site localizes the first binding site to the address target such that the first binding site influences effector target signaling in the target tissue or cell, and wherein the first binding site does not substantially influence effector target signaling in the absence of localization by the second binding site, wherein the macromolecule is linked to a small molecule.

In some embodiments, the pharmaceutical composition is an RNA pharmaceutical composition.

In some embodiments, the pharmaceutical composition further comprises a carrier.

In some embodiments, the carrier is a lipid nanoparticle.

In some embodiments, the carrier is a viral vector.

In some embodiments, the carrier is a membrane-based carrier.

In some embodiments, the membrane-based carrier is a cell.

In some embodiments, the membrane-based carrier is a vesicle.

In another aspect, the present disclosure provides a method for modulating activity of an effector target in the skin of a subject, the method comprising administering to the subject a macromolecule comprising a first binding site and a second binding site, wherein (a) the first binding site is specific for an effector target in the subject, and (b) the second binding site is specific for desmoglein-1 (DSG-1), wherein the macromolecule is linked to a small molecule.

In another aspect, the present disclosure provides a method for modulating activity of an effector target in the lung of a subject, the method comprising administering to the subject a macromolecule comprising a first binding site and a second binding site, wherein (a) the first binding site is specific for an effector target in the subject, and (b) the second binding site is specific for RAGE, wherein the macromolecule is linked to a small molecule.

In another aspect, the present disclosure provides a method for modulating activity of an effector target in the kidney of a subject, the method comprising administering to the subject a macromolecule comprising a first binding site and a second binding site, wherein (a) the first binding site is specific for an effector target in the subject, and (b) the second binding site is specific for cadherin 16 (CDH16), wherein the macromolecule is linked to a small molecule.

In another aspect, the present disclosure provides a method for modulating activity of an effector target in the intestine of a subject, the method comprising administering to the subject a macromolecule comprising a first binding site and a second binding site, wherein (a) the first binding site is specific for an effector target in the subject, and (b) the second binding site is specific for cadherin 17 (CDH17), wherein the macromolecule is linked to a small molecule.

In another aspect, the present disclosure provides a method of localizing a macromolecule at a target tissue or cell of a subject, the method comprising administering to the subject a macromolecule comprising a first binding site and a second binding site, wherein (a) the first binding site is specific for an effector target in the subject, and (b) the second binding site is specific for an address target expressed in the target tissue or cell in the subject; wherein: (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and allowing the macromolecule to localize at the target tissue or cell of the subject, wherein the macromolecule is linked to a small molecule.

In another aspect, the present disclosure provides a method of concentrating a macromolecule in a target tissue or cell in a subject, the method comprising administering to the subject a macromolecule comprising a first binding site and a second binding site, wherein (a) the first binding site is specific for an effector target in a subject, and (b) the second binding site is specific for an address target expressed in a target tissue or cell in the subject; wherein (i) the second binding site localizes the first binding site to the address target such that the first binding site influences effector target signaling in the target tissue or cell; (ii) the second binding site does not substantially influence signaling upon binding the address target; and (iii) the first binding site does not substantially influence effector target signaling in the absence of localization by the second binding site; and allowing the macromolecule to concentrate at the target tissue or cell of the subject, wherein at least 25% of the macromolecule detectable in the subject is detected at the target tissue or cell at a time point between 1 and 7 days following administration of the macromolecule to the subject, wherein the macromolecule is linked to a small molecule.

In some embodiments, the potency of the first binding site at the target tissue or cell is substantially increased relative to a reference macromolecule lacking the second binding site.

In some embodiments, effector target signaling by the macromolecule in a non-target tissue or cell of the subject is substantially decreased relative to a reference macromolecule lacking the second binding site.

In some embodiments, the macromolecule is a macromolecule of any one of the above embodiments.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 is a schematic illustrating exemplary ANDbody™ molecules and their use as logic-gated medicines. FIG. 1 shows broad distribution of a therapeutic target (right side), such as an effector target, in a human subject with no address targeting, and a localized and restricted distribution with address targeting (left side), which is provided by an address target binding domain. FIG. 1. also provides a representative bipartite structure of an ANDbody with an address target binding domain linked to an effector target binding domain, which includes a functional moiety, e.g., a moiety that modulates, e.g., agonizes or antagonizes, a target effector in an address targeted cell or tissue. The address target binding domain directs the ANDbody to a desired location, such as a targeted cell or tissue, allowing for the effector target binding domain to engage the therapeutic effector target in the localized and restricted distribution area. In some embodiments, high affinity of the effector domain for the target effector may not be required; localization of the effector target binding domain by the address target binding domain enables the effector target binding domain to bind the effector target sufficiently to elicit an influence on signaling by the effector target in the target cell or tissue despite low affinity of the effector domain for the effector target. The address target binding domain can alternatively be used to transport molecular or cellular cargos to a desired address.

FIG. 2 is a schematic map showing activity of exemplary effector targets that can be restricted to tissues or cells of interest by developing ANDbody therapeutics comprised of an effector targeting domain and an address targeting domain. These ANDbody biologics represent potent, address-restricted medicines according to the present technology.

FIG. 3 provides exemplary structures of ANDbody biologics that can be engineered according to the present technology, including (but not limited to): an asymmetric antibody, an dual-affinity re-targeting protein (DART), a tandem diabody (TandAb), a diabody, an Fab2, IgG(L,H)-Fv or a BiTE.

FIG. 4 demonstrates an EC₅₀ curve of an exemplary single effector targeting domain (dashed line), such as a monospecific biologic (for example scFv) having a single binding domain to an effector target compared to an EC₅₀ of an exemplary bispecific ANDbody biologic (for example di-scFc) with an address target binding domain and an effector target binding domain (solid line), such that single effector targeting domain (usually broadly expressed) is targeted/restricted to local, address target-specific tissues and/or cells, thus effectively increasing affinity of the effector target binding domain for the effector target binding site, as evidenced by a shift of the curve to the left (lower EC₅₀, higher affinity).

FIG. 5A is a bar graph showing the level of fluorescence intensity detected in the indicated tissues in mice treated with the anti-DSG1 antibody PRO003 conjugated to IRDYE® 800CW. Data are shown as average of three mice. To control for differences in labeling efficiency, values are shown with the strongest signal set to 1.

FIG. 5B is a bar graph showing the level of fluorescence intensity detected in the indicated tissues in mice treated with the anti-DSG1 antibody PRO004 conjugated to IRDYE® 800CW or with a vehicle control (untreated). Data are shown as average of three mice. To control for differences in labeling efficiency, values are shown with the strongest signal set to 1.

FIG. 6A is a bar graph showing the level of fluorescence intensity detected in the indicated tissues in mice treated with the anti-RAGE antibody PRO001 conjugated to IRDYE® 800CW or with a vehicle control. Data are shown as average of three mice. To control for differences in labeling efficiency, values are shown with the strongest signal set to 1.

FIG. 6B is a bar graph showing the level of fluorescence intensity detected in the indicated tissues in mice treated with the anti-RAGE antibody PRO002 conjugated to IRDYE® 800CW or with a vehicle control. Data are shown as average of three mice. To control for differences in labeling efficiency, values are shown with the strongest signal set to 1.

FIG. 7 is a pair of photomicrographs showing representative IHC staining of an anti-human secondary antibody conjugated to horseradish peroxidase in lung tissue of Balb/C mice that were treated by tail vein injection with 3 mg/kg of the anti-RAGE antibody PRO002 (left panel) as compared to untreated mice (right panel). PRO002 comprises a human IgG1 backbone.

FIG. 8 is a bar graph showing the level of fluorescence intensity detected in the indicated tissues in mice treated with the anti-CDH16 antibody PRO056 conjugated to IRDYE® 800CW or with a vehicle control. Data are shown as average of three mice. To control for differences in labeling efficiency, values are shown with the strongest signal set to 1.

FIG. 9 is a bar graph showing the level of fluorescence intensity detected in the indicated tissues in mice treated with the anti-CDH17 antibody PRO061 conjugated to IRDYE® 800CW or with a vehicle control. Data are shown as average of three mice. To control for differences in labeling efficiency, values are shown with the strongest signal set to 1.

FIG. 10 is a set of photomicrographs showing staining for the Notch2 antagonistic mAbs PRO034, PRO035, and PRO036 and the corresponding RAGE-targeting ANDbodies PRO051, PRO052, and PRO053 on fresh frozen healthy mouse tissue microarray (FF TMA) sections. Lung sections are indicated by boxes.

FIG. 11 is a plot showing the concentration of PRO052, a control antibody that binds RAGE and respiratory syncytial virus (RSV) glycoprotein F (RAGE XT-4/Motavizumab), and a control antibody that binds Notch2 and RSV glycoprotein F (Notch2-2/Motavizumab), as detected by sandwich ELISA. Points show the average of three mice. Error bars show the standard deviation.

FIG. 12 is a set of schematic diagrams showing the design of the PRO023, PRO025, PRO024, PRO027, and PRO026 IL-10/DSG1 ANDbodies.

FIG. 13A is a bar graph showing the level of tumor necrosis factor alpha (TNFα) in peripheral blood mononuclear cell (PBMC) cell culture after pre-stimulation with hrIL-10 followed by treatment with lipopolysaccharide (LPS) for the indicated lengths of time.

FIG. 13B is a bar graph showing the level of TNFα in PBMC cell culture after pre-stimulation with an anti-DSG1 monoclonal antibody (mAb) followed by treatment with LPS for the indicated lengths of time.

FIG. 13C is a bar graph showing the level of TNFα in PBMC cell culture after pre-stimulation with the IL-10/DSG1 ANDbody PRO024 followed by treatment with LPS for the indicated lengths of time.

FIG. 13D is a bar graph showing the level of TNFα in PBMC cell culture after pre-stimulation with the IL-10/DSG1 ANDbody PRO026 followed by treatment with LPS for the indicated lengths of time.

FIG. 13E is a bar graph showing the level of TNFα in PBMC cell culture after pre-stimulation with the IL-10/DSG1 ANDbody PRO023 followed by treatment with LPS for the indicated lengths of time.

FIG. 13F is a bar graph showing the level of TNFα in PBMC cell culture after pre-stimulation with the IL-10/DSG1 ANDbody PRO025 followed by treatment with LPS for the indicated lengths of time.

FIG. 13G is a bar graph showing the level of TNFα in PBMC cell culture after pre-stimulation with the IL-10/DSG1 ANDbody PRO027 followed by treatment with LPS for the indicated lengths of time.

FIG. 14A is a bar graph showing the level of TNFα in primary macrophage cell culture cell culture after pre-stimulation with hrIL-10 followed by treatment with LPS for the indicated lengths of time.

FIG. 14B is a bar graph showing the level of TNFα in primary macrophage cell culture after pre-stimulation with the anti-DSG1 monoclonal antibody (mAb) PRO003 followed by treatment with LPS for the indicated lengths of time.

FIG. 14C is a bar graph showing the level of TNFα in primary macrophage cell culture after pre-stimulation with the IL-10/DSG1 ANDbody PRO024 followed by treatment with LPS for the indicated lengths of time.

FIG. 14D is a bar graph showing the level of TNFα in primary macrophage cell culture after pre-stimulation with the IL-10/DSG1 ANDbody PRO026 followed by treatment with LPS for the indicated lengths of time.

FIG. 14E is a bar graph showing the level of TNFα in primary macrophage cell culture after pre-stimulation with the IL-10/DSG1 ANDbody PRO023 followed by treatment with LPS for the indicated lengths of time.

FIG. 14F is a bar graph showing the level of TNFα in primary macrophage cell culture after pre-stimulation with the IL-10/DSG1 ANDbody PRO025 followed by treatment with LPS for the indicated lengths of time.

FIG. 14G is a bar graph showing the level of TNFα in primary macrophage cell culture after pre-stimulation with the IL-10/DSG1 ANDbody PRO027 followed by treatment with LPS for the indicated lengths of time.

FIG. 15 is a plot showing the level of IL-10 signaling detected in parental HEK-BLUE™ IL-10 cells or HEK-BLUE™ IL-10 cells stably expressing DSG1 (+DSG1 expression) that were treated overnight with the IL-10/DSG1 ANDbody PRO058 (functionally equivalent to PRO026) or a control antibody that comprises IL-10 and binds RSV glycoprotein F (IL-10/Motavizumab) at the indicated concentrations. IL-10 was measured using a colorimetric assay to detect expression of secreted embryonic alkaline phosphatase (SEAP). OD630: optical density at 630 nm. Curve fitting was performed using GraphPad Prism 9 to fit a 4-parameter log(agonist) vs. response.

FIG. 16A is a plot showing the concentration (ng/mL) of PRO003, PRO024, and PRO058 over time in serum samples from BALB/c mice dosed by tail vein injection with 3 mg/kg of the indicated antibody or ANDbody. The concentration of circulating molecules was measured by ELISA. Average concentration and standard deviation are shown. N=3.

FIG. 16B is a plot showing the concentration (ng of target protein per mg of total protein) of PRO003, PRO024, and PRO058 (functionally equivalent to PRO026) over time in skin tissue samples from BALB/c mice dosed by tail vein injection with 3 mg/kg of the indicated antibody or ANDbody. Skin samples were collected at the indicated time points and homogenized to extract proteins. Concentration was measured by ELISA. Average concentration and standard deviation are shown. N=3.

FIG. 17 is a set of schematic diagrams showing the design of the PRO070, PRO074, PRO075, and PRO077 TNFα-blocking anti-DSG1 ANDbodies.

FIG. 18A is a plot showing the level of IL-10 signaling detected in parental HEK-BLUE™ IL-10 cells that were treated overnight with PRO003, recombinant human IL-10 (rhIL-10), or recombinant human IL-10 fused to a human Fc domain (IL-10-Fc). IL-10 was measured using a colorimetric assay to detect expression of SEAP. OD630: optical density at 630 nm. Curve fitting was performed using GraphPad Prism 9 to fit a 4-parameter log(agonist) vs. response.

FIG. 18B is a plot showing the level of IL-10 signaling detected in parental HEK-BLUE™ IL-10 cells that were treated overnight with the IL-10/DSG1 ANDbodies PRO023, PRO024, PRO025, PRO026, and PRO027. IL-10 was measured using a colorimetric assay to detect expression of SEAP. OD630: optical density at 630 nm. Curve fitting was performed using GraphPad Prism 9 to fit a 4-parameter log(agonist) vs. response.

### DETAILED DESCRIPTION OF THE INVENTION

Provided herein are ANDbody™ molecules that include a therapeutic effector target binding domain and an address target binding domain. The ANDbody molecule is linked to a small molecule or more than one small molecule. The therapeutic effector target on the ANDbody molecule productively engages its therapeutic effector target only if the address target binding domain also engages an address target on a target tissue or cell to localize the effector target to the targeted cell or tissue, e.g., to form an AND-gate type of logic gate. For example, in some embodiments, an ANDbody is a macromolecule comprising at least (a) a first binding site specific for a therapeutic effector target that is expressed, e.g., broadly expressed, on a mammalian subject, e.g., on a cell surface; and (b) a second binding site specific for an address target. In embodiments, expression of the address target is restricted in vivo in a subject. In some embodiments, the binding of a first binding site to a therapeutic effector target is weaker than the binding of the second binding site to the address marker. The effector and address targets may be on the same cell, or in different cells or compartments within the same tissue.

In some embodiments, at least 25% of the macromolecule (e.g., ANDbody) detectable in the subject is detected at the target tissue or cell at a time point between 1 and 7 days (e.g., at 1 day, 2 days, 3 days, 4 days, 5, days, 6 days, and/or 7 days) following administration of the macromolecule (e.g., ANDbody) to the subject. For example, in some embodiments, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, or 100% (e.g., 25-30%, 30-35%, 35-40%, 40-45%, 45-50%, 50-55%, 55-60%, 60-65%, 65-70%, 70-75%, 75-80%, 80-85%, 85-90%, 90-95%, or 95-100%) of the macromolecule detectable in the subject is detected at the target tissue or cell at a time point between 1 and 7 days following administration of the macromolecule the subject.

### Effector Target

An ANDbody™ of the invention comprises an effector that modulates a therapeutic effector target in a subject, e.g., a mammalian subject such as a human, in need thereof. As used herein, an “effector target” is a discrete structure (e.g., a cell surface protein, a transmembrane protein, a receptor) of a cell or tissue of a subject, to which a therapeutic effector binding domain of an ANDbody can bind and exert a modulating effect, such as a therapeutic effect, on the subject. The ANDbody described herein has a binding site specific for an effector target. Upon binding of the effector binding domain to the effector target, the effector modulates the target cell or tissue to produce a biological response, such as a therapeutic effect, on the subject. However, in some embodiments, the effector target binding domain provided herein may not elicit a biological effect unless it is provided in conjunction with an address targeting domain to localize the effector to the desired target address in a targeted cell or tissue. In some embodiments, such therapeutic signaling may require the binding of multiple effector targets by multiple macromolecules according to the invention.

In some embodiments, an effector target binding domain may produce a small/weak biological effect when provided alone and provide a larger/stronger biological effect when provided in conjunction with an address targeting domain that localizes and concentrates/focuses the effector to the desired target address in a targeted cell or tissue. In some embodiments, an effector target binding domain may produce an acceptable biological effect when provided alone and provide an even larger/stronger biological effect when provided in conjunction with an address targeting domain to localize the effector target binding domain to a targeted cell or tissue. In some embodiments, an effector target binding domain may produce a strong biological effect when provided alone and provide a strong, or stronger, targeted effect when provided in conjunction with an address targeting domain to localize the effector target binding domain to a targeted cell or tissue. In some embodiments, an effector target binding domain may produce a biological effect with undesirable off target biological effects when provided alone, but can be targeted, concentrated, and focused to desired addresses in a targeted cell or tissue when provided in conjunction with an address targeting domain in order to decrease or eliminate undesirable off-target biological effects. Accordingly, effector target binding domains of the present technology provide superior therapeutic agents that provide stronger, targeted biological effects with less side effects, including less unintended off-target biological effects, when provided in conjunction with address target binding domains as described herein.

Examples of such therapeutic signaling effects include, but are not limited to:
(i) blocking a signal transduction pathway that promotes or maintains a disease state;
(ii) activating a signal transduction pathway that reduces or prevents a disease state;
(iii) promoting antibody-dependent cellular cytotoxicity (ADCC);
(iv) inducing complement activation on the target cell or tissue;
(v) promoting phagocytosis;
(vi) blocking or activating a signal transduction pathway that promotes differentiation of a cell; and
(vii) inducing tissue remodeling to reduce or prevent fibrosis.

In some embodiments, the therapeutic effector target is more broadly expressed than the address target in the subject. In some embodiments, the therapeutic effector target is expressed systemically, regionally, or locally in the organism. “Systemic expression” of a therapeutic effector target means that the therapeutic effector target is expressed at substantially the same levels in most parts of a subject organism body. Systemic expression involves a plurality of tissues. “Regional expression” of a therapeutic effector target means that the therapeutic target is expressed in an area less than systemic expression but more than local expression. Regional expression is not limited to a single tissue but can occur in a plurality of different tissues. “Local expression” of a therapeutic effector target means that the therapeutic target is expressed in single or few tissue areas. Local expression is not limited to a single tissue but can occur in a plurality of different tissues.

In some embodiments, the effector target binding domain has a low affinity for the effector target. For example, a low affinity may be an affinity of greater than 10 nM (e.g., an affinity between 10 nM-1 μM, e.g., an affinity between 10 nM and 100 nM).

In some embodiments, the effector target binding domain has a low avidity for the effector target. Non-limiting examples of therapeutic effector targets that can be targeted with ANDbodies disclosed herein are listed in Table 1, along with the exemplary function for the effector targets.

TABLE 1
Exemplary Effector Targets

| | Sequence | |
|---|---|---|
| | Accession | Exemplary effector function |
| Target | number | for therapeutic effect |
| Notch 2 | Q04721 | Blocking Notch2 signaling, e.g., to treat |
| | | COPD, cancer |
| IL11RA | Q14626 | Blocking IL11RA signaling, e.g., to treat |
| | | fibrosis |
| Endothelin | P25101 | Blocking Endothelin Receptor A, e.g., to |
| Receptor A | | treat IPF |
| b3 adrenergic | P13945 | Agonizing b3AR, e.g., to treat obesity |
| receptor | | |
| FASR | P25445 | agonize FASR mediated apoptotic |
| | | signaling cascades eg. To treat cancer |
| Integrin | | Blocking Integrin alpha 4 to treat IBD |
| alpha4 | | |
| GLP1R | P43220 | Agonizing GLP1R to treat T2D or |
| | | obesity |
| LeptinR | P48357 | block leptin signaling to suppress |
| (peptide) | | appetite, eg. To treat weight gain |
| PTHR | U6CS43 | Activate PTH with intermittent agonizing |
| (GPCR) | | peptide. Eg. exposure to activating |
| | | peptide to stimulate bone growth in |
| | | osteoporosis |
| A4b7 (Integrin | P26010 | Antagonize its interactions with |
| B7) | | MAdCAM to prevent inflammation |
| | | associated with gut disease (eg. IBD) |

### Address Target

An ANDbody of the invention also comprises an address target binder that binds to an address target to provide targeted delivery of the effector. As used herein, an “address target” is a structure on a cell or tissue whose expression is sufficiently restricted in an organism to allow it to identify an organ, tissue, cell, or cell state of interest in an organism. The address target can be, e.g., a cell surface protein, or a structure localizing to the extracellular matrix. As used herein, “restricted” expression of an address target means that the address target has a differential, e.g., less broad, in vivo expression, as opposed to systemic expression. In certain embodiments, the address target is expressed, for example, in a single cell type, tissue or cell state in a mammalian subject, such as a human subject.

In some embodiments, the currently provided address target binding domains do not substantially influence biological signaling upon binding to the address target, e.g., do not modulate a signal transduction pathway or other biological response in the target cell or tissue. For example, the address target binder can be inert or inactive, in which it lacks any additional activity (other than binding), including lacking catalytic activity, after binding to the address target. For example, the address target binder binds a non-signaling site or motif of the address target. “Signal” is used herein to indicate a conformational, enzymatic, and/or electrical consequence occurs as a result of target binding. Accordingly, as described herein, address target binding domains do not signal upon address target binding. A domain that does not “substantially” influence biological signaling, as used herein, is a domain that modulates a signal transduction pathway or other biological response in the target cell or tissue to which it binds by no more than 25% relative to a control condition, e.g., relative to signaling in the absence of the domain. For example, the domain may modulate (e.g., increase or decrease) the signal transduction pathway or other biological response by less than 20%, less than 15%, less than 10%, less than 5%, less than 2%, or less than 1% (e.g., 20-25%, 15-20%, 10-15%, 5-10%, 2-5%, or 1-2%).

Similarly, an effector target binding domain may not substantially signal, or may not signal at all, when it is not localized by an address target binding domain. In embodiments, an effector target binding domain signals with higher potency (e.g., has higher avidity) when it is localized by an address target binding domain compared to the signal when it is not localized by an address target binding domain. When an effector target binding domain is localized to a targeted cell or tissue by an address target binding domain as part of the same macromolecule, effector target signaling can be influenced as discussed above.

In some embodiments, the address target is used for organ-specific addressing, tissue-specific addressing, or cell-specific addressing.

The specificity of address target binding domains for a cell or tissue can be detected using methods known in the art. In one embodiment, a Gini coefficient (GC) score, which is a method for assessing the expression variation of a particular gene in a data set, is used. (See O'Hagan et al., GeneGini: assessment via the Gini coefficient of reference “housekeeping” genes and diverse human transporter expression profiles. Cell systems 6, 230-244, https://doi.org/10.1016/j. cels.2018.01.003 (2018); Wright Muelas et al., The role and robustness of the Gini coefficient as an unbiased tool for the selection of Gini genes for normalising expression profiling data. Sci Rep 9, 17960 (2019). https://doi.org/10.1038/s41598-019-54288-7). Address target binders can be identified using cell expression data generated for address target binders as described herein (Table 2A and 2B). In some embodiments, address target markers exhibit Gini scores of greater than 0.4, such as between 0.74 and 1.00. Conversely, non-address markers that are expressed more systemically may exhibit Gini Scores of between 0.15 to 0.19.

In one embodiment, a Tau score, which represents the expression variation of a particular gene in a data set, is used. Calculating Tau uses the information of expression of a gene in each tissue and its maximal expression over all tissues while also taking into account the number of tissues where expression is measured (see Itai Yanai, et al., Genome-wide midrange transcription profiles reveal expression level relationships in human tissue specification, Bioinformatics, Volume 21, Issue 5, 1 Mar. 2005, Pages 650-659; Kryuchkova-Mostacci N, Robinson-Rechavi M. A benchmark of gene expression tissue-specificity metrics. Brief Bioinform. 2017 Mar. 1; 18(2):205-214. doi: 10.1093/bib/bbw008). In some embodiments, address target markers exhibit Tau scores of greater than 0.6, such as between 0.74 and 1.00. Conversely, non-address markers that are expressed more systemically may exhibit Tau Scores of below 0.3, such as 0.15 to 0.19.

In some embodiments, specificity of address target binding domains for a particular cell or tissue, such as that indicated by an appropriate Gini and/or Tau score, is determined with a tissue based analysis that does not include tissues having a natural biological separation barrier (i.e., blood-brain barrier). For example, in some embodiments, Gini and/or Tau scores may be calculated without data from tissues such as (but not limited to): central nervous system, brain, eye, and/or testis tissues. In some embodiments, an address target as provided herein identifies a cell state. As used herein a “cell state” refers to a given physiological condition of a cell. A cell state may be, e.g., a disease state (relative to a non-disease state or normal state of a cell or tissue); or an activated state (relative to a non-activated state of a cell). Exemplary disease states include inflammation, infection (e.g., bacterial, viral, or fungal infection), and states relating to cancer (e.g., precancerous or cancerous cell states). In some aspects, cell state reflects the fact that cells of a particular type can exhibit variability with regard to one or more features and/or can exist in a variety of different conditions, while retaining the features of their particular cell type and not gain features that would cause them to be classified as a different cell type. The different states or conditions in which a cell can exist may be characteristic of a particular cell type (e.g., may involve properties or characteristics exhibited only by that cell type and/or involve functions performed only or primarily by that cell type) or may occur in multiple different cell types. In some embodiments, a cell state reflects the capability of a cell to respond to a particular stimulus or environmental condition (e.g., whether or not the cell will respond, or the type of response that will be elicited) or is a condition of the cell brought about by a stimulus or environmental condition. Cells in different cell states may be distinguished from one another in a variety of ways. For example, they may express, produce, or secrete one or more different genes, proteins, or other molecules (“markers”, such as the address targets provided herein), exhibit differences in protein modifications such as phosphorylation, acetylation, etc., or may exhibit differences in appearance. Thus a cell state may be a condition of the cell in which the cell expresses, produces, or secretes one or more markers, exhibits particular protein modification(s), has a particular appearance, and/or will or will not exhibit one or more biological response(s) to a stimulus or environmental condition.

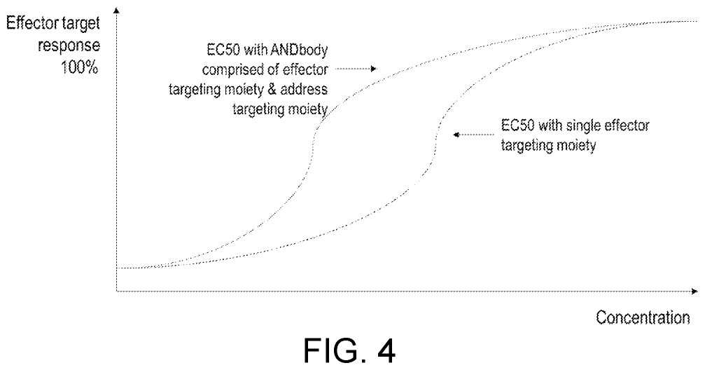

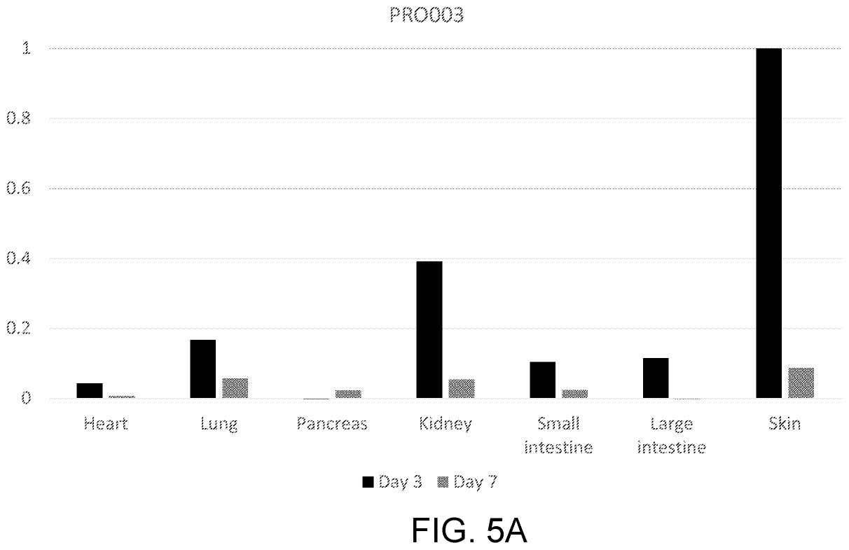

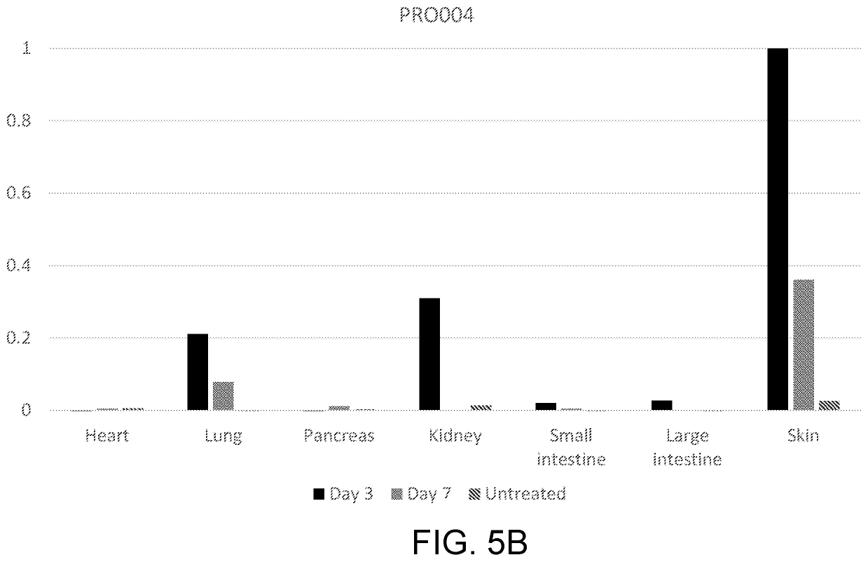

In some embodiments, the address target is CD20 (e.g., the address target binding domain binds to CD20 (e.g., human CD20)). The target cell may be, e.g., an immune cell. In some embodiments, the address target binding domain comprises rituximab (DrugBank Accession Number: DB00073) or a fragment, derivative, or variant thereof. In certain embodiments, the address target binding domain comprises the antigen binding fragment of rituximab, e.g., the CDRs (or full variable domains) disclosed in U.S. Pat. No. 7,381,560, including FIGS. 4 and 5, which are incorporated by reference. In other embodiments, the address target binding domain comprises a variant of the antigen binding fragment of rituximab, e.g., a humanized variant or a variant otherwise comprising one or more amino acid substitutions, e.g., 1, 2, 3, 4, 5, 6, 7, 8, 9, 10, or more substitutions, e.g., about: 1, 2, 3, 4, 5, 6, 7, 8, 9, 10, 11, 12, 13, 14, 15, 16, 17, 18, 19, or 20%, or more, divergence from the rituximab sequence; in certain embodiments, any substitutions are in the CDRs while in other embodiments any substitutions are outside the CDRs, while in other embodiments, the substitution may be in both CDR and non-CDR sequences. Substitutions, in some embodiments may be non-conservative, conservative, highly conservative, or a combination thereof, e.g., conservative or highly conservative substitutions in the CDRs (particularly in paratopic residues, while in some embodiments, any substitutions are outside of paratopic residues) and non-conservative, conservative, highly conservative, or a combination thereof in non-CDR residues. In some embodiments, the address target is CD20, and the small molecule is fluocinolone, ibrutinib, or tofacitinib.

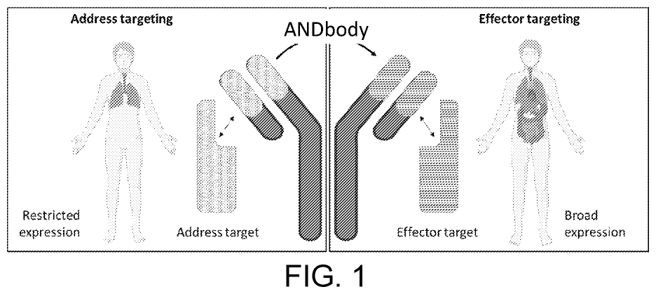

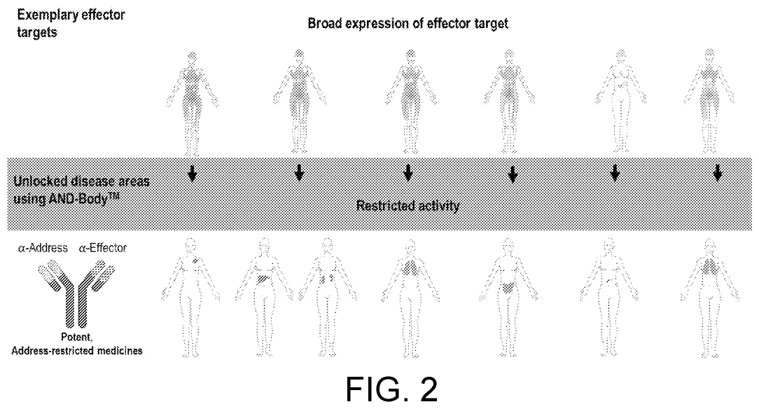

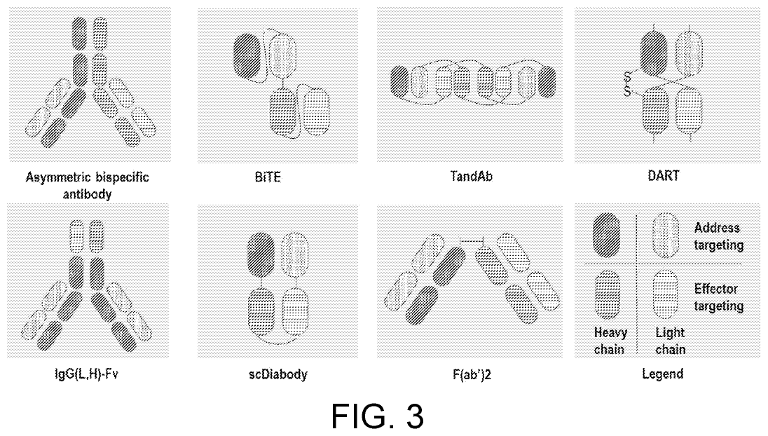

In some embodiments, the address target is CD33 (e.g., the address target binding domain binds to CD33 (e.g., human CD33)). The target cell may be, e.g., an immune cell. In some embodiments, the address target binding domain comprises gemtuzumab or a fragment, derivative, or variant thereof. In certain embodiments, the address target binding domain comprises the antigen binding fragment of gemtuzumab, e.g., the CDRs (or full variable domains) disclosed in US:<<8556, including FIGS. 1 and 2 (SEQ ID Nos: 2 and 4), which are incorporated by reference. In other embodiments, the address target binding domain comprises a variant of the antigen binding fragment of gemtuzumab, e.g., a humanized variant or a variant otherwise comprising one or more amino acid substitutions, e.g., 1, 2, 3, 4, 5, 6, 7, 8, 9, 10, or more substitutions, e.g., about: 1, 2, 3, 4, 5, 6, 7, 8, 9, 10, 11, 12, 13, 14, 15, 16, 17, 18, 19, or 20%, or more, divergence from the gemtuzumab sequence; in certain embodiments, any substitutions are in the CDRs while in other embodiments any substitutions are outside the CDRs, while in other embodiments, the substitution may be in both CDR and non-CDR sequences. Substitutions, in some embodiments may be non-conservative, conservative, highly conservative, or a combination thereof, e.g., conservative or highly conservative substitutions in the CDRs (particularly in paratopic residues, while in some embodiments, any substitutions are outside of paratopic residues) and non-conservative, conservative, highly conservative, or a combination thereof in non-CDR residues. In some embodiments, the address target is CD33, and the small molecule is fluocinolone, ibrutinib, or tofacitinib.

In some embodiments, the address target is a glucocorticoid address target. In some embodiments, the address target is a glucocorticoid address target, and the target cell is an immune cell.

In some embodiments, the target cell is a myeloid cell, and the glucocorticoid address target is HRH2, KCNE3, TLR4, MCTP1, CLEC4A, SIRPB1, FGCGRT, RNF130, CSFR2, SIRPB2, TYROBP, TBXAS1, CD300LF, LRRC25, FCER1G, TNFSF13, FES, CHST13, CLEC1B or CD302.

In some embodiments, the target cell is a T cell, and the glucocorticoid address target is PRKCH, CD2, IL7R, GIMAP5, CD6, KCNA3, CLEC2D, CD3D, CD3G, LCK, CD3E, CD247, SKAP1, LAT, SIRPG, TRAC, TRBV25-1, CD5, TRBC1 or IL23A.

In some embodiments, the target cell is a monocyte or dendritic cell, and the glucocorticoid address target is OSCAR, SIGLEC9, TNFSF13B, TLR8, FGL2, TLR2, IGSF6, LILRA1, CSF1R, GPBAR1, MS4A4A, UPK3A, CD3000, CCD86, CD68, MPEG1, FCN1, TNFSF13, MS4A6A or CST3.

In some embodiments, the target cell is a monocyte or dendritic cell or T cell, and the glucocorticoid target address is GPA33, ALG3, ENO1, IL1 ORA, STING1, LRRC8C, LPAR6, C1QL3, SIGMAR1, SLC3A2, GIMAP5, GIMAP1, TNFSF8, CACNA2D4, IL27RA, SCL9A6, HCST, CD48, SPN or ADA.

In some embodiments, the target cell is a monocyte or myeloid dendritic cell, and the glucocorticoid address target is SEZ6L, NFAM1, CLEC7A, TLR8, S100Z, FGL2, CD3000, CD86, CD68, SLC24A4, GPBAR1, SLCA7, CD300E, SIGLEC9, TLR5, CTSS, FCN1, 0033, MPEG1 or CST3.

In some embodiments, the target cell is a monocyte or myeloid dendritic cell or T cell, and the glucocorticoid address target is CD44, STING1, LRRC8C, LPAR6, ENO1, CACNA2D4, SERPINB9, IL10RA, IL27RA, IL12RB1, SLC9A6, MYO1G, CD48, S100A4, CRTAM, CELA1, GIMAP5, GIMAP1, TNFSF8 or GPA33.

Further exemplary address targets of the present technology are provided in Tables 2A (HPA database analysis) and 2B (Gtex database analysis), below.

TABLE 2A
Exemplary Address Targets (HPA database analysis)

| | | | | | Exemplary |
|---|---|---|---|---|---|
| | | | | | tissue/cellular |
| Gene | | | | | localization of |
| symbol | Ensembl | Uniprot ID | Tau score | Gini score | address target |
| AADACL4 | ENSG00000204518 | Q5VUY2 | 0.9194 | 0.7852 | epididymis |
| AATK | ENSG00000181409 | Q6ZMQ8 | 0.7783 | 0.546 | cerebral cortex |
| ABCA10 | ENSG00000154263 | Q8WWZ4 | 0.6792 | 0.4586 | ovary |
| ABCA12 | ENSG00000144452 | Q86UK0 | 0.9539 | 0.8965 | skin |
| ABCA13 | ENSG00000179869 | Q86UQ4 | 0.9202 | 0.6745 | bone marrow |
| ABCA4 | ENSG00000198691 | P78363 | 0.8841 | 0.7214 | epididymis |
| ABCB11 | ENSG00000073734 | O95342 | 0.9914 | 0.9671 | liver |
| ABCB4 | ENSG00000005471 | P21439 | 0.8274 | 0.5034 | liver |
| ABCB5 | ENSG00000004846 | Q2M3G0 | 0.9258 | 0.7265 | epididymis |
| ABCC11 | ENSG00000121270 | Q96J66 | 0.9664 | 0.8631 | breast |
| ABCC12 | ENSG00000140798 | Q96J65 | 0.974 | 0.97 | breast |
| ABCC2 | ENSG00000023839 | Q92887 | 0.8356 | 0.6441 | liver |
| ABCC6 | ENSG00000091262 | O95255 | 0.7146 | 0.4741 | liver |
| ABCC8 | ENSG00000006071 | Q09428 | 0.8101 | 0.6626 | cerebral cortex |
| ABCG4 | ENSG00000172350 | Q9H172 | 0.8977 | 0.6697 | cerebral cortex |
| ABCG5 | ENSG00000138075 | Q9H222 | 0.9457 | 0.929 | small intestine |
| ABCG8 | ENSG00000143921 | Q9H221 | 0.9273 | 0.8325 | small intestine |
| ABHD16A | ENSG00000204427 | O95870 | 0.875 | 0.7778 | gallbladder |
| ACKR4 | ENSG00000129048 | Q9NPB9 | 0.6411 | 0.4466 | duodenum |
| ACP4 | ENSG00000142513 | Q9BZG2 | 0.9708 | 0.9329 | skin |
| ACVR1C | ENSG00000123612 | Q8NER5 | 0.8594 | 0.5953 | adipose tissue |
| ADAM11 | ENSG00000073670 | O75078 | 0.9234 | 0.7693 | cerebral cortex |
| ADAM12 | ENSG00000148848 | O43184 | 0.8419 | 0.6349 | placenta |
| ADAM2 | ENSG00000104755 | Q99965 | 1 | 1 | breast |
| ADAM20 | ENSG00000134007 | O43506 | 0.9268 | 0.9268 | cerebral cortex |
| ADAM21 | ENSG00000139985 | Q9UKJ8 | 0.9202 | 0.7532 | cerebral cortex |
| ADAM22 | ENSG00000008277 | Q9POK1 | 0.7283 | 0.4446 | cerebral cortex |
| ADAM23 | ENSG00000114948 | O75077 | 0.7735 | 0.4184 | parathyroid gland |
| ADAM29 | ENSG00000168594 | Q9UKF5 | 0.9508 | 0.919 | endometrium |
| ADAM32 | ENSG00000197140 | Q8TC27 | 0.6516 | 0.4608 | skin |
| ADAM33 | ENSG00000149451 | Q9BZ11 | 0.5621 | 0.4 | endometrium |
| ADAM7 | ENSG00000069206 | Q9H2U9 | 0.99 | 0.9835 | epididymis |
| ADCY1 | ENSG00000164742 | Q08828 | 0.8168 | 0.516 | cerebral cortex |
| ADCY2 | ENSG00000078295 | Q08462 | 0.8139 | 0.5967 | cerebral cortex |
| ADCY4 | ENSG00000129467 | Q8NFM4 | 0.6858 | 0.406 | placenta |
| ADCY8 | ENSG00000155897 | P40145 | 0.9347 | 0.8854 | epididymis |
| ADCYAP1R1 | ENSG00000078549 | P41586 | 0.8129 | 0.5539 | cerebral cortex |
| ADGRA1 | ENSG00000197177 | Q86SQ6 | 0.9659 | 0.9383 | cerebral cortex |
| ADGRB1 | ENSG00000181790 | O14514 | 0.9121 | 0.6453 | cerebral cortex |
| ADGRB2 | ENSG00000121753 | O60241 | 0.8582 | 0.5438 | cerebral cortex |
| ADGRB3 | ENSG00000135298 | O60242 | 0.8675 | 0.5458 | cerebral cortex |
| ADGRD1 | ENSG00000111452 | Q6QNK2 | 0.6088 | 0.4 | placenta |
| ADGRE1 | ENSG00000174837 | Q14246 | 0.8457 | 0.6051 | granulocytes |
| ADGRE2 | ENSG00000127507 | Q9UHX3 | 0.7344 | 0.4061 | monocytes |
| ADGRE3 | ENSG00000131355 | Q9BY15 | 0.8976 | 0.6734 | granulocytes |
| ADGRF1 | ENSG00000153292 | Q5T601 | 0.837 | 0.7738 | urinary bladder |
| ADGRF3 | ENSG00000173567 | Q8IZF5 | 0.7837 | 0.534 | kidney |
| ADGRF4 | ENSG00000153294 | Q8IZF3 | 0.9132 | 0.7968 | skin |
| ADGRG2 | ENSG00000173698 | Q8IZP9 | 0.8866 | 0.6442 | epididymis |
| ADGRG3 | ENSG00000182885 | Q86Y34 | 0.8231 | 0.634 | granulocytes |
| ADGRG4 | ENSG00000156920 | Q8IZF6 | 0.9554 | 0.9216 | duodenum |
| ADGRG5 | ENSG00000159618 | Q8IZF4 | 0.8129 | 0.6296 | granulocytes |
| ADGRG7 | ENSG00000144820 | Q96K78 | 0.9117 | 0.8692 | small intestine |
| ADGRL3 | ENSG00000150471 | Q9HAR2 | 0.7403 | 0.4781 | cerebral cortex |
| ADGRV1 | ENSG00000164199 | Q8WXG9 | 0.8353 | 0.5653 | adrenal gland |
| ADIG | ENSG00000182035 | Q0VDE8 | 0.9659 | 0.9504 | epididymis |
| ADORA1 | ENSG00000163485 | P30542 | 0.7977 | 0.5357 | cerebral cortex |
| ADORA2B | ENSG00000170425 | P29275 | 0.7032 | 0.5341 | granulocytes |
| ADRA1A | ENSG00000120907 | P35348 | 0.7744 | 0.5909 | liver |
| ADRA1B | ENSG00000170214 | P35368 | 0.7861 | 0.5465 | spleen |
| ADRA1D | ENSG00000171873 | P25100 | 0.8478 | 0.733 | prostate |
| ADRA2A | ENSG00000150594 | P08913 | 0.5927 | 0.4122 | cervix, uterine |
| ADRA2C | ENSG00000184160 | P18825 | 0.769 | 0.5862 | seminal vesicle |
| ADRB3 | ENSG00000188778 | P13945 | 0.9072 | 0.7684 | ovary |
| ADTRP | ENSG00000111863 | Q96IZ2 | 0.6807 | 0.4974 | small intestine |
| AGER | ENSG00000204305 | Q15109 | 0.942 | 0.6711 | lung |
| (RAGE) | | | | | |
| AGTR1 | ENSG00000144891 | P30556 | 0.7202 | 0.4443 | placenta |
| AGTR2 | ENSG00000180772 | P50052 | 0.8982 | 0.8656 | smooth muscle |
| AJAP1 | ENSG00000196581 | Q9UKB5 | 0.8534 | 0.6208 | cerebral cortex |
| ALK | ENSG00000171094 | Q9UM73 | 0.9357 | 0.7784 | cerebral cortex |
| ALPP | ENSG00000163283 | P05187 | 0.9534 | 0.8933 | placenta |
| AMHR2 | ENSG00000135409 | Q16671 | 0.9219 | 0.8097 | ovary |
| AMN | ENSG00000166126 | Q9BXJ7 | 0.8765 | 0.7804 | small intestine |
| ANKAR | ENSG00000151687 | Q7Z5J8 | 0.7159 | 0.4233 | parathyroid gland |
| ANO2 | ENSG00000047617 | Q9NQ90 | 0.6832 | 0.4454 | placenta |
| ANO3 | ENSG00000134343 | Q9BYT9 | 0.9296 | 0.7809 | epididymis |
| ANO4 | ENSG00000151572 | Q32M45 | 0.8429 | 0.7696 | adrenal gland |
| ANO5 | ENSG00000171714 | Q75V66 | 0.7448 | 0.4641 | parathyroid gland |
| ANO7 | ENSG00000146205 | Q6IWH7 | 0.8989 | 0.7931 | prostate |
| ANO9 | ENSG00000185101 | A1A5B4 | 0.6923 | 0.4909 | duodenum |
| APCDD1L | ENSG00000198768 | Q8NCL9 | 0.8607 | 0.6756 | salivary gland |
| APLNR | ENSG00000134817 | P35414 | 0.6624 | 0.4032 | spleen |
| APLP1 | ENSG00000105290 | P51693 | 0.8158 | 0.4277 | cerebral cortex |
| AQP10 | ENSG00000143595 | Q96PS8 | 0.9493 | 0.8894 | duodenum |
| AQP11 | ENSG00000178301 | Q8NBQ7 | 0.7626 | 0.4971 | duodenum |
| AQP12A | ENSG00000184945 | Q8IXF9 | 0.9956 | 0.9894 | pancreas |
| AQP12B | ENSG00000185176 | A6NM10 | 0.9956 | 0.9904 | pancreas |
| AQP2 | ENSG00000167580 | P41181 | 0.9629 | 0.9304 | kidney |
| AQP4 | ENSG00000171885 | P55087 | 0.9052 | 0.7745 | cerebral cortex |
| AQP5 | ENSG00000161798 | P55064 | 0.8702 | 0.7198 | salivary gland |
| AQP7 | ENSG00000165269 | O14520 | 0.7575 | 0.4682 | adipose tissue |
| AQP9 | ENSG00000103569 | O43315 | 0.762 | 0.5304 | granulocytes |
| AREG | ENSG00000109321 | P15514 | 0.6962 | 0.4279 | placenta |
| ARMCX4 | ENSG00000196440 | Q5H9R4 | 0.6554 | 0.4114 | parathyroid gland |
| ARSH | ENSG00000205667 | Q5FYA8 | 0.9463 | 0.9109 | epididymis |
| ASAH2 | ENSG00000188611 | Q9NR71 | 0.8775 | 0.558 | duodenum |
| ASGR1 | ENSG00000141505 | P07306 | 0.8114 | 0.461 | liver |
| ASGR2 | ENSG00000161944 | P07307 | 0.8595 | 0.6116 | liver |
| ASIC1 | ENSG00000110881 | P78348 | 0.8174 | 0.4887 | cerebral cortex |
| ASIC2 | ENSG00000108684 | Q16515 | 0.9146 | 0.8482 | cerebral cortex |
| ASIC3 | ENSG00000213199 | Q9UHC3 | 0.7375 | 0.4063 | cerebral cortex |
| ASIC4 | ENSG00000072182 | Q96FT7 | 0.9852 | 0.9606 | cerebral cortex |
| ASPHD1 | ENSG00000174939 | Q5U4P2 | 0.8855 | 0.6676 | cerebral cortex |
| ASPRV1 | ENSG00000244617 | Q53RT3 | 0.8511 | 0.4144 | skin |
| ASTN1 | ENSG00000152092 | O14525 | 0.8921 | 0.6976 | cerebral cortex |
| ATP12A | ENSG00000075673 | P54707 | 0.8957 | 0.8698 | tonsil |
| ATP13A4 | ENSG00000127249 | Q4VNC1 | 0.7862 | 0.6789 | parathyroid gland |
| ATP13A5 | ENSG00000187527 | Q4VNC0 | 0.9243 | 0.7622 | breast |
| ATP1A2 | ENSG00000018625 | P50993 | 0.7206 | 0.4823 | cerebral cortex |
| ATP1A3 | ENSG00000105409 | P13637 | 0.9301 | 0.78 | cerebral cortex |
| ATP1A4 | ENSG00000132681 | Q13733 | 0.8721 | 0.7453 | placenta |
| ATP1B2 | ENSG00000129244 | P14415 | 0.7556 | 0.4022 | cerebral cortex |
| ATP2B2 | ENSG00000157087 | Q01814 | 0.9158 | 0.8334 | cerebral cortex |
| ATP2B3 | ENSG00000067842 | Q16720 | 0.9545 | 0.9245 | cerebral cortex |
| ATP2C2 | ENSG00000064270 | O75185 | 0.699 | 0.5908 | rectum |
| ATP4A | ENSG00000105675 | P20648 | 0.9883 | 0.958 | stomach |
| ATP4B | ENSG00000186009 | P51164 | 0.9822 | 0.9467 | stomach |
| ATP6AP1L | ENSG00000205464 | Q52LC2 | 0.7776 | 0.5805 | skin |
| ATP6V0A4 | ENSG00000105929 | Q9HBG4 | 0.8975 | 0.7973 | kidney |
| ATP8A2 | ENSG00000132932 | Q9NT12 | 0.9234 | 0.6846 | cerebral cortex |
| ATP8B4 | ENSG00000104043 | Q8TF62 | 0.7421 | 0.4466 | bone marrow |
| ATRNL1 | ENSG00000107518 | Q5VV63 | 0.7727 | 0.572 | cerebral cortex |
| AVPR1A | ENSG00000166148 | P37288 | 0.7499 | 0.4988 | adrenal gland |
| AVPR1B | ENSG00000198049 | P47901 | 0.938 | 0.86 | stomach |
| AVPR2 | ENSG00000126895 | P30518 | 0.7322 | 0.4842 | adipose tissue |
| B3GAT1 | ENSG00000109956 | Q9P2W7 | 0.8956 | 0.6452 | cerebral cortex |
| BAMBI | ENSG00000095739 | Q13145 | 0.6256 | 0.4095 | ovary |
| BDKRB1 | ENSG00000100739 | P46663 | 0.8144 | 0.6334 | gallbladder |
| BDKRB2 | ENSG00000168398 | P30411 | 0.619 | 0.458 | gallbladder |
| BEAN1 | ENSG00000166546 | Q3B7T3 | 0.7434 | 0.407 | cerebral cortex |
| BEST2 | ENSG00000039987 | Q8NFU1 | 0.7958 | 0.4578 | colon |
| BEST3 | ENSG00000127325 | Q8N1M1 | 0.88 | 0.6157 | cerebral cortex |
| BEST4 | ENSG00000142959 | Q8NFU0 | 0.8121 | 0.5532 | colon |
| BMPR1B | ENSG00000138696 | O00238 | 0.7647 | 0.6117 | cervix, uterine |
| BRS3 | ENSG00000102239 | P32247 | 0.9899 | 0.9704 | epididymis |
| BSND | ENSG00000162399 | Q8WZ55 | 0.9777 | 0.9343 | kidney |
| BTBD11 | ENSG00000151136 | A6QL63 | 0.789 | 0.4599 | parathyroid gland |
| BTC | ENSG00000174808 | P35070 | 0.5619 | 0.4328 | skin |
| BTLA | ENSG00000186265 | Q7Z6A9 | 0.8055 | 0.5853 | b-cells |
| BTN1A1 | ENSG00000124557 | Q13410 | 0.9711 | 0.8582 | breast |
| BTNL2 | ENSG00000204290 | Q9UIR0 | 0.949 | 0.9175 | prostate |
| BTNL3 | ENSG00000168903 | Q6UXE8 | 0.9075 | 0.8702 | duodenum |
| BTNL8 | ENSG00000113303 | Q6UX41 | 0.8409 | 0.7188 | granulocytes |
| BVES | ENSG00000112276 | Q8NE79 | 0.6818 | 0.4488 | smooth muscle |
| C10orf105 | ENSG00000214688 | Q8TEF2 | 0.8618 | 0.664 | spleen |
| C11orf87 | ENSG00000185742 | Q6NUJ2 | 0.9758 | 0.9342 | cerebral cortex |
| C14orf132 | ENSG00000227051 | Q9NPU4 | 0.6557 | 0.4353 | cerebral cortex |
| C16orf54 | ENSG00000185905 | Q6UWD8 | 0.7006 | 0.4573 | granulocytes |
| C1orf185 | ENSG00000204006 | Q5T7R7 | 0.9512 | 0.9512 | adipose tissue |
| C1orf210 | ENSG00000253313 | Q8IVY1 | 0.5899 | 0.4638 | duodenum |
| C20orf141 | ENSG00000258713 | Q9NUB4 | 0.9552 | 0.9318 | placenta |
| C3orf20 | ENSG00000131379 | Q8ND61 | 0.8184 | 0.6937 | lymph node |
| C3orf80 | ENSG00000180044 | F5H4A9 | 0.8604 | 0.6126 | cerebral cortex |
| C5AR2 | ENSG00000134830 | Q9P296 | 0.7691 | 0.4073 | granulocytes |
| C8A | ENSG00000157131 | P07357 | 0.9864 | 0.9694 | liver |
| C9 | ENSG00000113600 | P02748 | 0.9899 | 0.9808 | liver |
| C9orf135 | ENSG00000204711 | Q5VTT2 | 0.9477 | 0.8632 | fallopian tube |
| C9orf57 | ENSG00000204669 | Q5W0N0 | 1 | 1 | parathyroid gland |
| CA12 | ENSG00000074410 | O43570 | 0.6732 | 0.4937 | kidney |
| CA14 | ENSG00000118298 | Q9ULX7 | 0.8243 | 0.5757 | seminal vesicle |
| CA9 | ENSG00000107159 | Q16790 | 0.9256 | 0.856 | stomach |
| CABP7 | ENSG00000100314 | Q86V35 | 0.9258 | 0.8336 | cerebral cortex |
| CACNA1A | ENSG00000141837 | O00555 | 0.8763 | 0.5395 | cerebral cortex |
| CACNA1B | ENSG00000148408 | Q00975 | 0.9309 | 0.8096 | cerebral cortex |
| CACNA1C | ENSG00000151067 | Q13936 | 0.7159 | 0.4702 | smooth muscle |
| CACNA1D | ENSG00000157388 | Q01668 | 0.6777 | 0.4827 | fallopian tube |
| CACNA1E | ENSG00000198216 | Q15878 | 0.9383 | 0.8194 | cerebral cortex |
| CACNA1G | ENSG00000006283 | O43497 | 0.8372 | 0.5872 | cerebral cortex |
| CACNA1H | ENSG00000196557 | O95180 | 0.6199 | 0.4231 | ovary |
| CACNA1I | ENSG00000100346 | Q9P0X4 | 0.901 | 0.8161 | cerebral cortex |
| CACNA1S | ENSG00000081248 | Q13698 | 0.9826 | 0.953 | skeletal muscle |
| CACNA2D3 | ENSG00000157445 | Q8IZS8 | 0.8617 | 0.6321 | cerebral cortex |
| CACNA2D4 | ENSG00000151062 | Q7Z3S7 | 0.9147 | 0.8188 | t-cells |
| CACNG1 | ENSG00000108878 | Q06432 | 0.9779 | 0.9486 | skeletal muscle |
| CACNG2 | ENSG00000166862 | Q9Y698 | 0.9906 | 0.9792 | cerebral cortex |
| CACNG3 | ENSG00000006116 | O60359 | 0.9935 | 0.9887 | cerebral cortex |
| CACNG4 | ENSG00000075461 | Q9UBN1 | 0.8845 | 0.7714 | cerebral cortex |
| CACNG5 | ENSG00000075429 | Q9UF02 | 0.9665 | 0.946 | cerebral cortex |
| CACNG6 | ENSG00000130433 | Q9BXT2 | 0.9353 | 0.8716 | skeletal muscle |
| CACNG7 | ENSG00000105605 | P62955 | 0.9961 | 0.9904 | cerebral cortex |
| CADM2 | ENSG00000175161 | Q8N3J6 | 0.9024 | 0.6175 | cerebral cortex |
| CADM3 | ENSG00000162706 | Q8N126 | 0.6988 | 0.4311 | cerebral cortex |
| CALCR | ENSG00000004948 | P30988 | 0.9218 | 0.7574 | kidney |
| CALHM1 | ENSG00000185933 | Q8IU99 | 0.9283 | 0.7211 | cerebral cortex |
| CALHM3 | ENSG00000183128 | Q86XJ0 | 0.9119 | 0.8789 | dendritic cells |
| CALHM4 | ENSG00000164451 | Q5JW98 | 0.9932 | 0.9669 | placenta |
| CALHM5 | ENSG00000178033 | Q8N5C1 | 0.6974 | 0.4378 | placenta |
| CALN1 | ENSG00000183166 | Q9BXU9 | 0.9455 | 0.8658 | cerebral cortex |
| CALY | ENSG00000130643 | Q9NYX4 | 0.9377 | 0.8051 | cerebral cortex |
| CASR | ENSG00000036828 | P41180 | 0.9558 | 0.7864 | parathyroid gland |
| CATSPER1 | ENSG00000175294 | Q8NEC5 | 0.9399 | 0.8835 | granulocytes |
| CATSPERE | ENSG00000179397 | Q5SY80 | 0.7595 | 0.5126 | epididymis |
| CATSPERG | ENSG00000099338 | Q6ZRH7 | 0.6892 | 0.4463 | skin |
| CBARP | ENSG00000099625 | Q8N350 | 0.9515 | 0.8475 | cerebral cortex |
| CCDC188 | ENSG00000234409 | H7C350 | 0.7238 | 0.5424 | spleen |
| CCKAR | ENSG00000163394 | P32238 | 0.9647 | 0.9456 | gallbladder |
| CCKBR | ENSG00000110148 | P32239 | 0.9403 | 0.9019 | stomach |
| CCR10 | ENSG00000184451 | P46092 | 0.8477 | 0.5315 | t-cells |
| CCR3 | ENSG00000183625 | P51677 | 0.9375 | 0.6845 | granulocytes |
| CCR4 | ENSG00000183813 | P51679 | 0.8177 | 0.4888 | t-cells |
| CCR6 | ENSG00000112486 | P51684 | 0.7454 | 0.4983 | t-cells |
| CCR7 | ENSG00000126353 | P32248 | 0.7939 | 0.5403 | t-cells |
| CCR8 | ENSG00000179934 | P51685 | 0.9344 | 0.7641 | t-cells |
| CCR9 | ENSG00000173585 | P51686 | 0.9086 | 0.7792 | b-cells |
| CD101 | ENSG00000134256 | Q93033 | 0.7972 | 0.506 | granulocytes |
| CD163L1 | ENSG00000177675 | Q9NR16 | 0.6974 | 0.4893 | spleen |
| CD164L2 | ENSG00000174950 | Q6UWJ8 | 0.9023 | 0.7942 | fallopian tube |
| CD180 | ENSG00000134061 | Q99467 | 0.7324 | 0.4903 | b-cells |
| CD19 | ENSG00000177455 | P15391 | 0.8386 | 0.67 | b-cells |
| CD1A | ENSG00000158477 | P06126 | 0.8649 | 0.7269 | skin |
| CD1B | ENSG00000158485 | P29016 | 0.8813 | 0.7032 | dendritic cells |
| CD1D | ENSG00000158473 | P15813 | 0.7375 | 0.4226 | dendritic cells |
| CD200R1L | ENSG00000206531 | Q6Q8B3 | 0.9983 | 0.9937 | granulocytes |
| CD207 | ENSG00000116031 | Q9UJ71 | 0.8544 | 0.5934 | skin |
| CD209 | ENSG00000090659 | Q9NNX6 | 0.628 | 0.4093 | adipose tissue |
| CD22 | ENSG00000012124 | P20273 | 0.6788 | 0.4436 | lymph node |
| CD244 | ENSG00000122223 | Q9BZW8 | 0.809 | 0.5361 | granulocytes |
| CD27 | ENSG00000139193 | P26842 | 0.6947 | 0.4212 | t-cells |
| CD28 | ENSG00000178562 | P10747 | 0.7684 | 0.4953 | t-cells |
| CD300C | ENSG00000167850 | Q08708 | 0.7864 | 0.4942 | monocytes |
| CD300E | ENSG00000186407 | Q496F6 | 0.7976 | 0.5542 | monocytes |
| CD300LB | ENSG00000178789 | A8K4G0 | 0.8274 | 0.5806 | granulocytes |
| CD300LD | ENSG00000204345 | Q6UXZ3 | 0.8802 | 0.5851 | granulocytes |
| CD300LF | ENSG00000186074 | Q8TDQ1 | 0.7616 | 0.5162 | granulocytes |
| CD300LG | ENSG00000161649 | Q6UXG3 | 0.7808 | 0.4994 | adipose tissue |
| CD3G | ENSG00000160654 | P09693 | 0.804 | 0.4154 | t-cells |
| CD40LG | ENSG00000102245 | P29965 | 0.8186 | 0.5313 | t-cells |
| CD5 | ENSG00000110448 | P06127 | 0.7439 | 0.4332 | t-cells |
| CD6 | ENSG00000013725 | P30203 | 0.7028 | 0.4657 | lymph node |
| CD7 | ENSG00000173762 | P09564 | 0.7469 | 0.4697 | nk-cells |
| CD70 | ENSG00000125726 | P32970 | 0.867 | 0.6187 | t-cells |
| CD72 | ENSG00000137101 | P21854 | 0.7585 | 0.4113 | b-cells |
| CD79A | ENSG00000105369 | P11912 | 0.6686 | 0.4153 | b-cells |
| CD79B | ENSG00000007312 | P40259 | 0.7013 | 0.4123 | b-cells |
| CD80 | ENSG00000121594 | P33681 | 0.7816 | 0.6058 | appendix |
| CDH10 | ENSG00000040731 | Q9Y6N8 | 0.9679 | 0.9117 | cerebral cortex |
| CDH12 | ENSG00000154162 | P55289 | 0.8881 | 0.8058 | cervix, uterine |
| CDH15 | ENSG00000129910 | P55291 | 0.9719 | 0.9079 | skeletal muscle |
| CDH16 | ENSG00000166589 | O75309 | 0.9351 | 0.8659 | kidney |
| CDH17 | ENSG00000079112 | Q12864 | 0.7727 | 0.5189 | duodenum |
| CDH18 | ENSG00000145526 | Q13634 | 0.968 | 0.9189 | cerebral cortex |
| CDH19 | ENSG00000071991 | Q9H159 | 0.6206 | 0.4289 | heart muscle |
| CDH2 | ENSG00000170558 | P19022 | 0.7051 | 0.5105 | parathyroid gland |
| CDH20 | ENSG00000101542 | Q9HBT6 | 0.9554 | 0.8643 | cerebral cortex |
| CDH22 | ENSG00000149654 | Q9UJ99 | 0.9283 | 0.8231 | cerebral cortex |
| CDH26 | ENSG00000124215 | Q8IXH8 | 0.8039 | 0.4825 | prostate |
| CDH3 | ENSG00000062038 | P22223 | 0.6662 | 0.523 | skin |
| CDH4 | ENSG00000179242 | P55283 | 0.9085 | 0.7865 | cerebral cortex |
| CDH6 | ENSG00000113361 | P55285 | 0.6697 | 0.4549 | kidney |
| CDH7 | ENSG00000081138 | Q9ULB5 | 0.9643 | 0.9296 | cerebral cortex |
| CDH8 | ENSG00000150394 | P55286 | 0.9384 | 0.7675 | cerebral cortex |
| CDH9 | ENSG00000113100 | Q9ULB4 | 0.8018 | 0.4357 | cerebral cortex |
| CDHR1 | ENSG00000148600 | Q96JP9 | 0.8326 | 0.6175 | skin |
| CDHR2 | ENSG00000074276 | Q9BYE9 | 0.8837 | 0.8041 | duodenum |
| CDHR3 | ENSG00000128536 | Q6ZTQ4 | 0.838 | 0.4979 | fallopian tube |
| CDHR4 | ENSG00000187492 | A6H8M9 | 0.9317 | 0.7328 | fallopian tube |
| CDHR5 | ENSG00000099834 | Q9HBB8 | 0.8542 | 0.806 | duodenum |
| CDON | ENSG00000064309 | Q4KMG0 | 0.6369 | 0.4068 | thyroid gland |
| CEACAM3 | ENSG00000170956 | P40198 | 0.9229 | 0.8046 | granulocytes |
| CEACAM4 | ENSG00000105352 | O75871 | 0.8559 | 0.6729 | granulocytes |
| CELSR1 | ENSG00000075275 | Q9NYQ6 | 0.6755 | 0.4933 | fallopian tube |
| CELSR2 | ENSG00000143126 | Q9HCU4 | 0.7034 | 0.4656 | cerebral cortex |
| CELSR3 | ENSG00000008300 | Q9NYQ7 | 0.9027 | 0.5699 | cerebral cortex |
| CEND1 | ENSG00000184524 | Q8N111 | 0.9462 | 0.764 | cerebral cortex |
| CFAP65 | ENSG00000181378 | Q6ZU64 | 0.9513 | 0.841 | fallopian tube |
| CFTR | ENSG00000001626 | P13569 | 0.7716 | 0.6409 | gallbladder |
| CHODL | ENSG00000154645 | Q9H9P2 | 0.778 | 0.4593 | spleen |
| CHRFAM7A | ENSG00000166664 | Q494W8 | 0.7671 | 0.4566 | parathyroid gland |
| CHRM1 | ENSG00000168539 | P11229 | 0.8881 | 0.7732 | prostate |
| CHRM2 | ENSG00000181072 | P08172 | 0.8564 | 0.7383 | heart muscle |
| CHRM3 | ENSG00000133019 | P20309 | 0.7266 | 0.4916 | cerebral cortex |
| CHRM4 | ENSG00000180720 | P08173 | 0.9191 | 0.8248 | spleen |
| CHRM5 | ENSG00000184984 | P08912 | 0.8419 | 0.5754 | cerebral cortex |
| CHRNA1 | ENSG00000138435 | P02708 | 0.8978 | 0.7471 | skeletal muscle |
| CHRNA10 | ENSG00000129749 | Q9GZZ6 | 0.9216 | 0.4942 | skeletal muscle |
| CHRNA2 | ENSG00000120903 | Q15822 | 0.9722 | 0.868 | prostate |
| CHRNA3 | ENSG00000080644 | P32297 | 0.9161 | 0.7668 | adrenal gland |
| CHRNA4 | ENSG00000101204 | P43681 | 0.9421 | 0.9079 | parathyroid gland |
| CHRNA6 | ENSG00000147434 | Q15825 | 0.8564 | 0.764 | t-cells |
| CHRNA7 | ENSG00000175344 | P36544 | 0.7674 | 0.5196 | small intestine |
| CHRNA9 | ENSG00000174343 | Q9UGM1 | 0.9653 | 0.926 | fallopian tube |
| CHRNB2 | ENSG00000160716 | P17787 | 0.9776 | 0.8975 | cerebral cortex |
| CHRNB3 | ENSG00000147432 | Q05901 | 0.9675 | 0.9478 | cerebral cortex |
| CHRNB4 | ENSG00000117971 | P30926 | 0.8944 | 0.6441 | adrenal gland |
| CHRND | ENSG00000135902 | Q07001 | 0.992 | 0.9835 | skeletal muscle |
| CHRNE | ENSG00000108556 | Q04844 | 0.9206 | 0.5626 | heart muscle |
| CHRNG | ENSG00000196811 | P07510 | 0.9922 | 0.9772 | skeletal muscle |
| CHST9 | ENSG00000154080 | Q7L1S5 | 0.7933 | 0.6416 | fallopian tube |
| CLCA2 | ENSG00000137975 | Q9UQC9 | 0.9003 | 0.8079 | esophagus |
| CLCA4 | ENSG00000016602 | Q14CN2 | 0.8632 | 0.7863 | esophagus |
| CLCN1 | ENSG00000188037 | P35523 | 0.9641 | 0.8257 | skeletal muscle |
| CLCNKA | ENSG00000186510 | P51800 | 0.9169 | 0.7845 | kidney |
| CLCNKB | ENSG00000184908 | P51801 | 0.9068 | 0.7137 | kidney |
| CLDN1 | ENSG00000163347 | O95832 | 0.6574 | 0.4902 | skin |
| CLDN10 | ENSG00000134873 | P78369 | 0.7168 | 0.5876 | kidney |
| CLDN11 | ENSG00000013297 | O75508 | 0.8002 | 0.5644 | cerebral cortex |
| CLDN14 | ENSG00000159261 | O95500 | 0.9525 | 0.8433 | liver |
| CLDN17 | ENSG00000156282 | P56750 | 0.9872 | 0.9773 | esophagus |
| CLDN18 | ENSG00000066405 | P56856 | 0.9317 | 0.7657 | stomach |
| CLDN19 | ENSG00000164007 | Q8N6F1 | 0.9528 | 0.9125 | kidney |
| CLDN2 | ENSG00000165376 | P57739 | 0.8305 | 0.7468 | kidney |
| CLDN20 | ENSG00000171217 | P56880 | 0.8244 | 0.7053 | skin |
| CLDN22 | ENSG00000177300 | Q8N7P3 | 0.9869 | 0.9699 | fallopian tube |
| CLDN23 | ENSG00000253958 | Q96B33 | 0.6963 | 0.4432 | stomach |
| CLDN24 | ENSG00000185758 | A6NM45 | 0.9842 | 0.952 | kidney |
| CLDN3 | ENSG00000165215 | O15551 | 0.7346 | 0.585 | small intestine |
| CLDN4 | ENSG00000189143 | O14493 | 0.5271 | 0.4219 | colon |
| CLDN6 | ENSG00000184697 | P56747 | 0.9285 | 0.7482 | placenta |
| CLDN8 | ENSG00000156284 | P56748 | 0.8092 | 0.7417 | breast |
| CLDN9 | ENSG00000213937 | O95484 | 0.8802 | 0.7045 | parathyroid gland |
| CLEC12A | ENSG00000172322 | Q5QGZ9 | 0.7177 | 0.4406 | granulocytes |
| CLEC12B | ENSG00000256660 | Q2HXU8 | 0.9188 | 0.7737 | skin |
| CLEC17A | ENSG00000187912 | Q6ZS10 | 0.8431 | 0.6669 | b-cells |
| CLEC1B | ENSG00000165682 | Q9P126 | 0.9127 | 0.7923 | liver |
| CLEC2A | ENSG00000188393 | Q6UVW9 | 0.9857 | 0.9748 | skin |
| CLEC2L | ENSG00000236279 | POC7M8 | 0.9206 | 0.7872 | cerebral cortex |
| CLEC4C | ENSG00000198178 | Q8WTT0 | 0.9151 | 0.6812 | dendritic cells |
| CLEC4D | ENSG00000166527 | Q8WX18 | 0.8592 | 0.7041 | granulocytes |
| CLEC4E | ENSG00000166523 | Q9ULY5 | 0.7384 | 0.4538 | granulocytes |
| CLEC4F | ENSG00000152672 | Q8N1N0 | 0.6906 | 0.4672 | small intestine |
| CLEC4G | ENSG00000182566 | Q6UXB4 | 0.7874 | 0.5732 | liver |
| CLEC4M | ENSG00000104938 | Q9H2X3 | 0.9053 | 0.8054 | liver |
| CLEC5A | ENSG00000258227 | Q9NY25 | 0.8159 | 0.5412 | bone marrow |
| CLEC6A | ENSG00000205846 | Q6EIG7 | 0.9216 | 0.8336 | monocytes |
| CLEC9A | ENSG00000197992 | Q6UXN8 | 0.8203 | 0.5195 | dendritic cells |
| CLECL1 | ENSG00000184293 | Q8IZS7 | 0.7301 | 0.4551 | b-cells |
| CLIC3 | ENSG00000169583 | O95833 | 0.8114 | 0.5761 | dendritic cells |
| CLIC5 | ENSG00000112782 | Q9NZA1 | 0.6392 | 0.4525 | lung |
| CLIC6 | ENSG00000159212 | Q96NY7 | 0.7244 | 0.5014 | stomach |
| CLRN1 | ENSG00000163646 | P58418 | 0.9812 | 0.9558 | adrenal gland |
| CLRN2 | ENSG00000249581 | AOPK11 | 1 | 1 | kidney |
| CLRN3 | ENSG00000180745 | Q8NCR9 | 0.8423 | 0.8068 | small intestine |
| CLSTN2 | ENSG00000158258 | Q9H4D0 | 0.7843 | 0.5489 | ovary |
| CLTRN | ENSG00000147003 | Q9HBJ8 | 0.869 | 0.5739 | kidney |
| CMTM2 | ENSG00000140932 | Q8TAZ6 | 0.8479 | 0.7435 | granulocytes |
| CMTM5 | ENSG00000166091 | Q96DZ9 | 0.8645 | 0.4956 | cerebral cortex |
| CNGA1 | ENSG00000198515 | P29973 | 0.6269 | 0.4383 | liver |
| CNGA3 | ENSG00000144191 | Q16281 | 0.8049 | 0.7604 | esophagus |
| CNGA4 | ENSG00000132259 | Q8IV77 | 0.9381 | 0.6384 | fallopian tube |
| CNGB1 | ENSG00000070729 | Q14028 | 0.8773 | 0.6916 | adrenal gland |
| CNGB3 | ENSG00000170289 | Q9NQW8 | 0.8828 | 0.703 | bone marrow |
| CNIH2 | ENSG00000174871 | Q6PI25 | 0.9444 | 0.762 | cerebral cortex |
| CNIH3 | ENSG00000143786 | Q8TBE1 | 0.8792 | 0.538 | cerebral cortex |
| CNMD | ENSG00000136110 | O75829 | 0.9375 | 0.9018 | thyroid gland |
| CNNM1 | ENSG00000119946 | Q9NRU3 | 0.852 | 0.6167 | cerebral cortex |
| CNR1 | ENSG00000118432 | P21554 | 0.7306 | 0.4309 | cerebral cortex |
| CNR2 | ENSG00000188822 | P34972 | 0.851 | 0.7168 | granulocytes |
| CNTNAP2 | ENSG00000174469 | Q9UHC6 | 0.9154 | 0.7375 | cerebral cortex |
| CNTNAP3 | ENSG00000106714 | Q9BZ76 | 0.6749 | 0.452 | esophagus |
| CNTNAP4 | ENSG00000152910 | Q9C0A0 | 0.9864 | 0.9545 | cerebral cortex |
| CNTNAP5 | ENSG00000155052 | Q8WYK1 | 0.9775 | 0.9582 | cerebral cortex |
| COL13A1 | ENSG00000197467 | Q5TAT6 | 0.7004 | 0.4352 | epididymis |
| COL17A1 | ENSG00000065618 | Q9UMD9 | 0.7774 | 0.5608 | skin |
| COL23A1 | ENSG00000050767 | Q86Y22 | 0.7673 | 0.4674 | thyroid gland |
| COL25A1 | ENSG00000188517 | Q9BXS0 | 0.7203 | 0.5199 | adipose tissue |
| COLEC12 | ENSG00000158270 | Q5KU26 | 0.6676 | 0.4039 | placenta |
| CORIN | ENSG00000145244 | Q9Y5Q5 | 0.8754 | 0.7183 | heart muscle |
| CPT1C | ENSG00000169169 | Q8TCG5 | 0.7878 | 0.5129 | cerebral cortex |
| CR2 | ENSG00000117322 | P20023 | 0.8222 | 0.6599 | lymph node |
| CRB1 | ENSG00000134376 | P82279 | 0.9456 | 0.7913 | cerebral cortex |
| CRB2 | ENSG00000148204 | Q5IJ48 | 0.9209 | 0.7644 | cerebral cortex |
| CRHR1 | ENSG00000120088 | P34998 | 0.9543 | 0.8524 | cerebral cortex |
| CRHR2 | ENSG00000106113 | Q13324 | 0.8712 | 0.663 | seminal vesicle |
| CRLF2 | ENSG00000205755 | Q9HC73 | 0.8401 | 0.6627 | appendix |
| CRTAM | ENSG00000109943 | O95727 | 0.8311 | 0.5369 | nk-cells |
| CSF3R | ENSG00000119535 | Q99062 | 0.7128 | 0.4267 | granulocytes |
| CSMD1 | ENSG00000183117 | Q96PZ7 | 0.943 | 0.8207 | cerebral cortex |
| CSMD2 | ENSG00000121904 | Q7Z408 | 0.9081 | 0.7275 | cerebral cortex |
| CSMD3 | ENSG00000164796 | Q7Z407 | 0.98 | 0.9399 | cerebral cortex |
| CSPG4 | ENSG00000173546 | Q6UVK1 | 0.6146 | 0.4138 | adipose tissue |
| CSPG5 | ENSG00000114646 | O95196 | 0.9551 | 0.7032 | cerebral cortex |
| CT83 | ENSG00000204019 | Q5H943 | 0.9896 | 0.9743 | salivary gland |
| CTLA4 | ENSG00000163599 | P16410 | 0.7876 | 0.58 | t-cells |
| CTXN1 | ENSG00000178531 | P60606 | 0.844 | 0.624 | cerebral cortex |
| CTXN2 | ENSG00000233932 | P0C2S0 | 0.9893 | 0.9669 | cerebral cortex |
| CTXN3 | ENSG00000205279 | Q4LDR2 | 0.9775 | 0.9544 | kidney |
| CWH43 | ENSG00000109182 | Q9H720 | 0.8095 | 0.7094 | prostate |
| CXCR1 | ENSG00000163464 | P25024 | 0.8736 | 0.6096 | granulocytes |
| CXCR2 | ENSG00000180871 | P25025 | 0.8166 | 0.4876 | granulocytes |
| CXCR3 | ENSG00000186810 | P49682 | 0.8056 | 0.565 | dendritic cells |
| CXCR5 | ENSG00000160683 | P32302 | 0.8476 | 0.6776 | b-cells |
| CXorf66 | ENSG00000203933 | Q5JRM2 | 1 | 1 | liver |
| CYP26C1 | ENSG00000187553 | Q6V0L0 | 0.9617 | 0.9404 | spleen |
| CYP46A1 | ENSG00000036530 | Q9Y6A2 | 0.8925 | 0.5676 | cerebral cortex |
| DBH | ENSG00000123454 | P09172 | 0.9353 | 0.7033 | adrenal gland |
| DCC | ENSG00000187323 | P43146 | 0.9127 | 0.7725 | cerebral cortex |
| DCHS2 | ENSG00000197410 | Q6V1P9 | 0.8847 | 0.7649 | endometrium |
| DCST1 | ENSG00000163357 | Q5T197 | 0.9423 | 0.7328 | skin |
| DCST2 | ENSG00000163354 | Q5T1A1 | 0.8439 | 0.5085 | skin |
| DCSTAMP | ENSG00000164935 | Q9H295 | 0.9527 | 0.8384 | lung |
| DIO2 | ENSG00000211448 | Q92813 | 0.7722 | 0.5472 | thyroid gland |
| DIO3 | ENSG00000197406 | P55073 | 0.8138 | 0.6283 | cervix, uterine |
| DISP2 | ENSG00000140323 | A7MBM2 | 0.8604 | 0.5382 | cerebral cortex |
| DLK1 | ENSG00000185559 | P80370 | 0.8856 | 0.6954 | placenta |
| DLK2 | ENSG00000171462 | Q6UY11 | 0.779 | 0.506 | skin |
| DLL3 | ENSG00000090932 | Q9NYJ7 | 0.9855 | 0.9576 | cerebral cortex |
| DNAJC22 | ENSG00000178401 | Q8N4W6 | 0.6981 | 0.5753 | thyroid gland |
| DNER | ENSG00000187957 | Q8NFT8 | 0.8426 | 0.6116 | cerebral cortex |
| DPP10 | ENSG00000175497 | Q8N608 | 0.9034 | 0.763 | cerebral cortex |
| DPP6 | ENSG00000130226 | P42658 | 0.8432 | 0.6837 | cerebral cortex |
| DPY19L2 | ENSG00000177990 | Q6NUT2 | 0.5998 | 0.4219 | parathyroid gland |
| DRD1 | ENSG00000184845 | P21728 | 0.9075 | 0.686 | cerebral cortex |
| DRD2 | ENSG00000149295 | P14416 | 0.9091 | 0.7389 | adrenal gland |
| DRD4 | ENSG00000069696 | P21917 | 0.9664 | 0.9315 | dendritic cells |
| DRD5 | ENSG00000169676 | P21918 | 0.9548 | 0.9264 | stomach |
| DSC1 | ENSG00000134765 | Q08554 | 0.9557 | 0.883 | skin |
| DSC3 | ENSG00000134762 | Q14574 | 0.8758 | 0.719 | skin |
| DSCAM | ENSG00000171587 | O60469 | 0.9788 | 0.947 | cerebral cortex |
| DSCAML1 | ENSG00000177103 | Q8TD84 | 0.834 | 0.4943 | cerebral cortex |
| DSG1 | ENSG00000134760 | Q02413 | 0.9463 | 0.8739 | skin |
| DSG3 | ENSG00000134757 | P32926 | 0.9166 | 0.8278 | esophagus |
| DSG4 | ENSG00000175065 | Q86SJ6 | 0.9484 | 0.9276 | duodenum |
| DUOX1 | ENSG00000137857 | Q9NRD9 | 0.7017 | 0.5701 | skin |
| DUOX2 | ENSG00000140279 | Q9NRD8 | 0.8314 | 0.6573 | gallbladder |
| DUOXA1 | ENSG00000140254 | Q1HG43 | 0.7335 | 0.5998 | esophagus |
| ECEL1 | ENSG00000171551 | O95672 | 0.9213 | 0.7051 | ovary |
| EDA2R | ENSG00000131080 | Q9HAV5 | 0.5572 | 0.4466 | thyroid gland |
| EDAR | ENSG00000135960 | Q9UNE0 | 0.8266 | 0.6016 | t-cells |
| EDNRA | ENSG00000151617 | P25101 | 0.6316 | 0.4122 | seminal vesicle |
| EFNB3 | ENSG00000108947 | Q15768 | 0.8052 | 0.5291 | cerebral cortex |
| EGF | ENSG00000138798 | P01133 | 0.8815 | 0.7105 | kidney |
| ELFN1 | ENSG00000225968 | P0CZU0 | 0.8076 | 0.614 | liver |
| ELFN2 | ENSG00000166897 | Q5R3F8 | 0.9753 | 0.9224 | cerebral cortex |
| ENPEP | ENSG00000138792 | Q07075 | 0.742 | 0.5254 | small intestine |
| ENPP3 | ENSG00000154269 | O14638 | 0.7351 | 0.5526 | small intestine |
| ENTPD2 | ENSG00000054179 | Q9Y5L3 | 0.6289 | 0.4853 | duodenum |
| ENTPD8 | ENSG00000188833 | Q5MY95 | 0.837 | 0.7208 | small intestine |
| EPCAM | ENSG00000119888 | P16422 | 0.5686 | 0.4487 | small intestine |
| EPGN | ENSG00000182585 | Q6UW88 | 0.9242 | 0.7649 | esophagus |
| EPHA1 | ENSG00000146904 | P21709 | 0.8606 | 0.5333 | parathyroid gland |
| EPHA10 | ENSG00000183317 | Q5JZY3 | 0.7811 | 0.6473 | colon |
| EPHA3 | ENSG00000044524 | P29320 | 0.6787 | 0.4576 | prostate |
| EPHA5 | ENSG00000145242 | P54756 | 0.9169 | 0.7947 | cerebral cortex |
| EPHA6 | ENSG00000080224 | Q9UF33 | 0.8422 | 0.6941 | ovary |
| EPHA7 | ENSG00000135333 | Q15375 | 0.8538 | 0.5811 | parathyroid gland |
| EPHA8 | ENSG00000070886 | P29322 | 0.955 | 0.9321 | adrenal gland |
| EPHB1 | ENSG00000154928 | P54762 | 0.7204 | 0.4531 | cerebral cortex |
| EPHB2 | ENSG00000133216 | P29323 | 0.6051 | 0.431 | rectum |
| EPHB3 | ENSG00000182580 | P54753 | 0.6416 | 0.4106 | skin |
| EPHX4 | ENSG00000172031 | Q8IUS5 | 0.8357 | 0.5897 | cerebral cortex |
| ERBB4 | ENSG00000178568 | Q15303 | 0.8478 | 0.7284 | fallopian tube |
| EREG | ENSG00000124882 | O14944 | 0.8495 | 0.6461 | bone marrow |
| ERVFRD-1 | ENSG00000244476 | P60508 | 0.9243 | 0.6874 | placenta |
| ERVMER34-1 | ENSG00000226887 | Q9H9K5 | 0.8898 | 0.603 | parathyroid gland |
| ERVW-1 | ENSG00000242950 | Q9UQF0 | 0.9025 | 0.4919 | placenta |
| ESR1 | ENSG00000091831 | P03372 | 0.7662 | 0.5445 | endometrium |
| ESYT3 | ENSG00000158220 | A0FGR9 | 0.8418 | 0.724 | thyroid gland |
| EVC2 | ENSG00000173040 | Q86UK5 | 0.6373 | 0.4229 | ovary |
| F2RL2 | ENSG00000164220 | O00254 | 0.7398 | 0.459 | gallbladder |
| FAIM2 | ENSG00000135472 | Q9BWQ8 | 0.7616 | 0.4478 | cerebral cortex |
| FAM151A | ENSG00000162391 | Q8WW52 | 0.9047 | 0.7183 | kidney |
| FAM155A | ENSG00000204442 | B1AL88 | 0.8962 | 0.6476 | cerebral cortex |
| FAM155B | ENSG00000130054 | O75949 | 0.8664 | 0.6844 | thyroid gland |
| FAM162B | ENSG00000183807 | Q5T6X4 | 0.7169 | 0.4473 | placenta |
| FAM163A | ENSG00000143340 | Q96GL9 | 0.8844 | 0.6974 | adrenal gland |
| FAM163B | ENSG00000196990 | POC2L3 | 0.9408 | 0.8145 | cerebral cortex |
| FAM171A2 | ENSG00000161682 | A8MVW0 | 0.8564 | 0.6362 | cerebral cortex |
| FAM171B | ENSG00000144369 | Q6P995 | 0.7462 | 0.4187 | cerebral cortex |
| FAM187B | ENSG00000177558 | Q17R55 | 0.9745 | 0.9626 | spleen |
| FAM189A1 | ENSG00000104059 | O60320 | 0.9036 | 0.8075 | cerebral cortex |
| FAM189A2 | ENSG00000135063 | Q15884 | 0.6824 | 0.4629 | thyroid gland |
| FAM205A | ENSG00000205108 | Q6ZU69 | 1 | 1 | skin |
| FAM209A | ENSG00000124103 | Q5JX71 | 0.8871 | 0.5849 | bone marrow |
| FAM209B | ENSG00000213714 | Q5JX69 | 0.8429 | 0.4616 | bone marrow |
| FAP | ENSG00000078098 | Q12884 | 0.6894 | 0.5067 | endometrium |
| FASLG | ENSG00000117560 | P48023 | 0.8422 | 0.5348 | t-cells |
| FAT2 | ENSG00000086570 | Q9NYQ8 | 0.8973 | 0.7615 | skin |
| FAT3 | ENSG00000165323 | Q8TDW7 | 0.9323 | 0.8573 | cerebral cortex |
| FAXC | ENSG00000146267 | Q5TGI0 | 0.695 | 0.5034 | cerebral cortex |
| FCAR | ENSG00000186431 | P24071 | 0.9731 | 0.9395 | bone marrow |
| FCER2 | ENSG00000104921 | P06734 | 0.8403 | 0.6096 | b-cells |
| FCGR1A | ENSG00000150337 | P12314 | 0.635 | 0.4073 | epididymis |
| FCGR1B | ENSG00000198019 | Q92637 | 0.7229 | 0.4751 | epididymis |
| FCMR | ENSG00000162894 | O60667 | 0.679 | 0.403 | b-cells |
| FCRL1 | ENSG00000163534 | Q96LA6 | 0.8784 | 0.7758 | b-cells |
| FCRL2 | ENSG00000132704 | Q96LA5 | 0.8088 | 0.675 | lymph node |
| FCRL3 | ENSG00000160856 | Q96P31 | 0.8183 | 0.6984 | lymph node |
| FCRL4 | ENSG00000163518 | Q96PJ5 | 0.9465 | 0.9099 | tonsil |
| FCRL5 | ENSG00000143297 | Q96RD9 | 0.7691 | 0.622 | tonsil |
| FCRL6 | ENSG00000181036 | Q6DN72 | 0.8083 | 0.4367 | t-cells |
| FER1L6 | ENSG00000214814 | Q2WGJ9 | 0.919 | 0.8418 | stomach |
| FFAR1 | ENSG00000126266 | O14842 | 0.8923 | 0.7677 | bone marrow |
| FFAR2 | ENSG00000126262 | O15552 | 0.8519 | 0.5841 | granulocytes |
| FFAR3 | ENSG00000185897 | O14843 | 0.8565 | 0.6858 | appendix |
| FFAR4 | ENSG00000186188 | Q5NUL3 | 0.8454 | 0.6118 | rectum |
| FGFR3 | ENSG00000068078 | P22607 | 0.7475 | 0.4729 | skin |
| FGFR4 | ENSG00000160867 | P22455 | 0.6746 | 0.4789 | lung |
| FIBCD1 | ENSG00000130720 | Q8N539 | 0.8997 | 0.7961 | parathyroid gland |
| FLRT3 | ENSG00000125848 | Q9NZU0 | 0.6166 | 0.4585 | lung |
| FLT3 | ENSG00000122025 | P36888 | 0.848 | 0.5553 | dendritic cells |
| FMR1NB | ENSG00000176988 | Q8N0W7 | 1 | 1 | epididymis |
| FNDC10 | ENSG00000228594 | F2Z333 | 0.6928 | 0.4154 | adrenal gland |
| FNDC4 | ENSG00000115226 | Q9H6D8 | 0.6724 | 0.4405 | adrenal gland |
| FNDC5 | ENSG00000160097 | Q8NAU1 | 0.7674 | 0.5168 | skeletal muscle |
| FNDC9 | ENSG00000172568 | Q8TBE3 | 0.9628 | 0.889 | cerebral cortex |
| FOLH1 | ENSG00000086205 | Q04609 | 0.7895 | 0.5517 | duodenum |
| FPR1 | ENSG00000171051 | P21462 | 0.7182 | 0.4011 | granulocytes |
| FPR2 | ENSG00000171049 | P25090 | 0.8206 | 0.5174 | granulocytes |
| FRAS1 | ENSG00000138759 | Q86XX4 | 0.7806 | 0.5116 | thyroid gland |
| FRMD5 | ENSG00000171877 | Q7Z6J6 | 0.888 | 0.6285 | heart muscle |
| FUT6 | ENSG00000156413 | P51993 | 0.7662 | 0.6658 | esophagus |
| FXYD2 | ENSG00000137731 | P54710 | 0.806 | 0.4767 | kidney |
| FXYD3 | ENSG00000089356 | Q14802 | 0.6079 | 0.4579 | rectum |
| FXYD4 | ENSG00000150201 | P59646 | 0.9563 | 0.8703 | kidney |
| FXYD7 | ENSG00000221946 | P58549 | 0.8706 | 0.6034 | cerebral cortex |
| FZD10 | ENSG00000111432 | Q9ULW2 | 0.7846 | 0.6284 | cervix, uterine |
| FZD8 | ENSG00000177283 | Q9H461 | 0.6885 | 0.4081 | spleen |
| FZD9 | ENSG00000188763 | O00144 | 0.8597 | 0.7432 | skeletal muscle |
| GABBR1 | ENSG00000204681 | Q9UBS5 | 0.717 | 0.4883 | cerebral cortex |
| GABBR2 | ENSG00000136928 | O75899 | 0.964 | 0.8549 | cerebral cortex |
| GABRA1 | ENSG00000022355 | P14867 | 0.99 | 0.9732 | cerebral cortex |
| GABRA2 | ENSG00000151834 | P47869 | 0.8797 | 0.6296 | cerebral cortex |
| GABRA3 | ENSG00000011677 | P34903 | 0.9595 | 0.868 | cerebral cortex |
| GABRA4 | ENSG00000109158 | P48169 | 0.983 | 0.9548 | cerebral cortex |
| GABRA5 | ENSG00000186297 | P31644 | 0.9786 | 0.9144 | cerebral cortex |
| GABRA6 | ENSG00000145863 | Q16445 | 1 | 1 | cerebral cortex |
| GABRB1 | ENSG00000163288 | P18505 | 0.9774 | 0.901 | cerebral cortex |
| GABRB2 | ENSG00000145864 | P47870 | 0.9149 | 0.703 | cerebral cortex |
| GABRB3 | ENSG00000166206 | P28472 | 0.8101 | 0.5443 | cerebral cortex |
| GABRD | ENSG00000187730 | O14764 | 0.9652 | 0.8236 | cerebral cortex |
| GABRE | ENSG00000102287 | P78334 | 0.798 | 0.578 | adipose tissue |
| GABRG1 | ENSG00000163285 | Q8N1C3 | 0.9864 | 0.9539 | cerebral cortex |
| GABRG2 | ENSG00000113327 | P18507 | 0.8558 | 0.4767 | cerebral cortex |
| GABRG3 | ENSG00000182256 | Q99928 | 0.9072 | 0.7995 | prostate |
| GABRR1 | ENSG00000146276 | P24046 | 0.9557 | 0.9056 | placenta |
| GABRR2 | ENSG00000111886 | P28476 | 0.8049 | 0.8049 | adrenal gland |
| GALR1 | ENSG00000166573 | P47211 | 0.8884 | 0.5863 | adrenal gland |
| GALR2 | ENSG00000182687 | O43603 | 0.9673 | 0.9038 | smooth muscle |
| GALR3 | ENSG00000128310 | O60755 | 0.9872 | 0.9833 | cerebral cortex |
| GAPT | ENSG00000175857 | Q8N292 | 0.7573 | 0.4356 | granulocytes |
| GCGR | ENSG00000215644 | P47871 | 0.929 | 0.8826 | liver |
| GDPD2 | ENSG00000130055 | Q9HCC8 | 0.8694 | 0.7595 | spleen |
| GDPD4 | ENSG00000178795 | Q6W3E5 | 0.9476 | 0.9104 | placenta |
| GGT6 | ENSG00000167741 | Q6P531 | 0.6224 | 0.5347 | colon |
| GHRHR | ENSG00000106128 | Q02643 | 0.974 | 0.9693 | adrenal gland |
| GHSR | ENSG00000121853 | Q92847 | 0.9672 | 0.9543 | nk-cells |
| GJA3 | ENSG00000121743 | Q9Y6H8 | 0.9227 | 0.8719 | parathyroid gland |
| GJA8 | ENSG00000121634 | P48165 | 0.9756 | 0.9756 | kidney |
| GJB1 | ENSG00000169562 | P08034 | 0.7185 | 0.5841 | liver |
| GJB2 | ENSG00000165474 | P29033 | 0.7914 | 0.5439 | esophagus |
| GJB3 | ENSG00000188910 | O75712 | 0.8277 | 0.6428 | skin |
| GJB4 | ENSG00000189433 | Q9NTQ9 | 0.9322 | 0.8421 | skin |
| GJB5 | ENSG00000189280 | O95377 | 0.8712 | 0.7508 | skin |
| GJB6 | ENSG00000121742 | O95452 | 0.8647 | 0.7504 | esophagus |
| GJB7 | ENSG00000164411 | Q6PEY0 | 0.9611 | 0.8877 | fallopian tube |
| GJC2 | ENSG00000198835 | Q5T442 | 0.7813 | 0.4853 | cerebral cortex |
| GJC3 | ENSG00000176402 | Q8NFK1 | 0.8521 | 0.6084 | breast |
| GJD2 | ENSG00000159248 | Q9UKL4 | 0.9694 | 0.9223 | adrenal gland |
| GJD3 | ENSG00000183153 | Q8N144 | 0.7774 | 0.537 | spleen |
| GJD4 | ENSG00000177291 | Q96KN9 | 0.8018 | 0.5009 | cerebral cortex |
| GLDN | ENSG00000186417 | Q6ZMI3 | 0.7713 | 0.5463 | cerebral cortex |
| GLP1R | ENSG00000112164 | P43220 | 0.8591 | 0.8071 | cerebral cortex |
| GLP2R | ENSG00000065325 | O95838 | 0.8001 | 0.6416 | gallbladder |
| GLRA1 | ENSG00000145888 | P23415 | 0.9931 | 0.9892 | adrenal gland |
| GLRA2 | ENSG00000101958 | P23416 | 0.9522 | 0.898 | cerebral cortex |
| GLRA3 | ENSG00000145451 | O75311 | 0.9665 | 0.9115 | cerebral cortex |
| GLRB | ENSG00000109738 | P48167 | 0.7101 | 0.4729 | parathyroid gland |
| GLT6D1 | ENSG00000204007 | Q7Z4J2 | 1 | 1 | epididymis |
| GNRHR | ENSG00000109163 | P30968 | 0.9173 | 0.818 | adrenal gland |
| GP1BA | ENSG00000185245 | P07359 | 0.832 | 0.5932 | lymph node |
| GP1BB | ENSG00000203618 | P13224 | 0.9402 | 0.8534 | granulocytes |
| GP5 | ENSG00000178732 | P40197 | 0.8588 | 0.7206 | lymph node |
| GP6 | ENSG00000088053 | Q9HCN6 | 0.8762 | 0.7097 | skin |
| GP9 | ENSG00000169704 | P14770 | 0.9436 | 0.8717 | granulocytes |
| GPA33 | ENSG00000143167 | Q99795 | 0.8312 | 0.7357 | rectum |
| GPBAR1 | ENSG00000179921 | Q8TDU6 | 0.859 | 0.5696 | monocytes |
| GPM6A | ENSG00000150625 | P51674 | 0.7844 | 0.4163 | cerebral cortex |
| GPR1 | ENSG00000183671 | P46091 | 0.8154 | 0.5967 | placenta |
| GPR101 | ENSG00000165370 | Q96P66 | 1 | 1 | cerebral cortex |
| GPR119 | ENSG00000147262 | Q8TDV5 | 0.91 | 0.8962 | pancreas |
| GPR12 | ENSG00000132975 | P47775 | 0.9499 | 0.8619 | cerebral cortex |
| GPR135 | ENSG00000181619 | Q8IZ08 | 0.6903 | 0.4918 | fallopian tube |
| GPR139 | ENSG00000180269 | Q6DWJ6 | 0.9911 | 0.987 | endometrium |
| GPR141 | ENSG00000187037 | Q7Z602 | 0.7095 | 0.4316 | bone marrow |
| GPR142 | ENSG00000257008 | Q7Z601 | 1 | 1 | appendix |
| GPR143 | ENSG00000101850 | P51810 | 0.6504 | 0.4686 | skin |
| GPR148 | ENSG00000173302 | Q8TDV2 | 1 | 1 | stomach |
| GPR149 | ENSG00000174948 | Q86SP6 | 0.9983 | 0.9968 | seminal vesicle |
| GPR15 | ENSG00000154165 | P49685 | 0.7845 | 0.6345 | rectum |
| GPR150 | ENSG00000178015 | Q8NGU9 | 0.8726 | 0.6653 | parathyroid gland |
| GPR152 | ENSG00000175514 | Q8TDT2 | 1 | 1 | spleen |
| GPR156 | ENSG00000175697 | Q8NFN8 | 0.8933 | 0.7445 | fallopian tube |
| GPR158 | ENSG00000151025 | Q5T848 | 0.9772 | 0.8937 | cerebral cortex |
| GPR161 | ENSG00000143147 | Q8N6U8 | 0.6565 | 0.4207 | smooth muscle |
| GPR17 | ENSG00000144230 | Q13304 | 0.7134 | 0.5483 | spleen |
| GPR171 | ENSG00000174946 | O14626 | 0.7299 | 0.4017 | t-cells |
| GPR174 | ENSG00000147138 | Q9BXC1 | 0.7059 | 0.4536 | lymph node |
| GPR18 | ENSG00000125245 | Q14330 | 0.7504 | 0.5883 | nk-cells |
| GPR182 | ENSG00000166856 | O15218 | 0.9283 | 0.7541 | spleen |
| GPR19 | ENSG00000183150 | Q15760 | 0.8279 | 0.5477 | cerebral cortex |
| GPR20 | ENSG00000204882 | Q99678 | 0.8085 | 0.5781 | monocytes |
| GPR21 | ENSG00000188394 | Q99679 | 0.816 | 0.5364 | cerebral cortex |
| GPR22 | ENSG00000172209 | Q99680 | 0.9266 | 0.9104 | heart muscle |
| GPR25 | ENSG00000170128 | O00155 | 0.9611 | 0.8981 | t-cells |
| GPR26 | ENSG00000154478 | Q8NDV2 | 1 | 1 | cerebral cortex |
| GPR27 | ENSG00000170837 | Q9NS67 | 0.6935 | 0.421 | parathyroid gland |
| GPR3 | ENSG00000181773 | P46089 | 0.8521 | 0.572 | cerebral cortex |
| GPR31 | ENSG00000120436 | O00270 | 0.9285 | 0.8677 | lymph node |
| GPR32 | ENSG00000142511 | O75388 | 0.9944 | 0.9909 | parathyroid gland |
| GPR35 | ENSG00000178623 | Q9HC97 | 0.6515 | 0.4276 | small intestine |
| GPR37 | ENSG00000170775 | O15354 | 0.8774 | 0.6418 | cerebral cortex |
| GPR37L1 | ENSG00000170075 | O60883 | 0.9761 | 0.8041 | cerebral cortex |
| GPR39 | ENSG00000183840 | O43194 | 0.7671 | 0.572 | parathyroid gland |
| GPR4 | ENSG00000177464 | P46093 | 0.67 | 0.405 | adipose tissue |
| GPR42 | ENSG00000126251 | O15529 | 0.8996 | 0.8869 | appendix |
| GPR45 | ENSG00000135973 | Q9Y5Y3 | 0.9977 | 0.9959 | cerebral cortex |
| GPR50 | ENSG00000102195 | Q13585 | 0.9662 | 0.9264 | placenta |
| GPR52 | ENSG00000203737 | Q9Y2T5 | 0.9877 | 0.9595 | cerebral cortex |
| GPR55 | ENSG00000135898 | Q9Y2T6 | 0.7792 | 0.5548 | spleen |
| GPR6 | ENSG00000146360 | P46095 | 1 | 1 | cerebral cortex |
| GPR61 | ENSG00000156097 | Q9BZJ8 | 0.9662 | 0.9266 | cerebral cortex |
| GPR62 | ENSG00000180929 | Q9BZJ7 | 0.974 | 0.8468 | cerebral cortex |
| GPR65 | ENSG00000140030 | Q8IYL9 | 0.6899 | 0.427 | t-cells |
| GPR75 | ENSG00000119737 | O95800 | 0.7644 | 0.4088 | cerebral cortex |
| GPR82 | ENSG00000171657 | Q96P67 | 0.8403 | 0.5502 | granulocytes |
| GPR83 | ENSG00000123901 | Q9NYM4 | 0.8845 | 0.5479 | thyroid gland |
| GPR84 | ENSG00000139572 | Q9NQS5 | 0.7767 | 0.6001 | bone marrow |
| GPR87 | ENSG00000138271 | Q9BY21 | 0.8488 | 0.7889 | skin |
| GPR88 | ENSG00000181656 | Q9GZNO | 0.8875 | 0.7724 | spleen |
| GPRC5A | ENSG00000013588 | Q8NFJ5 | 0.6952 | 0.4713 | lung |
| GPRC5D | ENSG00000111291 | Q9NZD1 | 0.8103 | 0.5153 | b-cells |
| GPRC6A | ENSG00000173612 | Q5T6X5 | 0.9604 | 0.9422 | kidney |
| GRAMD2A | ENSG00000175318 | Q8IUY3 | 0.8197 | 0.6383 | placenta |
| GREB1 | ENSG00000196208 | Q4ZG55 | 0.7766 | 0.5349 | ovary |
| GREB1L | ENSG00000141449 | Q9C091 | 0.7258 | 0.5669 | thyroid gland |
| GRIA1 | ENSG00000155511 | P42261 | 0.9521 | 0.8156 | cerebral cortex |
| GRIA2 | ENSG00000120251 | P42262 | 0.8926 | 0.7656 | cerebral cortex |
| GRIA3 | ENSG00000125675 | P42263 | 0.8277 | 0.5568 | cerebral cortex |
| GRIA4 | ENSG00000152578 | P48058 | 0.9046 | 0.7143 | cerebral cortex |
| GRID1 | ENSG00000182771 | Q9ULK0 | 0.8491 | 0.6075 | cerebral cortex |
| GRID2 | ENSG00000152208 | O43424 | 0.9422 | 0.8808 | cerebral cortex |
| GRIK1 | ENSG00000171189 | P39086 | 0.9038 | 0.7519 | adrenal gland |
| GRIK2 | ENSG00000164418 | Q13002 | 0.821 | 0.4661 | cerebral cortex |
| GRIK3 | ENSG00000163873 | Q13003 | 0.8602 | 0.5852 | cerebral cortex |
| GRIK4 | ENSG00000149403 | Q16099 | 0.9149 | 0.7465 | cerebral cortex |
| GRIK5 | ENSG00000105737 | Q16478 | 0.7345 | 0.4966 | cerebral cortex |
| GRIN1 | ENSG00000176884 | Q05586 | 0.9873 | 0.9287 | cerebral cortex |
| GRIN2A | ENSG00000183454 | Q12879 | 0.9232 | 0.7076 | cerebral cortex |
| GRIN2B | ENSG00000273079 | Q13224 | 0.9866 | 0.954 | cerebral cortex |
| GRIN2C | ENSG00000161509 | Q14957 | 0.8804 | 0.6899 | thyroid gland |
| GRIN2D | ENSG00000105464 | O15399 | 0.8795 | 0.7124 | cerebral cortex |
| GRIN3A | ENSG00000198785 | Q8TCU5 | 0.9175 | 0.6785 | cerebral cortex |
| GRIN3B | ENSG00000116032 | O60391 | 0.9215 | 0.801 | fallopian tube |
| GRM1 | ENSG00000152822 | Q13255 | 0.976 | 0.951 | cerebral cortex |
| GRM2 | ENSG00000164082 | Q14416 | 0.9852 | 0.9288 | cerebral cortex |
| GRM3 | ENSG00000198822 | Q14832 | 0.9833 | 0.9132 | cerebral cortex |
| GRM4 | ENSG00000124493 | Q14833 | 0.9815 | 0.9072 | cerebral cortex |
| GRM5 | ENSG00000168959 | P41594 | 0.9941 | 0.9737 | cerebral cortex |
| GRM6 | ENSG00000113262 | O15303 | 0.8325 | 0.6712 | cerebral cortex |
| GRM7 | ENSG00000196277 | Q14831 | 0.9033 | 0.8068 | cerebral cortex |
| GRM8 | ENSG00000179603 | O00222 | 0.8539 | 0.7253 | cerebral cortex |
| GRPR | ENSG00000126010 | P30550 | 0.8554 | 0.6632 | pancreas |
| GSDMA | ENSG00000167914 | Q96QA5 | 0.8564 | 0.536 | skin |
| GSDMB | ENSG00000073605 | Q8TAX9 | 0.631 | 0.4023 | small intestine |
| GSDMC | ENSG00000147697 | Q9BYG8 | 0.9207 | 0.8956 | skin |
| GSG1L | ENSG00000169181 | Q6UXU4 | 0.8902 | 0.6614 | cerebral cortex |
| GSG1L2 | ENSG00000214978 | A8MUP6 | 1 | 1 | cerebral cortex |
| GUCY2C | ENSG00000070019 | P25092 | 0.8489 | 0.6619 | small intestine |
| GUCY2D | ENSG00000132518 | Q02846 | 0.9883 | 0.9413 | dendritic cells |
| GUCY2F | ENSG00000101890 | P51841 | 0.9799 | 0.9704 | fallopian tube |
| GYPA | ENSG00000170180 | P02724 | 0.9768 | 0.9409 | bone marrow |
| GYPB | ENSG00000250361 | P06028 | 0.9821 | 0.9704 | bone marrow |
| GYPE | ENSG00000197465 | P15421 | 0.7884 | 0.4775 | bone marrow |
| HAS1 | ENSG00000105509 | Q92839 | 0.8454 | 0.6662 | adipose tissue |
| HAS2 | ENSG00000170961 | Q92819 | 0.739 | 0.5094 | adipose tissue |
| HAS3 | ENSG00000103044 | O00219 | 0.7289 | 0.475 | urinary bladder |
| HAVCR1 | ENSG00000113249 | Q96D42 | 0.8724 | 0.5896 | kidney |
| HCAR1 | ENSG00000196917 | Q9BXC0 | 0.8615 | 0.5031 | parathyroid gland |
| HCAR2 | ENSG00000182782 | Q8TDS4 | 0.7461 | 0.5438 | granulocytes |
| HCAR3 | ENSG00000255398 | P49019 | 0.8063 | 0.6268 | granulocytes |
| HCN1 | ENSG00000164588 | O60741 | 0.983 | 0.9586 | cerebral cortex |
| HCN2 | ENSG00000099822 | Q9UL51 | 0.947 | 0.7854 | cerebral cortex |
| HCN3 | ENSG00000143630 | Q9P1Z3 | 0.6334 | 0.4024 | cerebral cortex |
| HCN4 | ENSG00000138622 | Q9Y3Q4 | 0.933 | 0.889 | heart muscle |
| HCRTR1 | ENSG00000121764 | O43613 | 0.8759 | 0.7364 | adrenal gland |
| HCRTR2 | ENSG00000137252 | O43614 | 0.9394 | 0.929 | kidney |
| HEPACAM | ENSG00000165478 | Q14CZ8 | 0.944 | 0.7786 | cerebral cortex |
| HEPACAM2 | ENSG00000188175 | A8MVW5 | 0.8469 | 0.7401 | rectum |
| HEPHL1 | ENSG00000181333 | Q6MZM0 | 0.976 | 0.9571 | tonsil |
| HHLA2 | ENSG00000114455 | Q9UM44 | 0.8117 | 0.6586 | small intestine |
| HIGD1C | ENSG00000214511 | A8MV81 | 0.775 | 0.5121 | parathyroid gland |
| HLA-DQA2 | ENSG00000237541 | P01906 | 0.9653 | 0.9494 | tonsil |
| HLA-DQB2 | ENSG00000232629 | P05538 | 0.8479 | 0.6557 | skin |
| HLA-G | ENSG00000204632 | P17693 | 0.9752 | 0.8866 | placenta |
| HPN | ENSG00000105707 | P05981 | 0.7623 | 0.5742 | liver |
| HRCT1 | ENSG00000196196 | Q6UXD1 | 0.6863 | 0.4662 | adipose tissue |
| HRH3 | ENSG00000101180 | Q9Y5N1 | 0.9863 | 0.9609 | cerebral cortex |
| HRH4 | ENSG00000134489 | Q9H3N8 | 0.962 | 0.6497 | granulocytes |
| HRK | ENSG00000135116 | O00198 | 0.9208 | 0.8061 | cerebral cortex |
| HS6ST2 | ENSG00000171004 | Q96MM7 | 0.7604 | 0.5888 | ovary |
| HS6ST3 | ENSG00000185352 | Q8IZP7 | 0.8866 | 0.723 | cerebral cortex |
| HSD17B2 | ENSG00000086696 | P37059 | 0.7534 | 0.5811 | placenta |
| HTR1A | ENSG00000178394 | P08908 | 0.9834 | 0.9803 | ovary |
| HTR1B | ENSG00000135312 | P28222 | 0.899 | 0.7407 | placenta |
| HTR1D | ENSG00000179546 | P28221 | 0.9135 | 0.7041 | duodenum |
| HTR1E | ENSG00000168830 | P28566 | 0.923 | 0.8651 | ovary |
| HTR1F | ENSG00000179097 | P30939 | 0.843 | 0.5887 | placenta |
| HTR2A | ENSG00000102468 | P28223 | 0.9113 | 0.6449 | cerebral cortex |
| HTR2B | ENSG00000135914 | P41595 | 0.7424 | 0.4967 | cervix, uterine |
| HTR3A | ENSG00000166736 | P46098 | 0.8703 | 0.7821 | dendritic cells |
| HTR3B | ENSG00000149305 | O95264 | 0.8486 | 0.4438 | cerebral cortex |
| HTR3C | ENSG00000178084 | Q8WXA8 | 0.9841 | 0.9682 | lung |
| HTR3E | ENSG00000186038 | A5X5Y0 | 0.9455 | 0.9339 | duodenum |
| HTR4 | ENSG00000164270 | Q13639 | 0.8898 | 0.8039 | small intestine |
| HTR5A | ENSG00000157219 | P47898 | 1 | 1 | cerebral cortex |
| HTR6 | ENSG00000158748 | P50406 | 0.977 | 0.9482 | cerebral cortex |
| HTR7 | ENSG00000148680 | P34969 | 0.9044 | 0.5659 | parathyroid gland |
| HYAL4 | ENSG00000106302 | Q2M3T9 | 0.867 | 0.497 | placenta |
| ICAM4 | ENSG00000105371 | Q14773 | 0.8454 | 0.4759 | monocytes |
| ICAM5 | ENSG00000105376 | Q9UMF0 | 0.9843 | 0.9579 | cerebral cortex |
| ICOS | ENSG00000163600 | Q9Y6W8 | 0.8134 | 0.6544 | t-cells |
| IER3 | ENSG00000137331 | P46695 | 0.8201 | 0.7179 | appendix |
| IFITM10 | ENSG00000244242 | A6NMD0 | 0.9543 | 0.8357 | adrenal gland |
| IFITM5 | ENSG00000206013 | A6NNB3 | 0.9239 | 0.8699 | bone marrow |
| IGDCC3 | ENSG00000174498 | Q8IVU1 | 0.917 | 0.8218 | parathyroid gland |
| IGDCC4 | ENSG00000103742 | Q8TDY8 | 0.7176 | 0.5429 | ovary |
| IGSF1 | ENSG00000147255 | Q8N6C5 | 0.7444 | 0.5376 | adrenal gland |
| IGSF11 | ENSG00000144847 | Q5DX21 | 0.8331 | 0.6085 | cerebral cortex |
| IGSF23 | ENSG00000216588 | A1L1A6 | 0.9158 | 0.8474 | small intestine |
| IGSF5 | ENSG00000183067 | Q9NSI5 | 0.7132 | 0.4364 | placenta |
| IGSF6 | ENSG00000140749 | O95976 | 0.824 | 0.5122 | granulocytes |
| IGSF9 | ENSG00000085552 | Q9P2J2 | 0.7633 | 0.5713 | skin |
| IGSF9B | ENSG00000080854 | Q9UPX0 | 0.7092 | 0.4747 | cerebral cortex |
| IL12RB2 | ENSG00000081985 | Q99665 | 0.8055 | 0.4751 | nk-cells |
| IL13RA2 | ENSG00000123496 | Q14627 | 0.7211 | 0.5956 | prostate |
| IL17RD | ENSG00000144730 | Q8NFM7 | 0.6713 | 0.5023 | parathyroid gland |
| IL17RE | ENSG00000163701 | Q8NFR9 | 0.6001 | 0.4393 | small intestine |
| IL18RAP | ENSG00000115607 | O95256 | 0.7749 | 0.5374 | nk-cells |
| IL1R2 | ENSG00000115590 | P27930 | 0.6745 | 0.4111 | granulocytes |
| IL1RAPL1 | ENSG00000169306 | Q9NZN1 | 0.9518 | 0.7378 | cerebral cortex |
| IL1RAPL2 | ENSG00000189108 | Q9NP60 | 0.9314 | 0.8204 | parathyroid gland |
| IL1RL1 | ENSG00000115602 | Q01638 | 0.7907 | 0.469 | placenta |
| IL1RL2 | ENSG00000115598 | Q9HB29 | 0.7646 | 0.5332 | skin |
| IL20RA | ENSG00000016402 | Q9UHF4 | 0.6787 | 0.5026 | skin |
| IL20RB | ENSG00000174564 | Q6UXL0 | 0.927 | 0.8353 | skin |
| IL21R | ENSG00000103522 | Q9HBE5 | 0.7339 | 0.5311 | lymph node |
| IL22RA1 | ENSG00000142677 | Q8N6P7 | 0.784 | 0.6542 | skin |
| IL23R | ENSG00000162594 | Q5VWK5 | 0.9487 | 0.7894 | t-cells |
| IL2RA | ENSG00000134460 | P01589 | 0.7733 | 0.4982 | t-cells |
| IL31RA | ENSG00000164509 | Q8NI17 | 0.8857 | 0.625 | bone marrow |
| IL5RA | ENSG00000091181 | Q01344 | 0.9003 | 0.5793 | granulocytes |
| IL9R | ENSG00000124334 | Q01113 | 0.8254 | 0.5829 | urinary bladder |
| ILDR1 | ENSG00000145103 | Q86SU0 | 0.6806 | 0.475 | parathyroid gland |
| IMPG2 | ENSG00000081148 | Q9BZV3 | 0.9714 | 0.7483 | fallopian tube |
| INSRR | ENSG00000027644 | P14616 | 0.9281 | 0.8545 | adrenal gland |
| ISLR2 | ENSG00000167178 | Q6UXK2 | 0.843 | 0.4714 | cerebral cortex |
| ITGA10 | ENSG00000143127 | O75578 | 0.668 | 0.407 | cervix, uterine |
| ITGA11 | ENSG00000137809 | Q9UKX5 | 0.6997 | 0.4556 | smooth muscle |
| ITGA2B | ENSG00000005961 | P08514 | 0.8468 | 0.5862 | granulocytes |
| ITGA8 | ENSG00000077943 | P53708 | 0.6175 | 0.4242 | prostate |
| ITGAD | ENSG00000156886 | Q13349 | 0.9298 | 0.772 | spleen |
| ITGAE | ENSG00000083457 | P38570 | 0.7801 | 0.5601 | lung |
| ITGB3 | ENSG00000259207 | P05106 | 0.6934 | 0.4142 | thyroid gland |
| ITGB6 | ENSG00000115221 | P18564 | 0.6863 | 0.5334 | lung |
| IYD | ENSG00000009765 | Q6PHW0 | 0.8931 | 0.6823 | thyroid gland |
| IZUMO1 | ENSG00000182264 | Q8IYV9 | 0.8728 | 0.7902 | lung |
| IZUMO2 | ENSG00000161652 | Q6UXV1 | 0.9385 | 0.9367 | prostate |
| JAG2 | ENSG00000184916 | Q9Y219 | 0.7347 | 0.4239 | skin |
| JPH1 | ENSG00000104369 | Q9HDC5 | 0.8392 | 0.5298 | skeletal muscle |
| JPH2 | ENSG00000149596 | Q9BR39 | 0.6739 | 0.5031 | skeletal muscle |
| JPH3 | ENSG00000154118 | Q8WXH2 | 0.9738 | 0.8806 | cerebral cortex |
| JPH4 | ENSG00000092051 | Q96JJ6 | 0.7876 | 0.5622 | cerebral cortex |
| KCNA1 | ENSG00000111262 | Q09470 | 0.9692 | 0.9064 | cerebral cortex |
| KCNA10 | ENSG00000143105 | Q16322 | 1 | 1 | spleen |
| KCNA2 | ENSG00000177301 | P16389 | 0.8905 | 0.5561 | cerebral cortex |
| KCNA3 | ENSG00000177272 | P22001 | 0.672 | 0.437 | t-cells |
| KCNA4 | ENSG00000182255 | P22459 | 0.9417 | 0.8738 | adrenal gland |
| KCNA5 | ENSG00000130037 | P22460 | 0.8081 | 0.5462 | heart muscle |
| KCNA6 | ENSG00000151079 | P17658 | 0.9512 | 0.9512 | appendix |
| KCNA7 | ENSG00000104848 | Q96RP8 | 0.9616 | 0.6927 | skeletal muscle |
| KCNB1 | ENSG00000158445 | Q14721 | 0.8187 | 0.618 | cerebral cortex |
| KCNB2 | ENSG00000182674 | Q92953 | 0.9306 | 0.8475 | spleen |
| KCNC1 | ENSG00000129159 | P48547 | 0.9087 | 0.5839 | cerebral cortex |
| KCNC2 | ENSG00000166006 | Q96PR1 | 0.9801 | 0.9638 | cerebral cortex |
| KCNC3 | ENSG00000131398 | Q14003 | 0.723 | 0.4768 | parathyroid gland |
| KCND2 | ENSG00000184408 | Q9NZV8 | 0.8556 | 0.6652 | cerebral cortex |
| KCND3 | ENSG00000171385 | Q9UK17 | 0.607 | 0.4304 | cerebral cortex |
| KCNE1 | ENSG00000180509 | P15382 | 0.8903 | 0.6834 | fallopian tube |
| KCNE2 | ENSG00000159197 | Q9Y6J6 | 0.935 | 0.5809 | stomach |
| KCNE5 | ENSG00000176076 | Q9UJ90 | 0.8582 | 0.7018 | cerebral cortex |
| KCNF1 | ENSG00000162975 | Q9H3M0 | 0.9708 | 0.7972 | cerebral cortex |
| KCNG1 | ENSG00000026559 | Q9UIX4 | 0.7559 | 0.5741 | endometrium |
| KCNG2 | ENSG00000178342 | Q9UJ96 | 0.9655 | 0.9142 | cerebral cortex |
| KCNG3 | ENSG00000171126 | Q8TAE7 | 0.9217 | 0.8349 | cerebral cortex |
| KCNG4 | ENSG00000168418 | Q8TDN1 | 0.8521 | 0.6923 | adrenal gland |
| KCNH1 | ENSG00000143473 | O95259 | 0.9604 | 0.7711 | cerebral cortex |
| KCNH2 | ENSG00000055118 | Q12809 | 0.7084 | 0.5165 | bone marrow |
| KCNH3 | ENSG00000135519 | Q9ULD8 | 0.8975 | 0.6586 | cerebral cortex |
| KCNH4 | ENSG00000089558 | Q9UQ05 | 0.9278 | 0.7565 | cerebral cortex |
| KCNH5 | ENSG00000140015 | Q8NCM2 | 0.9803 | 0.9702 | cerebral cortex |
| KCNH6 | ENSG00000173826 | Q9H252 | 0.8972 | 0.797 | kidney |
| KCNH7 | ENSG00000184611 | Q9NS40 | 0.9699 | 0.8941 | cerebral cortex |
| KCNH8 | ENSG00000183960 | Q96L42 | 0.8522 | 0.5871 | cerebral cortex |
| KCNJ1 | ENSG00000151704 | P48048 | 0.9755 | 0.8648 | kidney |
| KCNJ10 | ENSG00000177807 | P78508 | 0.9133 | 0.7408 | cerebral cortex |
| KCNJ12 | ENSG00000184185 | Q14500 | 0.8194 | 0.5279 | skeletal muscle |
| KCNJ13 | ENSG00000115474 | O60928 | 0.8975 | 0.7549 | small intestine |
| KCNJ15 | ENSG00000157551 | Q99712 | 0.6824 | 0.4665 | kidney |
| KCNJ16 | ENSG00000153822 | Q9NPI9 | 0.7928 | 0.6131 | parathyroid gland |
| KCNJ3 | ENSG00000162989 | P48549 | 0.8023 | 0.6127 | parathyroid gland |
| KCNJ4 | ENSG00000168135 | P48050 | 0.9682 | 0.9402 | cerebral cortex |
| KCNJ5 | ENSG00000120457 | P48544 | 0.7996 | 0.4801 | adrenal gland |
| KCNJ6 | ENSG00000157542 | P48051 | 0.9366 | 0.7923 | cerebral cortex |
| KCNJ9 | ENSG00000162728 | Q92806 | 0.9974 | 0.9942 | cerebral cortex |
| KCNK10 | ENSG00000100433 | P57789 | 0.873 | 0.7631 | dendritic cells |
| KCNK12 | ENSG00000184261 | Q9HB15 | 0.9856 | 0.9436 | cerebral cortex |
| KCNK13 | ENSG00000152315 | Q9HB14 | 0.8666 | 0.5491 | parathyroid gland |
| KCNK16 | ENSG00000095981 | Q96T55 | 0.9377 | 0.8945 | stomach |
| KCNK17 | ENSG00000124780 | Q96T54 | 0.8649 | 0.5428 | dendritic cells |
| KCNK18 | ENSG00000186795 | Q7Z418 | 1 | 1 | cerebral cortex |
| KCNK2 | ENSG00000082482 | O95069 | 0.8437 | 0.629 | adrenal gland |
| KCNK3 | ENSG00000171303 | O14649 | 0.801 | 0.53 | adrenal gland |
| KCNK4 | ENSG00000182450 | Q9NYG8 | 0.988 | 0.9601 | cerebral cortex |
| KCNK5 | ENSG00000164626 | O95279 | 0.6158 | 0.4046 | small intestine |
| KCNK7 | ENSG00000173338 | Q9Y2U2 | 0.8857 | 0.5634 | skin |
| KCNK9 | ENSG00000169427 | Q9NPC2 | 0.9595 | 0.9396 | cerebral cortex |
| KCNMB2 | ENSG00000197584 | Q9Y691 | 0.8134 | 0.6289 | epididymis |
| KCNMB4 | ENSG00000135643 | Q86W47 | 0.7679 | 0.4047 | cerebral cortex |
| KCNN1 | ENSG00000105642 | Q92952 | 0.9589 | 0.7325 | cerebral cortex |
| KCNN2 | ENSG00000080709 | Q9H2S1 | 0.7821 | 0.5713 | adrenal gland |
| KCNQ2 | ENSG00000075043 | O43526 | 0.9735 | 0.9336 | cerebral cortex |
| KCNQ3 | ENSG00000184156 | O43525 | 0.9184 | 0.5983 | cerebral cortex |
| KCNQ4 | ENSG00000117013 | P56696 | 0.7381 | 0.5498 | smooth muscle |
| KCNQ5 | ENSG00000185760 | Q9NR82 | 0.8415 | 0.5809 | cerebral cortex |
| KCNS1 | ENSG00000124134 | Q96KK3 | 0.9308 | 0.8631 | cerebral cortex |
| KCNS2 | ENSG00000156486 | Q9ULS6 | 0.9041 | 0.7179 | cerebral cortex |
| KCNT1 | ENSG00000107147 | Q5JUK3 | 0.917 | 0.8021 | cerebral cortex |
| KCNT2 | ENSG00000162687 | Q6UVM3 | 0.7776 | 0.5365 | ovary |
| KCNU1 | ENSG00000215262 | A8MYU2 | 0.9618 | 0.9541 | adipose tissue |
| KCNV1 | ENSG00000164794 | Q6PIU1 | 0.9429 | 0.5772 | cerebral cortex |
| KCNV2 | ENSG00000168263 | Q8TDN2 | 0.9756 | 0.9756 | b-cells |
| KEL | ENSG00000197993 | P23276 | 0.9256 | 0.8338 | bone marrow |
| KIAA0319 | ENSG00000137261 | Q5VV43 | 0.935 | 0.7682 | cerebral cortex |
| KIAA1549 | ENSG00000122778 | Q9HCM3 | 0.6803 | 0.4504 | seminal vesicle |
| KIAA1549L | ENSG00000110427 | Q6ZVL6 | 0.9459 | 0.8124 | parathyroid gland |
| KIR2DL4 | ENSG00000189013 | Q99706 | 1 | 1 | nk-cells |
| KIR3DL1 | ENSG00000167633 | P43629 | 0.9892 | 0.9822 | t-cells |
| KIR3DL2 | ENSG00000240403 | P43630 | 1 | 1 | t-cells |
| KIRREL3 | ENSG00000149571 | Q8IZU9 | 0.9044 | 0.6553 | cerebral cortex |
| KISS1R | ENSG00000116014 | Q969F8 | 0.9664 | 0.9119 | cerebral cortex |
| KL | ENSG00000133116 | Q9UEF7 | 0.8335 | 0.5447 | parathyroid gland |
| KLB | ENSG00000134962 | Q86Z14 | 0.9039 | 0.6768 | adipose tissue |
| KLHDC7A | ENSG00000179023 | Q5VTJ3 | 0.864 | 0.7067 | kidney |
| KLRB1 | ENSG00000111796 | Q12918 | 0.7677 | 0.4149 | t-cells |
| KLRC1 | ENSG00000134545 | P26715 | 0.8204 | 0.4208 | nk-cells |
| KLRC3 | ENSG00000205810 | Q07444 | 0.9011 | 0.6979 | nk-cells |
| KLRC4 | ENSG00000183542 | O43908 | 0.7286 | 0.4793 | spleen |
| KLRF1 | ENSG00000150045 | Q9NZS2 | 0.8489 | 0.4614 | nk-cells |
| KLRF2 | ENSG00000256797 | D3WOD1 | 0.94 | 0.8161 | skin |
| KLRG2 | ENSG00000188883 | A4D1S0 | 0.8656 | 0.8029 | thyroid gland |
| KREMEN2 | ENSG00000131650 | Q8NCW0 | 0.927 | 0.748 | skin |
| L1CAM | ENSG00000198910 | P32004 | 0.7264 | 0.4563 | cerebral cortex |
| LAG3 | ENSG00000089692 | P18627 | 0.7239 | 0.5025 | spleen |
| LAMP5 | ENSG00000125869 | Q9UJQ1 | 0.7748 | 0.4879 | dendritic cells |
| LAX1 | ENSG00000122188 | Q8IWV1 | 0.6998 | 0.5126 | tonsil |
| LCT | ENSG00000115850 | P09848 | 0.9743 | 0.9664 | duodenum |
| LDLRAD1 | ENSG00000203985 | Q5T700 | 0.9372 | 0.752 | fallopian tube |
| LDLRAD2 | ENSG00000187942 | Q5SZI1 | 0.741 | 0.4888 | adipose tissue |
| LEMD1 | ENSG00000186007 | Q68G75 | 0.9453 | 0.7786 | epididymis |
| LGR5 | ENSG00000139292 | O75473 | 0.7737 | 0.5888 | placenta |
| LGR6 | ENSG00000133067 | Q9HBX8 | 0.7406 | 0.5152 | t-cells |
| LHCGR | ENSG00000138039 | P22888 | 0.9155 | 0.8824 | ovary |
| LHFPL1 | ENSG00000182508 | Q86WI0 | 0.8287 | 0.6423 | cerebral cortex |
| LHFPL4 | ENSG00000156959 | Q7Z7J7 | 0.9777 | 0.9204 | cerebral cortex |
| LHFPL5 | ENSG00000197753 | Q8TAF8 | 0.7749 | 0.4209 | epididymis |
| LILRA1 | ENSG00000104974 | O75019 | 0.9511 | 0.8844 | monocytes |
| LILRA5 | ENSG00000187116 | A6NI73 | 0.8829 | 0.7109 | monocytes |
| LIM2 | ENSG00000105370 | P55344 | 0.981 | 0.9464 | t-cells |
| LIME1 | ENSG00000203896 | Q9H400 | 0.814 | 0.612 | dendritic cells |
| LINGO1 | ENSG00000169783 | Q96FE5 | 0.817 | 0.4679 | cerebral cortex |
| LINGO2 | ENSG00000174482 | Q7L985 | 0.8559 | 0.748 | smooth muscle |
| LINGO3 | ENSG00000220008 | P0C6S8 | 0.8984 | 0.8303 | spleen |
| LINGO4 | ENSG00000213171 | Q6UY18 | 0.8889 | 0.6497 | skeletal muscle |
| LMTK3 | ENSG00000142235 | Q96Q04 | 0.8839 | 0.6242 | cerebral cortex |
| LPAR3 | ENSG00000171517 | Q9UBY5 | 0.8097 | 0.5882 | fallopian tube |
| LPAR4 | ENSG00000147145 | Q99677 | 0.822 | 0.5977 | ovary |
| LPCAT1 | ENSG00000153395 | Q8NF37 | 0.7879 | 0.4599 | lung |
| LRFN1 | ENSG00000128011 | Q9P244 | 0.8453 | 0.4576 | cerebral cortex |
| LRFN2 | ENSG00000156564 | Q9ULH4 | 0.9674 | 0.9055 | cerebral cortex |
| LRFN5 | ENSG00000165379 | Q96NI6 | 0.8195 | 0.6066 | parathyroid gland |
| LRIT2 | ENSG00000204033 | A6NDA9 | 0.9814 | 0.9602 | skin |
| LRIT3 | ENSG00000183423 | Q3SXY7 | 0.8689 | 0.6618 | kidney |
| LRP1B | ENSG00000168702 | Q9NZR2 | 0.9029 | 0.7876 | cerebral cortex |
| LRP2 | ENSG00000081479 | P98164 | 0.913 | 0.8631 | parathyroid gland |
| LRP4 | ENSG00000134569 | O75096 | 0.7087 | 0.4445 | skin |
| LRP8 | ENSG00000157193 | Q14114 | 0.7881 | 0.4515 | thyroid gland |
| LRRC15 | ENSG00000172061 | Q8TF66 | 0.6834 | 0.5809 | cervix, uterine |
| LRRC19 | ENSG00000184434 | Q9H756 | 0.863 | 0.8065 | kidney |
| LRRC26 | ENSG00000184709 | Q210M4 | 0.9209 | 0.8594 | dendritic cells |
| LRRC37A | ENSG00000176681 | A6NMS7 | 0.7528 | 0.4355 | skeletal muscle |
| LRRC38 | ENSG00000162494 | Q5VT99 | 0.9533 | 0.8611 | adrenal gland |
| LRRC3B | ENSG00000179796 | Q96PB8 | 0.9222 | 0.7338 | cerebral cortex |
| LRRC3C | ENSG00000204913 | A6NJW4 | 0.9591 | 0.9178 | parathyroid gland |
| LRRC4 | ENSG00000128594 | Q9HBW1 | 0.7186 | 0.4284 | cerebral cortex |
| LRRC4B | ENSG00000131409 | Q9NT99 | 0.8376 | 0.568 | cerebral cortex |
| LRRC4C | ENSG00000148948 | Q9HCJ2 | 0.8489 | 0.5469 | cerebral cortex |
| LRRC52 | ENSG00000162763 | Q8N7C0 | 0.9678 | 0.9506 | parathyroid gland |
| LRRC55 | ENSG00000183908 | Q6ZSA7 | 0.8646 | 0.6325 | cerebral cortex |
| LRRC66 | ENSG00000188993 | Q68CR7 | 0.8603 | 0.7096 | duodenum |
| LRRN1 | ENSG00000175928 | Q6UXK5 | 0.7324 | 0.4796 | cerebral cortex |
| LRRN2 | ENSG00000170382 | O75325 | 0.6768 | 0.4438 | cerebral cortex |
| LRRN3 | ENSG00000173114 | Q9H3W5 | 0.6934 | 0.4958 | thyroid gland |
| LRRN4 | ENSG00000125872 | Q8WUT4 | 0.9767 | 0.9317 | lung |
| LRRN4CL | ENSG00000177363 | Q8ND94 | 0.6509 | 0.4579 | endometrium |
| LRRTM1 | ENSG00000162951 | Q86UE6 | 0.8861 | 0.6817 | cerebral cortex |
| LRRTM2 | ENSG00000146006 | O43300 | 0.943 | 0.7453 | cerebral cortex |
| LRRTM3 | ENSG00000198739 | Q86VH5 | 0.9607 | 0.9108 | cerebral cortex |
| LRTM1 | ENSG00000144771 | Q9HBL6 | 0.8814 | 0.6793 | skeletal muscle |
| LRTM2 | ENSG00000166159 | Q8N967 | 1 | 1 | cerebral cortex |
| LTB | ENSG00000227507 | Q06643 | 0.7932 | 0.6701 | t-cells |
| LTK | ENSG00000062524 | P29376 | 0.6759 | 0.5109 | placenta |
| LVRN | ENSG00000172901 | Q6Q4G3 | 0.9498 | 0.833 | placenta |
| LY6G6F | ENSG00000204424 | Q5SQ64 | 0.9401 | 0.9017 | bone marrow |
| LY9 | ENSG00000122224 | Q9HBG7 | 0.7229 | 0.4772 | t-cells |
| MADCAM1 | ENSG00000099866 | Q13477 | 0.8279 | 0.5918 | appendix |
| MAG | ENSG00000105695 | P20916 | 0.9472 | 0.8486 | cerebral cortex |
| MARCO | ENSG00000019169 | Q9UEW3 | 0.7384 | 0.4998 | lung |
| MARVELD2 | ENSG00000152939 | Q8N4S9 | 0.6804 | 0.4561 | thyroid gland |
| MAS1 | ENSG00000130368 | P04201 | 0.9377 | 0.7909 | cerebral cortex |
| MAS1L | ENSG00000204687 | P35410 | 0.8744 | 0.7078 | cervix, uterine |
| MC2R | ENSG00000185231 | Q01718 | 0.9865 | 0.9659 | adrenal gland |
| MC4R | ENSG00000166603 | P32245 | 0.9571 | 0.8937 | fallopian tube |
| MC5R | ENSG00000176136 | P33032 | 0.9843 | 0.961 | epididymis |
| MCEMP1 | ENSG00000183019 | Q8IX19 | 0.8506 | 0.7534 | lung |
| MCHR1 | ENSG00000128285 | Q99705 | 0.9341 | 0.7882 | cerebral cortex |
| MCHR2 | ENSG00000152034 | Q969V1 | 0.9979 | 0.9943 | cerebral cortex |
| MCOLN2 | ENSG00000153898 | Q8IZK6 | 0.6509 | 0.4273 | dendritic cells |
| MCOLN3 | ENSG00000055732 | Q8TDD5 | 0.7659 | 0.5313 | adrenal gland |
| MEGF10 | ENSG00000145794 | Q96KG7 | 0.9212 | 0.7039 | cerebral cortex |
| MEGF11 | ENSG00000157890 | A6BM72 | 0.9427 | 0.8741 | cerebral cortex |
| MEP1A | ENSG00000112818 | Q16819 | 0.9082 | 0.8432 | small intestine |
| MEP1B | ENSG00000141434 | Q16820 | 0.9003 | 0.6451 | small intestine |
| MFAP3L | ENSG00000198948 | O75121 | 0.6233 | 0.4148 | epididymis |
| MFRP | ENSG00000235718 | Q9BY79 | 1 | 1 | parathyroid gland |
| MFSD2A | ENSG00000168389 | Q8NA29 | 0.6258 | 0.4075 | epididymis |
| MFSD2B | ENSG00000205639 | A6NFX1 | 0.9714 | 0.923 | bone marrow |
| MFSD4A | ENSG00000174514 | Q8N468 | 0.7568 | 0.5524 | stomach |
| MGAM | ENSG00000257335 | O43451 | 0.8462 | 0.7228 | small intestine |
| MGAM2 | ENSG00000257743 | Q2M2H8 | 0.915 | 0.8281 | duodenum |
| MICB | ENSG00000204516 | Q29980 | 0.7297 | 0.4835 | lymph node |
| MIP | ENSG00000135517 | P30301 | 0.8991 | 0.8565 | cerebral cortex |
| MLC1 | ENSG00000100427 | Q15049 | 0.8843 | 0.6848 | cerebral cortex |
| MLNR | ENSG00000102539 | O43193 | 0.9693 | 0.9106 | thyroid gland |
| MME | ENSG00000196549 | P08473 | 0.6103 | 0.4207 | duodenum |
| MMEL1 | ENSG00000142606 | Q495T6 | 0.7688 | 0.6414 | granulocytes |
| MMP16 | ENSG00000156103 | P51512 | 0.8139 | 0.5655 | cerebral cortex |
| MMP23B | ENSG00000189409 | O75900 | 0.7057 | 0.4499 | dendritic cells |
| MMP24 | ENSG00000125966 | Q9Y5R2 | 0.7529 | 0.5117 | cerebral cortex |
| MOG | ENSG00000204655 | Q16653 | 1 | 1 | cerebral cortex |
| MPIG6B | ENSG00000204420 | O95866 | 0.9075 | 0.7976 | nk-cells |
| MPL | ENSG00000117400 | P40238 | 0.7722 | 0.4893 | ovary |
| MPZ | ENSG00000158887 | P25189 | 0.6402 | 0.4013 | seminal vesicle |
| MRAP | ENSG00000170262 | Q8TCY5 | 0.911 | 0.7895 | adrenal gland |
| MRAP2 | ENSG00000135324 | Q96G30 | 0.6705 | 0.4606 | cerebral cortex |
| MRGPRD | ENSG00000172938 | Q8TDS7 | 0.9468 | 0.883 | seminal vesicle |
| MRGPRE | ENSG00000184350 | Q86SM8 | 0.8762 | 0.7484 | cervix, uterine |
| MRGPRF | ENSG00000172935 | Q96AM1 | 0.5848 | 0.4214 | smooth muscle |
| MRGPRX2 | ENSG00000183695 | Q96LB1 | 0.9138 | 0.8541 | breast |
| MRGPRX3 | ENSG00000179826 | Q96LB0 | 0.9028 | 0.7178 | salivary gland |
| MROH7 | ENSG00000184313 | Q68CQ1 | 0.74 | 0.55 | ovary |
| MS4A1 | ENSG00000156738 | P11836 | 0.7323 | 0.5145 | tonsil |
| MS4A10 | ENSG00000172689 | Q96PG2 | 0.9594 | 0.8651 | small intestine |
| MS4A12 | ENSG00000071203 | Q9NXJ0 | 0.9481 | 0.9211 | rectum |
| MS4A14 | ENSG00000166928 | Q96JA4 | 0.6946 | 0.4546 | spleen |
| MS4A15 | ENSG00000166961 | Q8N5U1 | 0.8253 | 0.5051 | lung |
| MS4A18 | ENSG00000214782 | Q3C1V0 | 0.966 | 0.9538 | duodenum |
| MS4A2 | ENSG00000149534 | Q01362 | 0.803 | 0.5073 | granulocytes |
| MS4A5 | ENSG00000166930 | Q9H3V2 | 0.9872 | 0.9833 | duodenum |
| MS4A6E | ENSG00000166926 | Q96DS6 | 0.9437 | 0.876 | appendix |
| MS4A8 | ENSG00000166959 | Q9BY19 | 0.8039 | 0.7146 | fallopian tube |
| MSLNL | ENSG00000162006 | Q96KJ4 | 0.9806 | 0.952 | epididymis |
| MST1R | ENSG00000164078 | Q04912 | 0.7262 | 0.5155 | skin |
| MTNR1A | ENSG00000168412 | P48039 | 0.9224 | 0.8431 | kidney |
| MTNR1B | ENSG00000134640 | P49286 | 0.9778 | 0.9525 | placenta |
| MUC1 | ENSG00000185499 | P15941 | 0.608 | 0.4207 | stomach |
| MUC12 | ENSG00000205277 | Q9UKN1 | 0.9557 | 0.9286 | rectum |
| MUC13 | ENSG00000173702 | Q9H3R2 | 0.8227 | 0.7339 | duodenum |
| MUC15 | ENSG00000169550 | Q8N387 | 0.8744 | 0.7307 | epididymis |
| MUC16 | ENSG00000181143 | Q8WX17 | 0.9503 | 0.9121 | cervix, uterine |
| MUC17 | ENSG00000169876 | Q685J3 | 0.9551 | 0.9336 | small intestine |
| MUC21 | ENSG00000204544 | Q5SSG8 | 0.9874 | 0.9765 | esophagus |
| MUC22 | ENSG00000261272 | E2RYF6 | 0.9992 | 0.9985 | esophagus |
| MUC4 | ENSG00000145113 | Q99102 | 0.9556 | 0.8949 | colon |
| MUSK | ENSG00000030304 | O15146 | 0.7693 | 0.6893 | rectum |
| MYADML2 | ENSG00000185105 | A6NDP7 | 0.9741 | 0.9273 | skeletal muscle |
| MYMK | ENSG00000187616 | A6NI61 | 0.9329 | 0.9203 | cerebral cortex |
| MYRFL | ENSG00000166268 | Q96LU7 | 0.9248 | 0.7562 | small intestine |
| NAALAD2 | ENSG00000077616 | Q9Y3Q0 | 0.6922 | 0.5092 | adrenal gland |
| NAALADL2 | ENSG00000177694 | Q58DX5 | 0.7304 | 0.4312 | parathyroid gland |
| NALCN | ENSG00000102452 | Q8IZF0 | 0.7967 | 0.4599 | cerebral cortex |
| NAT8L | ENSG00000185818 | Q8N9F0 | 0.8855 | 0.6792 | cerebral cortex |
| NCAM1 | ENSG00000149294 | P13591 | 0.6599 | 0.4035 | cerebral cortex |
| NCAM2 | ENSG00000154654 | O15394 | 0.8649 | 0.5689 | cerebral cortex |
| NCMAP | ENSG00000184454 | Q5T1S8 | 0.7744 | 0.5474 | gallbladder |
| NCR1 | ENSG00000189430 | O76036 | 0.9802 | 0.9454 | spleen |
| NCR2 | ENSG00000096264 | O95944 | 0.9632 | 0.9079 | dendritic cells |
| NCR3 | ENSG00000204475 | O14931 | 0.9371 | 0.8624 | t-cells |
| NCR3LG1 | ENSG00000188211 | Q68D85 | 0.7446 | 0.4619 | parathyroid gland |
| NECTIN4 | ENSG00000143217 | Q96NY8 | 0.7706 | 0.5933 | skin |
| NETO1 | ENSG00000166342 | Q8TDF5 | 0.948 | 0.8266 | cerebral cortex |
| NFAM1 | ENSG00000235568 | Q8NET5 | 0.7162 | 0.4392 | granulocytes |
| NIPAL1 | ENSG00000163293 | Q6NVV3 | 0.6965 | 0.5014 | skin |
| NIPAL4 | ENSG00000172548 | Q0D2K0 | 0.8609 | 0.6806 | skin |
| NKAIN1 | ENSG00000084628 | Q4KMZ8 | 0.8874 | 0.7963 | adrenal gland |
| NKAIN2 | ENSG00000188580 | Q5VXU1 | 0.9441 | 0.6405 | cerebral cortex |
| NKAIN3 | ENSG00000185942 | Q8N8D7 | 0.9264 | 0.8723 | adrenal gland |
| NKAIN4 | ENSG00000101198 | Q8IVV8 | 0.951 | 0.8012 | cerebral cortex |
| NKPD1 | ENSG00000179846 | Q17RQ9 | 0.9695 | 0.8498 | skin |
| NLGN1 | ENSG00000169760 | Q8N2Q7 | 0.7962 | 0.5518 | cerebral cortex |
| NLGN3 | ENSG00000196338 | Q9NZ94 | 0.8258 | 0.5652 | cerebral cortex |
| NLGN4X | ENSG00000146938 | Q8NOW4 | 0.7084 | 0.4705 | cerebral cortex |
| NLGN4Y | ENSG00000165246 | Q8NFZ3 | 0.8089 | 0.6538 | seminal vesicle |
| NMBR | ENSG00000135577 | P28336 | 0.9486 | 0.8103 | fallopian tube |
| NMUR1 | ENSG00000171596 | Q9HB89 | 0.7528 | 0.44 | t-cells |
| NMUR2 | ENSG00000132911 | Q9GZQ4 | 0.9256 | 0.8519 | stomach |
| NOMO1 | ENSG00000103512 | Q15155 | 0.5756 | 0.4604 | thyroid gland |
| NOTCH4 | ENSG00000204301 | Q99466 | 0.8648 | 0.6619 | lung |
| NOX1 | ENSG00000007952 | Q9Y5S8 | 0.9057 | 0.7242 | rectum |
| NOX3 | ENSG00000074771 | Q9HBY0 | 1 | 1 | adrenal gland |
| NOX4 | ENSG00000086991 | Q9NPH5 | 0.8662 | 0.5719 | kidney |
| NOX5 | ENSG00000255346 | Q96PH1 | 0.9359 | 0.7661 | spleen |
| NPBWR2 | ENSG00000125522 | P48146 | 0.9756 | 0.9756 | adrenal gland |
| NPC1L1 | ENSG00000015520 | Q9UHC9 | 0.9233 | 0.8565 | small intestine |
| NPFFR2 | ENSG00000056291 | Q9Y5X5 | 0.9176 | 0.7584 | seminal vesicle |
| NPHS1 | ENSG00000161270 | O60500 | 0.9451 | 0.9045 | kidney |
| NPR1 | ENSG00000169418 | P16066 | 0.6483 | 0.4094 | adipose tissue |
| NPSR1 | ENSG00000187258 | Q6W5P4 | 0.9828 | 0.9597 | stomach |
| NPY2R | ENSG00000185149 | P49146 | 0.9279 | 0.8467 | cerebral cortex |
| NPY5R | ENSG00000164129 | Q15761 | 0.7905 | 0.5786 | spleen |
| NRCAM | ENSG00000091129 | Q92823 | 0.7837 | 0.5128 | cerebral cortex |
| NRG2 | ENSG00000158458 | O14511 | 0.7334 | 0.5251 | parathyroid gland |
| NRG3 | ENSG00000185737 | P56975 | 0.9264 | 0.7613 | cerebral cortex |
| NRG4 | ENSG00000169752 | Q8WWWG1 | 0.8997 | 0.8148 | fallopian tube |
| NRSN1 | ENSG00000152954 | Q8IZ57 | 0.9525 | 0.7085 | cerebral cortex |
| NRXN1 | ENSG00000179915 | Q9ULB1 | 0.857 | 0.5042 | cerebral cortex |
| NRXN2 | ENSG00000110076 | Q9P2S2 | 0.8504 | 0.5482 | cerebral cortex |
| NRXN3 | ENSG00000021645 | Q9Y4C0 | 0.7146 | 0.4647 | cerebral cortex |
| NSG1 | ENSG00000168824 | P42857 | 0.6397 | 0.4208 | skin |
| NSG2 | ENSG00000170091 | Q9Y328 | 0.9278 | 0.8113 | cerebral cortex |
| NTRK1 | ENSG00000198400 | P04629 | 0.9497 | 0.7575 | granulocytes |
| NTRK2 | ENSG00000148053 | Q16620 | 0.7032 | 0.4316 | cerebral cortex |
| NTRK3 | ENSG00000140538 | Q16288 | 0.7303 | 0.4476 | cerebral cortex |
| NTSR1 | ENSG00000101188 | P30989 | 0.941 | 0.9093 | cerebral cortex |
| NTSR2 | ENSG00000169006 | O95665 | 0.992 | 0.982 | cerebral cortex |
| NUP210L | ENSG00000143552 | Q5VU65 | 0.9098 | 0.8718 | seminal vesicle |
| NXPE2 | ENSG00000204361 | Q96DL1 | 0.9059 | 0.803 | epididymis |
| OCLN | ENSG00000197822 | Q16625 | 0.6232 | 0.4107 | thyroid gland |
| OCSTAMP | ENSG00000149635 | Q9BR26 | 0.9931 | 0.9892 | dendritic cells |
| OLR1 | ENSG00000173391 | P78380 | 0.7517 | 0.4572 | placenta |
| OPALIN | ENSG00000197430 | Q96PE5 | 1 | 1 | cerebral cortex |
| OPN1SW | ENSG00000128617 | P03999 | 0.6951 | 0.5377 | heart muscle |
| OPN4 | ENSG00000122375 | Q9UHM6 | 0.9364 | 0.8682 | cerebral cortex |
| OPN5 | ENSG00000124818 | Q6U736 | 1 | 1 | heart muscle |
| OPRD1 | ENSG00000116329 | P41143 | 0.8954 | 0.5128 | cerebral cortex |
| OPRK1 | ENSG00000082556 | P41145 | 0.9538 | 0.8863 | cerebral cortex |
| OPRL1 | ENSG00000125510 | P41146 | 0.802 | 0.504 | cerebral cortex |
| OPRM1 | ENSG00000112038 | P35372 | 0.7979 | 0.5593 | adrenal gland |
| OR10A3 | ENSG00000170683 | P58181 | 0.9742 | 0.9663 | granulocytes |
| OR10A4 | ENSG00000170782 | Q9H209 | 1 | 1 | dendritic cells |
| OR10G3 | ENSG00000169208 | Q8NGC4 | 0.9265 | 0.8037 | granulocytes |
| OR10G4 | ENSG00000254737 | Q8NGN3 | 0.8366 | 0.6068 | bone marrow |
| OR10J1 | ENSG00000196184 | P30954 | 0.9724 | 0.9352 | granulocytes |
| OR10J3 | ENSG00000196266 | Q5JRS4 | 1 | 1 | placenta |
| OR10P1 | ENSG00000175398 | Q8NGE3 | 0.9831 | 0.98 | t-cells |
| OR10S1 | ENSG00000196248 | Q8NGN2 | 1 | 1 | thyroid gland |
| OR10Z1 | ENSG00000198967 | Q8NGY1 | 0.9974 | 0.9953 | bone marrow |
| OR13A1 | ENSG00000256574 | Q8NGR1 | 0.9302 | 0.8759 | urinary bladder |
| OR13C8 | ENSG00000186943 | Q8NGS7 | 1 | 1 | t-cells |
| OR14C36 | ENSG00000177174 | Q8NHC7 | 1 | 1 | prostate |
| OR14I1 | ENSG00000189181 | A6ND48 | 0.9907 | 0.9843 | cerebral cortex |
| OR14K1 | ENSG00000153230 | Q8NGZ2 | 1 | 1 | fallopian tube |
| OR1F1 | ENSG00000168124 | O43749 | 0.9402 | 0.914 | cerebral cortex |
| OR1J2 | ENSG00000197233 | Q8NGS2 | 0.9872 | 0.9833 | urinary bladder |
| OR1N1 | ENSG00000171505 | Q8NGS0 | 1 | 1 | cervix, uterine |
| OR2A5 | ENSG00000221836 | Q96R48 | 0.9024 | 0.9024 | appendix |
| OR2AG2 | ENSG00000188124 | A6NM03 | 0.878 | 0.878 | appendix |
| OR2AP1 | ENSG00000179615 | Q8NGE2 | 0.9024 | 0.9024 | cerebral cortex |
| OR2B11 | ENSG00000177535 | Q5JQS5 | 0.9246 | 0.9122 | appendix |
| OR2B6 | ENSG00000124657 | P58173 | 0.9246 | 0.9008 | placenta |
| OR2H2 | ENSG00000204657 | O95918 | 0.9697 | 0.8626 | epididymis |
| OR2L13 | ENSG00000196071 | Q8N349 | 0.934 | 0.831 | cerebral cortex |
| OR2L2 | ENSG00000203663 | Q8NH16 | 0.6724 | 0.4039 | bone marrow |
| OR2L3 | ENSG00000198128 | Q8NG85 | 0.9448 | 0.8091 | prostate |
| OR2L5 | ENSG00000197454 | Q8NG80 | 0.9756 | 0.9756 | bone marrow |
| OR2T10 | ENSG00000184022 | Q8NGZ9 | 0.9929 | 0.9774 | kidney |
| OR2T33 | ENSG00000177212 | Q8NG76 | 0.811 | 0.7268 | bone marrow |
| OR2V2 | ENSG00000182613 | Q96R30 | 0.8548 | 0.6345 | granulocytes |
| OR2W3 | ENSG00000238243 | Q7Z3T1 | 0.8785 | 0.6888 | thyroid gland |
| OR3A1 | ENSG00000180090 | P47881 | 0.9182 | 0.8532 | t-cells |
| OR3A2 | ENSG00000221882 | P47893 | 0.8088 | 0.6479 | cerebral cortex |
| OR3A3 | ENSG00000159961 | P47888 | 0.9848 | 0.9026 | dendritic cells |
| OR4A47 | ENSG00000237388 | Q6IF82 | 1 | 1 | t-cells |
| OR4C6 | ENSG00000181903 | Q8NH72 | 0.9512 | 0.9512 | b-cells |
| OR4D1 | ENSG00000141194 | Q15615 | 0.9219 | 0.819 | nk-cells |
| OR4D9 | ENSG00000172742 | Q8NGE8 | 0.7751 | 0.464 | granulocytes |
| OR4F15 | ENSG00000182854 | Q8NGB8 | 0.9599 | 0.8786 | granulocytes |
| OR4P4 | ENSG00000181927 | Q8NGL7 | 0.8875 | 0.8604 | bone marrow |
| OR51B4 | ENSG00000183251 | Q9Y5P0 | 0.9024 | 0.9024 | breast |
| OR51E2 | ENSG00000167332 | Q9H255 | 0.9085 | 0.6836 | prostate |
| OR51T1 | ENSG00000176900 | Q8NGJ9 | 1 | 1 | prostate |
| OR52A1 | ENSG00000182070 | Q9UKL2 | 0.7079 | 0.5011 | granulocytes |
| OR52I1 | ENSG00000232268 | Q8NGK6 | 0.739 | 0.481 | skin |
| OR52I2 | ENSG00000226288 | Q8NH67 | 1 | 1 | epididymis |
| OR52K1 | ENSG00000196778 | Q8NGK4 | 0.7 | 0.417 | bone marrow |
| OR52K2 | ENSG00000181963 | Q8NGK3 | 0.9869 | 0.9803 | granulocytes |
| OR52N1 | ENSG00000181001 | Q8NH53 | 0.9024 | 0.9024 | adipose tissue |
| OR52N4 | ENSG00000181074 | Q8NGI2 | 0.728 | 0.5138 | spleen |
| OR52W1 | ENSG00000175485 | Q6IF63 | 1 | 1 | bone marrow |
| OR56A4 | ENSG00000183389 | Q8NGH8 | 0.9745 | 0.9626 | granulocytes |
| OR5AN1 | ENSG00000176495 | Q8NGI8 | 0.9745 | 0.9626 | bone marrow |
| OR5AU1 | ENSG00000169327 | Q8NGC0 | 0.7646 | 0.5563 | bone marrow |
| OR5B21 | ENSG00000198283 | A6NL26 | 1 | 1 | urinary bladder |
| OR5P2 | ENSG00000183303 | Q8WZ92 | 1 | 1 | skin |
| OR5P3 | ENSG00000182334 | Q8WZ94 | 0.9756 | 0.9756 | epididymis |
| OR5T2 | ENSG00000181718 | Q8NGG2 | 0.9872 | 0.9833 | skin |
| OR6B3 | ENSG00000178586 | Q8NGW1 | 0.9978 | 0.996 | epididymis |
| OR6C2 | ENSG00000179695 | Q9NZP2 | 1 | 1 | epididymis |
| OR6C4 | ENSG00000179626 | Q8NGE1 | 0.7696 | 0.5384 | granulocytes |
| OR6N1 | ENSG00000197403 | Q8NGY5 | 0.9935 | 0.9898 | granulocytes |
| OR6N2 | ENSG00000188340 | Q8NGY6 | 0.9814 | 0.9789 | bone marrow |
| OR6T1 | ENSG00000181499 | Q8NGN1 | 1 | 1 | pancreas |
| OR6X1 | ENSG00000221931 | Q8NH79 | 1 | 1 | epididymis |
| OR7A17 | ENSG00000185385 | O14581 | 0.7494 | 0.5889 | granulocytes |
| OR7A5 | ENSG00000188269 | Q15622 | 0.8984 | 0.7465 | epididymis |
| OR7C1 | ENSG00000127530 | O76099 | 0.8511 | 0.5418 | parathyroid gland |
| OR7D4 | ENSG00000174667 | Q8NG98 | 0.9112 | 0.6384 | granulocytes |
| OR8A1 | ENSG00000196119 | Q8NGG7 | 0.7554 | 0.4787 | parathyroid gland |
| OR8B8 | ENSG00000197125 | Q15620 | 0.7567 | 0.5074 | skin |
| OR8D1 | ENSG00000196341 | Q8WZ84 | 0.9756 | 0.9756 | epididymis |
| OR9A4 | ENSG00000258083 | Q8NGU2 | 0.9756 | 0.9756 | bone marrow |
| OR9Q1 | ENSG00000186509 | Q8NGQ5 | 0.9756 | 0.9756 | skeletal muscle |
| OTOF | ENSG00000115155 | Q9HC10 | 0.9348 | 0.7627 | bone marrow |
| OTOP1 | ENSG00000163982 | Q7RTM1 | 0.9872 | 0.9833 | skin |
| OTOP2 | ENSG00000183034 | Q7RTS6 | 0.9586 | 0.9242 | colon |
| OXGR1 | ENSG00000165621 | Q96P68 | 0.7366 | 0.5499 | breast |
| OXTR | ENSG00000180914 | P30559 | 0.9111 | 0.6361 | breast |
| P2RX1 | ENSG00000108405 | P51575 | 0.6004 | 0.41 | seminal vesicle |
| P2RX2 | ENSG00000187848 | Q9UBL9 | 0.8616 | 0.7141 | epididymis |
| P2RX3 | ENSG00000109991 | P56373 | 0.9502 | 0.8705 | heart muscle |
| P2RX5 | ENSG00000083454 | Q93086 | 0.7794 | 0.6037 | lymph node |
| P2RX6 | ENSG00000099957 | O15547 | 0.8347 | 0.6838 | cerebral cortex |
| P2RY10 | ENSG00000078589 | O00398 | 0.7558 | 0.5012 | granulocytes |
| P2RY2 | ENSG00000175591 | P41231 | 0.6975 | 0.4126 | granulocytes |
| P2RY4 | ENSG00000186912 | P51582 | 0.9553 | 0.8394 | small intestine |
| P2RY6 | ENSG00000171631 | Q15077 | 0.778 | 0.4329 | dendritic cells |
| PANX2 | ENSG00000073150 | Q96RD6 | 0.8492 | 0.5647 | cerebral cortex |
| PANX3 | ENSG00000154143 | Q96QZ0 | 1 | 1 | placenta |
| PAQR5 | ENSG00000137819 | Q9NXK6 | 0.729 | 0.4852 | kidney |
| PAQR6 | ENSG00000160781 | Q6TCH4 | 0.8427 | 0.4182 | cerebral cortex |
| PAQR9 | ENSG00000188582 | Q6ZVX9 | 0.9276 | 0.907 | liver |
| PCDH10 | ENSG00000138650 | Q9P2E7 | 0.8557 | 0.7177 | cerebral cortex |
| PCDH11X | ENSG00000102290 | Q9BZA7 | 0.9198 | 0.8332 | ovary |
| PCDH11Y | ENSG00000099715 | Q9BZA8 | 0.9642 | 0.8435 | cerebral cortex |
| PCDH12 | ENSG00000113555 | Q9NPG4 | 0.687 | 0.4012 | placenta |
| PCDH15 | ENSG00000150275 | Q96QU1 | 0.9119 | 0.7677 | adrenal gland |
| PCDH17 | ENSG00000118946 | O14917 | 0.7611 | 0.4945 | spleen |
| PCDH19 | ENSG00000165194 | Q8TAB3 | 0.8636 | 0.6075 | cerebral cortex |
| PCDH7 | ENSG00000169851 | O60245 | 0.5813 | 0.4438 | cerebral cortex |
| PCDH8 | ENSG00000136099 | O95206 | 0.9919 | 0.9751 | cerebral cortex |
| PCDH9 | ENSG00000184226 | Q9HC56 | 0.8268 | 0.4676 | cerebral cortex |
| PCDHA1 | ENSG00000204970 | Q9Y513 | 0.9804 | 0.9344 | cerebral cortex |
| PCDHA10 | ENSG00000250120 | Q9Y512 | 0.8342 | 0.6027 | cerebral cortex |
| PCDHA11 | ENSG00000249158 | Q9Y511 | 0.9056 | 0.7359 | cerebral cortex |
| PCDHA12 | ENSG00000251664 | Q9UN75 | 0.8544 | 0.747 | lung |
| PCDHA13 | ENSG00000239389 | Q9Y510 | 0.9476 | 0.7811 | parathyroid gland |
| PCDHA2 | ENSG00000204969 | Q9Y5H9 | 0.901 | 0.7794 | cerebral cortex |
| PCDHA3 | ENSG00000255408 | Q9Y5H8 | 0.767 | 0.577 | endometrium |
| PCDHA4 | ENSG00000204967 | Q9UN74 | 0.8016 | 0.4153 | cerebral cortex |
| PCDHA5 | ENSG00000204965 | Q9Y5H7 | 0.9742 | 0.8747 | cerebral cortex |
| PCDHA6 | ENSG00000081842 | Q9UN73 | 0.893 | 0.6789 | cerebral cortex |
| PCDHA7 | ENSG00000204963 | Q9UN72 | 0.9297 | 0.6986 | cerebral cortex |
| PCDHA8 | ENSG00000204962 | Q9Y5H6 | 0.9872 | 0.9775 | cerebral cortex |
| PCDHA9 | ENSG00000204961 | Q9Y5H5 | 0.9308 | 0.7294 | cerebral cortex |
| PCDHAC1 | ENSG00000248383 | Q9H158 | 0.9366 | 0.73 | parathyroid gland |
| PCDHAC2 | ENSG00000243232 | Q9Y514 | 0.8931 | 0.6482 | parathyroid gland |
| PCDHB1 | ENSG00000171815 | Q9Y5F3 | 0.7003 | 0.4239 | fallopian tube |
| PCDHB10 | ENSG00000120324 | Q9UN67 | 0.7177 | 0.4821 | cerebral cortex |
| PCDHB11 | ENSG00000197479 | Q9Y5F2 | 0.7826 | 0.403 | parathyroid gland |
| PCDHB12 | ENSG00000120328 | Q9Y5F1 | 0.6989 | 0.4872 | endometrium |
| PCDHB14 | ENSG00000120327 | Q9Y5E9 | 0.6367 | 0.4303 | parathyroid gland |
| PCDHB15 | ENSG00000113248 | Q9Y5E8 | 0.6583 | 0.4206 | cerebral cortex |
| PCDHB2 | ENSG00000112852 | Q9Y5E7 | 0.7427 | 0.4815 | cerebral cortex |
| PCDHB3 | ENSG00000113205 | Q9Y5E6 | 0.6095 | 0.4663 | endometrium |
| PCDHB4 | ENSG00000081818 | Q9Y5E5 | 0.6242 | 0.4281 | endometrium |
| PCDHB7 | ENSG00000113212 | Q9Y5E2 | 0.7388 | 0.5031 | endometrium |
| PCDHB8 | ENSG00000120322 | Q9UN66 | 0.7449 | 0.5383 | endometrium |
| PCDHGA1 | ENSG00000204956 | Q9Y5H4 | 0.8128 | 0.4355 | cerebral cortex |
| PCDHGA10 | ENSG00000253846 | Q9Y5H3 | 0.7948 | 0.4234 | cerebral cortex |
| PCDHGA11 | ENSG00000253873 | Q9Y5H2 | 0.7177 | 0.4483 | cerebral cortex |
| PCDHGA12 | ENSG00000253159 | O60330 | 0.6848 | 0.4452 | cerebral cortex |
| PCDHGA3 | ENSG00000254245 | Q9Y5H0 | 0.7762 | 0.5422 | cerebral cortex |
| PCDHGA4 | ENSG00000262576 | Q9Y5G9 | 0.7258 | 0.4894 | cerebral cortex |
| PCDHGA5 | ENSG00000253485 | Q9Y5G8 | 0.6832 | 0.4648 | cerebral cortex |
| PCDHGA6 | ENSG00000253731 | Q9Y5G7 | 0.5792 | 0.4047 | endometrium |
| PCDHGA7 | ENSG00000253537 | Q9Y5G6 | 0.8012 | 0.5292 | cerebral cortex |
| PCDHGA8 | ENSG00000253767 | Q9Y5G5 | 0.7213 | 0.4271 | granulocytes |
| PCDHGB2 | ENSG00000253910 | Q9Y5G2 | 0.6751 | 0.4516 | cerebral cortex |
| PCDHGB4 | ENSG00000253953 | Q9UN71 | 0.6459 | 0.4344 | placenta |
| PCDHGC5 | ENSG00000240764 | Q9Y5F6 | 0.9689 | 0.8024 | cerebral cortex |
| PCSK4 | ENSG00000115257 | Q6UW60 | 0.7894 | 0.4729 | fallopian tube |
| PDCD1 | ENSG00000188389 | Q15116 | 0.8737 | 0.7137 | lymph node |
| PHEX | ENSG00000102174 | P78562 | 0.7949 | 0.6459 | dendritic cells |
| PIANP | ENSG00000139200 | Q81YJ0 | 0.85 | 0.6013 | cerebral cortex |
| PIEZO2 | ENSG00000154864 | Q9H515 | 0.6755 | 0.4761 | lung |
| PIGR | ENSG00000162896 | P01833 | 0.6844 | 0.5599 | duodenum |
| PIRT | ENSG00000233670 | P0C851 | 0.8546 | 0.821 | adrenal gland |
| PKD1L1 | ENSG00000158683 | Q8TDX9 | 0.8081 | 0.5315 | adipose tissue |
| PKD2L1 | ENSG00000107593 | Q9P0L9 | 0.8892 | 0.7104 | spleen |
| PKDREJ | ENSG00000130943 | Q9NTG1 | 0.8672 | 0.565 | skin |
| PKHD1 | ENSG00000170927 | P08F94 | 0.9385 | 0.8592 | kidney |
| PKHD1L1 | ENSG00000205038 | Q86WI1 | 0.8606 | 0.6404 | thyroid gland |
| PLA2R1 | ENSG00000153246 | Q13018 | 0.6517 | 0.4002 | thyroid gland |
| PLB1 | ENSG00000163803 | Q6P1J6 | 0.7647 | 0.4021 | small intestine |
| PLD5 | ENSG00000180287 | Q8N7P1 | 0.7947 | 0.6287 | seminal vesicle |
| PLN | ENSG00000198523 | P26678 | 0.6573 | 0.4228 | heart muscle |
| PLP1 | ENSG00000123560 | P60201 | 0.8036 | 0.4933 | cerebral cortex |
| PLPP2 | ENSG00000141934 | O43688 | 0.5639 | 0.4073 | fallopian tube |
| PLPP4 | ENSG00000203805 | Q5VZY2 | 0.8344 | 0.6443 | cerebral cortex |
| PLPP7 | ENSG00000160539 | Q8NBV4 | 0.7813 | 0.4492 | skeletal muscle |
| PLPPR1 | ENSG00000148123 | Q8TBJ4 | 0.9424 | 0.8988 | cerebral cortex |
| PLPPR3 | ENSG00000129951 | Q6T4P5 | 0.9264 | 0.882 | cerebral cortex |
| PLPPR4 | ENSG00000117600 | Q7Z2D5 | 0.8553 | 0.532 | cerebral cortex |
| PLPPR5 | ENSG00000117598 | Q32ZL2 | 0.9873 | 0.9602 | cerebral cortex |
| PLSCR2 | ENSG00000163746 | Q9NRY7 | 0.7914 | 0.4692 | epididymis |
| PLXNA4 | ENSG00000221866 | Q9HCM2 | 0.7313 | 0.4975 | adipose tissue |
| PLXNB3 | ENSG00000198753 | Q9ULL4 | 0.733 | 0.4715 | cerebral cortex |
| PMEL | ENSG00000185664 | P40967 | 0.82 | 0.4213 | skin |
| PNPLA3 | ENSG00000100344 | Q9NST1 | 0.8733 | 0.6059 | liver |
| POPDC2 | ENSG00000121577 | Q9HBU9 | 0.76 | 0.4634 | heart muscle |
| POPDC3 | ENSG00000132429 | Q9HBV1 | 0.8935 | 0.7327 | skeletal muscle |
| PPP1R3A | ENSG00000154415 | Q16821 | 0.9667 | 0.956 | skeletal muscle |
| PRIMA1 | ENSG00000175785 | Q86XR5 | 0.6391 | 0.478 | smooth muscle |
| PRLHR | ENSG00000119973 | P49683 | 0.927 | 0.9092 | adrenal gland |
| PRLR | ENSG00000113494 | P16471 | 0.7021 | 0.5318 | placenta |
| PROKR1 | ENSG00000169618 | Q8TCW9 | 0.9658 | 0.9425 | epididymis |
| PROKR2 | ENSG00000101292 | Q8NFJ6 | 0.964 | 0.9251 | cerebral cortex |
| PROM1 | ENSG00000007062 | O43490 | 0.624 | 0.4688 | cervix, uterine |
| PROM2 | ENSG00000155066 | Q8N271 | 0.6316 | 0.4945 | skin |
| PRR7 | ENSG00000131188 | Q8TB68 | 0.7546 | 0.5063 | cerebral cortex |
| PRRG3 | ENSG00000130032 | Q9BZD7 | 0.7336 | 0.4765 | cerebral cortex |
| PRRT1 | ENSG00000204314 | Q99946 | 0.9829 | 0.9506 | cerebral cortex |
| PRRT4 | ENSG00000224940 | C9JH25 | 0.8911 | 0.6251 | bone marrow |
| PRSS8 | ENSG00000052344 | Q16651 | 0.5284 | 0.439 | small intestine |
| PRTG | ENSG00000166450 | Q2VWP7 | 0.8416 | 0.6137 | thyroid gland |
| PSD2 | ENSG00000146005 | Q9BQ17 | 0.9699 | 0.8254 | cerebral cortex |
| PTCHD1 | ENSG00000165186 | Q96NR3 | 0.8119 | 0.665 | seminal vesicle |
| PTCHD4 | ENSG00000244694 | Q6ZW05 | 0.7537 | 0.556 | seminal vesicle |
| PTCRA | ENSG00000171611 | Q6ISU1 | 0.9503 | 0.8011 | dendritic cells |
| PTGDR | ENSG00000168229 | Q13258 | 0.8153 | 0.4775 | nk-cells |
| PTGDR2 | ENSG00000183134 | Q9Y5Y4 | 0.8665 | 0.6091 | granulocytes |
| PTGER1 | ENSG00000160951 | P34995 | 0.9001 | 0.7788 | kidney |
| PTGER3 | ENSG00000050628 | P43115 | 0.6911 | 0.4703 | endometrium |
| PTGFR | ENSG00000122420 | P43088 | 0.68 | 0.4826 | endometrium |
| PTH1R | ENSG00000160801 | Q03431 | 0.7502 | 0.4656 | kidney |
| PTH2R | ENSG00000144407 | P49190 | 0.8088 | 0.7026 | bone marrow |
| PTPRCAP | ENSG00000213402 | Q14761 | 0.7318 | 0.4997 | t-cells |
| PTPRD | ENSG00000153707 | P23468 | 0.7603 | 0.5186 | parathyroid gland |
| PTPRH | ENSG00000080031 | Q9HD43 | 0.7928 | 0.6938 | small intestine |
| PTPRN | ENSG00000054356 | Q16849 | 0.9033 | 0.7277 | cerebral cortex |
| PTPRO | ENSG00000151490 | Q16827 | 0.6578 | 0.4226 | rectum |
| PTPRR | ENSG00000153233 | Q15256 | 0.8522 | 0.7643 | cerebral cortex |
| PTPRT | ENSG00000196090 | O14522 | 0.8989 | 0.7632 | cerebral cortex |
| PTPRZ1 | ENSG00000106278 | P23471 | 0.888 | 0.629 | cerebral cortex |
| PVRIG | ENSG00000213413 | Q6DKI7 | 0.8516 | 0.6584 | nk-cells |
| QRFPR | ENSG00000186867 | Q96P65 | 0.8795 | 0.8335 | heart muscle |
| RAET1E | ENSG00000164520 | Q8TD07 | 0.8824 | 0.6399 | esophagus |
| RAET1G | ENSG00000203722 | Q6H3X3 | 0.8283 | 0.5924 | esophagus |
| RARRES1 | ENSG00000118849 | P49788 | 0.543 | 0.4023 | appendix |
| RDH8 | ENSG00000080511 | Q9NYR8 | 0.9971 | 0.9947 | kidney |
| REEP1 | ENSG00000068615 | Q9H902 | 0.6543 | 0.4109 | cerebral cortex |
| REEP2 | ENSG00000132563 | Q9BRK0 | 0.7712 | 0.4602 | cerebral cortex |
| RET | ENSG00000165731 | P07949 | 0.8487 | 0.451 | parathyroid gland |
| RGR | ENSG00000148604 | P47804 | 0.9543 | 0.6467 | cerebral cortex |
| RGS9BP | ENSG00000186326 | Q6ZS82 | 0.9538 | 0.8087 | skeletal muscle |
| RGSL1 | ENSG00000121446 | A5PLK6 | 0.9816 | 0.9336 | epididymis |
| RHAG | ENSG00000112077 | Q02094 | 0.9792 | 0.9235 | bone marrow |
| RHBDL1 | ENSG00000103269 | O75783 | 0.7795 | 0.4256 | cerebral cortex |
| RHBDL2 | ENSG00000158315 | Q9NX52 | 0.6567 | 0.4468 | skin |
| RHBDL3 | ENSG00000141314 | P58872 | 0.881 | 0.6614 | cerebral cortex |
| RHCG | ENSG00000140519 | Q9UBD6 | 0.928 | 0.8177 | esophagus |
| RHD | ENSG00000187010 | Q02161 | 0.8331 | 0.4732 | bone marrow |
| RHO | ENSG00000163914 | P08100 | 1 | 1 | cerebral cortex |
| RNF112 | ENSG00000128482 | Q9ULX5 | 0.7934 | 0.5037 | cerebral cortex |
| RNF148 | ENSG00000235631 | Q8N7C7 | 0.8243 | 0.6246 | lung |
| RNF175 | ENSG00000145428 | Q8N4F7 | 0.7948 | 0.5438 | cerebral cortex |
| RNF182 | ENSG00000180537 | Q8N6D2 | 0.8605 | 0.6404 | cerebral cortex |
| RNF222 | ENSG00000189051 | A6NCQ9 | 0.952 | 0.8976 | esophagus |
| RNF223 | ENSG00000237330 | E7ERA6 | 0.8817 | 0.6938 | esophagus |
| RNF225 | ENSG00000269855 | M0QZC1 | 0.946 | 0.9147 | esophagus |
| ROBO2 | ENSG00000185008 | Q9HCK4 | 0.7569 | 0.5148 | cerebral cortex |
| ROBO3 | ENSG00000154134 | Q96MS0 | 0.8747 | 0.7313 | smooth muscle |
| ROR2 | ENSG00000169071 | Q01974 | 0.6343 | 0.4484 | parathyroid gland |
| ROS1 | ENSG00000047936 | P08922 | 0.9529 | 0.9008 | epididymis |
| RPRM | ENSG00000177519 | Q9NS64 | 0.7764 | 0.687 | endometrium |
| RPRML | ENSG00000179673 | Q8N4K4 | 0.9866 | 0.9637 | cerebral cortex |
| RRH | ENSG00000180245 | O14718 | 0.7327 | 0.566 | breast |
| RTL1 | ENSG00000254656 | A6NKG5 | 0.9774 | 0.9698 | placenta |
| RTP1 | ENSG00000175077 | P59025 | 0.9867 | 0.972 | cerebral cortex |
| RTP2 | ENSG00000198471 | Q5QGT7 | 1 | 1 | skeletal muscle |
| RTP3 | ENSG00000163825 | Q9BQQ7 | 0.9941 | 0.9836 | liver |
| RTP5 | ENSG00000188011 | Q14D33 | 0.9897 | 0.9732 | cerebral cortex |
| RXFP1 | ENSG00000171509 | Q9HBX9 | 0.8424 | 0.669 | cerebral cortex |
| RXFP2 | ENSG00000133105 | Q8WXD0 | 0.9493 | 0.9222 | monocytes |
| RXFP3 | ENSG00000182631 | Q9NSD7 | 1 | 1 | adrenal gland |
| RXFP4 | ENSG00000173080 | Q8TDU9 | 0.9129 | 0.8636 | rectum |
| RYR2 | ENSG00000198626 | Q92736 | 0.8859 | 0.6388 | heart muscle |
| RYR3 | ENSG00000198838 | Q15413 | 0.8296 | 0.567 | parathyroid gland |
| S1PR4 | ENSG00000125910 | O95977 | 0.7406 | 0.5629 | granulocytes |
| S1PR5 | ENSG00000180739 | Q9H228 | 0.8568 | 0.5896 | t-cells |
| SCN11A | ENSG00000168356 | Q9UI33 | 0.8821 | 0.6541 | spleen |
| SCN1A | ENSG00000144285 | P35498 | 0.974 | 0.9633 | cerebral cortex |
| SCN2A | ENSG00000136531 | Q99250 | 0.9473 | 0.7604 | cerebral cortex |
| SCN2B | ENSG00000149575 | O60939 | 0.8563 | 0.5637 | cerebral cortex |
| SCN3A | ENSG00000153253 | Q9NY46 | 0.8293 | 0.4866 | cerebral cortex |
| SCN3B | ENSG00000166257 | Q9NY72 | 0.8692 | 0.4782 | cerebral cortex |
| SCN4A | ENSG00000007314 | P35499 | 0.9349 | 0.793 | skeletal muscle |
| SCN4B | ENSG00000177098 | Q8IWT1 | 0.6202 | 0.4058 | adipose tissue |
| SCN5A | ENSG00000183873 | Q14524 | 0.9437 | 0.7035 | heart muscle |
| SCN7A | ENSG00000136546 | Q01118 | 0.6636 | 0.4402 | ovary |
| SCN8A | ENSG00000196876 | Q9UQD0 | 0.9037 | 0.5596 | cerebral cortex |
| SCN9A | ENSG00000169432 | Q15858 | 0.7881 | 0.4445 | dendritic cells |
| SCNN1A | ENSG00000111319 | P37088 | 0.5277 | 0.4318 | cervix, uterine |
| SCNN1B | ENSG00000168447 | P51168 | 0.707 | 0.5506 | cervix, uterine |
| SCNN1G | ENSG00000166828 | P51170 | 0.8182 | 0.6521 | kidney |
| SCTR | ENSG00000080293 | P47872 | 0.878 | 0.7221 | duodenum |
| SDK2 | ENSG00000069188 | Q58EX2 | 0.7795 | 0.5188 | cervix, uterine |
| SEL1L2 | ENSG00000101251 | Q5TEA6 | 0.9617 | 0.9404 | endometrium |
| SELE | ENSG00000007908 | P16581 | 0.6892 | 0.5071 | prostate |
| SEMA5B | ENSG00000082684 | Q9P283 | 0.7593 | 0.4843 | cerebral cortex |
| SEMA6B | ENSG00000167680 | Q9H3T3 | 0.7045 | 0.4161 | cerebral cortex |
| SERP2 | ENSG00000151778 | Q8N6R1 | 0.7751 | 0.4271 | cerebral cortex |
| SERTM1 | ENSG00000180440 | A2A2V5 | 0.8446 | 0.7758 | cerebral cortex |
| SEZ6 | ENSG00000063015 | Q53EL9 | 0.958 | 0.7217 | cerebral cortex |
| SEZ6L2 | ENSG00000174938 | Q6UXD5 | 0.6797 | 0.4507 | cerebral cortex |
| SFT2D3 | ENSG00000173349 | Q58719 | 0.6691 | 0.5922 | parathyroid gland |
| SGCA | ENSG00000108823 | Q16586 | 0.7014 | 0.4472 | skeletal muscle |
| SGCD | ENSG00000170624 | Q92629 | 0.5904 | 0.4037 | thyroid gland |
| SGCG | ENSG00000102683 | Q13326 | 0.8438 | 0.6823 | heart muscle |
| SGCZ | ENSG00000185053 | Q96LD1 | 0.9608 | 0.9027 | ovary |
| SHISA6 | ENSG00000188803 | Q6ZSJ9 | 0.8349 | 0.6684 | cervix, uterine |
| SHISA7 | ENSG00000187902 | A6NL88 | 0.9922 | 0.9823 | cerebral cortex |
| SHISA8 | ENSG00000234965 | B8ZZ34 | 0.9321 | 0.853 | adrenal gland |
| SHISAL1 | ENSG00000138944 | Q3SXP7 | 0.7804 | 0.6151 | smooth muscle |
| SHISAL2A | ENSG00000182183 | Q6UWV7 | 0.7595 | 0.6059 | b-cells |
| SHISAL2B | ENSG00000145642 | A6NKW6 | 0.981 | 0.9632 | stomach |
| SI | ENSG00000090402 | P14410 | 0.9324 | 0.8998 | duodenum |
| SIGLEC11 | ENSG00000161640 | Q96RL6 | 0.8723 | 0.5709 | ovary |
| SIGLEC12 | ENSG00000254521 | Q96PQ1 | 0.8527 | 0.6937 | spleen |
| SIGLEC14 | ENSG00000254415 | Q08ET2 | 0.7217 | 0.4751 | granulocytes |
| SIGLEC15 | ENSG00000197046 | Q6ZMC9 | 0.9505 | 0.891 | monocytes |
| SIGLEC5 | ENSG00000105501 | O15389 | 0.8471 | 0.6196 | granulocytes |
| SIGLEC6 | ENSG00000105492 | O43699 | 0.8527 | 0.5153 | placenta |
| SIGLEC7 | ENSG00000168995 | Q9Y286 | 0.7482 | 0.4953 | granulocytes |
| SIGLEC8 | ENSG00000105366 | Q9NYZ4 | 0.8619 | 0.4837 | granulocytes |
| SIGLEC9 | ENSG00000129450 | Q9Y336 | 0.702 | 0.4291 | monocytes |
| SIGLECL1 | ENSG00000179213 | Q8N7X8 | 0.9411 | 0.9048 | cervix, uterine |
| SIRPB1 | ENSG00000101307 | O00241 | 0.7715 | 0.4569 | granulocytes |
| SIRPB2 | ENSG00000196209 | Q5JXA9 | 0.7054 | 0.4564 | granulocytes |
| SIRPG | ENSG00000089012 | Q9P1W8 | 0.816 | 0.5225 | t-cells |
| SIT1 | ENSG00000137078 | Q9Y3P8 | 0.7848 | 0.533 | t-cells |
| SLAMF1 | ENSG00000117090 | Q13291 | 0.7622 | 0.5084 | t-cells |
| SLAMF9 | ENSG00000162723 | Q96A28 | 0.9497 | 0.8349 | dendritic cells |
| SLC10A1 | ENSG00000100652 | Q14973 | 0.9905 | 0.9167 | liver |
| SLC10A2 | ENSG00000125255 | Q12908 | 0.9535 | 0.9283 | small intestine |
| SLC10A4 | ENSG00000145248 | Q96EP9 | 0.9021 | 0.7547 | adrenal gland |
| SLC10A5 | ENSG00000253598 | Q5PT55 | 0.7628 | 0.5802 | liver |
| SLC10A6 | ENSG00000145283 | Q3KNW5 | 0.8449 | 0.6271 | skin |
| SLC11A1 | ENSG00000018280 | P49279 | 0.7876 | 0.5626 | lung |
| SLC12A1 | ENSG00000074803 | Q13621 | 0.9907 | 0.9446 | kidney |
| SLC12A3 | ENSG00000070915 | P55017 | 0.9631 | 0.8474 | kidney |
| SLC12A5 | ENSG00000124140 | Q9H2X9 | 0.8701 | 0.4743 | cerebral cortex |
| SLC12A8 | ENSG00000221955 | AOAV02 | 0.721 | 0.4524 | thyroid gland |
| SLC13A1 | ENSG00000081800 | Q9BZW2 | 0.9694 | 0.956 | kidney |
| SLC13A2 | ENSG00000007216 | Q13183 | 0.8838 | 0.8296 | duodenum |
| SLC13A3 | ENSG00000158296 | Q8WWT9 | 0.8376 | 0.4803 | kidney |
| SLC13A4 | ENSG00000164707 | Q9UKG4 | 0.9799 | 0.9409 | placenta |
| SLC13A5 | ENSG00000141485 | Q86YT5 | 0.9527 | 0.8733 | liver |
| SLC14A1 | ENSG00000141469 | Q13336 | 0.7642 | 0.4272 | prostate |
| SLC14A2 | ENSG00000132874 | Q15849 | 0.8762 | 0.6362 | kidney |
| SLC15A1 | ENSG00000088386 | P46059 | 0.8252 | 0.6239 | small intestine |
| SLC15A5 | ENSG00000188991 | A6NIM6 | 0.9629 | 0.9604 | adipose tissue |
| SLC16A11 | ENSG00000174326 | Q8NCK7 | 0.775 | 0.5095 | parathyroid gland |
| SLC16A12 | ENSG00000152779 | Q6ZSM3 | 0.8255 | 0.6336 | kidney |
| SLC16A8 | ENSG00000100156 | O95907 | 0.7056 | 0.558 | prostate |
| SLC16A9 | ENSG00000165449 | Q7RTY1 | 0.6897 | 0.494 | kidney |
| SLC17A1 | ENSG00000124568 | Q14916 | 0.9798 | 0.9596 | kidney |
| SLC17A2 | ENSG00000112337 | O00624 | 0.9978 | 0.996 | liver |
| SLC17A3 | ENSG00000124564 | O00476 | 0.9792 | 0.9549 | kidney |
| SLC17A4 | ENSG00000146039 | Q9Y2C5 | 0.8613 | 0.8429 | small intestine |
| SLC17A6 | ENSG00000091664 | Q9P2U8 | 1 | 1 | cerebral cortex |
| SLC17A7 | ENSG00000104888 | Q9P2U7 | 0.9353 | 0.6051 | cerebral cortex |
| SLC17A8 | ENSG00000179520 | Q8NDX2 | 0.9553 | 0.8749 | small intestine |
| SLC17A9 | ENSG00000101194 | Q9BYT1 | 0.591 | 0.4005 | stomach |
| SLC18A1 | ENSG00000036565 | P54219 | 0.9507 | 0.8657 | adrenal gland |
| SLC18A3 | ENSG00000187714 | Q16572 | 0.9587 | 0.9372 | placenta |
| SLC19A3 | ENSG00000135917 | Q9BZV2 | 0.7471 | 0.4282 | adipose tissue |
| SLC1A1 | ENSG00000106688 | P43005 | 0.6164 | 0.4487 | small intestine |
| SLC1A2 | ENSG00000110436 | P43004 | 0.9075 | 0.5781 | cerebral cortex |
| SLC1A6 | ENSG00000105143 | P48664 | 0.8808 | 0.5326 | cerebral cortex |
| SLC1A7 | ENSG00000162383 | O00341 | 0.8381 | 0.6539 | gallbladder |
| SLC22A1 | ENSG00000175003 | O15245 | 0.9661 | 0.6918 | liver |
| SLC22A10 | ENSG00000184999 | Q63ZE4 | 0.997 | 0.9925 | liver |
| SLC22A11 | ENSG00000168065 | Q9NSA0 | 0.9595 | 0.9449 | placenta |
| SLC22A12 | ENSG00000197891 | Q96S37 | 0.9964 | 0.9904 | kidney |
| SLC22A13 | ENSG00000172940 | Q9Y226 | 0.9965 | 0.9922 | kidney |
| SLC22A14 | ENSG00000144671 | Q9Y267 | 0.9226 | 0.7935 | kidney |
| SLC22A16 | ENSG00000004809 | Q86VW1 | 0.8944 | 0.8282 | parathyroid gland |
| SLC22A2 | ENSG00000112499 | O15244 | 0.9806 | 0.897 | kidney |
| SLC22A24 | ENSG00000197658 | Q8N4F4 | 1 | 1 | kidney |
| SLC22A25 | ENSG00000196600 | Q6T423 | 1 | 1 | liver |
| SLC22A3 | ENSG00000146477 | O75751 | 0.5788 | 0.4105 | seminal vesicle |
| SLC22A6 | ENSG00000197901 | Q4U2R8 | 0.9926 | 0.9867 | kidney |
| SLC22A7 | ENSG00000137204 | Q9Y694 | 0.9738 | 0.9529 | liver |
| SLC22A8 | ENSG00000149452 | Q8TCC7 | 0.9964 | 0.991 | kidney |
| SLC22A9 | ENSG00000149742 | Q8IVM8 | 0.969 | 0.7552 | liver |
| SLC23A3 | ENSG00000213901 | Q6PIS1 | 0.8547 | 0.5553 | kidney |
| SLC24A2 | ENSG00000155886 | Q9UI40 | 0.9694 | 0.8602 | cerebral cortex |
| SLC24A4 | ENSG00000140090 | Q8NFF2 | 0.8397 | 0.5774 | monocytes |
| SLC26A1 | ENSG00000145217 | Q9H2B4 | 0.6166 | 0.4052 | adrenal gland |
| SLC26A10 | ENSG00000135502 | Q8NG04 | 0.7903 | 0.598 | endometrium |
| SLC26A3 | ENSG00000091138 | P40879 | 0.8347 | 0.7579 | colon |
| SLC26A4 | ENSG00000091137 | O43511 | 0.9293 | 0.6932 | thyroid gland |
| SLC26A5 | ENSG00000170615 | P58743 | 0.7588 | 0.637 | breast |
| SLC26A8 | ENSG00000112053 | Q96RN1 | 0.944 | 0.8715 | bone marrow |
| SLC26A9 | ENSG00000174502 | Q7LBE3 | 0.9203 | 0.8883 | salivary gland |
| SLC27A2 | ENSG00000140284 | O14975 | 0.6902 | 0.4576 | kidney |
| SLC27A6 | ENSG00000113396 | Q9Y2P4 | 0.7566 | 0.5881 | fallopian tube |
| SLC28A1 | ENSG00000156222 | O00337 | 0.8985 | 0.8388 | small intestine |
| SLC28A2 | ENSG00000137860 | O43868 | 0.8688 | 0.7571 | small intestine |
| SLC28A3 | ENSG00000197506 | Q9HAS3 | 0.7837 | 0.6299 | gallbladder |
| SLC2A12 | ENSG00000146411 | Q8TD20 | 0.699 | 0.4579 | prostate |
| SLC2A14 | ENSG00000173262 | Q8TDB8 | 0.7432 | 0.4994 | bone marrow |
| SLC2A2 | ENSG00000163581 | P11168 | 0.9418 | 0.9175 | liver |
| SLC2A4 | ENSG00000181856 | P14672 | 0.7253 | 0.4887 | skeletal muscle |
| SLC30A10 | ENSG00000196660 | Q6XR72 | 0.9328 | 0.9103 | liver |
| SLC30A3 | ENSG00000115194 | Q99726 | 0.9512 | 0.9046 | epididymis |
| SLC30A8 | ENSG00000164756 | Q8IWU4 | 0.8977 | 0.7099 | pancreas |
| SLC34A1 | ENSG00000131183 | Q06495 | 0.9914 | 0.9633 | kidney |
| SLC34A2 | ENSG00000157765 | O95436 | 0.8382 | 0.6518 | lung |
| SLC34A3 | ENSG00000198569 | Q8N130 | 0.9429 | 0.8274 | kidney |
| SLC35D3 | ENSG00000182747 | Q5M8T2 | 0.9532 | 0.9034 | adrenal gland |
| SLC35F1 | ENSG00000196376 | Q5T1Q4 | 0.8889 | 0.5963 | cerebral cortex |
| SLC35F3 | ENSG00000183780 | Q81Y50 | 0.8666 | 0.6339 | cerebral cortex |
| SLC35F4 | ENSG00000151812 | A4IF30 | 0.9534 | 0.9027 | seminal vesicle |
| SLC35G1 | ENSG00000176273 | Q2M3R5 | 0.8073 | 0.4033 | duodenum |
| SLC35G5 | ENSG00000177710 | Q96KT7 | 0.8088 | 0.6445 | dendritic cells |
| SLC36A2 | ENSG00000186335 | Q495M3 | 0.9483 | 0.8046 | kidney |
| SLC38A11 | ENSG00000169507 | Q08A16 | 0.7182 | 0.4802 | epididymis |
| SLC38A4 | ENSG00000139209 | Q96916 | 0.8838 | 0.5506 | liver |
| SLC38A8 | ENSG00000166558 | A6NNN8 | 0.9681 | 0.9534 | cerebral cortex |
| SLC39A12 | ENSG00000148482 | Q504Y0 | 0.9914 | 0.9706 | cerebral cortex |
| SLC39A2 | ENSG00000165794 | Q9NP94 | 0.8727 | 0.7699 | seminal vesicle |
| SLC39A4 | ENSG00000147804 | Q6P5W5 | 0.8882 | 0.7091 | duodenum |
| SLC39A5 | ENSG00000139540 | Q6ZMH5 | 0.8324 | 0.7644 | small intestine |
| SLC3A1 | ENSG00000138079 | Q07837 | 0.8372 | 0.6064 | kidney |
| SLC44A4 | ENSG00000204385 | Q53GD3 | 0.7177 | 0.6109 | duodenum |
| SLC44A5 | ENSG00000137968 | Q8NCS7 | 0.7016 | 0.4532 | skin |
| SLC45A3 | ENSG00000158715 | Q96JT2 | 0.8204 | 0.4595 | prostate |
| SLC46A2 | ENSG00000119457 | Q9BY10 | 0.8515 | 0.7783 | cervix, uterine |
| SLC47A1 | ENSG00000142494 | Q96FL8 | 0.6863 | 0.4253 | adrenal gland |
| SLC47A2 | ENSG00000180638 | Q86VL8 | 0.9503 | 0.892 | kidney |
| SLC4A1 | ENSG00000004939 | P02730 | 0.8985 | 0.5254 | bone marrow |
| SLC4A10 | ENSG00000144290 | Q6U841 | 0.915 | 0.7725 | t-cells |
| SLC4A11 | ENSG00000088836 | Q8NBS3 | 0.7342 | 0.5459 | thyroid gland |
| SLC4A3 | ENSG00000114923 | P48751 | 0.771 | 0.4927 | heart muscle |
| SLC4A4 | ENSG00000080493 | Q9Y6R1 | 0.6589 | 0.5026 | kidney |
| SLC4A8 | ENSG00000050438 | Q2Y0W8 | 0.7924 | 0.4355 | cerebral cortex |
| SLC4A9 | ENSG00000113073 | Q96Q91 | 0.992 | 0.9786 | kidney |
| SLC51A | ENSG00000163959 | Q86UW1 | 0.8253 | 0.5723 | small intestine |
| SLC51B | ENSG00000186198 | Q86UW2 | 0.7683 | 0.5757 | small intestine |
| SLC52A1 | ENSG00000132517 | Q9NWF4 | 0.8553 | 0.6142 | duodenum |
| SLC52A3 | ENSG00000101276 | Q9NQ40 | 0.6844 | 0.4693 | small intestine |
| SLC5A1 | ENSG00000100170 | P13866 | 0.8145 | 0.6105 | duodenum |
| SLC5A10 | ENSG00000154025 | A0PJK1 | 0.9509 | 0.8327 | kidney |
| SLC5A11 | ENSG00000158865 | Q8WWWX8 | 0.9184 | 0.8684 | small intestine |
| SLC5A12 | ENSG00000148942 | Q1EHB4 | 0.9238 | 0.7677 | kidney |
| SLC5A2 | ENSG00000140675 | P31639 | 0.9861 | 0.9364 | kidney |
| SLC5A4 | ENSG00000100191 | Q9NY91 | 0.8808 | 0.6648 | small intestine |
| SLC5A5 | ENSG00000105641 | Q92911 | 0.9136 | 0.7865 | stomach |
| SLC5A7 | ENSG00000115665 | Q9GZV3 | 0.7973 | 0.6496 | thyroid gland |
| SLC5A8 | ENSG00000256870 | Q8N695 | 0.936 | 0.908 | cervix, uterine |
| SLC5A9 | ENSG00000117834 | Q2M3M2 | 0.8998 | 0.7956 | small intestine |
| SLC6A1 | ENSG00000157103 | P30531 | 0.8446 | 0.5297 | cerebral cortex |
| SLC6A11 | ENSG00000132164 | P48066 | 0.9295 | 0.8563 | cerebral cortex |
| SLC6A12 | ENSG00000111181 | P48065 | 0.7957 | 0.577 | liver |
| SLC6A13 | ENSG00000010379 | Q9NSD5 | 0.9324 | 0.8286 | kidney |
| SLC6A15 | ENSG00000072041 | Q9H2J7 | 0.9083 | 0.7324 | cerebral cortex |
| SLC6A17 | ENSG00000197106 | Q9H1V8 | 0.9417 | 0.8011 | cerebral cortex |
| SLC6A18 | ENSG00000164363 | Q96N87 | 1 | 1 | kidney |
| SLC6A19 | ENSG00000174358 | Q695T7 | 0.9183 | 0.8875 | small intestine |
| SLC6A2 | ENSG00000103546 | P23975 | 0.8853 | 0.769 | adrenal gland |
| SLC6A20 | ENSG00000163817 | Q9NP91 | 0.8472 | 0.6098 | duodenum |
| SLC6A3 | ENSG00000142319 | Q01959 | 0.9734 | 0.9491 | thyroid gland |
| SLC6A4 | ENSG00000108576 | P31645 | 0.8909 | 0.7967 | small intestine |
| SLC6A5 | ENSG00000165970 | Q9Y345 | 0.9973 | 0.9952 | lung |
| SLC6A7 | ENSG00000011083 | Q99884 | 0.9483 | 0.9017 | cerebral cortex |
| SLC7A10 | ENSG00000130876 | Q9NS82 | 0.9694 | 0.9141 | adipose tissue |
| SLC7A11 | ENSG00000151012 | Q9UPY5 | 0.8173 | 0.5644 | cerebral cortex |
| SLC7A13 | ENSG00000164893 | Q8TCU3 | 0.997 | 0.9881 | kidney |
| SLC7A3 | ENSG00000165349 | Q8WY07 | 0.8603 | 0.7607 | prostate |
| SLC7A4 | ENSG00000099960 | O43246 | 0.705 | 0.529 | esophagus |
| SLC7A9 | ENSG00000021488 | P82251 | 0.9203 | 0.8334 | small intestine |
| SLC8A2 | ENSG00000118160 | Q9UPR5 | 0.9233 | 0.7824 | cerebral cortex |
| SLC8A3 | ENSG00000100678 | P57103 | 0.9136 | 0.666 | cerebral cortex |
| SLC9A2 | ENSG00000115616 | Q9UBY0 | 0.7636 | 0.6095 | stomach |
| SLC9A4 | ENSG00000180251 | Q6AI14 | 0.9138 | 0.6322 | stomach |
| SLC9A5 | ENSG00000135740 | Q14940 | 0.8546 | 0.5252 | spleen |
| SLC9C1 | ENSG00000172139 | Q4GON8 | 0.9843 | 0.9563 | skin |
| SLC9C2 | ENSG00000162753 | Q5TAH2 | 0.9657 | 0.9192 | fallopian tube |
| SLCO1A2 | ENSG00000084453 | P46721 | 0.8637 | 0.5882 | cerebral cortex |
| SLCO1B1 | ENSG00000134538 | Q9Y6L6 | 0.993 | 0.9794 | liver |
| SLCO1B3 | ENSG00000111700 | Q9NPD5 | 0.9723 | 0.9195 | liver |
| SLCO1C1 | ENSG00000139155 | Q9NYB5 | 0.9569 | 0.7548 | cerebral cortex |
| SLCO4C1 | ENSG00000173930 | Q6ZQN7 | 0.826 | 0.5574 | kidney |
| SLCO5A1 | ENSG00000137571 | Q9H2Y9 | 0.8387 | 0.6516 | prostate |
| SLCO6A1 | ENSG00000205359 | Q86UG4 | 1 | 1 | placenta |
| SLITRK1 | ENSG00000178235 | Q96PX8 | 0.9945 | 0.9774 | cerebral cortex |
| SLITRK2 | ENSG00000185985 | Q9H156 | 0.9077 | 0.7097 | cerebral cortex |
| SLITRK3 | ENSG00000121871 | O94933 | 0.8165 | 0.6982 | cerebral cortex |
| SLITRK4 | ENSG00000179542 | Q8IW52 | 0.7253 | 0.5213 | adrenal gland |
| SLITRK5 | ENSG00000165300 | O94991 | 0.8948 | 0.7924 | cerebral cortex |
| SLITRK6 | ENSG00000184564 | Q9H5Y7 | 0.7513 | 0.5855 | urinary bladder |
| SMCO2 | ENSG00000165935 | A6NFE2 | 0.8786 | 0.8161 | skin |
| SMCO3 | ENSG00000179256 | A2RU48 | 0.8201 | 0.6598 | cervix, uterine |
| SMIM18 | ENSG00000253457 | PODKX4 | 0.9462 | 0.8582 | cerebral cortex |
| SMIM2 | ENSG00000139656 | Q9BVW6 | 0.783 | 0.526 | epididymis |
| SMIM22 | ENSG00000267795 | K7EJ46 | 0.5943 | 0.4667 | colon |
| SMIM23 | ENSG00000185662 | A6NLE4 | 1 | 1 | spleen |
| SMIM24 | ENSG00000095932 | O75264 | 0.7921 | 0.6177 | epididymis |
| SMIM28 | ENSG00000262543 | A0A1B0GU | 0.9147 | 0.8985 | appendix |
| | | 29 | | | |
| SMIM5 | ENSG00000204323 | Q71RC9 | 0.7747 | 0.4264 | dendritic cells |
| SMIM6 | ENSG00000259120 | P0DI80 | 0.7505 | 0.5941 | fallopian tube |
| SMLR1 | ENSG00000256162 | H3BR10 | 0.9504 | 0.9357 | liver |
| SNORC | ENSG00000182600 | Q6UX34 | 0.8447 | 0.5738 | breast |
| SOGA3 | ENSG00000214338 | Q5TF21 | 0.8808 | 0.6291 | cerebral cortex |
| SORCS1 | ENSG00000108018 | Q8WY21 | 0.767 | 0.4845 | thyroid gland |
| SORCS2 | ENSG00000184985 | Q96PQ0 | 0.6845 | 0.4427 | cerebral cortex |
| SORCS3 | ENSG00000156395 | Q9UPU3 | 0.9562 | 0.8303 | cerebral cortex |
| SPACA3 | ENSG00000141316 | Q8IXA5 | 0.9161 | 0.5714 | pancreas |
| SPATA9 | ENSG00000145757 | Q9BWV2 | 0.8682 | 0.6293 | adipose tissue |
| SPNS3 | ENSG00000182557 | Q6ZMD2 | 0.9197 | 0.7735 | granulocytes |
| SSMEM1 | ENSG00000165120 | Q8WWF3 | 0.9756 | 0.9756 | lung |
| SSTR1 | ENSG00000139874 | P30872 | 0.7857 | 0.5928 | stomach |
| SSTR4 | ENSG00000132671 | P31391 | 0.9921 | 0.9821 | cerebral cortex |
| SSTR5 | ENSG00000162009 | P35346 | 0.9403 | 0.8624 | adrenal gland |
| STAB2 | ENSG00000136011 | Q8WWQ8 | 0.8967 | 0.6423 | spleen |
| STEAP1B | ENSG00000105889 | Q6NZ63 | 0.7731 | 0.5256 | b-cells |
| STOML3 | ENSG00000133115 | Q8TAV4 | 0.9811 | 0.9529 | fallopian tube |
| STRA6 | ENSG00000137868 | Q9BX79 | 0.8446 | 0.7104 | cervix, uterine |
| STUM | ENSG00000203685 | Q69YW2 | 0.7731 | 0.4636 | cerebral cortex |
| STX1A | ENSG00000106089 | Q16623 | 0.7997 | 0.407 | cerebral cortex |
| STX1B | ENSG00000099365 | P61266 | 0.8793 | 0.4947 | cerebral cortex |
| STYK1 | ENSG00000060140 | Q6J9G0 | 0.6552 | 0.4952 | rectum |
| SUCNR1 | ENSG00000198829 | Q9BXA5 | 0.8119 | 0.5613 | kidney |
| SUN3 | ENSG00000164744 | Q8TAQ9 | 0.9045 | 0.7731 | placenta |
| SUSD2 | ENSG00000099994 | Q9UGT4 | 0.7536 | 0.4576 | lung |
| SUSD4 | ENSG00000143502 | Q5VX71 | 0.6452 | 0.46 | esophagus |
| SUSD5 | ENSG00000173705 | O60279 | 0.6387 | 0.4572 | cerebral cortex |
| SV2A | ENSG00000159164 | Q7L0J3 | 0.7621 | 0.4298 | cerebral cortex |
| SV2B | ENSG00000185518 | Q7L112 | 0.9342 | 0.5981 | cerebral cortex |
| SV2C | ENSG00000122012 | Q496J9 | 0.9086 | 0.7211 | cerebral cortex |
| SVOP | ENSG00000166111 | Q8N4V2 | 0.9738 | 0.8783 | cerebral cortex |
| SVOPL | ENSG00000157703 | Q8N434 | 0.8985 | 0.6747 | parathyroid gland |
| SYNDIG1 | ENSG00000101463 | Q9H7V2 | 0.7711 | 0.5726 | cerebral cortex |
| SYNDIG1L | ENSG00000183379 | A6NDD5 | 0.9543 | 0.7651 | epididymis |
| SYNGR3 | ENSG00000127561 | O43761 | 0.8556 | 0.5475 | cerebral cortex |
| SYNGR4 | ENSG00000105467 | O95473 | 0.9564 | 0.8782 | parathyroid gland |
| SYNPR | ENSG00000163630 | Q8TBG9 | 0.9824 | 0.9232 | cerebral cortex |
| SYP | ENSG00000102003 | P08247 | 0.8395 | 0.4297 | cerebral cortex |
| SYPL2 | ENSG00000143028 | Q5VXT5 | 0.8005 | 0.5181 | skeletal muscle |
| SYT1 | ENSG00000067715 | P21579 | 0.8511 | 0.5748 | cerebral cortex |
| SYT12 | ENSG00000173227 | Q8IV01 | 0.905 | 0.7293 | parathyroid gland |
| SYT13 | ENSG00000019505 | Q7L8C5 | 0.8262 | 0.7027 | cerebral cortex |
| SYT14 | ENSG00000143469 | Q8NB59 | 0.9598 | 0.8916 | thyroid gland |
| SYT2 | ENSG00000143858 | Q8N910 | 0.8725 | 0.6758 | cerebral cortex |
| SYT3 | ENSG00000213023 | Q9BQG1 | 0.9652 | 0.7863 | cerebral cortex |
| SYT5 | ENSG00000129990 | O00445 | 0.9264 | 0.6625 | cerebral cortex |
| SYT6 | ENSG00000134207 | Q5T7P8 | 0.9314 | 0.8261 | cerebral cortex |
| SYT7 | ENSG00000011347 | O43581 | 0.7122 | 0.4306 | cerebral cortex |
| SYT8 | ENSG00000149043 | Q8NBV8 | 0.8668 | 0.7587 | skin |
| SYT9 | ENSG00000170743 | Q86SS6 | 0.856 | 0.7636 | cerebral cortex |
| TAAR1 | ENSG00000146399 | Q96RJ0 | 0.9745 | 0.9279 | stomach |
| TAAR6 | ENSG00000146383 | Q96RI8 | 1 | 1 | kidney |
| TACR1 | ENSG00000115353 | P25103 | 0.7228 | 0.5325 | cervix, uterine |
| TACR2 | ENSG00000075073 | P21452 | 0.7753 | 0.5562 | smooth muscle |
| TACR3 | ENSG00000169836 | P29371 | 0.9573 | 0.9401 | cerebral cortex |
| TACSTD2 | ENSG00000184292 | P09758 | 0.6326 | 0.4719 | esophagus |
| TAS1R1 | ENSG00000173662 | Q7RTX1 | 0.7229 | 0.5737 | gallbladder |
| TAS1R3 | ENSG00000169962 | Q7RTX0 | 0.8835 | 0.6039 | epididymis |
| TAS2R1 | ENSG00000169777 | Q9NYW7 | 0.9734 | 0.9491 | fallopian tube |
| TAS2R10 | ENSG00000121318 | Q9NYW0 | 0.8991 | 0.8565 | breast |
| TAS2R14 | ENSG00000212127 | Q9NYV8 | 0.8207 | 0.457 | epididymis |
| TAS2R19 | ENSG00000212124 | P59542 | 0.9617 | 0.9404 | ovary |
| TAS2R20 | ENSG00000255837 | P59543 | 0.8718 | 0.6638 | skin |
| TAS2R3 | ENSG00000127362 | Q9NYW6 | 0.8488 | 0.7448 | ovary |
| TAS2R30 | ENSG00000256188 | P59541 | 0.7738 | 0.6885 | endometrium |
| TAS2R31 | ENSG00000256436 | P59538 | 0.862 | 0.7756 | endometrium |
| TAS2R38 | ENSG00000257138 | P59533 | 0.9498 | 0.9445 | rectum |
| TAS2R4 | ENSG00000127364 | Q9NYW5 | 0.7348 | 0.4364 | skin |
| TAS2R5 | ENSG00000127366 | Q9NYW4 | 0.7351 | 0.4339 | skin |
| TCP11 | ENSG00000124678 | Q8WWU5 | 0.869 | 0.6076 | fallopian tube |
| TECRL | ENSG00000205678 | Q5HYJ1 | 0.9462 | 0.7504 | heart muscle |
| TEDDM1 | ENSG00000203730 | Q5T9Z0 | 0.9895 | 0.9292 | epididymis |
| TENM1 | ENSG00000009694 | Q9UKZ4 | 0.7365 | 0.5526 | prostate |
| TENM2 | ENSG00000145934 | Q9NT68 | 0.8751 | 0.6913 | heart muscle |
| TENM3 | ENSG00000218336 | Q9P273 | 0.6999 | 0.4995 | placenta |
| TENM4 | ENSG00000149256 | Q6N022 | 0.8458 | 0.617 | parathyroid gland |
| TEX29 | ENSG00000153495 | Q8N6K0 | 0.9195 | 0.591 | cerebral cortex |
| TEX38 | ENSG00000186118 | Q6PEX7 | 0.8944 | 0.6462 | parathyroid gland |
| TEX51 | ENSG00000237524 | A0A1B0GUA7 | 1 | 1 | kidney |
| TFR2 | ENSG00000106327 | Q9UP52 | 0.9662 | 0.9341 | liver |
| TGFBR3L | ENSG00000260001 | H3BV60 | 0.8695 | 0.7942 | small intestine |
| THSD7A | ENSG00000005108 | Q9UPZ6 | 0.6632 | 0.4927 | kidney |
| THSD7B | ENSG00000144229 | Q9C014 | 0.886 | 0.7052 | epididymis |
| TIGIT | ENSG00000181847 | Q495A1 | 0.7953 | 0.4813 | t-cells |
| TIMD4 | ENSG00000145850 | Q96H15 | 0.8194 | 0.6305 | lymph node |
| TLR10 | ENSG00000174123 | Q9BXR5 | 0.7734 | 0.5508 | lymph node |
| TM4SF19 | ENSG00000145107 | Q96DZ7 | 0.8432 | 0.4504 | t-cells |
| TM4SF20 | ENSG00000168955 | Q53R12 | 0.9418 | 0.8281 | duodenum |
| TM4SF4 | ENSG00000169903 | P48230 | 0.8854 | 0.7801 | gallbladder |
| TM4SF5 | ENSG00000142484 | O14894 | 0.8803 | 0.8327 | duodenum |
| TMC1 | ENSG00000165091 | Q8TDI8 | 0.9215 | 0.8429 | cervix, uterine |
| TMC2 | ENSG00000149488 | Q8TDI7 | 0.937 | 0.8576 | lung |
| TMC3 | ENSG00000188869 | Q7Z5M5 | 0.9456 | 0.6093 | parathyroid gland |
| TMC5 | ENSG00000103534 | Q6UXY8 | 0.6831 | 0.5594 | small intestine |
| TMC7 | ENSG00000170537 | Q7Z402 | 0.6989 | 0.4468 | cerebral cortex |
| TMCO2 | ENSG00000188800 | Q7Z6W1 | 1 | 1 | urinary bladder |
| TMCO5A | ENSG00000166069 | Q8N6Q1 | 0.9646 | 0.9553 | fallopian tube |
| TMEFF1 | ENSG00000241697 | Q81YR6 | 0.9649 | 0.8661 | cerebral cortex |
| TMEFF2 | ENSG00000144339 | Q9UIK5 | 0.8949 | 0.7835 | prostate |
| TMEM100 | ENSG00000166292 | Q9NV29 | 0.6522 | 0.4322 | lung |
| TMEM108 | ENSG00000144868 | Q6UXF1 | 0.6842 | 0.4661 | cerebral cortex |
| TMEM114 | ENSG00000232258 | B3SHH9 | 0.9969 | 0.9933 | seminal vesicle |
| TMEM121 | ENSG00000184986 | Q9BTD3 | 0.8174 | 0.4963 | cerebral cortex |
| TMEM125 | ENSG00000179178 | Q96AQ2 | 0.6355 | 0.4566 | lung |
| TMEM132B | ENSG00000139364 | Q14DG7 | 0.9324 | 0.7198 | cerebral cortex |
| TMEM132C | ENSG00000181234 | Q8N3T6 | 0.7671 | 0.5363 | adipose tissue |
| TMEM132D | ENSG00000151952 | Q14C87 | 0.9839 | 0.9112 | cerebral cortex |
| TMEM132E | ENSG00000181291 | Q6IEE7 | 0.9061 | 0.7371 | cerebral cortex |
| TMEM139 | ENSG00000178826 | Q8IV31 | 0.6635 | 0.4612 | kidney |
| TMEM150B | ENSG00000180061 | A6NC51 | 0.7577 | 0.5222 | duodenum |
| TMEM151A | ENSG00000179292 | Q8N4L1 | 0.978 | 0.9391 | cerebral cortex |
| TMEM151B | ENSG00000178233 | Q8IW70 | 0.9642 | 0.8543 | cerebral cortex |
| TMEM156 | ENSG00000121895 | Q8N614 | 0.7119 | 0.4753 | b-cells |
| TMEM158 | ENSG00000249992 | Q8WZ71 | 0.7111 | 0.5516 | endometrium |
| TMEM163 | ENSG00000152128 | Q8TC26 | 0.6972 | 0.5342 | lung |
| TMEM169 | ENSG00000163449 | Q96HH4 | 0.7746 | 0.402 | cerebral cortex |
| TMEM171 | ENSG00000157111 | Q8WVE6 | 0.7486 | 0.6289 | thyroid gland |
| TMEM178B | ENSG00000261115 | H3BS89 | 0.8316 | 0.6226 | parathyroid gland |
| TMEM179 | ENSG00000258986 | Q6ZVK1 | 0.9425 | 0.8058 | cerebral cortex |
| TMEM184A | ENSG00000164855 | Q6ZMB5 | 0.6607 | 0.4951 | skin |
| TMEM190 | ENSG00000160472 | Q8WZ59 | 0.9519 | 0.8179 | fallopian tube |
| TMEM196 | ENSG00000173452 | Q5HYL7 | 0.9489 | 0.8654 | cerebral cortex |
| TMEM200A | ENSG00000164484 | Q86VY9 | 0.6372 | 0.4026 | endometrium |
| TMEM200B | ENSG00000253304 | Q69YZ2 | 0.6313 | 0.435 | endometrium |
| TMEM200C | ENSG00000206432 | A6NKL6 | 0.8334 | 0.6523 | cerebral cortex |
| TMEM207 | ENSG00000198398 | Q6UWW9 | 1 | 1 | kidney |
| TMEM210 | ENSG00000185863 | A6NLX4 | 0.8708 | 0.7003 | dendritic cells |
| TMEM211 | ENSG00000206069 | Q6ICI0 | 0.9168 | 0.8483 | cervix, uterine |
| TMEM213 | ENSG00000214128 | A2RRL7 | 0.8895 | 0.5834 | kidney |
| TMEM215 | ENSG00000188133 | Q68D42 | 0.9679 | 0.9477 | endometrium |
| TMEM217 | ENSG00000172738 | Q8N7C4 | 0.8097 | 0.6135 | adrenal gland |
| TMEM229A | ENSG00000234224 | B2RXF0 | 0.8993 | 0.8089 | duodenum |
| TMEM232 | ENSG00000186952 | C9JQ17 | 0.8214 | 0.5365 | fallopian tube |
| TMEM233 | ENSG00000224982 | B4DJY2 | 0.8755 | 0.6497 | thyroid gland |
| TMEM235 | ENSG00000204278 | A6NFC5 | 0.9992 | 0.9984 | cerebral cortex |
| TMEM236 | ENSG00000148483 | Q5WOB7 | 0.8488 | 0.5784 | small intestine |
| TMEM239 | ENSG00000198326 | Q8WW34 | 0.9512 | 0.9512 | endometrium |
| TMEM240 | ENSG00000205090 | Q5SV17 | 0.7488 | 0.4689 | cerebral cortex |
| TMEM244 | ENSG00000203756 | Q5VVB8 | 0.9039 | 0.7619 | cerebral cortex |
| TMEM252 | ENSG00000181778 | Q8N6L7 | 0.8339 | 0.632 | kidney |
| TMEM253 | ENSG00000232070 | P0C7T8 | 0.8508 | 0.6319 | duodenum |
| TMEM255A | ENSG00000125355 | Q5JRV8 | 0.7117 | 0.42 | ovary |
| TMEM26 | ENSG00000196932 | Q6ZUK4 | 0.7492 | 0.5397 | spleen |
| TMEM262 | ENSG00000187066 | E9PQX1 | 0.9745 | 0.9626 | cervix, uterine |
| TMEM266 | ENSG00000169758 | Q2M3C6 | 0.9063 | 0.8506 | cerebral cortex |
| TMEM270 | ENSG00000175877 | Q6UE05 | 1 | 1 | duodenum |
| TMEM31 | ENSG00000179363 | Q5JXX7 | 0.745 | 0.5571 | ovary |
| TMEM40 | ENSG00000088726 | Q8WWA1 | 0.8467 | 0.6879 | esophagus |
| TMEM45B | ENSG00000151715 | Q96B21 | 0.6284 | 0.4348 | small intestine |
| TMEM52 | ENSG00000178821 | Q8NDY8 | 0.8125 | 0.5194 | skeletal muscle |
| TMEM52B | ENSG00000165685 | Q4KMG9 | 0.9367 | 0.6494 | kidney |
| TMEM61 | ENSG00000143001 | Q8N0U2 | 0.7421 | 0.5896 | parathyroid gland |
| TMEM63C | ENSG00000165548 | Q9P1W3 | 0.7858 | 0.5453 | cerebral cortex |
| TMEM72 | ENSG00000187783 | A0PK05 | 0.9511 | 0.8824 | kidney |
| TMEM74B | ENSG00000125895 | Q9NUR3 | 0.6474 | 0.4362 | small intestine |
| TMEM82 | ENSG00000162460 | A0PJX8 | 0.9196 | 0.8958 | duodenum |
| TMEM88B | ENSG00000205116 | A6NKF7 | 0.9928 | 0.9593 | cerebral cortex |
| TMEM92 | ENSG00000167105 | Q6UXU6 | 0.8017 | 0.6433 | small intestine |
| TMIE | ENSG00000181585 | Q8NEW7 | 0.7843 | 0.455 | adrenal gland |
| TMIGD1 | ENSG00000182271 | Q6UXZ0 | 0.9385 | 0.9193 | small intestine |
| TMIGD2 | ENSG00000167664 | Q96BF3 | 0.8416 | 0.64 | t-cells |
| TMIGD3 | ENSG00000121933 | P0DMS9 | 0.862 | 0.5243 | granulocytes |
| TMPRSS11B | ENSG00000185873 | Q86T26 | 0.9709 | 0.9227 | esophagus |
| TMPRSS11D | ENSG00000153802 | O60235 | 0.9714 | 0.9602 | esophagus |
| TMPRSS11E | ENSG00000087128 | Q9UL52 | 0.9229 | 0.8539 | esophagus |
| TMPRSS11F | ENSG00000198092 | Q6ZWK6 | 0.9533 | 0.9353 | esophagus |
| TMPRSS12 | ENSG00000186452 | Q86WS5 | 0.9713 | 0.9209 | epididymis |
| TMPRSS13 | ENSG00000137747 | Q9BYE2 | 0.806 | 0.5436 | skin |
| TMPRSS15 | ENSG00000154646 | P98073 | 0.9787 | 0.9274 | duodenum |
| TMPRSS2 | ENSG00000184012 | O15393 | 0.6939 | 0.5219 | prostate |
| TMPRSS4 | ENSG00000137648 | Q9NRS4 | 0.7007 | 0.6161 | urinary bladder |
| TMPRSS5 | ENSG00000166682 | Q9H3S3 | 0.8756 | 0.5459 | cerebral cortex |
| TMPRSS6 | ENSG00000187045 | Q8IU80 | 0.9215 | 0.72 | liver |
| TMPRSS7 | ENSG00000176040 | Q7RTY8 | 0.9234 | 0.8185 | fallopian tube |
| TMPRSS9 | ENSG00000178297 | Q7Z410 | 0.903 | 0.6829 | spleen |
| TNF | ENSG00000232810 | P01375 | 0.8625 | 0.6544 | monocytes |
| TNFRSF11A | ENSG00000141655 | Q9Y6Q6 | 0.6834 | 0.4509 | duodenum |
| TNFRSF13B | ENSG00000240505 | O14836 | 0.8115 | 0.6221 | b-cells |
| TNFRSF13C | ENSG00000159958 | Q96RJ3 | 0.7895 | 0.569 | tonsil |
| TNFRSF17 | ENSG00000048462 | Q02223 | 0.7029 | 0.5256 | dendritic cells |
| TNFRSF18 | ENSG00000186891 | Q9Y5U5 | 0.7812 | 0.5109 | nk-cells |
| TNFRSF19 | ENSG00000127863 | Q9NS68 | 0.6707 | 0.4033 | skin |
| TNFRSF4 | ENSG00000186827 | P43489 | 0.7814 | 0.5591 | t-cells |
| TNFRSF8 | ENSG00000120949 | P28908 | 0.8899 | 0.6601 | monocytes |
| TNFRSF9 | ENSG00000049249 | Q07011 | 0.7643 | 0.5105 | t-cells |
| TNFSF11 | ENSG00000120659 | O14788 | 0.8312 | 0.6901 | lymph node |
| TNFSF14 | ENSG00000125735 | O43557 | 0.7948 | 0.4852 | granulocytes |
| TNFSF15 | ENSG00000181634 | O95150 | 0.6607 | 0.4575 | duodenum |
| TNFSF18 | ENSG00000120337 | Q9UNG2 | 0.8426 | 0.6081 | gallbladder |
| TNFSF9 | ENSG00000125657 | P41273 | 0.7236 | 0.5092 | cerebral cortex |
| TNMD | ENSG00000000005 | Q9H2S6 | 0.9008 | 0.7151 | seminal vesicle |
| TPBGL | ENSG00000261594 | PODKB5 | 0.8978 | 0.7203 | cerebral cortex |
| TPO | ENSG00000115705 | P07202 | 0.9389 | 0.7179 | thyroid gland |
| TRABD2A | ENSG00000186854 | Q86V40 | 0.7433 | 0.5337 | t-cells |
| TRABD2B | ENSG00000269113 | A6NFA1 | 0.7693 | 0.4913 | kidney |
| TRAT1 | ENSG00000163519 | Q6PIZ9 | 0.8336 | 0.5594 | granulocytes |
| TRDN | ENSG00000186439 | Q13061 | 0.8852 | 0.744 | skeletal muscle |
| TREM1 | ENSG00000124731 | Q9NP99 | 0.7737 | 0.5353 | granulocytes |
| TREM2 | ENSG00000095970 | Q9NZC2 | 0.6811 | 0.4447 | lung |
| TREML1 | ENSG00000161911 | Q86YW5 | 0.8783 | 0.6138 | granulocytes |
| TREML2 | ENSG00000112195 | Q5T2D2 | 0.794 | 0.6796 | granulocytes |
| TRHDE | ENSG00000072657 | Q9UKU6 | 0.7191 | 0.5526 | cerebral cortex |
| TRHR | ENSG00000174417 | P34981 | 0.9872 | 0.9833 | thyroid gland |
| TRPA1 | ENSG00000104321 | O75762 | 0.834 | 0.6815 | urinary bladder |
| TRPC3 | ENSG00000138741 | Q13507 | 0.8522 | 0.6691 | smooth muscle |
| TRPC4 | ENSG00000133107 | Q9UBN4 | 0.8046 | 0.6247 | endometrium |
| TRPC5 | ENSG00000072315 | Q9UL62 | 0.9918 | 0.9845 | cerebral cortex |
| TRPC6 | ENSG00000137672 | Q9Y210 | 0.7054 | 0.4515 | placenta |
| TRPC7 | ENSG00000069018 | Q9HCX4 | 0.9667 | 0.949 | adrenal gland |
| TRPM1 | ENSG00000134160 | Q7Z4N2 | 0.9987 | 0.9975 | skin |
| TRPM3 | ENSG00000083067 | Q9HCF6 | 0.8849 | 0.6614 | kidney |
| TRPM5 | ENSG00000070985 | Q9NZQ8 | 0.8895 | 0.8472 | duodenum |
| TRPM6 | ENSG00000119121 | Q9BX84 | 0.7984 | 0.5741 | rectum |
| TRPM8 | ENSG00000144481 | Q7Z2W7 | 0.9746 | 0.9062 | prostate |
| TRPV3 | ENSG00000167723 | Q8NET8 | 0.8549 | 0.6042 | skin |
| TRPV4 | ENSG00000111199 | Q9HBA0 | 0.6971 | 0.5086 | kidney |
| TSHR | ENSG00000165409 | P16473 | 0.9193 | 0.478 | thyroid gland |
| TSPAN11 | ENSG00000110900 | A1L157 | 0.5707 | 0.4003 | cerebral cortex |
| TSPAN16 | ENSG00000130167 | Q9UKR8 | 0.9657 | 0.8961 | granulocytes |
| TSPAN19 | ENSG00000231738 | P0C672 | 0.7643 | 0.4476 | lung |
| TSPAN32 | ENSG00000064201 | Q96QS1 | 0.772 | 0.5576 | nk-cells |
| TSPAN8 | ENSG00000127324 | P19075 | 0.6163 | 0.4484 | rectum |
| TSPO2 | ENSG00000112212 | Q5TGU0 | 0.9632 | 0.8681 | bone marrow |
| TTYH1 | ENSG00000167614 | Q9H313 | 0.9773 | 0.9012 | cerebral cortex |
| TVP23A | ENSG00000166676 | A6NH52 | 0.7612 | 0.4407 | cerebral cortex |
| UGT2A3 | ENSG00000135220 | Q6UWM9 | 0.849 | 0.8059 | small intestine |
| UGT3A1 | ENSG00000145626 | Q6NUS8 | 0.9396 | 0.9042 | kidney |
| UGT3A2 | ENSG00000168671 | Q3SY77 | 0.9323 | 0.8459 | skin |
| UGT8 | ENSG00000174607 | Q16880 | 0.7181 | 0.4897 | cerebral cortex |
| UMODL1 | ENSG00000177398 | Q5DID0 | 0.9862 | 0.9525 | fallopian tube |
| UNC5A | ENSG00000113763 | Q6ZN44 | 0.9416 | 0.7625 | cerebral cortex |
| UNC5C | ENSG00000182168 | O95185 | 0.6989 | 0.4543 | thyroid gland |
| UNC5CL | ENSG00000124602 | Q8IV45 | 0.7915 | 0.5306 | duodenum |
| UNC5D | ENSG00000156687 | Q6UXZ4 | 0.8519 | 0.7835 | cerebral cortex |
| UNC79 | ENSG00000133958 | Q9P2D8 | 0.9738 | 0.9173 | cerebral cortex |
| UNC80 | ENSG00000144406 | Q8N2C7 | 0.9325 | 0.8017 | cerebral cortex |
| UNC93A | ENSG00000112494 | Q86WB7 | 0.8877 | 0.81 | skin |
| UPK1A | ENSG00000105668 | O00322 | 0.926 | 0.7878 | urinary bladder |
| UPK1B | ENSG00000114638 | O75841 | 0.8245 | 0.7324 | urinary bladder |
| UPK2 | ENSG00000110375 | O00526 | 0.974 | 0.8701 | urinary bladder |
| UPK3B | ENSG00000243566 | Q9BT76 | 0.9267 | 0.8361 | lung |
| USH2A | ENSG00000042781 | O75445 | 0.9843 | 0.9665 | liver |
| UTS2R | ENSG00000181408 | Q9UKP6 | 0.9568 | 0.8471 | thyroid gland |
| VIPR2 | ENSG00000106018 | P41587 | 0.6874 | 0.4614 | seminal vesicle |
| VN1R1 | ENSG00000178201 | Q9GZP7 | 0.6791 | 0.4448 | epididymis |
| VSIG1 | ENSG00000101842 | Q86XK7 | 0.9155 | 0.7056 | stomach |
| VSIG10L | ENSG00000186806 | Q86VR7 | 0.9317 | 0.8632 | esophagus |
| VSIG2 | ENSG00000019102 | Q96IQ7 | 0.7055 | 0.4608 | stomach |
| VSIG8 | ENSG00000243284 | P0DPA2 | 0.9382 | 0.8247 | skin |
| VSTM1 | ENSG00000189068 | Q6UX27 | 0.9617 | 0.9334 | granulocytes |
| VSTM2B | ENSG00000187135 | A6NLU5 | 0.9854 | 0.96 | cerebral cortex |
| VSTM5 | ENSG00000214376 | A8MXK1 | 0.8483 | 0.6388 | placenta |
| VTCN1 | ENSG00000134258 | Q7Z7D3 | 0.8472 | 0.7231 | breast |
| WSCD1 | ENSG00000179314 | Q658N2 | 0.7597 | 0.4749 | cerebral cortex |
| WSCD2 | ENSG00000075035 | Q2TBF2 | 0.8152 | 0.656 | thyroid gland |
| XCR1 | ENSG00000173578 | P46094 | 0.6896 | 0.4338 | lymph node |
| XG | ENSG00000124343 | P55808 | 0.8478 | 0.663 | skin |
| XK | ENSG00000047597 | P51811 | 0.6198 | 0.4369 | rectum |
| XKR3 | ENSG00000172967 | Q5GH77 | 0.9933 | 0.9842 | granulocytes |
| XKR4 | ENSG00000206579 | Q5GH76 | 0.9082 | 0.6061 | cerebral cortex |
| XKR6 | ENSG00000171044 | Q5GH73 | 0.6236 | 0.4395 | cerebral cortex |
| XKR7 | ENSG00000260903 | Q5GH72 | 0.9929 | 0.9845 | cerebral cortex |
| XKR9 | ENSG00000221947 | Q5GH70 | 0.7604 | 0.4506 | small intestine |
| XKRX | ENSG00000182489 | Q6PP77 | 0.835 | 0.5806 | skin |
| ZDHHC11B | ENSG00000206077 | P0C7U3 | 0.6605 | 0.4713 | cerebral cortex |
| ZDHHC15 | ENSG00000102383 | Q96MV8 | 0.6646 | 0.4601 | cerebral cortex |
| ZP1 | ENSG00000149506 | P60852 | 0.9899 | 0.9687 | dendritic cells |
| ZP2 | ENSG00000103310 | Q05996 | 0.9395 | 0.9097 | placenta |
| ZP4 | ENSG00000116996 | Q12836 | 0.9745 | 0.9626 | tonsil |
| ZPLD1 | ENSG00000170044 | Q8TCW7 | 0.958 | 0.8929 | gallbladder |

Table 2B contains address targets based on a Gtex database analysis:

TABLE 2B
Exemplary Address Targets (Gtex database analysis)

| | | | | | Exemplary |
|---|---|---|---|---|---|
| | | | | | tissue/cellular |
| Gene | | | | | localization of |
| symbol | Ensembl | Uniprot ID | Tau score | Gini score | address target |
| AADACL4 | ENSG00000204518 | Q5VUY2 | 0.8989 | 0.835 | vagina |
| ABCA12 | ENSG00000144452 | Q86UK0 | 0.9572 | 0.9191 | skin |
| ABCA13 | ENSG00000179869 | Q86UQ4 | 0.8778 | 0.7422 | thyroid gland |
| ABCA4 | ENSG00000198691 | P78363 | 0.8822 | 0.6752 | kidney |
| ABCB11 | ENSG00000073734 | 095342 | 0.9699 | 0.9393 | liver |
| ABCB4 | ENSG00000005471 | P21439 | 0.8807 | 0.5882 | liver |
| ABCB5 | ENSG00000004846 | Q2M3G0 | 0.8633 | 0.8033 | cervix, uterine |
| ABCC11 | ENSG00000121270 | Q96J66 | 0.9143 | 0.8074 | breast |
| ABCC12 | ENSG00000140798 | Q96J65 | 0.9753 | 0.9471 | cerebral cortex |
| ABCC2 | ENSG00000023839 | Q92887 | 0.8753 | 0.5552 | liver |
| ABCC3 | ENSG00000108846 | O15438 | 0.6722 | 0.4456 | adrenal gland |
| ABCC6 | ENSG00000091262 | O95255 | 0.7202 | 0.4101 | liver |
| ABCC8 | ENSG00000006071 | Q09428 | 0.7232 | 0.4722 | cerebellum |
| ABCG4 | ENSG00000172350 | Q9H172 | 0.8477 | 0.6202 | cerebellum |
| ABCG5 | ENSG00000138075 | Q9H222 | 0.9915 | 0.9853 | liver |
| ABCG8 | ENSG00000143921 | Q9H221 | 0.9782 | 0.9514 | liver |
| ACKR2 | ENSG00000144648 | O00590 | 0.7425 | 0.5222 | adipose tissue |
| ACKR4 | ENSG00000129048 | Q9NPB9 | 0.6548 | 0.4434 | adipose tissue |
| ACP4 | ENSG00000142513 | Q9BZG2 | 1 | 1 | skin |
| ACVR1C | ENSG00000123612 | Q8NER5 | 0.7767 | 0.5035 | adipose tissue |
| ADAM11 | ENSG00000073670 | 075078 | 0.7977 | 0.5251 | cerebellum |
| ADAM12 | ENSG00000148848 | O43184 | 0.735 | 0.4805 | ovary |
| ADAM20 | ENSG00000134007 | O43506 | 0.8511 | 0.6232 | cerebellum |
| ADAM21 | ENSG00000139985 | Q9UKJ8 | 0.7415 | 0.6204 | adrenal gland |
| ADAM29 | ENSG00000168594 | Q9UKF5 | 0.9339 | 0.9072 | cervix, uterine |
| ADCY1 | ENSG00000164742 | Q08828 | 0.6979 | 0.4014 | cerebellum |
| ADCY2 | ENSG00000078295 | Q08462 | 0.6206 | 0.4319 | cerebral cortex |
| ADCY8 | ENSG00000155897 | P40145 | 0.7714 | 0.7388 | cerebellum |
| ADGRA1 | ENSG00000197177 | Q86SQ6 | 0.8153 | 0.7342 | cerebral cortex |
| ADGRB1 | ENSG00000181790 | O14514 | 0.69 | 0.4802 | cerebral cortex |
| ADGRB2 | ENSG00000121753 | O60241 | 0.718 | 0.5019 | cerebral cortex |
| ADGRB3 | ENSG00000135298 | O60242 | 0.6602 | 0.4405 | cerebellum |
| ADGRE1 | ENSG00000174837 | Q14246 | 0.9433 | 0.728 | spleen |
| ADGRE2 | ENSG00000127507 | Q9UHX3 | 0.8197 | 0.4098 | spleen |
| ADGRE3 | ENSG00000131355 | Q9BY15 | 0.9253 | 0.9025 | spleen |
| ADGRF1 | ENSG00000153292 | Q5T601 | 0.7994 | 0.7451 | esophagus |
| ADGRF3 | ENSG00000173567 | Q8IZF5 | 0.7853 | 0.5041 | cerebellum |
| ADGRF4 | ENSG00000153294 | Q8IZF3 | 0.8768 | 0.7624 | skin |
| ADGRG2 | ENSG00000173698 | Q8IZP9 | 0.7417 | 0.4908 | fallopian tube |
| ADGRG3 | ENSG00000182885 | Q86Y34 | 0.822 | 0.5524 | spleen |
| ADGRG5 | ENSG00000159618 | Q8IZF4 | 0.8999 | 0.6892 | spleen |
| ADGRG7 | ENSG00000144820 | Q96K78 | 0.9151 | 0.7474 | small intestine |
| ADGRV1 | ENSG00000164199 | Q8WXG9 | 0.8204 | 0.6182 | adrenal gland |
| ADIG | ENSG00000182035 | QOVDE8 | 0.9763 | 0.9274 | thyroid gland |
| ADORA1 | ENSG00000163485 | P30542 | 0.6015 | 0.4359 | spinal cord |
| ADORA2B | ENSG00000170425 | P29275 | 0.6191 | 0.4465 | skin |
| ADRA1A | ENSG00000120907 | P35348 | 0.7609 | 0.4698 | liver |
| ADRA1D | ENSG00000171873 | P25100 | 0.6935 | 0.4701 | cervix, uterine |
| ADRB3 | ENSG00000188778 | P13945 | 0.8966 | 0.8098 | ovary |
| ADTRP | ENSG00000111863 | Q96IZ2 | 0.8265 | 0.7087 | colon |
| AGTR2 | ENSG00000180772 | P50052 | 0.9248 | 0.849 | lung |
| AJAP1 | ENSG00000196581 | Q9UKB5 | 0.6332 | 0.4242 | cerebral cortex |
| ALK | ENSG00000171094 | Q9UM73 | 0.8644 | 0.7105 | pituitary gland |
| ALPP | ENSG00000163283 | P05187 | 0.9749 | 0.9592 | lung |
| AMHR2 | ENSG00000135409 | Q16671 | 0.9163 | 0.8041 | adrenal gland |
| AMN | ENSG00000166126 | Q9BXJ7 | 0.8347 | 0.6656 | small intestine |
| ANO3 | ENSG00000134343 | Q9BYT9 | 0.922 | 0.742 | basal ganglia |
| ANO4 | ENSG00000151572 | Q32M45 | 0.6904 | 0.6144 | cervix, uterine |
| ANO7 | ENSG00000146205 | Q6IWH7 | 0.882 | 0.6368 | prostate |
| ANO9 | ENSG00000185101 | A1A5B4 | 0.7384 | 0.56 | small intestine |
| APCDD1L | ENSG00000198768 | Q8NCL9 | 0.8561 | 0.7015 | salivary gland |
| APLNR | ENSG00000134817 | P35414 | 0.7974 | 0.5614 | spinal cord |
| APLP1 | ENSG00000105290 | P51693 | 0.6626 | 0.4667 | spinal cord |
| AQP10 | ENSG00000143595 | Q96PS8 | 0.9596 | 0.9353 | fallopian tube |
| AQP12A | ENSG00000184945 | Q8IXF9 | 0.9975 | 0.9938 | pancreas |
| AQP12B | ENSG00000185176 | A6NM10 | 0.9949 | 0.983 | pancreas |
| AQP2 | ENSG00000167580 | P41181 | 0.9798 | 0.9418 | kidney |
| AQP4 | ENSG00000171885 | P55087 | 0.72 | 0.6407 | basal ganglia |
| AQP5 | ENSG00000161798 | P55064 | 0.7966 | 0.5051 | salivary gland |
| AQP7 | ENSG00000165269 | O14520 | 0.7466 | 0.473 | adipose tissue |
| AQP9 | ENSG00000103569 | O43315 | 0.8013 | 0.5358 | liver |
| AREG | ENSG00000109321 | P15514 | 0.6191 | 0.4843 | esophagus |
| ARSH | ENSG00000205667 | Q5FYA8 | 0.9688 | 0.9688 | esophagus |
| ASGR2 | ENSG00000161944 | P07307 | 0.9297 | 0.5961 | liver |
| ASIC1 | ENSG00000110881 | P78348 | 0.6609 | 0.4246 | cerebellum |
| ASIC2 | ENSG00000108684 | Q16515 | 0.7764 | 0.7059 | cerebellum |
| ASIC4 | ENSG00000072182 | Q96FT7 | 0.8687 | 0.7419 | pituitary gland |
| ASPHD1 | ENSG00000174939 | Q5U4P2 | 0.6268 | 0.5052 | cerebellum |
| ASTN1 | ENSG00000152092 | O14525 | 0.7137 | 0.6117 | cerebral cortex |
| ATP12A | ENSG00000075673 | P54707 | 0.9503 | 0.919 | skin |
| ATP13A4 | ENSG00000127249 | Q4VNC1 | 0.6941 | 0.4676 | thyroid gland |
| ATP13A5 | ENSG00000187527 | Q4VNC0 | 0.8887 | 0.7293 | skin |
| ATP1A3 | ENSG00000105409 | P13637 | 0.7338 | 0.6684 | cerebral cortex |
| ATP1A4 | ENSG00000132681 | Q13733 | 0.9581 | 0.8285 | urinary bladder |
| ATP2B2 | ENSG00000157087 | Q01814 | 0.8001 | 0.6662 | cerebellum |
| ATP2B3 | ENSG00000067842 | Q16720 | 0.8402 | 0.7707 | cerebellum |
| ATP2C2 | ENSG00000064270 | O75185 | 0.6722 | 0.5135 | colon |
| ATP4A | ENSG00000105675 | P20648 | 0.9779 | 0.9161 | stomach |
| ATP4B | ENSG00000186009 | P51164 | 0.9749 | 0.8349 | stomach |
| ATP6V0A4 | ENSG00000105929 | Q9HBG4 | 0.9177 | 0.8026 | kidney |
| ATP8A2 | ENSG00000132932 | Q9NTI2 | 0.8268 | 0.7218 | cerebellum |
| ATP8B4 | ENSG00000104043 | Q8TF62 | 0.8819 | 0.6168 | fallopian tube |
| ATRNL1 | ENSG00000107518 | Q5VV63 | 0.7608 | 0.493 | cerebral cortex |
| AVPR1A | ENSG00000166148 | P37288 | 0.7667 | 0.5661 | adrenal gland |
| AVPR1B | ENSG00000198049 | P47901 | 0.9935 | 0.9849 | pituitary gland |
| AVPR2 | ENSG00000126895 | P30518 | 0.6887 | 0.4753 | adipose tissue |
| B3GAT1 | ENSG00000109956 | Q9P2W7 | 0.7055 | 0.5761 | spinal cord |
| BDKRB1 | ENSG00000100739 | P46663 | 0.6817 | 0.5196 | esophagus |
| BDKRB2 | ENSG00000168398 | P30411 | 0.6259 | 0.4487 | cervix, uterine |
| BEST2 | ENSG00000039987 | Q8NFU1 | 0.94 | 0.9193 | colon |
| BEST3 | ENSG00000127325 | Q8N1M1 | 0.8897 | 0.7282 | skeletal muscle |
| BEST4 | ENSG00000142959 | Q8NFU0 | 0.789 | 0.5119 | colon |
| BMPR1B | ENSG00000138696 | O00238 | 0.6286 | 0.4094 | prostate |
| BRS3 | ENSG00000102239 | P32247 | 0.8802 | 0.8064 | fallopian tube |
| BSND | ENSG00000162399 | Q8WZ55 | 0.9678 | 0.9248 | kidney |
| BTBD11 | ENSG00000151136 | A6QL63 | 0.6655 | 0.4431 | esophagus |
| BTC | ENSG00000174808 | P35070 | 0.7263 | 0.4532 | colon |
| BTLA | ENSG00000186265 | Q7Z6A9 | 0.9309 | 0.7922 | spleen |
| BTN1A1 | ENSG00000124557 | Q13410 | 0.9504 | 0.9049 | breast |
| BTNL2 | ENSG00000204290 | Q9UIRO | 0.8395 | 0.7937 | hypothalamus |
| BTNL3 | ENSG00000168903 | Q6UXE8 | 0.9575 | 0.9205 | small intestine |
| BTNL8 | ENSG00000113303 | Q6UX41 | 0.9053 | 0.7554 | small intestine |
| C10orf105 | ENSG00000214688 | Q8TEF2 | 0.7844 | 0.6569 | hypothalamus |
| C11orf87 | ENSG00000185742 | Q6NUJ2 | 0.8892 | 0.809 | cerebral cortex |
| C16orf54 | ENSG00000185905 | Q6UWD8 | 0.8507 | 0.5709 | spleen |
| C1orf210 | ENSG00000253313 | Q8IVY1 | 0.601 | 0.5385 | colon |
| C20orf141 | ENSG00000258713 | Q9NUB4 | 0.9334 | 0.8893 | endometrium |
| C3orf80 | ENSG00000180044 | F5H4A9 | 0.8005 | 0.5354 | cerebral cortex |
| C8A | ENSG00000157131 | P07357 | 0.9978 | 0.9948 | liver |
| C9 | ENSG00000113600 | P02748 | 0.9938 | 0.982 | liver |
| C9orf135 | ENSG00000204711 | Q5VTT2 | 0.8841 | 0.8073 | pituitary gland |
| CA14 | ENSG00000118298 | Q9ULX7 | 0.7572 | 0.5196 | spinal cord |
| CA9 | ENSG00000107159 | Q16790 | 0.9013 | 0.6825 | stomach |
| CABP7 | ENSG00000100314 | Q86V35 | 0.8373 | 0.6335 | hippocampal |
| | | | | | formation |
| CACNA1A | ENSG00000141837 | O00555 | 0.814 | 0.5316 | cerebellum |
| CACNA1B | ENSG00000148408 | Q00975 | 0.8321 | 0.7204 | cerebellum |
| CACNA1E | ENSG00000198216 | Q15878 | 0.8597 | 0.7723 | basal ganglia |
| CACNA1F | ENSG00000102001 | 060840 | 0.7955 | 0.4964 | cerebellum |
| CACNA1G | ENSG00000006283 | O43497 | 0.7611 | 0.5529 | cerebellum |
| CACNA11 | ENSG00000100346 | Q9P0X4 | 0.7978 | 0.6741 | cerebellum |
| CACNA1S | ENSG00000081248 | Q13698 | 0.9861 | 0.9524 | skeletal muscle |
| CACNA2D4 | ENSG00000151062 | Q7Z3S7 | 0.7702 | 0.6706 | cervix, uterine |
| CACNG1 | ENSG00000108878 | Q06432 | 0.9585 | 0.832 | skeletal muscle |
| CACNG2 | ENSG00000166862 | Q9Y698 | 0.905 | 0.8191 | cerebellum |
| CACNG3 | ENSG00000006116 | O60359 | 0.8861 | 0.8491 | cerebral cortex |
| CACNG4 | ENSG00000075461 | Q9UBN1 | 0.7424 | 0.5817 | basal ganglia |
| CACNG5 | ENSG00000075429 | Q9UF02 | 0.8959 | 0.8298 | cerebral cortex |
| CACNG6 | ENSG00000130433 | Q9BXT2 | 0.9262 | 0.8218 | skeletal muscle |
| CACNG7 | ENSG00000105605 | P62955 | 0.7979 | 0.7687 | cerebellum |
| CADM2 | ENSG00000175161 | Q8N3J6 | 0.7098 | 0.5567 | cerebral cortex |
| CALCR | ENSG00000004948 | P30988 | 0.9673 | 0.9007 | hypothalamus |
| CALHM1 | ENSG00000185933 | Q8IU99 | 0.9243 | 0.8662 | cerebral cortex |
| CALHM3 | ENSG00000183128 | Q86XJO | 0.7812 | 0.7813 | basal ganglia |
| CALHM4 | ENSG00000164451 | Q5JW98 | 0.9929 | 0.9796 | skin |
| CALN1 | ENSG00000183166 | Q9BXU9 | 0.8276 | 0.6864 | cerebellum |
| CALY | ENSG00000130643 | Q9NYX4 | 0.7262 | 0.6046 | hypothalamus |
| CASR | ENSG00000036828 | P41180 | 0.9491 | 0.9043 | kidney |
| CATSPER1 | ENSG00000175294 | Q8NEC5 | 0.9659 | 0.9348 | lung |
| CATSPERB | ENSG00000133962 | Q9H7TO | 0.8538 | 0.6277 | pancreas |
| CATSPERG | ENSG00000099338 | Q6ZRH7 | 0.8309 | 0.4479 | cerebellum |
| CBARP | ENSG00000099625 | Q8N350 | 0.734 | 0.4897 | pituitary gland |
| CCKAR | ENSG00000163394 | P32238 | 0.9791 | 0.9379 | stomach |
| CCKBR | ENSG00000110148 | P32239 | 0.8408 | 0.7516 | stomach |
| CCR2 | ENSG00000121807 | P41597 | 0.8123 | 0.5303 | spleen |
| CCR3 | ENSG00000183625 | P51677 | 0.9223 | 0.8592 | skin |
| CCR4 | ENSG00000183813 | P51679 | 0.8585 | 0.6996 | spleen |
| CCR5 | ENSG00000160791 | P51681 | 0.7574 | 0.4025 | spleen |
| CCR6 | ENSG00000112486 | P51684 | 0.929 | 0.7695 | spleen |
| CCR7 | ENSG00000126353 | P32248 | 0.7273 | 0.4091 | spleen |
| CCR8 | ENSG00000179934 | P51685 | 1 | 1 | spleen |
| CCR9 | ENSG00000173585 | P51686 | 0.9832 | 0.9654 | small intestine |
| CD101 | ENSG00000134256 | Q93033 | 0.7331 | 0.4345 | lung |
| CD163L1 | ENSG00000177675 | Q9NR16 | 0.8456 | 0.488 | spleen |
| CD164L2 | ENSG00000174950 | Q6UWJ8 | 0.79 | 0.6586 | fallopian tube |
| CD180 | ENSG00000134061 | Q99467 | 0.8481 | 0.4806 | spleen |
| CD19 | ENSG00000177455 | P15391 | 0.9325 | 0.8014 | spleen |
| CD1A | ENSG00000158477 | P06126 | 0.9497 | 0.8817 | skin |
| CD1B | ENSG00000158485 | P29016 | 0.9144 | 0.8504 | skin |
| CD1C | ENSG00000158481 | P29017 | 0.8465 | 0.6094 | spleen |
| CD1D | ENSG00000158473 | P15813 | 0.7386 | 0.44 | spleen |
| CD2 | ENSG00000116824 | P06729 | 0.7241 | 0.445 | spleen |
| CD207 | ENSG00000116031 | Q9UJ71 | 0.9176 | 0.7564 | skin |
| CD209 | ENSG00000090659 | Q9NNX6 | 0.7338 | 0.4689 | adipose tissue |
| CD22 | ENSG00000012124 | P20273 | 0.6676 | 0.4227 | spleen |
| CD244 | ENSG00000122223 | Q9BZW8 | 0.8937 | 0.5888 | spleen |
| CD27 | ENSG00000139193 | P26842 | 0.8136 | 0.5699 | spleen |
| CD28 | ENSG00000178562 | P10747 | 0.7989 | 0.5927 | spleen |
| CD300C | ENSG00000167850 | Q08708 | 0.7726 | 0.4033 | spleen |
| CD300E | ENSG00000186407 | Q496F6 | 0.9673 | 0.9521 | lung |
| CD300LB | ENSG00000178789 | A8K4G0 | 0.8427 | 0.6226 | spleen |
| CD300LD | ENSG00000204345 | Q6UXZ3 | 1 | 1 | spleen |
| CD300LF | ENSG00000186074 | Q8TDQ1 | 0.8462 | 0.4747 | spleen |
| CD300LG | ENSG00000161649 | Q6UXG3 | 0.7617 | 0.501 | adipose tissue |
| CD3D | ENSG00000167286 | P04234 | 0.7248 | 0.4255 | spleen |
| CD3E | ENSG00000198851 | P07766 | 0.6996 | 0.4103 | spleen |
| CD3G | ENSG00000160654 | P09693 | 0.8741 | 0.6646 | spleen |
| CD40LG | ENSG00000102245 | P29965 | 0.83 | 0.6454 | small intestine |
| CD5 | ENSG00000110448 | P06127 | 0.7783 | 0.4795 | spleen |
| CD6 | ENSG00000013725 | P30203 | 0.795 | 0.4477 | spleen |
| CD70 | ENSG00000125726 | P32970 | 0.8157 | 0.67 | spleen |
| CD79A | ENSG00000105369 | P11912 | 0.8114 | 0.5571 | spleen |
| CD79B | ENSG00000007312 | P40259 | 0.7786 | 0.4463 | spleen |
| CD80 | ENSG00000121594 | P33681 | 0.9101 | 0.7655 | lung |
| CD8B | ENSG00000172116 | P10966 | 0.6817 | 0.4102 | cervix, uterine |
| CD96 | ENSG00000153283 | P40200 | 0.6722 | 0.4813 | spleen |
| CDCP1 | ENSG00000163814 | Q9H5V8 | 0.6613 | 0.4714 | esophagus |
| CDH1 | ENSG00000039068 | P12830 | 0.5228 | 0.4193 | esophagus |
| CDH10 | ENSG00000040731 | Q9Y6N8 | 0.8758 | 0.7872 | cerebellum |
| CDH12 | ENSG00000154162 | P55289 | 0.7807 | 0.7068 | pituitary gland |
| CDH15 | ENSG00000129910 | P55291 | 0.9365 | 0.8157 | cerebellum |
| CDH16 | ENSG00000166589 | 075309 | 0.9707 | 0.9466 | kidney |
| CDH17 | ENSG00000079112 | Q12864 | 0.9548 | 0.9 | colon |
| CDH18 | ENSG00000145526 | Q13634 | 0.9024 | 0.8297 | cerebellum |
| CDH20 | ENSG00000101542 | Q9HBT6 | 0.7348 | 0.6582 | spinal cord |
| CDH22 | ENSG00000149654 | Q9UJ99 | 0.84 | 0.6067 | cerebellum |
| CDH3 | ENSG00000062038 | P22223 | 0.618 | 0.4292 | ovary |
| CDH4 | ENSG00000179242 | P55283 | 0.7382 | 0.5687 | basal ganglia |
| CDH7 | ENSG00000081138 | Q9ULB5 | 0.9614 | 0.8965 | cerebellum |
| CDH8 | ENSG00000150394 | P55286 | 0.8131 | 0.7176 | cerebral cortex |
| CDH9 | ENSG00000113100 | Q9ULB4 | 0.9137 | 0.8766 | cerebral cortex |
| CDHR1 | ENSG00000148600 | Q96JP9 | 0.8274 | 0.562 | skin |
| CDHR2 | ENSG00000074276 | Q9BYE9 | 0.9281 | 0.8493 | small intestine |
| CDHR3 | ENSG00000128536 | Q6ZTQ4 | 0.7865 | 0.4509 | fallopian tube |
| CDHR4 | ENSG00000187492 | A6H8M9 | 0.9178 | 0.7427 | fallopian tube |
| CDHR5 | ENSG00000099834 | Q9HBB8 | 0.9075 | 0.8039 | small intestine |
| CEACAM1 | ENSG00000079385 | P13688 | 0.6625 | 0.45 | colon |
| CEACAM3 | ENSG00000170956 | P40198 | 0.9395 | 0.8117 | spleen |
| CEACAM4 | ENSG00000105352 | O75871 | 0.8741 | 0.5218 | spleen |
| CELSR1 | ENSG00000075275 | Q9NYQ6 | 0.6442 | 0.4918 | skin |
| CELSR3 | ENSG00000008300 | Q9NYQ7 | 0.8998 | 0.4722 | pituitary gland |
| CEND1 | ENSG00000184524 | Q8N111 | 0.7386 | 0.6293 | cerebellum |
| CFAP65 | ENSG00000181378 | Q6ZU64 | 0.8943 | 0.7567 | fallopian tube |
| CFTR | ENSG00000001626 | P13569 | 0.8847 | 0.7268 | pancreas |
| CHODL | ENSG00000154645 | Q9H9P2 | 0.6737 | 0.4448 | hypothalamus |
| CHRM1 | ENSG00000168539 | P11229 | 0.8158 | 0.7203 | cerebral cortex |
| CHRM2 | ENSG00000181072 | P08172 | 0.9428 | 0.9143 | colon |
| CHRM3 | ENSG00000133019 | P20309 | 0.7198 | 0.4937 | urinary bladder |
| CHRM4 | ENSG00000180720 | P08173 | 0.8309 | 0.6842 | basal ganglia |
| CHRM5 | ENSG00000184984 | P08912 | 0.9148 | 0.7366 | spinal cord |
| CHRNA1 | ENSG00000138435 | P02708 | 0.9387 | 0.8197 | skeletal muscle |
| CHRNA2 | ENSG00000120903 | Q15822 | 0.9315 | 0.8106 | prostate |
| CHRNA3 | ENSG00000080644 | P32297 | 0.8346 | 0.6794 | adrenal gland |
| CHRNA4 | ENSG00000101204 | P43681 | 0.8178 | 0.703 | liver |
| CHRNA6 | ENSG00000147434 | Q15825 | 0.948 | 0.8738 | cerebellum |
| CHRNA9 | ENSG00000174343 | Q9UGM1 | 0.9579 | 0.9415 | skin |
| CHRNB2 | ENSG00000160716 | P17787 | 0.8098 | 0.7622 | cerebral cortex |
| CHRNB3 | ENSG00000147432 | Q05901 | 0.9521 | 0.9339 | midbrain |
| CHRNB4 | ENSG00000117971 | P30926 | 0.8743 | 0.766 | colon |
| CHRND | ENSG00000135902 | Q07001 | 0.9977 | 0.9957 | skeletal muscle |
| CHRNE | ENSG00000108556 | Q04844 | 0.8408 | 0.4426 | heart muscle |
| CHRNG | ENSG00000196811 | P07510 | 0.9969 | 0.9924 | skeletal muscle |
| CHST9 | ENSG00000154080 | Q7L1S5 | 0.7739 | 0.4625 | salivary gland |
| CLCA2 | ENSG00000137975 | Q9UQC9 | 0.861 | 0.7963 | esophagus |
| CLCA4 | ENSG00000016602 | Q14CN2 | 0.7943 | 0.6561 | esophagus |
| CLCN1 | ENSG00000188037 | P35523 | 0.9742 | 0.8952 | skeletal muscle |
| CLCNKA | ENSG00000186510 | P51800 | 0.8866 | 0.7024 | kidney |
| CLCNKB | ENSG00000184908 | P51801 | 0.8865 | 0.6748 | kidney |
| CLDN1 | ENSG00000163347 | 095832 | 0.6447 | 0.4024 | skin |
| CLDN10 | ENSG00000134873 | P78369 | 0.6156 | 0.4714 | pancreas |
| CLDN14 | ENSG00000159261 | O95500 | 0.961 | 0.908 | liver |
| CLDN17 | ENSG00000156282 | P56750 | 0.9271 | 0.9133 | vagina |
| CLDN18 | ENSG00000066405 | P56856 | 0.9418 | 0.7883 | stomach |
| CLDN19 | ENSG00000164007 | Q8N6F1 | 0.9432 | 0.8148 | kidney |
| CLDN2 | ENSG00000165376 | P57739 | 0.8898 | 0.8058 | kidney |
| CLDN22 | ENSG00000177300 | Q8N7P3 | 0.9068 | 0.5104 | salivary gland |
| CLDN23 | ENSG00000253958 | Q96B33 | 0.6008 | 0.4221 | colon |
| CLDN24 | ENSG00000185758 | A6NM45 | 0.9659 | 0.9348 | salivary gland |
| CLDN25 | ENSG00000228607 | C9JDP6 | 1 | 1 | pituitary gland |
| CLDN3 | ENSG00000165215 | O15551 | 0.6637 | 0.5255 | colon |
| CLDN4 | ENSG00000189143 | O14493 | 0.5427 | 0.4551 | esophagus |
| CLDN6 | ENSG00000184697 | P56747 | 0.907 | 0.8192 | cerebellum |
| CLDN7 | ENSG00000181885 | O95471 | 0.5513 | 0.4036 | colon |
| CLDN9 | ENSG00000213937 | O95484 | 0.8093 | 0.5125 | cerebellum |
| CLEC12A | ENSG00000172322 | Q5QGZ9 | 0.8172 | 0.4773 | spleen |
| CLEC12B | ENSG00000256660 | Q2HXU8 | 0.9356 | 0.8836 | spleen |
| CLEC17A | ENSG00000187912 | Q6ZS10 | 0.9475 | 0.8684 | spleen |
| CLEC1B | ENSG00000165682 | Q9P126 | 0.9576 | 0.9305 | liver |
| CLEC2A | ENSG00000188393 | Q6UVW9 | 0.993 | 0.9734 | skin |
| CLEC2L | ENSG00000236279 | POC7M8 | 0.8031 | 0.7071 | cerebral cortex |
| CLEC4C | ENSG00000198178 | Q8WTTO | 1 | 1 | spleen |
| CLEC4D | ENSG00000166527 | Q8WX18 | 0.9288 | 0.7879 | spleen |
| CLEC4F | ENSG00000152672 | Q8N1NO | 0.7414 | 0.5147 | spleen |
| CLEC4G | ENSG00000182566 | Q6UXB4 | 0.7468 | 0.4562 | cerebellum |
| CLEC4M | ENSG00000104938 | Q9H2X3 | 0.9138 | 0.749 | liver |
| CLEC6A | ENSG00000205846 | Q6EIG7 | 1 | 1 | lung |
| CLEC9A | ENSG00000197992 | Q6UXN8 | 0.8512 | 0.6867 | spinal cord |
| CLECL1 | ENSG00000184293 | Q8IZS7 | 0.8401 | 0.4617 | spleen |
| CLIC3 | ENSG00000169583 | 095833 | 0.7265 | 0.5223 | esophagus |
| CLIC5 | ENSG00000112782 | Q9NZA1 | 0.7142 | 0.4696 | skeletal muscle |
| CLRN1 | ENSG00000163646 | P58418 | 0.9937 | 0.986 | adrenal gland |
| CLRN3 | ENSG00000180745 | Q8NCR9 | 0.9148 | 0.8504 | small intestine |
| CLTRN | ENSG00000147003 | Q9HBJ8 | 0.8752 | 0.445 | kidney |
| CMTM5 | ENSG00000166091 | Q96DZ9 | 0.7382 | 0.4918 | spinal cord |
| CNGA1 | ENSG00000198515 | P29973 | 0.6656 | 0.4626 | urinary bladder |
| CNGA3 | ENSG00000144191 | Q16281 | 0.8959 | 0.7568 | pituitary gland |
| CNGA4 | ENSG00000132259 | Q8IV77 | 0.8702 | 0.5214 | fallopian tube |
| CNGB1 | ENSG00000070729 | Q14028 | 0.9262 | 0.8284 | hypothalamus |
| CNGB3 | ENSG00000170289 | Q9NQW8 | 0.9752 | 0.9511 | fallopian tube |
| CNIH2 | ENSG00000174871 | Q6PI25 | 0.7562 | 0.5911 | hippocampal |
| | | | | | formation |
| CNIH3 | ENSG00000143786 | Q8TBE1 | 0.7254 | 0.4483 | cerebral cortex |
| CNMD | ENSG00000136110 | 075829 | 0.8366 | 0.7254 | basal ganglia |
| CNNM1 | ENSG00000119946 | Q9NRU3 | 0.8366 | 0.6526 | cerebellum |
| CNR2 | ENSG00000188822 | P34972 | 0.9766 | 0.9672 | spleen |
| CNTNAP2 | ENSG00000174469 | Q9UHC6 | 0.8745 | 0.8133 | spinal cord |
| CNTNAP3B | ENSG00000154529 | Q96NUO | 0.7863 | 0.4851 | breast |
| CNTNAP4 | ENSG00000152910 | Q9C0A0 | 0.9269 | 0.8729 | cerebellum |
| CNTNAP5 | ENSG00000155052 | Q8WYK1 | 0.9008 | 0.8446 | cerebral cortex |
| COL13A1 | ENSG00000197467 | Q5TAT6 | 0.7396 | 0.41 | cerebellum |
| COL17A1 | ENSG00000065618 | Q9UMD9 | 0.7538 | 0.5497 | skin |
| COL25A1 | ENSG00000188517 | Q9BXS0 | 0.8455 | 0.5864 | pituitary gland |
| CORIN | ENSG00000145244 | Q9Y5Q5 | 0.8373 | 0.6117 | heart muscle |
| CR2 | ENSG00000117322 | P20023 | 0.9618 | 0.9157 | spleen |
| CRB1 | ENSG00000134376 | P82279 | 0.8149 | 0.7518 | cerebellum |
| CRB2 | ENSG00000148204 | Q5IJ48 | 0.7767 | 0.706 | basal ganglia |
| CRB3 | ENSG00000130545 | Q9BUF7 | 0.5596 | 0.4382 | esophagus |
| CRHR1 | ENSG00000120088 | P34998 | 0.8456 | 0.6816 | cerebellum |
| CRHR2 | ENSG00000106113 | Q13324 | 0.836 | 0.5029 | pituitary gland |
| CRLF2 | ENSG00000205755 | Q9HC73 | 0.843 | 0.6956 | lung |
| CRTAM | ENSG00000109943 | 095727 | 0.9618 | 0.7549 | cerebellum |
| CSMD1 | ENSG00000183117 | Q96PZ7 | 0.8515 | 0.7587 | cerebral cortex |
| CSMD2 | ENSG00000121904 | Q7Z408 | 0.774 | 0.6539 | cerebellum |
| CSMD3 | ENSG00000164796 | Q7Z407 | 0.8467 | 0.8064 | basal ganglia |
| CSPG5 | ENSG00000114646 | 095196 | 0.7452 | 0.6423 | cerebral cortex |
| CT83 | ENSG00000204019 | Q5H943 | 0.9972 | 0.9949 | salivary gland |
| CTLA4 | ENSG00000163599 | P16410 | 0.7908 | 0.5171 | small intestine |
| CTXN2 | ENSG00000233932 | POC2S0 | 0.8038 | 0.7611 | hypothalamus |
| CTXN3 | ENSG00000205279 | Q4LDR2 | 0.8686 | 0.8178 | cerebral cortex |
| CWH43 | ENSG00000109182 | Q9H720 | 0.7916 | 0.7203 | skin |
| CX3CR1 | ENSG00000168329 | P49238 | 0.7028 | 0.4454 | spinal cord |
| CXCR1 | ENSG00000163464 | P25024 | 0.8905 | 0.5619 | spleen |
| CXCR2 | ENSG00000180871 | P25025 | 0.7982 | 0.576 | spleen |
| CXCR3 | ENSG00000186810 | P49682 | 0.8596 | 0.6327 | spleen |
| CXCR5 | ENSG00000160683 | P32302 | 0.8137 | 0.5221 | spleen |
| CXCR6 | ENSG00000172215 | O00574 | 0.7626 | 0.4668 | spleen |
| CYP46A1 | ENSG00000036530 | Q9Y6A2 | 0.7299 | 0.4964 | basal ganglia |
| CYSLTR1 | ENSG00000173198 | Q9Y271 | 0.7058 | 0.4018 | spleen |
| CYSLTR2 | ENSG00000152207 | Q9NS75 | 0.7776 | 0.4897 | adrenal gland |
| DBH | ENSG00000123454 | P09172 | 0.9069 | 0.6122 | adrenal gland |
| DCC | ENSG00000187323 | P43146 | 0.8342 | 0.7006 | basal ganglia |
| DCHS2 | ENSG00000197410 | Q6V1P9 | 0.6957 | 0.5475 | colon |
| DCST1 | ENSG00000163357 | Q5T197 | 0.9499 | 0.8397 | skin |
| DCST2 | ENSG00000163354 | Q5T1A1 | 0.754 | 0.5165 | skin |
| DCSTAMP | ENSG00000164935 | Q9H295 | 0.9519 | 0.9188 | lung |
| DIO2 | ENSG00000211448 | Q92813 | 0.6367 | 0.4297 | thyroid gland |
| DIO3 | ENSG00000197406 | P55073 | 0.7628 | 0.5173 | cervix, uterine |
| DISP2 | ENSG00000140323 | A7MBM2 | 0.8262 | 0.5491 | cerebellum |
| DLK1 | ENSG00000185559 | P80370 | 0.8191 | 0.5867 | adrenal gland |
| DLK2 | ENSG00000171462 | Q6UY11 | 0.6504 | 0.4945 | prostate |
| DLL3 | ENSG00000090932 | Q9NYJ7 | 0.8117 | 0.7735 | basal ganglia |
| DNAJC22 | ENSG00000178401 | Q8N4W6 | 0.8014 | 0.5869 | liver |
| DNER | ENSG00000187957 | Q8NFT8 | 0.6092 | 0.4844 | hypothalamus |
| DPP10 | ENSG00000175497 | Q8N608 | 0.7894 | 0.6696 | cerebral cortex |
| DPP4 | ENSG00000197635 | P27487 | 0.6865 | 0.4677 | small intestine |
| DPP6 | ENSG00000130226 | P42658 | 0.6277 | 0.4764 | endometrium |
| DRD1 | ENSG00000184845 | P21728 | 0.9102 | 0.6467 | basal ganglia |
| DRD2 | ENSG00000149295 | P14416 | 0.8203 | 0.5983 | pituitary gland |
| DRD5 | ENSG00000169676 | P21918 | 0.8907 | 0.8184 | cerebral cortex |
| DSC1 | ENSG00000134765 | Q08554 | 0.9803 | 0.9427 | skin |
| DSC2 | ENSG00000134755 | Q02487 | 0.7146 | 0.4509 | esophagus |
| DSC3 | ENSG00000134762 | Q14574 | 0.8544 | 0.7665 | skin |
| DSCAM | ENSG00000171587 | O60469 | 0.7965 | 0.7756 | hypothalamus |
| DSG1 | ENSG00000134760 | Q02413 | 0.9193 | 0.8369 | skin |
| DSG2 | ENSG00000046604 | Q14126 | 0.5691 | 0.4327 | colon |
| DSG3 | ENSG00000134757 | P32926 | 0.8883 | 0.8524 | esophagus |
| DSG4 | ENSG00000175065 | Q86SJ6 | 0.9799 | 0.9545 | skin |
| DUOX1 | ENSG00000137857 | Q9NRD9 | 0.6518 | 0.5116 | lung |
| DUOX2 | ENSG00000140279 | Q9NRD8 | 0.7868 | 0.6292 | thyroid gland |
| DUOXA1 | ENSG00000140254 | Q1HG43 | 0.6971 | 0.5608 | esophagus |
| DYNAP | ENSG00000178690 | Q8N1N2 | 0.9761 | 0.9483 | esophagus |
| ECEL1 | ENSG00000171551 | O95672 | 0.7888 | 0.5018 | ovary |
| EDAR | ENSG00000135960 | Q9UNE0 | 0.8857 | 0.8186 | esophagus |
| EFNB3 | ENSG00000108947 | Q15768 | 0.6495 | 0.4066 | basal ganglia |
| EGF | ENSG00000138798 | P01133 | 0.8386 | 0.6819 | skeletal muscle |
| ELFN2 | ENSG00000166897 | Q5R3F8 | 0.8285 | 0.769 | cerebral cortex |
| ENPEP | ENSG00000138792 | Q07075 | 0.7491 | 0.4497 | small intestine |
| ENPP1 | ENSG00000197594 | P22413 | 0.6634 | 0.4243 | endometrium |
| ENPP3 | ENSG00000154269 | O14638 | 0.7105 | 0.5263 | salivary gland |
| ENTPD8 | ENSG00000188833 | Q5MY95 | 0.8489 | 0.731 | small intestine |
| EPGN | ENSG00000182585 | Q6UW88 | 0.8985 | 0.8211 | esophagus |
| EPHA1 | ENSG00000146904 | P21709 | 0.7307 | 0.5445 | esophagus |
| EPHA10 | ENSG00000183317 | Q5JZY3 | 0.7465 | 0.5994 | colon |
| EPHA5 | ENSG00000145242 | P54756 | 0.8268 | 0.6816 | cerebral cortex |
| EPHA6 | ENSG00000080224 | Q9UF33 | 0.7922 | 0.5514 | colon |
| EPHA7 | ENSG00000135333 | Q15375 | 0.7399 | 0.496 | colon |
| EPHA8 | ENSG00000070886 | P29322 | 0.9028 | 0.7866 | spleen |
| EPHB1 | ENSG00000154928 | P54762 | 0.7367 | 0.4094 | cerebellum |
| EPHX4 | ENSG00000172031 | Q8IUS5 | 0.8213 | 0.5259 | cerebral cortex |
| ERBB4 | ENSG00000178568 | Q15303 | 0.6627 | 0.5179 | fallopian tube |
| EREG | ENSG00000124882 | O14944 | 0.8382 | 0.7341 | skin |
| ERVFRD-1 | ENSG00000244476 | P60508 | 0.6812 | 0.6136 | adrenal gland |
| ERVMER34- | ENSG00000226887 | Q9H9K5 | 0.7591 | 0.5227 | ovary |
| 1 | | | | | |
| ERVW-1 | ENSG00000242950 | Q9UQF0 | 0.9183 | 0.8644 | cerebellum |
| ESR1 | ENSG00000091831 | P03372 | 0.7228 | 0.495 | cervix, uterine |
| ESYT3 | ENSG00000158220 | AOFGR9 | 0.6935 | 0.4867 | skin |
| EVC | ENSG00000072840 | P57679 | 0.7282 | 0.5347 | endometrium |
| F2RL2 | ENSG00000164220 | O00254 | 0.7389 | 0.5252 | colon |
| F2RL3 | ENSG00000127533 | Q96RI0 | 0.7305 | 0.4611 | lung |
| FAM151A | ENSG00000162391 | Q8WW52 | 0.9455 | 0.9038 | small intestine |
| FAM155A | ENSG00000204442 | B1AL88 | 0.7277 | 0.5824 | cerebral cortex |
| FAM155B | ENSG00000130054 | O75949 | 0.8259 | 0.569 | heart muscle |
| FAM163A | ENSG00000143340 | Q96GL9 | 0.9253 | 0.7271 | pituitary gland |
| FAM163B | ENSG00000196990 | POC2L3 | 0.8371 | 0.684 | cerebral cortex |
| FAM171A2 | ENSG00000161682 | A8MVWO | 0.681 | 0.4745 | cerebellum |
| FAM171B | ENSG00000144369 | Q6P995 | 0.6049 | 0.4019 | cerebral cortex |
| FAM187B | ENSG00000177558 | Q17R55 | 0.902 | 0.8341 | spleen |
| FAM189A1 | ENSG00000104059 | O60320 | 0.7129 | 0.5345 | hippocampal |
| | | | | | formation |
| FAM209A | ENSG00000124103 | Q5JX71 | 0.7554 | 0.4299 | spleen |
| FAP | ENSG00000078098 | Q12884 | 0.7726 | 0.513 | endometrium |
| FASLG | ENSG00000117560 | P48023 | 0.9253 | 0.6958 | spleen |
| FAT2 | ENSG00000086570 | Q9NYQ8 | 0.8516 | 0.6952 | cerebellum |
| FAT3 | ENSG00000165323 | Q8TDW7 | 0.7727 | 0.5779 | basal ganglia |
| FCAR | ENSG00000186431 | P24071 | 0.8301 | 0.5151 | spleen |
| FCER1A | ENSG00000179639 | P12319 | 0.7176 | 0.4535 | skin |
| FCER2 | ENSG00000104921 | P06734 | 0.9142 | 0.7048 | spleen |
| FCMR | ENSG00000162894 | 060667 | 0.8118 | 0.4981 | spleen |
| FCRL1 | ENSG00000163534 | Q96LA6 | 0.9534 | 0.9081 | spleen |
| FCRL2 | ENSG00000132704 | Q96LA5 | 0.936 | 0.8364 | spleen |
| FCRL3 | ENSG00000160856 | Q96P31 | 0.9569 | 0.8905 | spleen |
| FCRL4 | ENSG00000163518 | Q96PJ5 | 0.9848 | 0.9795 | small intestine |
| FCRL5 | ENSG00000143297 | Q96RD9 | 0.895 | 0.7421 | spleen |
| FCRL6 | ENSG00000181036 | Q6DN72 | 0.8649 | 0.4642 | spleen |
| FER1L6 | ENSG00000214814 | Q2WGJ9 | 0.9325 | 0.8828 | stomach |
| FFAR1 | ENSG00000126266 | 014842 | 0.8687 | 0.738 | ovary |
| FFAR2 | ENSG00000126262 | O15552 | 0.8748 | 0.6402 | spleen |
| FFAR3 | ENSG00000185897 | 014843 | 0.8504 | 0.6178 | adipose tissue |
| FFAR4 | ENSG00000186188 | Q5NUL3 | 0.8933 | 0.7082 | pituitary gland |
| FIBCD1 | ENSG00000130720 | Q8N539 | 0.8896 | 0.729 | hippocampal |
| | | | | | formation |
| FLT3 | ENSG00000122025 | P36888 | 0.8835 | 0.6222 | cerebellum |
| FNDC5 | ENSG00000160097 | Q8NAU1 | 0.7087 | 0.4366 | cerebellum |
| FNDC9 | ENSG00000172568 | Q8TBE3 | 0.8491 | 0.7807 | hypothalamus |
| FOLH1 | ENSG00000086205 | Q04609 | 0.7199 | 0.5144 | spinal cord |
| FPR2 | ENSG00000171049 | P25090 | 0.8596 | 0.5311 | spleen |
| FRAS1 | ENSG00000138759 | Q86XX4 | 0.8232 | 0.4932 | thyroid gland |
| FRMD5 | ENSG00000171877 | Q7Z6J6 | 0.7123 | 0.596 | spinal cord |
| FRRS1 | ENSG00000156869 | Q6ZNA5 | 0.6353 | 0.4465 | esophagus |
| FUT6 | ENSG00000156413 | P51993 | 0.8254 | 0.7699 | esophagus |
| FXYD2 | ENSG00000137731 | P54710 | 0.8746 | 0.5585 | kidney |
| FXYD3 | ENSG00000089356 | Q14802 | 0.5752 | 0.4063 | colon |
| FXYD4 | ENSG00000150201 | P59646 | 0.9483 | 0.8097 | kidney |
| FXYD7 | ENSG00000221946 | P58549 | 0.7272 | 0.5279 | cerebral cortex |
| FZD10 | ENSG00000111432 | Q9ULW2 | 0.7497 | 0.5184 | esophagus |
| FZD9 | ENSG00000188763 | O00144 | 0.6263 | 0.4699 | pituitary gland |
| GABBR2 | ENSG00000136928 | O75899 | 0.8414 | 0.774 | cerebral cortex |
| GABRA1 | ENSG00000022355 | P14867 | 0.8723 | 0.8224 | cerebellum |
| GABRA2 | ENSG00000151834 | P47869 | 0.7724 | 0.6364 | cerebral cortex |
| GABRA3 | ENSG00000011677 | P34903 | 0.8469 | 0.7788 | cerebral cortex |
| GABRA4 | ENSG00000109158 | P48169 | 0.8951 | 0.8263 | basal ganglia |
| GABRA5 | ENSG00000186297 | P31644 | 0.8825 | 0.8281 | basal ganglia |
| GABRA6 | ENSG00000145863 | Q16445 | 0.9874 | 0.9614 | cerebellum |
| GABRB1 | ENSG00000163288 | P18505 | 0.8566 | 0.7934 | basal ganglia |
| GABRB2 | ENSG00000145864 | P47870 | 0.8578 | 0.7178 | cerebellum |
| GABRB3 | ENSG00000166206 | P28472 | 0.6826 | 0.4658 | cerebral cortex |
| GABRD | ENSG00000187730 | O14764 | 0.8541 | 0.6607 | cerebellum |
| GABRE | ENSG00000102287 | P78334 | 0.8923 | 0.746 | hypothalamus |
| GABRG1 | ENSG00000163285 | Q8N1C3 | 0.8063 | 0.7803 | basal ganglia |
| GABRG2 | ENSG00000113327 | P18507 | 0.828 | 0.7812 | cerebral cortex |
| GABRG3 | ENSG00000182256 | Q99928 | 0.8362 | 0.7058 | pituitary gland |
| GABRP | ENSG00000094755 | O00591 | 0.7572 | 0.6659 | breast |
| GABRR1 | ENSG00000146276 | P24046 | 0.9375 | 0.9375 | esophagus |
| GALR1 | ENSG00000166573 | P47211 | 0.9152 | 0.7022 | pituitary gland |
| GALR2 | ENSG00000182687 | O43603 | 0.931 | 0.8083 | colon |
| GALR3 | ENSG00000128310 | O60755 | 0.9646 | 0.917 | cerebellum |
| GAPT | ENSG00000175857 | Q8N292 | 0.8353 | 0.4694 | spleen |
| GCGR | ENSG00000215644 | P47871 | 0.932 | 0.7647 | liver |
| GDPD2 | ENSG00000130055 | Q9HCC8 | 0.8428 | 0.6784 | spleen |
| GDPD4 | ENSG00000178795 | Q6W3E5 | 0.9361 | 0.9258 | adrenal gland |
| GGT6 | ENSG00000167741 | Q6P531 | 0.6893 | 0.6128 | skin |
| GHRHR | ENSG00000106128 | Q02643 | 0.9953 | 0.9814 | pituitary gland |
| GHSR | ENSG00000121853 | Q92847 | 0.9874 | 0.9735 | pituitary gland |
| GJA3 | ENSG00000121743 | Q9Y6H8 | 0.9627 | 0.8685 | heart muscle |
| GJB1 | ENSG00000169562 | P08034 | 0.673 | 0.507 | liver |
| GJB2 | ENSG00000165474 | P29033 | 0.776 | 0.5476 | esophagus |
| GJB3 | ENSG00000188910 | O75712 | 0.8432 | 0.6709 | skin |
| GJB4 | ENSG00000189433 | Q9NTQ9 | 0.9564 | 0.8901 | skin |
| GJB5 | ENSG00000189280 | 095377 | 0.8284 | 0.6748 | skin |
| GJB6 | ENSG00000121742 | O95452 | 0.7078 | 0.6111 | esophagus |
| GJB7 | ENSG00000164411 | Q6PEYO | 0.9692 | 0.9486 | pituitary gland |
| GJC3 | ENSG00000176402 | Q8NFK1 | 0.7789 | 0.4855 | salivary gland |
| GJD2 | ENSG00000159248 | Q9UKL4 | 0.912 | 0.8031 | pituitary gland |
| GJD3 | ENSG00000183153 | Q8N144 | 0.7456 | 0.5963 | urinary bladder |
| GJD4 | ENSG00000177291 | Q96KN9 | 0.9886 | 0.9833 | cerebral cortex |
| GLDN | ENSG00000186417 | Q6ZMI3 | 0.6649 | 0.4243 | spinal cord |
| GLP1R | ENSG00000112164 | P43220 | 0.934 | 0.8555 | pancreas |
| GLP2R | ENSG00000065325 | O95838 | 0.7814 | 0.6252 | colon |
| GLRA1 | ENSG00000145888 | P23415 | 0.9452 | 0.9279 | hypothalamus |
| GLRA2 | ENSG00000101958 | P23416 | 0.893 | 0.8292 | cerebellum |
| GLRA3 | ENSG00000145451 | 075311 | 0.8658 | 0.8234 | cerebral cortex |
| GNRHR | ENSG00000109163 | P30968 | 0.972 | 0.8217 | pituitary gland |
| GP5 | ENSG00000178732 | P40197 | 0.8871 | 0.8448 | fallopian tube |
| GP6 | ENSG00000088053 | Q9HCN6 | 0.7625 | 0.4339 | cerebellum |
| GP9 | ENSG00000169704 | P14770 | 0.9667 | 0.9286 | spleen |
| GPA33 | ENSG00000143167 | Q99795 | 0.9605 | 0.9499 | colon |
| GPBAR1 | ENSG00000179921 | Q8TDU6 | 0.7547 | 0.4867 | adipose tissue |
| GPM6A | ENSG00000150625 | P51674 | 0.6609 | 0.4441 | cerebellum |
| GPR1 | ENSG00000183671 | P46091 | 0.7682 | 0.5848 | vagina |
| GPR101 | ENSG00000165370 | Q96P66 | 0.9832 | 0.976 | basal ganglia |
| GPR119 | ENSG00000147262 | Q8TDV5 | 1 | 1 | pancreas |
| GPR12 | ENSG00000132975 | P47775 | 0.8467 | 0.7404 | cerebellum |
| GPR139 | ENSG00000180269 | Q6DWJ6 | 0.9831 | 0.978 | basal ganglia |
| GPR141 | ENSG00000187037 | Q7Z602 | 0.8654 | 0.5665 | spleen |
| GPR142 | ENSG00000257008 | Q7Z601 | 1 | 1 | cerebellum |
| GPR148 | ENSG00000173302 | Q8TDV2 | 0.9604 | 0.9483 | cerebellum |
| GPR149 | ENSG00000174948 | Q86SP6 | 0.9592 | 0.9361 | basal ganglia |
| GPR15 | ENSG00000154165 | P49685 | 0.8276 | 0.7181 | colon |
| GPR150 | ENSG00000178015 | Q8NGU9 | 0.7823 | 0.4746 | pancreas |
| GPR152 | ENSG00000175514 | Q8TDT2 | 0.9688 | 0.9688 | small intestine |
| GPR156 | ENSG00000175697 | Q8NFN8 | 0.8632 | 0.7378 | urinary bladder |
| GPR158 | ENSG00000151025 | Q5T848 | 0.9258 | 0.8411 | cerebellum |
| GPR171 | ENSG00000174946 | O14626 | 0.724 | 0.519 | spleen |
| GPR174 | ENSG00000147138 | Q9BXC1 | 0.8314 | 0.6287 | spleen |
| GPR18 | ENSG00000125245 | Q14330 | 0.9105 | 0.644 | spleen |
| GPR182 | ENSG00000166856 | O15218 | 0.9168 | 0.7009 | spleen |
| GPR19 | ENSG00000183150 | Q15760 | 0.7766 | 0.6709 | cerebellum |
| GPR20 | ENSG00000204882 | Q99678 | 0.7043 | 0.5179 | cervix, uterine |
| GPR21 | ENSG00000188394 | Q99679 | 0.7192 | 0.5098 | adipose tissue |
| GPR22 | ENSG00000172209 | Q99680 | 0.8477 | 0.7564 | cerebral cortex |
| GPR25 | ENSG00000170128 | O00155 | 0.8161 | 0.7352 | small intestine |
| GPR26 | ENSG00000154478 | Q8NDV2 | 0.9161 | 0.8789 | cerebral cortex |
| GPR37 | ENSG00000170775 | O15354 | 0.7701 | 0.5764 | spinal cord |
| GPR37L1 | ENSG00000170075 | O60883 | 0.7768 | 0.7402 | cerebral cortex |
| GPR39 | ENSG00000183840 | O43194 | 0.7468 | 0.5772 | urinary bladder |
| GPR42 | ENSG00000126251 | 015529 | 0.9233 | 0.874 | adipose tissue |
| GPR45 | ENSG00000135973 | Q9Y5Y3 | 0.892 | 0.8426 | hypothalamus |
| GPR50 | ENSG00000102195 | Q13585 | 0.9923 | 0.9828 | pituitary gland |
| GPR52 | ENSG00000203737 | Q9Y2T5 | 0.9607 | 0.8847 | basal ganglia |
| GPR55 | ENSG00000135898 | Q9Y2T6 | 0.8655 | 0.6735 | basal ganglia |
| GPR6 | ENSG00000146360 | P46095 | 0.9506 | 0.9037 | basal ganglia |
| GPR61 | ENSG00000156097 | Q9BZJ8 | 0.887 | 0.8248 | cerebellum |
| GPR62 | ENSG00000180929 | Q9BZJ7 | 0.8459 | 0.704 | spinal cord |
| GPR78 | ENSG00000155269 | Q96P69 | 0.9937 | 0.9895 | esophagus |
| GPR82 | ENSG00000171657 | Q96P67 | 0.8607 | 0.6348 | small intestine |
| GPR83 | ENSG00000123901 | Q9NYM4 | 0.9016 | 0.7456 | cerebellum |
| GPR85 | ENSG00000164604 | P60893 | 0.6896 | 0.5482 | cerebellum |
| GPR87 | ENSG00000138271 | Q9BY21 | 0.84 | 0.8133 | esophagus |
| GPR88 | ENSG00000181656 | Q9GZNO | 0.9049 | 0.6565 | basal ganglia |
| GPRC5A | ENSG00000013588 | Q8NFJ5 | 0.6311 | 0.4064 | lung |
| GPRC5D | ENSG00000111291 | Q9NZD1 | 0.8638 | 0.5143 | skin |
| GPRC6A | ENSG00000173612 | Q5T6X5 | 0.9927 | 0.9881 | salivary gland |
| GRAMD1B | ENSG00000023171 | Q3KR37 | 0.7086 | 0.4335 | cerebellum |
| GRAMD2A | ENSG00000175318 | Q8IUY3 | 0.7208 | 0.5538 | fallopian tube |
| GREB1 | ENSG00000196208 | Q4ZG55 | 0.7032 | 0.4087 | ovary |
| GREB1L | ENSG00000141449 | Q9C091 | 0.6365 | 0.4494 | fallopian tube |
| GRIA1 | ENSG00000155511 | P42261 | 0.7876 | 0.689 | cerebellum |
| GRIA2 | ENSG00000120251 | P42262 | 0.7131 | 0.6071 | cerebral cortex |
| GRIA3 | ENSG00000125675 | P42263 | 0.7195 | 0.5116 | cerebral cortex |
| GRIA4 | ENSG00000152578 | P48058 | 0.8019 | 0.6615 | cerebellum |
| GRID1 | ENSG00000182771 | Q9ULK0 | 0.7582 | 0.6447 | basal ganglia |
| GRID2 | ENSG00000152208 | O43424 | 0.9194 | 0.8062 | cerebellum |
| GRIK1 | ENSG00000171189 | P39086 | 0.7749 | 0.6756 | hypothalamus |
| GRIK2 | ENSG00000164418 | Q13002 | 0.8032 | 0.5469 | cerebellum |
| GRIK3 | ENSG00000163873 | Q13003 | 0.7189 | 0.5128 | pituitary gland |
| GRIK4 | ENSG00000149403 | Q16099 | 0.679 | 0.608 | cerebral cortex |
| GRIN1 | ENSG00000176884 | Q05586 | 0.8079 | 0.7595 | cerebral cortex |
| GRIN2A | ENSG00000183454 | Q12879 | 0.8674 | 0.7212 | cerebral cortex |
| GRIN2B | ENSG00000273079 | Q13224 | 0.896 | 0.8651 | cerebral cortex |
| GRIN2C | ENSG00000161509 | Q14957 | 0.832 | 0.6319 | cerebellum |
| GRIN2D | ENSG00000105464 | O15399 | 0.773 | 0.5131 | hypothalamus |
| GRIN3A | ENSG00000198785 | Q8TCU5 | 0.8259 | 0.5847 | cerebral cortex |
| GRIN3B | ENSG00000116032 | O60391 | 0.9012 | 0.6475 | fallopian tube |
| GRM1 | ENSG00000152822 | Q13255 | 0.9279 | 0.8348 | cerebellum |
| GRM2 | ENSG00000164082 | Q14416 | 0.8949 | 0.6648 | cerebral cortex |
| GRM3 | ENSG00000198822 | Q14832 | 0.8189 | 0.7909 | basal ganglia |
| GRM4 | ENSG00000124493 | Q14833 | 0.9429 | 0.8382 | cerebellum |
| GRM5 | ENSG00000168959 | P41594 | 0.8985 | 0.8237 | cerebral cortex |
| GRM6 | ENSG00000113262 | 015303 | 0.8504 | 0.6234 | pituitary gland |
| GRM7 | ENSG00000196277 | Q14831 | 0.8487 | 0.7194 | cerebral cortex |
| GRM8 | ENSG00000179603 | O00222 | 0.8196 | 0.6639 | cerebral cortex |
| GRPR | ENSG00000126010 | P30550 | 0.9489 | 0.8745 | pancreas |
| GSDMA | ENSG00000167914 | Q96QA5 | 0.9435 | 0.7313 | skin |
| GSDMC | ENSG00000147697 | Q9BYG8 | 0.8746 | 0.8415 | esophagus |
| GSG1L | ENSG00000169181 | Q6UXU4 | 0.7886 | 0.7249 | basal ganglia |
| GSG1L2 | ENSG00000214978 | A8MUP6 | 1 | 1 | cerebral cortex |
| GUCY2C | ENSG00000070019 | P25092 | 0.9576 | 0.9313 | small intestine |
| GUCY2D | ENSG00000132518 | Q02846 | 0.9688 | 0.9688 | esophagus |
| GUCY2F | ENSG00000101890 | P51841 | 1 | 1 | fallopian tube |
| GYPA | ENSG00000170180 | P02724 | 0.9617 | 0.8728 | spleen |
| GYPB | ENSG00000250361 | P06028 | 0.977 | 0.9135 | spleen |
| GYPE | ENSG00000197465 | P15421 | 0.8154 | 0.5311 | spleen |
| HAS1 | ENSG00000105509 | Q92839 | 0.7883 | 0.5166 | adipose tissue |
| HAS2 | ENSG00000170961 | Q92819 | 0.725 | 0.4638 | adipose tissue |
| HAS3 | ENSG00000103044 | O00219 | 0.6919 | 0.4244 | esophagus |
| HAVCR1 | ENSG00000113249 | Q96D42 | 0.9701 | 0.8838 | kidney |
| HCAR1 | ENSG00000196917 | Q9BXC0 | 0.9886 | 0.9833 | breast |
| HCAR2 | ENSG00000182782 | Q8TDS4 | 0.7293 | 0.5939 | esophagus |
| HCAR3 | ENSG00000255398 | P49019 | 0.7871 | 0.6554 | spleen |
| HCN1 | ENSG00000164588 | O60741 | 0.8665 | 0.7716 | cerebral cortex |
| HCN2 | ENSG00000099822 | Q9UL51 | 0.7282 | 0.65 | spinal cord |
| HCN4 | ENSG00000138622 | Q9Y3Q4 | 0.9412 | 0.8441 | heart muscle |
| HCRTR1 | ENSG00000121764 | O43613 | 0.8759 | 0.7665 | hypothalamus |
| HCRTR2 | ENSG00000137252 | 043614 | 0.9406 | 0.8672 | hypothalamus |
| HEPACAM | ENSG00000165478 | Q14CZ8 | 0.7574 | 0.7147 | cerebral cortex |
| HEPACAM2 | ENSG00000188175 | A8MVW5 | 0.8845 | 0.8389 | colon |
| HEPHL1 | ENSG00000181333 | Q6MZM0 | 0.9171 | 0.904 | esophagus |
| HHLA2 | ENSG00000114455 | Q9UM44 | 0.9206 | 0.8343 | colon |
| HIGD1C | ENSG00000214511 | A8MV81 | 0.853 | 0.7423 | cervix, uterine |
| HLA-DQB2 | ENSG00000232629 | P05538 | 0.7445 | 0.4372 | skin |
| HLA-G | ENSG00000204632 | P17693 | 0.8905 | 0.5237 | pituitary gland |
| HPN | ENSG00000105707 | P05981 | 0.7194 | 0.4136 | liver |
| HRH2 | ENSG00000113749 | P25021 | 0.6272 | 0.4281 | heart muscle |
| HRH3 | ENSG00000101180 | Q9Y5N1 | 0.8476 | 0.8071 | cerebellum |
| HRH4 | ENSG00000134489 | Q9H3N8 | 0.9062 | 0.9063 | adipose tissue |
| HRK | ENSG00000135116 | O00198 | 0.8603 | 0.7482 | hippocampal |
| | | | | | formation |
| HS6ST2 | ENSG00000171004 | Q96MM7 | 0.6839 | 0.4445 | basal ganglia |
| HS6ST3 | ENSG00000185352 | Q8IZP7 | 0.8278 | 0.5859 | cerebral cortex |
| HSD17B2 | ENSG00000086696 | P37059 | 0.7606 | 0.6499 | urinary bladder |
| HTR1A | ENSG00000178394 | P08908 | 0.9345 | 0.8929 | cerebral cortex |
| HTR1B | ENSG00000135312 | P28222 | 0.8025 | 0.5465 | basal ganglia |
| HTR1D | ENSG00000179546 | P28221 | 0.9696 | 0.957 | small intestine |
| HTR1E | ENSG00000168830 | P28566 | 0.8614 | 0.7582 | cerebral cortex |
| HTR1F | ENSG00000179097 | P30939 | 0.8265 | 0.6591 | adipose tissue |
| HTR2A | ENSG00000102468 | P28223 | 0.8761 | 0.6431 | cerebral cortex |
| HTR2B | ENSG00000135914 | P41595 | 0.765 | 0.5872 | endometrium |
| HTR3A | ENSG00000166736 | P46098 | 0.8713 | 0.7292 | salivary gland |
| HTR3B | ENSG00000149305 | O95264 | 0.9797 | 0.9102 | cerebral cortex |
| HTR3C | ENSG00000178084 | Q8WXA8 | 0.9933 | 0.978 | lung |
| HTR3E | ENSG00000186038 | A5X5Y0 | 0.9688 | 0.9687 | colon |
| HTR4 | ENSG00000164270 | Q13639 | 0.895 | 0.8241 | small intestine |
| HTR5A | ENSG00000157219 | P47898 | 0.8999 | 0.8409 | cerebellum |
| HTR6 | ENSG00000158748 | P50406 | 0.9656 | 0.915 | basal ganglia |
| HTR7 | ENSG00000148680 | P34969 | 0.7577 | 0.4721 | hypothalamus |
| HYAL4 | ENSG00000106302 | Q2M3T9 | 0.9624 | 0.9222 | skeletal muscle |
| ICAM4 | ENSG00000105371 | Q14773 | 0.8794 | 0.6411 | lung |
| ICAM5 | ENSG00000105376 | Q9UMFO | 0.8386 | 0.7234 | basal ganglia |
| ICOS | ENSG00000163600 | Q9Y6W8 | 0.8638 | 0.7395 | small intestine |
| IFITM10 | ENSG00000244242 | A6NMDO | 0.8211 | 0.4075 | adrenal gland |
| IFITM5 | ENSG00000206013 | A6NNB3 | 0.9589 | 0.8481 | pancreas |
| IGDCC3 | ENSG00000174498 | Q8IVU1 | 0.8506 | 0.6279 | cerebellum |
| IGDCC4 | ENSG00000103742 | Q8TDY8 | 0.6707 | 0.4077 | ovary |
| IGSF1 | ENSG00000147255 | Q8N6C5 | 0.8291 | 0.4977 | pituitary gland |
| IGSF11 | ENSG00000144847 | Q5DX21 | 0.6815 | 0.4547 | spinal cord |
| IGSF23 | ENSG00000216588 | A1L1A6 | 0.9636 | 0.9463 | liver |
| IGSF5 | ENSG00000183067 | Q9NSI5 | 0.7891 | 0.576 | heart muscle |
| IGSF9 | ENSG00000085552 | Q9P2J2 | 0.6721 | 0.5287 | skin |
| IL12RB1 | ENSG00000096996 | P42701 | 0.8087 | 0.4037 | spleen |
| IL12RB2 | ENSG00000081985 | Q99665 | 0.779 | 0.5077 | skeletal muscle |
| IL13RA2 | ENSG00000123496 | Q14627 | 0.7938 | 0.5348 | pituitary gland |
| IL17RE | ENSG00000163701 | Q8NFR9 | 0.7329 | 0.482 | skin |
| IL18RAP | ENSG00000115607 | O95256 | 0.8142 | 0.4937 | spleen |
| IL1R2 | ENSG00000115590 | P27930 | 0.6544 | 0.4119 | spleen |
| IL1RAPL1 | ENSG00000169306 | Q9NZN1 | 0.8728 | 0.7725 | spinal cord |
| IL1RAPL2 | ENSG00000189108 | Q9NP60 | 0.9197 | 0.9001 | cerebral cortex |
| IL1RL2 | ENSG00000115598 | Q9HB29 | 0.7549 | 0.4599 | skin |
| IL20RB | ENSG00000174564 | Q6UXLO | 0.8489 | 0.706 | skin |
| IL21R | ENSG00000103522 | Q9HBE5 | 0.8724 | 0.6412 | spleen |
| IL22RA1 | ENSG00000142677 | Q8N6P7 | 0.8178 | 0.6222 | pancreas |
| IL23R | ENSG00000162594 | Q5VWK5 | 1 | 1 | adrenal gland |
| IL2RA | ENSG00000134460 | P01589 | 0.8174 | 0.6083 | spleen |
| IL2RB | ENSG00000100385 | P14784 | 0.8081 | 0.4812 | spleen |
| IL31RA | ENSG00000164509 | Q8NI17 | 0.9443 | 0.9138 | skin |
| IL5RA | ENSG00000091181 | Q01344 | 0.8179 | 0.6165 | fallopian tube |
| IL7R | ENSG00000168685 | P16871 | 0.6827 | 0.4251 | lung |
| IL9R | ENSG00000124334 | Q01113 | 0.9354 | 0.9094 | spleen |
| ILDR1 | ENSG00000145103 | Q86SU0 | 0.6575 | 0.5789 | salivary gland |
| IMPG2 | ENSG00000081148 | Q9BZV3 | 0.9391 | 0.645 | fallopian tube |
| INSRR | ENSG00000027644 | P14616 | 0.803 | 0.6557 | kidney |
| ISLR2 | ENSG00000167178 | Q6UXK2 | 0.738 | 0.4434 | basal ganglia |
| ITGA11 | ENSG00000137809 | Q9UKX5 | 0.7224 | 0.4267 | endometrium |
| ITGA2B | ENSG00000005961 | P08514 | 0.6981 | 0.4019 | spleen |
| ITGA4 | ENSG00000115232 | P13612 | 0.7513 | 0.4335 | spleen |
| ITGAD | ENSG00000156886 | Q13349 | 0.9882 | 0.9413 | spleen |
| ITGAE | ENSG00000083457 | P38570 | 0.8396 | 0.6669 | lung |
| ITGB3 | ENSG00000259207 | P05106 | 0.6552 | 0.4053 | thyroid gland |
| ITGB6 | ENSG00000115221 | P18564 | 0.7393 | 0.6172 | urinary bladder |
| ITPRIPL1 | ENSG00000198885 | Q6GPH6 | 0.6527 | 0.407 | cerebellum |
| IYD | ENSG00000009765 | Q6PHWO | 0.9648 | 0.9057 | thyroid gland |
| IZUMO1 | ENSG00000182264 | Q8IYV9 | 0.8913 | 0.8269 | lung |
| IZUMO2 | ENSG00000161652 | Q6UXV1 | 0.9062 | 0.9063 | midbrain |
| JPH1 | ENSG00000104369 | Q9HDC5 | 0.7908 | 0.4363 | skeletal muscle |
| JPH2 | ENSG00000149596 | Q9BR39 | 0.5991 | 0.4408 | colon |
| JPH3 | ENSG00000154118 | Q8WXH2 | 0.7716 | 0.6968 | cerebral cortex |
| JPH4 | ENSG00000092051 | Q96JJ6 | 0.6412 | 0.4812 | cerebellum |
| KCNA1 | ENSG00000111262 | Q09470 | 0.9089 | 0.766 | cerebellum |
| KCNA10 | ENSG00000143105 | Q16322 | 1 | 1 | lung |
| KCNA2 | ENSG00000177301 | P16389 | 0.8284 | 0.6763 | cerebellum |
| KCNA3 | ENSG00000177272 | P22001 | 0.7563 | 0.4651 | lung |
| KCNA4 | ENSG00000182255 | P22459 | 0.8744 | 0.7585 | basal ganglia |
| KCNA7 | ENSG00000104848 | Q96RP8 | 0.9897 | 0.9748 | skeletal muscle |
| KCNB2 | ENSG00000182674 | Q92953 | 0.9062 | 0.8329 | cerebral cortex |
| KCNC1 | ENSG00000129159 | P48547 | 0.9012 | 0.7854 | cerebellum |
| KCNC2 | ENSG00000166006 | Q96PR1 | 0.888 | 0.8173 | cerebral cortex |
| KCND2 | ENSG00000184408 | Q9NZV8 | 0.8448 | 0.605 | cerebellum |
| KCNE1 | ENSG00000180509 | P15382 | 0.7597 | 0.5913 | lung |
| KCNE2 | ENSG00000159197 | Q9Y6J6 | 0.9248 | 0.4234 | stomach |
| KCNE4 | ENSG00000152049 | Q8WWG9 | 0.9104 | 0.7256 | endometrium |
| KCNE5 | ENSG00000176076 | Q9UJ90 | 0.7705 | 0.5849 | midbrain |
| KCNF1 | ENSG00000162975 | Q9H3M0 | 0.8289 | 0.7134 | cerebral cortex |
| KCNG1 | ENSG00000026559 | Q9UIX4 | 0.7613 | 0.4602 | endometrium |
| KCNG2 | ENSG00000178342 | Q9UJ96 | 0.8873 | 0.7214 | hippocampal |
| | | | | | formation |
| KCNG3 | ENSG00000171126 | Q8TAE7 | 0.8506 | 0.7951 | basal ganglia |
| KCNG4 | ENSG00000168418 | Q8TDN1 | 0.8584 | 0.8073 | midbrain |
| KCNH1 | ENSG00000143473 | O95259 | 0.9122 | 0.7757 | cerebellum |
| KCNH3 | ENSG00000135519 | Q9ULD8 | 0.7507 | 0.5695 | cerebral cortex |
| KCNH4 | ENSG00000089558 | Q9UQ05 | 0.9035 | 0.7533 | basal ganglia |
| KCNH5 | ENSG00000140015 | Q8NCM2 | 0.9479 | 0.8973 | cerebral cortex |
| KCNH6 | ENSG00000173826 | Q9H252 | 0.8628 | 0.783 | pituitary gland |
| KCNH7 | ENSG00000184611 | Q9NS40 | 0.9514 | 0.9078 | cerebellum |
| KCNH8 | ENSG00000183960 | Q96L42 | 0.7513 | 0.5737 | pituitary gland |
| KCNJ1 | ENSG00000151704 | P48048 | 0.9594 | 0.778 | kidney |
| KCNJ10 | ENSG00000177807 | P78508 | 0.7868 | 0.7152 | spinal cord |
| KCNJ12 | ENSG00000184185 | Q14500 | 0.8054 | 0.4361 | cerebellum |
| KCNJ13 | ENSG00000115474 | O60928 | 0.9437 | 0.6433 | small intestine |
| KCNJ15 | ENSG00000157551 | Q99712 | 0.6926 | 0.5362 | kidney |
| KCNJ16 | ENSG00000153822 | Q9NPI9 | 0.7824 | 0.5959 | thyroid gland |
| KCNJ3 | ENSG00000162989 | P48549 | 0.8308 | 0.63 | cerebellum |
| KCNJ4 | ENSG00000168135 | P48050 | 0.8502 | 0.7665 | cerebral cortex |
| KCNJ5 | ENSG00000120457 | P48544 | 0.8368 | 0.5615 | adrenal gland |
| KCNJ6 | ENSG00000157542 | P48051 | 0.97 | 0.9177 | pituitary gland |
| KCNJ9 | ENSG00000162728 | Q92806 | 0.8281 | 0.7842 | cerebellum |
| KCNK10 | ENSG00000100433 | P57789 | 0.7914 | 0.6725 | cerebellum |
| KCNK12 | ENSG00000184261 | Q9HB15 | 0.8822 | 0.796 | cerebellum |
| KCNK13 | ENSG00000152315 | Q9HB14 | 0.7547 | 0.5196 | thyroid gland |
| KCNK16 | ENSG00000095981 | Q96T55 | 0.9896 | 0.978 | pancreas |
| KCNK17 | ENSG00000124780 | Q96T54 | 0.8081 | 0.5573 | lung |
| KCNK18 | ENSG00000186795 | Q7Z418 | 1 | 1 | basal ganglia |
| KCNK2 | ENSG00000082482 | O95069 | 0.7791 | 0.4459 | adrenal gland |
| KCNK4 | ENSG00000182450 | Q9NYG8 | 0.8665 | 0.8379 | basal ganglia |
| KCNK5 | ENSG00000164626 | O95279 | 0.6005 | 0.4486 | small intestine |
| KCNK9 | ENSG00000169427 | Q9NPC2 | 0.9534 | 0.8363 | cerebellum |
| KCNMB1 | ENSG00000145936 | Q16558 | 0.6618 | 0.4612 | colon |
| KCNMB2 | ENSG00000197584 | Q9Y691 | 0.6791 | 0.4511 | ovary |
| KCNN1 | ENSG00000105642 | Q92952 | 0.8156 | 0.7028 | hippocampal |
| | | | | | formation |
| KCNN2 | ENSG00000080709 | Q9H2S1 | 0.6898 | 0.412 | adrenal gland |
| KCNN4 | ENSG00000104783 | O15554 | 0.6544 | 0.4194 | salivary gland |
| KCNQ1 | ENSG00000053918 | P51787 | 0.6744 | 0.4019 | adrenal gland |
| KCNQ2 | ENSG00000075043 | O43526 | 0.7884 | 0.7263 | cerebellum |
| KCNQ3 | ENSG00000184156 | O43525 | 0.7617 | 0.5624 | cerebral cortex |
| KCNQ4 | ENSG00000117013 | P56696 | 0.7056 | 0.4525 | colon |
| KCNQ5 | ENSG00000185760 | Q9NR82 | 0.8074 | 0.5949 | cerebral cortex |
| KCNS1 | ENSG00000124134 | Q96KK3 | 0.9432 | 0.719 | cerebral cortex |
| KCNS2 | ENSG00000156486 | Q9ULS6 | 0.8596 | 0.5625 | cerebral cortex |
| KCNT1 | ENSG00000107147 | Q5JUK3 | 0.8171 | 0.653 | cerebellum |
| KCNT2 | ENSG00000162687 | Q6UVM3 | 0.8046 | 0.4677 | ovary |
| KCNU1 | ENSG00000215262 | A8MYU2 | 1 | 1 | prostate |
| KCNV1 | ENSG00000164794 | Q6PIU1 | 0.9393 | 0.8616 | cerebral cortex |
| KCNV2 | ENSG00000168263 | Q8TDN2 | 0.9034 | 0.758 | cerebellum |
| KEL | ENSG00000197993 | P23276 | 0.7848 | 0.4941 | spleen |
| KIAA0319 | ENSG00000137261 | Q5VV43 | 0.788 | 0.6989 | cerebellum |
| KIAA1549 | ENSG00000122778 | Q9HCM3 | 0.6062 | 0.4282 | lung |
| KIAA1549L | ENSG00000110427 | Q6ZVL6 | 0.9258 | 0.8636 | cerebral cortex |
| KIR2DL3 | ENSG00000243772 | P43628 | 0.9791 | 0.9614 | spleen |
| KIR2DL4 | ENSG00000189013 | Q99706 | 0.963 | 0.8841 | spleen |
| KIR3DL1 | ENSG00000167633 | P43629 | 0.9859 | 0.9806 | spleen |
| KIR3DL2 | ENSG00000240403 | P43630 | 0.9665 | 0.9409 | spleen |
| KIRREL3 | ENSG00000149571 | Q8IZU9 | 0.7495 | 0.6258 | basal ganglia |
| KISS1R | ENSG00000116014 | Q969F8 | 0.8586 | 0.6747 | hypothalamus |
| KITLG | ENSG00000049130 | P21583 | 0.6473 | 0.4232 | urinary bladder |
| KL | ENSG00000133116 | Q9UEF7 | 0.7532 | 0.4399 | kidney |
| KLB | ENSG00000134962 | Q86Z14 | 0.8862 | 0.7648 | adipose tissue |
| KLHDC7A | ENSG00000179023 | Q5VTJ3 | 0.8447 | 0.6983 | kidney |
| KLRB1 | ENSG00000111796 | Q12918 | 0.8018 | 0.5329 | spleen |
| KLRC1 | ENSG00000134545 | P26715 | 0.7857 | 0.4381 | spleen |
| KLRC3 | ENSG00000205810 | Q07444 | 0.7624 | 0.6769 | amygdala |
| KLRC4 | ENSG00000183542 | O43908 | 0.7215 | 0.4204 | spleen |
| KLRD1 | ENSG00000134539 | Q13241 | 0.848 | 0.4559 | spleen |
| KLRF1 | ENSG00000150045 | Q9NZS2 | 0.969 | 0.952 | spleen |
| KLRF2 | ENSG00000256797 | D3WOD1 | 1 | 1 | skin |
| KLRG2 | ENSG00000188883 | A4D1S0 | 0.8622 | 0.7762 | thyroid gland |
| KREMEN2 | ENSG00000131650 | Q8NCW0 | 0.8559 | 0.6241 | skin |
| LAG3 | ENSG00000089692 | P18627 | 0.7344 | 0.5106 | ovary |
| LAMP5 | ENSG00000125869 | Q9UJQ1 | 0.7729 | 0.5246 | basal ganglia |
| LAX1 | ENSG00000122188 | Q8IWV1 | 0.8727 | 0.69 | spleen |
| LCT | ENSG00000115850 | P09848 | 0.9832 | 0.9782 | small intestine |
| LDLRAD1 | ENSG00000203985 | Q5T700 | 0.9468 | 0.8963 | fallopian tube |
| LEMD1 | ENSG00000186007 | Q68G75 | 0.8832 | 0.8108 | cerebellum |
| LGR5 | ENSG00000139292 | O75473 | 0.7277 | 0.4632 | skeletal muscle |
| LHCGR | ENSG00000138039 | P22888 | 0.9324 | 0.827 | ovary |
| LHFPL1 | ENSG00000182508 | Q86WI0 | 0.8639 | 0.7983 | basal ganglia |
| LHFPL4 | ENSG00000156959 | Q7Z7J7 | 0.8374 | 0.7801 | cerebellum |
| LHFPL5 | ENSG00000197753 | Q8TAF8 | 0.9173 | 0.8589 | pancreas |
| LILRA1 | ENSG00000104974 | 075019 | 0.9039 | 0.5353 | spleen |
| LILRA5 | ENSG00000187116 | A6NI73 | 0.8194 | 0.4406 | spleen |
| LIM2 | ENSG00000105370 | P55344 | 1 | 1 | spleen |
| LINGO2 | ENSG00000174482 | Q7L985 | 0.8828 | 0.687 | endometrium |
| LINGO3 | ENSG00000220008 | POC6S8 | 0.9056 | 0.8335 | basal ganglia |
| LINGO4 | ENSG00000213171 | Q6UY18 | 0.9037 | 0.7406 | skeletal muscle |
| LMTK3 | ENSG00000142235 | Q96Q04 | 0.6345 | 0.4375 | cerebral cortex |
| LPAR3 | ENSG00000171517 | Q9UBY5 | 0.6669 | 0.5385 | fallopian tube |
| LPAR4 | ENSG00000147145 | Q99677 | 0.8283 | 0.5001 | ovary |
| LRFN2 | ENSG00000156564 | Q9ULH4 | 0.8782 | 0.7794 | cerebral cortex |
| LRFN5 | ENSG00000165379 | Q96NI6 | 0.664 | 0.4403 | cervix, uterine |
| LRIT2 | ENSG00000204033 | A6NDA9 | 0.9226 | 0.8081 | skin |
| LRP1B | ENSG00000168702 | Q9NZR2 | 0.647 | 0.5573 | cerebral cortex |
| LRP2 | ENSG00000081479 | P98164 | 0.8131 | 0.7274 | thyroid gland |
| LRRC15 | ENSG00000172061 | Q8TF66 | 0.9614 | 0.8791 | skin |
| LRRC19 | ENSG00000184434 | Q9H756 | 0.946 | 0.9411 | colon |
| LRRC26 | ENSG00000184709 | Q210M4 | 0.8847 | 0.7514 | salivary gland |
| LRRC38 | ENSG00000162494 | Q5VT99 | 0.8952 | 0.7459 | skeletal muscle |
| LRRC3B | ENSG00000179796 | Q96PB8 | 0.7744 | 0.6552 | cerebellum |
| LRRC3C | ENSG00000204913 | A6NJW4 | 0.9688 | 0.9688 | cervix, uterine |
| LRRC4C | ENSG00000148948 | Q9HCJ2 | 0.6932 | 0.4728 | cerebral cortex |
| LRRC52 | ENSG00000162763 | Q8N7C0 | 0.9566 | 0.9022 | skeletal muscle |
| LRRC55 | ENSG00000183908 | Q6ZSA7 | 0.8304 | 0.7392 | hypothalamus |
| LRRC8E | ENSG00000171017 | Q6NSJ5 | 0.7422 | 0.5782 | skin |
| LRRN3 | ENSG00000173114 | Q9H3W5 | 0.6609 | 0.4542 | adrenal gland |
| LRRN4 | ENSG00000125872 | Q8WUT4 | 0.9221 | 0.8602 | lung |
| LRRN4CL | ENSG00000177363 | Q8ND94 | 0.5905 | 0.4325 | endometrium |
| LRRTM1 | ENSG00000162951 | Q86UE6 | 0.7547 | 0.5412 | basal ganglia |
| LRRTM2 | ENSG00000146006 | O43300 | 0.7881 | 0.6567 | cerebral cortex |
| LRRTM3 | ENSG00000198739 | Q86VH5 | 0.8111 | 0.7531 | cerebral cortex |
| LRRTM4 | ENSG00000176204 | Q86VH4 | 0.8543 | 0.698 | cerebral cortex |
| LRTM1 | ENSG00000144771 | Q9HBL6 | 0.9824 | 0.968 | spleen |
| LRTM2 | ENSG00000166159 | Q8N967 | 0.9029 | 0.8621 | cerebral cortex |
| LTB | ENSG00000227507 | Q06643 | 0.8217 | 0.4923 | spleen |
| LTB4R2 | ENSG00000213906 | Q9NPC1 | 0.7607 | 0.5077 | skin |
| LTK | ENSG00000062524 | P29376 | 0.7801 | 0.457 | lung |
| LVRN | ENSG00000172901 | Q6Q4G3 | 0.9315 | 0.8118 | adipose tissue |
| LY6G6F | ENSG00000204424 | Q5SQ64 | 0.9668 | 0.9641 | spleen |
| LY9 | ENSG00000122224 | Q9HBG7 | 0.8426 | 0.6146 | spleen |
| MAG | ENSG00000105695 | P20916 | 0.8025 | 0.6883 | spinal cord |
| MARCO | ENSG00000019169 | Q9UEW3 | 0.6975 | 0.4354 | lung |
| MARVELD2 | ENSG00000152939 | Q8N4S9 | 0.637 | 0.4159 | thyroid gland |
| MARVELD3 | ENSG00000140832 | Q96A59 | 0.7301 | 0.5439 | small intestine |
| MAS1 | ENSG00000130368 | P04201 | 0.9253 | 0.9175 | cerebral cortex |
| MAS1L | ENSG00000204687 | P35410 | 0.8488 | 0.7765 | adipose tissue |
| MBOAT1 | ENSG00000172197 | Q6ZNC8 | 0.6228 | 0.431 | fallopian tube |
| MC2R | ENSG00000185231 | Q01718 | 0.9938 | 0.9897 | adrenal gland |
| MC3R | ENSG00000124089 | P41968 | 1 | 1 | hypothalamus |
| MC4R | ENSG00000166603 | P32245 | 0.9136 | 0.8421 | hypothalamus |
| MC5R | ENSG00000176136 | P33032 | 0.9422 | 0.9222 | esophagus |
| MCEMP1 | ENSG00000183019 | Q8IX19 | 0.882 | 0.5879 | lung |
| MCHR1 | ENSG00000128285 | Q99705 | 0.7825 | 0.6211 | cerebral cortex |
| MCHR2 | ENSG00000152034 | Q969V1 | 0.9836 | 0.9584 | cerebral cortex |
| MCOLN2 | ENSG00000153898 | Q8IZK6 | 0.7471 | 0.558 | adrenal gland |
| MCOLN3 | ENSG00000055732 | Q8TDD5 | 0.7898 | 0.509 | adrenal gland |
| MCTP2 | ENSG00000140563 | Q6DN12 | 0.6535 | 0.4107 | spleen |
| MEGF10 | ENSG00000145794 | Q96KG7 | 0.7753 | 0.6594 | spinal cord |
| MEGF11 | ENSG00000157890 | A6BM72 | 0.8685 | 0.7207 | cerebellum |
| MEP1A | ENSG00000112818 | Q16819 | 0.9728 | 0.9544 | small intestine |
| MEP1B | ENSG00000141434 | Q16820 | 0.9909 | 0.9817 | small intestine |
| MFSD2B | ENSG00000205639 | A6NFX1 | 0.946 | 0.9354 | lung |
| MFSD4A | ENSG00000174514 | Q8N468 | 0.7339 | 0.5104 | stomach |
| MGAM | ENSG00000257335 | O43451 | 0.9255 | 0.7566 | small intestine |
| MGAM2 | ENSG00000257743 | Q2M2H8 | 0.9174 | 0.8786 | small intestine |
| MIP | ENSG00000135517 | P30301 | 0.902 | 0.8341 | liver |
| MLC1 | ENSG00000100427 | Q15049 | 0.7313 | 0.6646 | basal ganglia |
| MLNR | ENSG00000102539 | O43193 | 0.9773 | 0.9605 | stomach |
| MME | ENSG00000196549 | P08473 | 0.654 | 0.4729 | small intestine |
| MMP16 | ENSG00000156103 | P51512 | 0.6457 | 0.4364 | cerebral cortex |
| MMP23B | ENSG00000189409 | O75900 | 0.6536 | 0.4411 | cervix, uterine |
| MMP24 | ENSG00000125966 | Q9Y5R2 | 0.816 | 0.485 | cerebellum |
| MOG | ENSG00000204655 | Q16653 | 0.8399 | 0.7949 | spinal cord |
| MPIG6B | ENSG00000204420 | O95866 | 0.8347 | 0.5921 | skin |
| MPL | ENSG00000117400 | P40238 | 0.8317 | 0.6667 | lung |
| MRAP | ENSG00000170262 | Q8TCY5 | 0.876 | 0.6914 | adrenal gland |
| MRGPRD | ENSG00000172938 | Q8TDS7 | 0.8727 | 0.798 | colon |
| MRGPRE | ENSG00000184350 | Q86SM8 | 0.8976 | 0.8023 | cervix, uterine |
| MRGPRX2 | ENSG00000183695 | Q96LB1 | 0.9237 | 0.8639 | skin |
| MRGPRX3 | ENSG00000179826 | Q96LB0 | 0.9564 | 0.9276 | salivary gland |
| MS4A1 | ENSG00000156738 | P11836 | 0.8504 | 0.6232 | spleen |
| MS4A10 | ENSG00000172689 | Q96PG2 | 0.9954 | 0.9891 | small intestine |
| MS4A12 | ENSG00000071203 | Q9NXJO | 0.9965 | 0.9892 | colon |
| MS4A15 | ENSG00000166961 | Q8N5U1 | 0.9965 | 0.991 | lung |
| MS4A18 | ENSG00000214782 | Q3C1V0 | 1 | 1 | small intestine |
| MS4A2 | ENSG00000149534 | Q01362 | 0.7345 | 0.622 | lung |
| MS4A4E | ENSG00000214787 | Q96PG1 | 0.7206 | 0.5617 | adipose tissue |
| MS4A6E | ENSG00000166926 | Q96DS6 | 0.92 | 0.8902 | spleen |
| MS4A8 | ENSG00000166959 | Q9BY19 | 0.856 | 0.7624 | small intestine |
| MSLNL | ENSG00000162006 | Q96KJ4 | 0.9565 | 0.9192 | cerebral cortex |
| MST1R | ENSG00000164078 | Q04912 | 0.6714 | 0.4971 | skin |
| MTNR1A | ENSG00000168412 | P48039 | 0.9691 | 0.9367 | cerebellum |
| MTNR1B | ENSG00000134640 | P49286 | 1 | 1 | basal ganglia |
| MUC12 | ENSG00000205277 | Q9UKN1 | 0.971 | 0.959 | colon |
| MUC13 | ENSG00000173702 | Q9H3R2 | 0.9732 | 0.9711 | small intestine |
| MUC15 | ENSG00000169550 | Q8N387 | 0.8097 | 0.7161 | thyroid gland |
| MUC16 | ENSG00000181143 | Q8WX17 | 0.9092 | 0.8469 | cervix, uterine |
| MUC17 | ENSG00000169876 | Q685J3 | 0.9923 | 0.9863 | small intestine |
| MUC21 | ENSG00000204544 | Q5SSG8 | 0.9009 | 0.8207 | esophagus |
| MUC22 | ENSG00000261272 | E2RYF6 | 0.9665 | 0.962 | esophagus |
| MUC4 | ENSG00000145113 | Q99102 | 0.8067 | 0.7488 | colon |
| MUSK | ENSG00000030304 | O15146 | 0.85 | 0.7775 | skeletal muscle |
| MYADML2 | ENSG00000185105 | A6NDP7 | 0.9032 | 0.7632 | skeletal muscle |
| MYMK | ENSG00000187616 | A6NI61 | 0.9442 | 0.911 | skeletal muscle |
| MYRFL | ENSG00000166268 | Q96LU7 | 0.9084 | 0.6059 | small intestine |
| NALCN | ENSG00000102452 | Q8IZFO | 0.6951 | 0.539 | cerebellum |
| NAT8L | ENSG00000185818 | Q8N9F0 | 0.6882 | 0.5521 | cerebral cortex |
| NCAM2 | ENSG00000154654 | O15394 | 0.6635 | 0.4905 | cerebral cortex |
| NCMAP | ENSG00000184454 | Q5T1S8 | 0.7822 | 0.6116 | salivary gland |
| NCR1 | ENSG00000189430 | O76036 | 0.9745 | 0.9226 | spleen |
| NCR2 | ENSG00000096264 | 095944 | 1 | 1 | small intestine |
| NCR3 | ENSG00000204475 | O14931 | 0.9293 | 0.7449 | spleen |
| NECTIN4 | ENSG00000143217 | Q96NY8 | 0.7844 | 0.6951 | skin |
| NETO1 | ENSG00000166342 | Q8TDF5 | 0.8387 | 0.7363 | cerebral cortex |
| NIPAL1 | ENSG00000163293 | Q6NVV3 | 0.7922 | 0.626 | skin |
| NIPAL4 | ENSG00000172548 | Q0D2K0 | 0.7756 | 0.5606 | skin |
| NKAIN1 | ENSG00000084628 | Q4KMZ8 | 0.8836 | 0.7673 | cerebellum |
| NKAIN2 | ENSG00000188580 | Q5VXU1 | 0.8243 | 0.7041 | spinal cord |
| NKAIN3 | ENSG00000185942 | Q8N8D7 | 0.8113 | 0.7247 | amygdala |
| NKAIN4 | ENSG00000101198 | Q8IVV8 | 0.7368 | 0.6749 | basal ganglia |
| NKPD1 | ENSG00000179846 | Q17RQ9 | 0.9225 | 0.7191 | skin |
| NLGN1 | ENSG00000169760 | Q8N2Q7 | 0.6491 | 0.4314 | cerebellum |
| NLGN3 | ENSG00000196338 | Q9NZ94 | 0.6402 | 0.4813 | cerebellum |
| NLGN4Y | ENSG00000165246 | Q8NFZ3 | 0.6204 | 0.438 | cerebellum |
| NMBR | ENSG00000135577 | P28336 | 0.9314 | 0.874 | basal ganglia |
| NMUR1 | ENSG00000171596 | Q9HB89 | 0.6753 | 0.4881 | spleen |
| NMUR2 | ENSG00000132911 | Q9GZQ4 | 0.8741 | 0.8221 | spinal cord |
| NOX1 | ENSG00000007952 | Q9Y5S8 | 0.9707 | 0.8891 | colon |
| NOX4 | ENSG00000086991 | Q9NPH5 | 0.7777 | 0.5032 | kidney |
| NOX5 | ENSG00000255346 | Q96PH1 | 0.9474 | 0.6781 | spleen |
| NPBWR2 | ENSG00000125522 | P48146 | 0.9783 | 0.9744 | cerebral cortex |
| NPC1L1 | ENSG00000015520 | Q9UHC9 | 0.9191 | 0.612 | liver |
| NPFFR1 | ENSG00000148734 | Q9GZQ6 | 0.9187 | 0.7889 | cerebellum |
| NPFFR2 | ENSG00000056291 | Q9Y5X5 | 0.8887 | 0.8009 | urinary bladder |
| NPHS1 | ENSG00000161270 | O60500 | 0.9659 | 0.9336 | kidney |
| NPR3 | ENSG00000113389 | P17342 | 0.6036 | 0.4537 | kidney |
| NPSR1 | ENSG00000187258 | Q6W5P4 | 1 | 1 | hypothalamus |
| NPY2R | ENSG00000185149 | P49146 | 0.8241 | 0.7534 | hypothalamus |
| NPY5R | ENSG00000164129 | Q15761 | 0.7209 | 0.4803 | spleen |
| NRCAM | ENSG00000091129 | Q92823 | 0.6811 | 0.4793 | cerebral cortex |
| NRG3 | ENSG00000185737 | P56975 | 0.826 | 0.6709 | cerebral cortex |
| NRG4 | ENSG00000169752 | Q8WWG1 | 0.9832 | 0.9614 | cerebellum |
| NRSN1 | ENSG00000152954 | Q8IZ57 | 0.8056 | 0.7533 | cerebral cortex |
| NRXN1 | ENSG00000179915 | Q9ULB1 | 0.7261 | 0.5362 | cerebellum |
| NSG2 | ENSG00000170091 | Q9Y328 | 0.7634 | 0.675 | basal ganglia |
| NTRK1 | ENSG00000198400 | P04629 | 0.6964 | 0.5304 | basal ganglia |
| NTSR1 | ENSG00000101188 | P30989 | 0.9628 | 0.8823 | colon |
| NTSR2 | ENSG00000169006 | O95665 | 0.8173 | 0.7803 | basal ganglia |
| NUP210L | ENSG00000143552 | Q5VU65 | 0.896 | 0.7679 | pituitary gland |
| NXPE2 | ENSG00000204361 | Q96DL1 | 0.9528 | 0.9412 | colon |
| ODF4 | ENSG00000184650 | Q2M2E3 | 0.9673 | 0.9521 | esophagus |
| OLR1 | ENSG00000173391 | P78380 | 0.7508 | 0.4809 | lung |
| OPALIN | ENSG00000197430 | Q96PE5 | 0.817 | 0.8016 | hippocampal |
| | | | | | formation |
| OPN1SW | ENSG00000128617 | P03999 | 0.7796 | 0.4889 | endometrium |
| OPN4 | ENSG00000122375 | Q9UHM6 | 0.8161 | 0.6622 | basal ganglia |
| OPN5 | ENSG00000124818 | Q6U736 | 1 | 1 | heart muscle |
| OPRD1 | ENSG00000116329 | P41143 | 0.9706 | 0.9243 | cerebral cortex |
| OPRK1 | ENSG00000082556 | P41145 | 0.8806 | 0.8114 | basal ganglia |
| OPRM1 | ENSG00000112038 | P35372 | 0.9782 | 0.9532 | cerebellum |
| OR10A3 | ENSG00000170683 | P58181 | 0.9688 | 0.9688 | skin |
| OR10G3 | ENSG00000169208 | Q8NGC4 | 1 | 1 | pituitary gland |
| OR10G4 | ENSG00000254737 | Q8NGN3 | 1 | 1 | pancreas |
| OR10P1 | ENSG00000175398 | Q8NGE3 | 1 | 1 | heart muscle |
| OR13A1 | ENSG00000256574 | Q8NGR1 | 0.8983 | 0.773 | urinary bladder |
| OR1411 | ENSG00000189181 | A6ND48 | 0.9778 | 0.9532 | cerebral cortex |
| OR1F1 | ENSG00000168124 | O43749 | 0.9627 | 0.8976 | cerebellum |
| OR1G1 | ENSG00000183024 | P47890 | 1 | 1 | urinary bladder |
| OR2B11 | ENSG00000177535 | Q5JQS5 | 1 | 1 | adipose tissue |
| OR2B6 | ENSG00000124657 | P58173 | 0.9556 | 0.9352 | prostate |
| OR2H2 | ENSG00000204657 | 095918 | 0.8174 | 0.6287 | cerebellum |
| OR2K2 | ENSG00000171133 | Q8NGT1 | 0.9578 | 0.9268 | spinal cord |
| OR2L13 | ENSG00000196071 | Q8N349 | 0.8512 | 0.7924 | spinal cord |
| OR2L2 | ENSG00000203663 | Q8NH16 | 0.9456 | 0.8467 | spinal cord |
| OR2L3 | ENSG00000198128 | Q8NG85 | 0.9347 | 0.8943 | cerebellum |
| OR2T10 | ENSG00000184022 | Q8NGZ9 | 1 | 1 | kidney |
| OR2T33 | ENSG00000177212 | Q8NG76 | 0.9646 | 0.917 | thyroid gland |
| OR2V2 | ENSG00000182613 | Q96R30 | 1 | 1 | fallopian tube |
| OR2W3 | ENSG00000238243 | Q7Z3T1 | 0.8955 | 0.7434 | thyroid gland |
| OR3A2 | ENSG00000221882 | P47893 | 0.8667 | 0.7823 | pituitary gland |
| OR3A3 | ENSG00000159961 | P47888 | 0.9688 | 0.9688 | cerebellum |
| OR4F17 | ENSG00000176695 | Q8NGA8 | 0.9688 | 0.9688 | cervix, uterine |
| OR51B4 | ENSG00000183251 | Q9Y5P0 | 0.9688 | 0.9688 | cervix, uterine |
| OR51E2 | ENSG00000167332 | Q9H255 | 0.9305 | 0.8026 | prostate |
| OR5211 | ENSG00000232268 | Q8NGK6 | 1 | 1 | cerebellum |
| OR52N1 | ENSG00000181001 | Q8NH53 | 1 | 1 | adipose tissue |
| OR52N4 | ENSG00000181074 | Q8NGI2 | 0.7646 | 0.5145 | spleen |
| OR52N5 | ENSG00000181009 | Q8NH56 | 1 | 1 | adipose tissue |
| OR5M11 | ENSG00000255223 | Q96RB7 | 1 | 1 | urinary bladder |
| OR5P2 | ENSG00000183303 | Q8WZ92 | 0.9688 | 0.9688 | skin |
| OR5P3 | ENSG00000182334 | Q8WZ94 | 0.9837 | 0.9785 | skin |
| OR6B3 | ENSG00000178586 | Q8NGW1 | 0.951 | 0.9237 | spinal cord |
| OR6T1 | ENSG00000181499 | Q8NGN1 | 1 | 1 | pancreas |
| OR7A5 | ENSG00000188269 | Q15622 | 0.8354 | 0.7609 | midbrain |
| OR7C1 | ENSG00000127530 | O76099 | 0.9114 | 0.7981 | pituitary gland |
| OR9A2 | ENSG00000179468 | Q8NGT5 | 1 | 1 | spinal cord |
| OR9G4 | ENSG00000172457 | Q8NGQ1 | 1 | 1 | spleen |
| OTOF | ENSG00000115155 | Q9HC10 | 0.9148 | 0.6449 | basal ganglia |
| OTOP1 | ENSG00000163982 | Q7RTM1 | 0.9688 | 0.9688 | esophagus |
| OTOP2 | ENSG00000183034 | Q7RTS6 | 0.977 | 0.9507 | colon |
| OXGR1 | ENSG00000165621 | Q96P68 | 0.691 | 0.4619 | salivary gland |
| OXTR | ENSG00000180914 | P30559 | 0.8064 | 0.5619 | breast |
| P2RX1 | ENSG00000108405 | P51575 | 0.7186 | 0.4753 | urinary bladder |
| P2RX2 | ENSG00000187848 | Q9UBL9 | 0.7587 | 0.5725 | prostate |
| P2RX3 | ENSG00000109991 | P56373 | 0.973 | 0.9366 | heart muscle |
| P2RX5 | ENSG00000083454 | Q93086 | 0.8053 | 0.5566 | spleen |
| P2RX6 | ENSG00000099957 | O15547 | 0.8771 | 0.6345 | skeletal muscle |
| P2RY10 | ENSG00000078589 | O00398 | 0.8813 | 0.6755 | spleen |
| P2RY12 | ENSG00000169313 | Q9H244 | 0.742 | 0.5473 | spinal cord |
| P2RY13 | ENSG00000181631 | Q9BPV8 | 0.8286 | 0.4452 | spleen |
| P2RY2 | ENSG00000175591 | P41231 | 0.6519 | 0.4201 | esophagus |
| P2RY4 | ENSG00000186912 | P51582 | 0.9151 | 0.7566 | small intestine |
| P2RY8 | ENSG00000182162 | Q86VZ1 | 0.7504 | 0.4916 | spleen |
| PANX3 | ENSG00000154143 | Q96QZ0 | 0.9688 | 0.9688 | basal ganglia |
| PAQR5 | ENSG00000137819 | Q9NXK6 | 0.6807 | 0.4044 | kidney |
| PAQR6 | ENSG00000160781 | Q6TCH4 | 0.6795 | 0.4179 | spinal cord |
| PAQR9 | ENSG00000188582 | Q6ZVX9 | 0.9029 | 0.8086 | liver |
| PCDH10 | ENSG00000138650 | Q9P2E7 | 0.7298 | 0.58 | basal ganglia |
| PCDH11X | ENSG00000102290 | Q9BZA7 | 0.8861 | 0.7904 | basal ganglia |
| PCDH11Y | ENSG00000099715 | Q9BZA8 | 0.8923 | 0.8333 | hypothalamus |
| PCDH15 | ENSG00000150275 | Q96QU1 | 0.7674 | 0.726 | hypothalamus |
| PCDH19 | ENSG00000165194 | Q8TAB3 | 0.7349 | 0.5018 | hypothalamus |
| PCDH8 | ENSG00000136099 | O95206 | 0.8504 | 0.7786 | cerebral cortex |
| PCDH9 | ENSG00000184226 | Q9HC56 | 0.6771 | 0.48 | cerebral cortex |
| PCDHA1 | ENSG00000204970 | Q9Y513 | 0.7796 | 0.714 | cerebral cortex |
| PCDHA10 | ENSG00000250120 | Q9Y512 | 0.7553 | 0.5153 | cerebellum |
| PCDHA11 | ENSG00000249158 | Q9Y511 | 0.8909 | 0.6662 | cerebellum |
| PCDHA12 | ENSG00000251664 | Q9UN75 | 0.9013 | 0.7021 | cerebellum |
| PCDHA13 | ENSG00000239389 | Q9Y510 | 0.896 | 0.691 | cerebellum |
| PCDHA2 | ENSG00000204969 | Q9Y5H9 | 0.8499 | 0.5857 | cerebellum |
| PCDHA3 | ENSG00000255408 | Q9Y5H8 | 0.6995 | 0.4266 | cerebellum |
| PCDHA4 | ENSG00000204967 | Q9UN74 | 0.8115 | 0.5177 | cerebellum |
| PCDHA5 | ENSG00000204965 | Q9Y5H7 | 0.9149 | 0.7574 | cerebellum |
| PCDHA6 | ENSG00000081842 | Q9UN73 | 0.8321 | 0.6082 | cerebellum |
| PCDHA7 | ENSG00000204963 | Q9UN72 | 0.8732 | 0.5785 | cerebellum |
| PCDHA8 | ENSG00000204962 | Q9Y5H6 | 0.9577 | 0.8634 | cerebellum |
| PCDHA9 | ENSG00000204961 | Q9Y5H5 | 0.9608 | 0.9203 | cerebellum |
| PCDHAC1 | ENSG00000248383 | Q9H158 | 0.8091 | 0.6529 | cerebral cortex |
| PCDHAC2 | ENSG00000243232 | Q9Y514 | 0.8384 | 0.6097 | cerebellum |
| PCDHB1 | ENSG00000171815 | Q9Y5F3 | 0.9688 | 0.9688 | fallopian tube |
| PCDHGC5 | ENSG00000240764 | Q9Y5F6 | 0.7226 | 0.4376 | cerebral cortex |
| PDCD1 | ENSG00000188389 | Q15116 | 0.7717 | 0.513 | spleen |
| PDCD1LG2 | ENSG00000197646 | Q9BQ51 | 0.6972 | 0.4692 | spleen |
| PDZK1IP1 | ENSG00000162366 | Q13113 | 0.6818 | 0.4854 | kidney |
| PHEX | ENSG00000102174 | P78562 | 0.803 | 0.5587 | lung |
| PIANP | ENSG00000139200 | Q8IYJO | 0.6193 | 0.469 | basal ganglia |
| PIGR | ENSG00000162896 | P01833 | 0.7579 | 0.6337 | salivary gland |
| PIRT | ENSG00000233670 | POC851 | 0.7994 | 0.7286 | hypothalamus |
| PKD2L1 | ENSG00000107593 | Q9POL9 | 0.9205 | 0.7534 | spleen |
| PKDREJ | ENSG00000130943 | Q9NTG1 | 0.7059 | 0.4633 | ovary |
| PKHD1 | ENSG00000170927 | P08F94 | 0.9624 | 0.9358 | kidney |
| PKHD1L1 | ENSG00000205038 | Q86WI1 | 0.7997 | 0.5803 | thyroid gland |
| PLD5 | ENSG00000180287 | Q8N7P1 | 0.8055 | 0.5177 | cerebellum |
| PLP1 | ENSG00000123560 | P60201 | 0.6674 | 0.4246 | spinal cord |
| PLPP4 | ENSG00000203805 | Q5VZY2 | 0.7349 | 0.615 | hypothalamus |
| PLPPR1 | ENSG00000148123 | Q8TBJ4 | 0.8028 | 0.7395 | basal ganglia |
| PLPPR3 | ENSG00000129951 | Q6T4P5 | 0.793 | 0.7298 | basal ganglia |
| PLPPR4 | ENSG00000117600 | Q7Z2D5 | 0.6792 | 0.5234 | basal ganglia |
| PLPPR5 | ENSG00000117598 | Q32ZL2 | 0.8585 | 0.8181 | cerebral cortex |
| PLSCR2 | ENSG00000163746 | Q9NRY7 | 0.9375 | 0.9375 | fallopian tube |
| PMEL | ENSG00000185664 | P40967 | 0.8657 | 0.6452 | cervix, uterine |
| PNPLA3 | ENSG00000100344 | Q9NST1 | 0.8702 | 0.6163 | liver |
| PODXL | ENSG00000128567 | 000592 | 0.6626 | 0.4243 | lung |
| POPDC2 | ENSG00000121577 | Q9HBU9 | 0.7461 | 0.4752 | heart muscle |
| POPDC3 | ENSG00000132429 | Q9HBV1 | 0.8135 | 0.5652 | skeletal muscle |
| PPP1R3A | ENSG00000154415 | Q16821 | 0.9679 | 0.9599 | skeletal muscle |
| PRLHR | ENSG00000119973 | P49683 | 0.875 | 0.7717 | pituitary gland |
| PRLR | ENSG00000113494 | P16471 | 0.7547 | 0.5185 | cervix, uterine |
| PROKR1 | ENSG00000169618 | Q8TCW9 | 0.9661 | 0.9512 | adipose tissue |
| PROM1 | ENSG00000007062 | O43490 | 0.7574 | 0.4694 | salivary gland |
| PROM2 | ENSG00000155066 | Q8N271 | 0.634 | 0.4492 | esophagus |
| PRRG2 | ENSG00000126460 | O14669 | 0.5839 | 0.4027 | pituitary gland |
| PRRG3 | ENSG00000130032 | Q9BZD7 | 0.642 | 0.4095 | cerebral cortex |
| PRSS8 | ENSG00000052344 | Q16651 | 0.5791 | 0.491 | salivary gland |
| PRTG | ENSG00000166450 | Q2VWP7 | 0.8224 | 0.4546 | thyroid gland |
| PSD2 | ENSG00000146005 | Q9BQ17 | 0.7797 | 0.7091 | basal ganglia |
| PTCHD1 | ENSG00000165186 | Q96NR3 | 0.7997 | 0.5375 | cerebellum |
| PTCHD4 | ENSG00000244694 | Q6ZW05 | 0.7552 | 0.5261 | cervix, uterine |
| PTCRA | ENSG00000171611 | Q6ISU1 | 0.9588 | 0.9093 | spleen |
| PTGDR | ENSG00000168229 | Q13258 | 0.713 | 0.4487 | spleen |
| PTGDR2 | ENSG00000183134 | Q9Y5Y4 | 0.7024 | 0.4766 | colon |
| PTGER1 | ENSG00000160951 | P34995 | 0.7926 | 0.5093 | kidney |
| PTGER2 | ENSG00000125384 | P43116 | 0.6445 | 0.4674 | cervix, uterine |
| PTGER3 | ENSG00000050628 | P43115 | 0.7204 | 0.4691 | endometrium |
| PTGFR | ENSG00000122420 | P43088 | 0.6227 | 0.4171 | adipose tissue |
| PTH2R | ENSG00000144407 | P49190 | 0.8732 | 0.7647 | cerebral cortex |
| PTPRD | ENSG00000153707 | P23468 | 0.6791 | 0.4507 | cerebellum |
| PTPRH | ENSG00000080031 | Q9HD43 | 0.7303 | 0.5359 | small intestine |
| PTPRN | ENSG00000054356 | Q16849 | 0.7354 | 0.6422 | pituitary gland |
| PTPRO | ENSG00000151490 | Q16827 | 0.6773 | 0.434 | kidney |
| PTPRR | ENSG00000153233 | Q15256 | 0.856 | 0.7111 | cerebellum |
| PTPRT | ENSG00000196090 | O14522 | 0.8515 | 0.6733 | cerebral cortex |
| PTPRZ1 | ENSG00000106278 | P23471 | 0.6511 | 0.5129 | amygdala |
| PVRIG | ENSG00000213413 | Q6DKI7 | 0.8874 | 0.5625 | spleen |
| QRFPR | ENSG00000186867 | Q96P65 | 0.9068 | 0.7554 | hypothalamus |
| RAET1E | ENSG00000164520 | Q8TD07 | 0.828 | 0.6719 | esophagus |
| RAET1G | ENSG00000203722 | Q6H3X3 | 0.7884 | 0.5641 | esophagus |
| RDH8 | ENSG00000080511 | Q9NYR8 | 1 | 1 | kidney |
| RGR | ENSG00000148604 | P47804 | 0.8858 | 0.7879 | cerebral cortex |
| RGS9BP | ENSG00000186326 | Q6ZS82 | 0.9115 | 0.6528 | skeletal muscle |
| RHAG | ENSG00000112077 | Q02094 | 0.8669 | 0.7284 | spleen |
| RHBDL2 | ENSG00000158315 | Q9NX52 | 0.6836 | 0.5013 | esophagus |
| RHBDL3 | ENSG00000141314 | P58872 | 0.6482 | 0.4579 | cerebral cortex |
| RHCG | ENSG00000140519 | Q9UBD6 | 0.8103 | 0.545 | esophagus |
| RHD | ENSG00000187010 | Q02161 | 0.87 | 0.5627 | salivary gland |
| RHO | ENSG00000163914 | P08100 | 0.9424 | 0.9299 | cerebral cortex |
| RNF148 | ENSG00000235631 | Q8N7C7 | 0.7923 | 0.5921 | cerebellum |
| RNF175 | ENSG00000145428 | Q8N4F7 | 0.6971 | 0.4973 | cerebral cortex |
| RNF182 | ENSG00000180537 | Q8N6D2 | 0.7571 | 0.5487 | cerebellum |
| RNF222 | ENSG00000189051 | A6NCQ9 | 0.9192 | 0.8943 | esophagus |
| RNF223 | ENSG00000237330 | E7ERA6 | 0.8643 | 0.8014 | esophagus |
| RNF225 | ENSG00000269855 | MOQZC1 | 0.9065 | 0.8865 | esophagus |
| RNFT2 | ENSG00000135119 | Q96EX2 | 0.7117 | 0.5322 | cerebellum |
| ROBO2 | ENSG00000185008 | Q9HCK4 | 0.6406 | 0.4936 | cerebral cortex |
| ROR2 | ENSG00000169071 | Q01974 | 0.5841 | 0.4226 | colon |
| ROS1 | ENSG00000047936 | P08922 | 0.9881 | 0.9638 | lung |
| RPRM | ENSG00000177519 | Q9NS64 | 0.5866 | 0.417 | cervix, uterine |
| RPRML | ENSG00000179673 | Q8N4K4 | 0.8648 | 0.7156 | basal ganglia |
| RRH | ENSG00000180245 | 014718 | 0.8631 | 0.4221 | cerebellum |
| RTL1 | ENSG00000254656 | A6NKG5 | 0.9374 | 0.8457 | hypothalamus |
| RTP1 | ENSG00000175077 | P59025 | 0.9441 | 0.8899 | cerebral cortex |
| RTP2 | ENSG00000198471 | Q5QGT7 | 1 | 1 | skeletal muscle |
| RTP3 | ENSG00000163825 | Q9BQQ7 | 0.993 | 0.9768 | liver |
| RTP5 | ENSG00000188011 | Q14D33 | 0.8361 | 0.7338 | hypothalamus |
| RXFP1 | ENSG00000171509 | Q9HBX9 | 0.8567 | 0.7037 | cerebral cortex |
| RXFP2 | ENSG00000133105 | Q8WXD0 | 0.8799 | 0.8391 | fallopian tube |
| RXFP3 | ENSG00000182631 | Q9NSD7 | 0.9786 | 0.9659 | adrenal gland |
| RXFP4 | ENSG00000173080 | Q8TDU9 | 0.9643 | 0.9402 | colon |
| RYR2 | ENSG00000198626 | Q92736 | 0.9627 | 0.871 | heart muscle |
| RYR3 | ENSG00000198838 | Q15413 | 0.5951 | 0.4241 | basal ganglia |
| S1PR4 | ENSG00000125910 | O95977 | 0.8022 | 0.4641 | spleen |
| S1PR5 | ENSG00000180739 | Q9H228 | 0.6682 | 0.4864 | spinal cord |
| SCARA5 | ENSG00000168079 | Q6ZMJ2 | 0.5492 | 0.4026 | urinary bladder |
| SCIMP | ENSG00000161929 | Q6UWF3 | 0.8173 | 0.4138 | spleen |
| SCN11A | ENSG00000168356 | Q9UI33 | 0.8948 | 0.6915 | spleen |
| SCN1A | ENSG00000144285 | P35498 | 0.8186 | 0.735 | cerebral cortex |
| SCN2A | ENSG00000136531 | Q99250 | 0.8486 | 0.6979 | cerebellum |
| SCN2B | ENSG00000149575 | 060939 | 0.7773 | 0.5859 | cerebellum |
| SCN3A | ENSG00000153253 | Q9NY46 | 0.7189 | 0.5172 | cerebellum |
| SCN3B | ENSG00000166257 | Q9NY72 | 0.6808 | 0.438 | cerebral cortex |
| SCN4A | ENSG00000007314 | P35499 | 0.8932 | 0.7638 | skeletal muscle |
| SCN5A | ENSG00000183873 | Q14524 | 0.9173 | 0.6151 | heart muscle |
| SCN8A | ENSG00000196876 | Q9UQDO | 0.8201 | 0.5781 | cerebellum |
| SCN9A | ENSG00000169432 | Q15858 | 0.7594 | 0.4311 | hypothalamus |
| SCNN1A | ENSG00000111319 | P37088 | 0.548 | 0.4393 | salivary gland |
| SCNN1B | ENSG00000168447 | P51168 | 0.6311 | 0.5117 | colon |
| SCNN1G | ENSG00000166828 | P51170 | 0.6329 | 0.5589 | kidney |
| SCTR | ENSG00000080293 | P47872 | 0.9467 | 0.89 | pancreas |
| SDC1 | ENSG00000115884 | P18827 | 0.5779 | 0.4185 | skin |
| SDK2 | ENSG00000069188 | Q58EX2 | 0.8226 | 0.6812 | hypothalamus |
| SDR42E1 | ENSG00000184860 | Q8WUS8 | 0.7108 | 0.456 | skin |
| SERP2 | ENSG00000151778 | Q8N6R1 | 0.6979 | 0.5313 | cerebral cortex |
| SERTM1 | ENSG00000180440 | A2A2V5 | 0.7674 | 0.6298 | fallopian tube |
| SEZ6 | ENSG00000063015 | Q53EL9 | 0.8311 | 0.747 | cerebellum |
| SFT2D3 | ENSG00000173349 | Q58719 | 0.6865 | 0.405 | pancreas |
| SGCG | ENSG00000102683 | Q13326 | 0.832 | 0.6114 | heart muscle |
| SGCZ | ENSG00000185053 | Q96LD1 | 0.8585 | 0.6925 | ovary |
| SHISA6 | ENSG00000188803 | Q6ZSJ9 | 0.649 | 0.486 | cerebellum |
| SHISA7 | ENSG00000187902 | A6NL88 | 0.8127 | 0.7759 | basal ganglia |
| SHISA8 | ENSG00000234965 | B8ZZ34 | 0.9488 | 0.8338 | cerebellum |
| SHISA9 | ENSG00000237515 | B4DS77 | 0.8116 | 0.7384 | basal ganglia |
| SHISAL1 | ENSG00000138944 | Q3SXP7 | 0.681 | 0.4924 | endometrium |
| SHISAL2A | ENSG00000182183 | Q6UWV7 | 0.7881 | 0.5214 | spleen |
| SHISAL2B | ENSG00000145642 | A6NKW6 | 0.9603 | 0.9284 | hypothalamus |
| SI | ENSG00000090402 | P14410 | 0.9904 | 0.9854 | small intestine |
| SIGLEC11 | ENSG00000161640 | Q96RL6 | 0.8878 | 0.6231 | ovary |
| SIGLEC12 | ENSG00000254521 | Q96PQ1 | 0.9394 | 0.7528 | spleen |
| SIGLEC15 | ENSG00000197046 | Q6ZMC9 | 0.9911 | 0.9862 | urinary bladder |
| SIGLEC5 | ENSG00000105501 | O15389 | 0.9532 | 0.9368 | spleen |
| SIGLEC6 | ENSG00000105492 | O43699 | 0.8224 | 0.646 | small intestine |
| SIGLEC7 | ENSG00000168995 | Q9Y286 | 0.8046 | 0.4169 | spleen |
| SIGLEC8 | ENSG00000105366 | Q9NYZ4 | 0.743 | 0.4954 | spinal cord |
| SIRPG | ENSG00000089012 | Q9P1W8 | 0.8825 | 0.6602 | spleen |
| SIT1 | ENSG00000137078 | Q9Y3P8 | 0.8928 | 0.6672 | spleen |
| SLAMF1 | ENSG00000117090 | Q13291 | 0.7965 | 0.5653 | spleen |
| SLAMF6 | ENSG00000162739 | Q96DU3 | 0.8351 | 0.5582 | spleen |
| SLAMF7 | ENSG00000026751 | Q9NQ25 | 0.7907 | 0.5499 | spleen |
| SLAMF9 | ENSG00000162723 | Q96A28 | 0.9425 | 0.8772 | adipose tissue |
| SLC10A1 | ENSG00000100652 | Q14973 | 0.9978 | 0.9918 | liver |
| SLC10A2 | ENSG00000125255 | Q12908 | 0.9943 | 0.9893 | small intestine |
| SLC10A4 | ENSG00000145248 | Q96EP9 | 0.8394 | 0.614 | midbrain |
| SLC10A6 | ENSG00000145283 | Q3KNW5 | 0.841 | 0.6839 | skin |
| SLC12A1 | ENSG00000074803 | Q13621 | 0.9869 | 0.9366 | kidney |
| SLC12A3 | ENSG00000070915 | P55017 | 0.9958 | 0.9901 | kidney |
| SLC12A5 | ENSG00000124140 | Q9H2X9 | 0.8487 | 0.7987 | cerebellum |
| SLC13A1 | ENSG00000081800 | Q9BZW2 | 0.9683 | 0.9669 | small intestine |
| SLC13A2 | ENSG00000007216 | Q13183 | 0.9256 | 0.884 | small intestine |
| SLC13A4 | ENSG00000164707 | Q9UKG4 | 0.8562 | 0.7569 | cerebral cortex |
| SLC13A5 | ENSG00000141485 | Q86YT5 | 0.9022 | 0.6773 | liver |
| SLC14A1 | ENSG00000141469 | Q13336 | 0.6315 | 0.4145 | urinary bladder |
| SLC14A2 | ENSG00000132874 | Q15849 | 0.9361 | 0.9258 | adipose tissue |
| SLC15A1 | ENSG00000088386 | P46059 | 0.8795 | 0.7362 | small intestine |
| SLC15A5 | ENSG00000188991 | A6NIM6 | 0.9375 | 0.9375 | amygdala |
| SLC16A12 | ENSG00000152779 | Q6ZSM3 | 0.6857 | 0.4072 | thyroid gland |
| SLC17A1 | ENSG00000124568 | Q14916 | 0.9793 | 0.9721 | kidney |
| SLC17A2 | ENSG00000112337 | O00624 | 0.9982 | 0.9965 | liver |
| SLC17A3 | ENSG00000124564 | O00476 | 0.9728 | 0.9559 | kidney |
| SLC17A4 | ENSG00000146039 | Q9Y2C5 | 0.903 | 0.8915 | liver |
| SLC17A6 | ENSG00000091664 | Q9P2U8 | 0.9082 | 0.8513 | hypothalamus |
| SLC17A7 | ENSG00000104888 | Q9P2U7 | 0.7704 | 0.5291 | cerebral cortex |
| SLC17A8 | ENSG00000179520 | Q8NDX2 | 0.9479 | 0.8546 | small intestine |
| SLC18A1 | ENSG00000036565 | P54219 | 0.9637 | 0.8965 | adrenal gland |
| SLC18A3 | ENSG00000187714 | Q16572 | 0.8954 | 0.8381 | basal ganglia |
| SLC19A3 | ENSG00000135917 | Q9BZV2 | 0.7705 | 0.4211 | adipose tissue |
| SLC1A2 | ENSG00000110436 | P43004 | 0.7774 | 0.6405 | basal ganglia |
| SLC1A6 | ENSG00000105143 | P48664 | 0.8923 | 0.7955 | cerebellum |
| SLC22A1 | ENSG00000175003 | O15245 | 0.9571 | 0.6527 | liver |
| SLC22A10 | ENSG00000184999 | Q63ZE4 | 0.998 | 0.9962 | liver |
| SLC22A11 | ENSG00000168065 | Q9NSAO | 0.998 | 0.9943 | kidney |
| SLC22A12 | ENSG00000197891 | Q96S37 | 0.9979 | 0.9948 | kidney |
| SLC22A13 | ENSG00000172940 | Q9Y226 | 0.9789 | 0.944 | kidney |
| SLC22A14 | ENSG00000144671 | Q9Y267 | 0.8723 | 0.5431 | cerebellum |
| SLC22A16 | ENSG00000004809 | Q86VW1 | 0.9443 | 0.8789 | endometrium |
| SLC22A2 | ENSG00000112499 | O15244 | 0.9918 | 0.9606 | kidney |
| SLC22A24 | ENSG00000197658 | Q8N4F4 | 0.9837 | 0.9785 | kidney |
| SLC22A25 | ENSG00000196600 | Q6T423 | 1 | 1 | liver |
| SLC22A6 | ENSG00000197901 | Q4U2R8 | 0.9426 | 0.8424 | kidney |
| SLC22A7 | ENSG00000137204 | Q9Y694 | 0.9716 | 0.9457 | liver |
| SLC22A8 | ENSG00000149452 | Q8TCC7 | 0.987 | 0.9668 | kidney |
| SLC22A9 | ENSG00000149742 | Q8IVM8 | 0.9886 | 0.9808 | liver |
| SLC23A3 | ENSG00000213901 | Q6PIS1 | 0.8232 | 0.5588 | small intestine |
| SLC24A2 | ENSG00000155886 | Q9UI40 | 0.7915 | 0.7538 | cerebral cortex |
| SLC24A4 | ENSG00000140090 | Q8NFF2 | 0.8115 | 0.6285 | midbrain |
| SLC26A10 | ENSG00000135502 | Q8NG04 | 0.9414 | 0.8737 | cerebellum |
| SLC26A3 | ENSG00000091138 | P40879 | 0.9514 | 0.8919 | colon |
| SLC26A4 | ENSG00000091137 | O43511 | 0.9328 | 0.6563 | thyroid gland |
| SLC26A5 | ENSG00000170615 | P58743 | 0.8818 | 0.7215 | cerebellum |
| SLC26A8 | ENSG00000112053 | Q96RN1 | 0.9001 | 0.7564 | cerebellum |
| SLC26A9 | ENSG00000174502 | Q7LBE3 | 0.9174 | 0.86 | stomach |
| SLC27A2 | ENSG00000140284 | O14975 | 0.7567 | 0.459 | liver |
| SLC27A6 | ENSG00000113396 | Q9Y2P4 | 0.6736 | 0.4638 | adrenal gland |
| SLC28A1 | ENSG00000156222 | O00337 | 0.9411 | 0.9056 | liver |
| SLC28A2 | ENSG00000137860 | O43868 | 0.9682 | 0.9388 | small intestine |
| SLC28A3 | ENSG00000197506 | Q9HAS3 | 0.7948 | 0.685 | salivary gland |
| SLC2A14 | ENSG00000173262 | Q8TDB8 | 0.8233 | 0.5152 | adrenal gland |
| SLC2A2 | ENSG00000163581 | P11168 | 0.9653 | 0.9472 | liver |
| SLC30A10 | ENSG00000196660 | Q6XR72 | 0.9271 | 0.802 | liver |
| SLC30A3 | ENSG00000115194 | Q99726 | 0.8956 | 0.7413 | cerebral cortex |
| SLC30A8 | ENSG00000164756 | Q8IWU4 | 0.9525 | 0.7575 | pancreas |
| SLC34A1 | ENSG00000131183 | Q06495 | 0.997 | 0.9946 | kidney |
| SLC34A2 | ENSG00000157765 | O95436 | 0.8391 | 0.6956 | lung |
| SLC34A3 | ENSG00000198569 | Q8N130 | 0.9051 | 0.7644 | kidney |
| SLC35D3 | ENSG00000182747 | Q5M8T2 | 0.9487 | 0.9103 | basal ganglia |
| SLC35F1 | ENSG00000196376 | Q5T1Q4 | 0.7235 | 0.4976 | cerebral cortex |
| SLC35F2 | ENSG00000110660 | Q8IXU6 | 0.9183 | 0.8644 | skin |
| SLC35F3 | ENSG00000183780 | Q8IY50 | 0.7873 | 0.5775 | cerebellum |
| SLC35F4 | ENSG00000151812 | A4IF30 | 0.9186 | 0.7746 | cerebellum |
| SLC35G1 | ENSG00000176273 | Q2M3R5 | 0.7156 | 0.4045 | small intestine |
| SLC36A2 | ENSG00000186335 | Q495M3 | 0.9742 | 0.9648 | skeletal muscle |
| SLC38A4 | ENSG00000139209 | Q96916 | 0.9105 | 0.6666 | liver |
| SLC38A8 | ENSG00000166558 | A6NNN8 | 0.8913 | 0.8174 | amygdala |
| SLC39A12 | ENSG00000148482 | Q504Y0 | 0.8546 | 0.8098 | cerebral cortex |
| SLC39A2 | ENSG00000165794 | Q9NP94 | 0.8842 | 0.812 | skin |
| SLC39A4 | ENSG00000147804 | Q6P5W5 | 0.8474 | 0.6092 | small intestine |
| SLC39A5 | ENSG00000139540 | Q6ZMH5 | 0.8671 | 0.7687 | pancreas |
| SLC3A1 | ENSG00000138079 | Q07837 | 0.859 | 0.6149 | kidney |
| SLC44A4 | ENSG00000204385 | Q53GD3 | 0.6719 | 0.5517 | colon |
| SLC46A1 | ENSG00000076351 | Q96NT5 | 0.7397 | 0.4087 | adrenal gland |
| SLC46A2 | ENSG00000119457 | Q9BY10 | 0.9172 | 0.8199 | skin |
| SLC47A2 | ENSG00000180638 | Q86VL8 | 0.8774 | 0.7711 | kidney |
| SLC4A1 | ENSG00000004939 | P02730 | 0.946 | 0.8938 | kidney |
| SLC4A10 | ENSG00000144290 | Q6U841 | 0.8472 | 0.7795 | cerebral cortex |
| SLC4A11 | ENSG00000088836 | Q8NBS3 | 0.706 | 0.418 | thyroid gland |
| SLC4A4 | ENSG00000080493 | Q9Y6R1 | 0.6167 | 0.4004 | pancreas |
| SLC4A8 | ENSG00000050438 | Q2Y0W8 | 0.7774 | 0.602 | pituitary gland |
| SLC4A9 | ENSG00000113073 | Q96Q91 | 0.9813 | 0.8701 | kidney |
| SLC51A | ENSG00000163959 | Q86UW1 | 0.7634 | 0.4172 | small intestine |
| SLC51B | ENSG00000186198 | Q86UW2 | 0.8555 | 0.5823 | small intestine |
| SLC52A1 | ENSG00000132517 | Q9NWF4 | 0.89 | 0.744 | small intestine |
| SLC5A1 | ENSG00000100170 | P13866 | 0.8558 | 0.7111 | small intestine |
| SLC5A10 | ENSG00000154025 | AOPJK1 | 0.9252 | 0.7081 | kidney |
| SLC5A11 | ENSG00000158865 | Q8WWWX8 | 0.8487 | 0.7541 | spinal cord |
| SLC5A12 | ENSG00000148942 | Q1EHB4 | 0.9475 | 0.9131 | kidney |
| SLC5A2 | ENSG00000140675 | P31639 | 0.9936 | 0.9738 | kidney |
| SLC5A4 | ENSG00000100191 | Q9NY91 | 0.7273 | 0.4623 | small intestine |
| SLC5A5 | ENSG00000105641 | Q92911 | 0.9076 | 0.8553 | stomach |
| SLC5A7 | ENSG00000115665 | Q9GZV3 | 0.8539 | 0.7331 | colon |
| SLC5A8 | ENSG00000256870 | Q8N695 | 0.9279 | 0.8441 | thyroid gland |
| SLC5A9 | ENSG00000117834 | Q2M3M2 | 0.9427 | 0.8587 | small intestine |
| SLC6A1 | ENSG00000157103 | P30531 | 0.6343 | 0.4589 | cerebral cortex |
| SLC6A11 | ENSG00000132164 | P48066 | 0.8041 | 0.6978 | midbrain |
| SLC6A12 | ENSG00000111181 | P48065 | 0.6914 | 0.5438 | kidney |
| SLC6A13 | ENSG00000010379 | Q9NSD5 | 0.796 | 0.5954 | kidney |
| SLC6A15 | ENSG00000072041 | Q9H2J7 | 0.7513 | 0.5994 | cerebellum |
| SLC6A17 | ENSG00000197106 | Q9H1V8 | 0.7883 | 0.6932 | cerebral cortex |
| SLC6A18 | ENSG00000164363 | Q96N87 | 0.9949 | 0.9913 | kidney |
| SLC6A19 | ENSG00000174358 | Q695T7 | 0.9624 | 0.9351 | small intestine |
| SLC6A2 | ENSG00000103546 | P23975 | 0.859 | 0.7225 | adrenal gland |
| SLC6A20 | ENSG00000163817 | Q9NP91 | 0.8719 | 0.6457 | small intestine |
| SLC6A3 | ENSG00000142319 | Q01959 | 0.9714 | 0.8467 | midbrain |
| SLC6A4 | ENSG00000108576 | P31645 | 0.946 | 0.814 | lung |
| SLC6A5 | ENSG00000165970 | Q9Y345 | 0.9439 | 0.9368 | cerebellum |
| SLC6A7 | ENSG00000011083 | Q99884 | 0.899 | 0.8075 | cerebellum |
| SLC7A10 | ENSG00000130876 | Q9NS82 | 0.8027 | 0.6743 | adipose tissue |
| SLC7A11 | ENSG00000151012 | Q9UPY5 | 0.63 | 0.4702 | basal ganglia |
| SLC7A13 | ENSG00000164893 | Q8TCU3 | 1 | 1 | kidney |
| SLC7A3 | ENSG00000165349 | Q8WY07 | 0.845 | 0.6827 | endometrium |
| SLC7A9 | ENSG00000021488 | P82251 | 0.9136 | 0.5972 | small intestine |
| SLC8A1 | ENSG00000183023 | P32418 | 0.6777 | 0.4375 | heart muscle |
| SLC8A2 | ENSG00000118160 | Q9UPR5 | 0.8022 | 0.6044 | cerebellum |
| SLC8A3 | ENSG00000100678 | P57103 | 0.7723 | 0.6112 | skeletal muscle |
| SLC9A2 | ENSG00000115616 | Q9UBY0 | 0.8239 | 0.6318 | colon |
| SLC9A3 | ENSG00000066230 | P48764 | 0.8074 | 0.6262 | colon |
| SLC9A4 | ENSG00000180251 | Q6AI14 | 0.9693 | 0.9209 | stomach |
| SLC9A5 | ENSG00000135740 | Q14940 | 0.744 | 0.4137 | cerebellum |
| SLC9C2 | ENSG00000162753 | Q5TAH2 | 0.981 | 0.9527 | fallopian tube |
| SLCO1A2 | ENSG00000084453 | P46721 | 0.8043 | 0.7425 | hippocampal |
| | | | | | formation |
| SLCO1B1 | ENSG00000134538 | Q9Y6L6 | 0.9984 | 0.9969 | liver |
| SLCO1B3 | ENSG00000111700 | Q9NPD5 | 0.9687 | 0.9183 | liver |
| SLCO1C1 | ENSG00000139155 | Q9NYB5 | 0.8438 | 0.7821 | basal ganglia |
| SLCO4C1 | ENSG00000173930 | Q6ZQN7 | 0.9158 | 0.8424 | kidney |
| SLCO5A1 | ENSG00000137571 | Q9H2Y9 | 0.8988 | 0.7538 | skeletal muscle |
| SLITRK1 | ENSG00000178235 | Q96PX8 | 0.8578 | 0.7875 | cerebral cortex |
| SLITRK2 | ENSG00000185985 | Q9H156 | 0.7748 | 0.6447 | basal ganglia |
| SLITRK3 | ENSG00000121871 | 094933 | 0.7512 | 0.5757 | fallopian tube |
| SLITRK4 | ENSG00000179542 | Q8IW52 | 0.8654 | 0.7019 | adrenal gland |
| SLITRK5 | ENSG00000165300 | O94991 | 0.7519 | 0.5736 | cerebral cortex |
| SLITRK6 | ENSG00000184564 | Q9H5Y7 | 0.8474 | 0.6578 | urinary bladder |
| SMCO2 | ENSG00000165935 | A6NFE2 | 0.8776 | 0.7093 | esophagus |
| SMCO3 | ENSG00000179256 | A2RU48 | 0.7385 | 0.5721 | kidney |
| SMIM13 | ENSG00000224531 | PODJ93 | 0.847 | 0.5643 | skin |
| SMIM18 | ENSG00000253457 | PODKX4 | 0.8321 | 0.7796 | cerebellum |
| SMIM2 | ENSG00000139656 | Q9BVW6 | 0.9062 | 0.9063 | colon |
| SMIM22 | ENSG00000267795 | K7EJ46 | 0.6129 | 0.4946 | colon |
| SMIM23 | ENSG00000185662 | A6NLE4 | 1 | 1 | cervix, uterine |
| SMIM24 | ENSG00000095932 | O75264 | 0.8306 | 0.6255 | small intestine |
| SMIM28 | ENSG00000262543 | A0A1BOGU29 | 0.991 | 0.986 | colon |
| SMIM3 | ENSG00000256235 | Q9BZL3 | 0.708 | 0.4962 | adipose tissue |
| SMIM6 | ENSG00000259120 | PODI80 | 0.7229 | 0.5605 | kidney |
| SMLR1 | ENSG00000256162 | H3BR10 | 0.9746 | 0.9539 | liver |
| SNORC | ENSG00000182600 | Q6UX34 | 0.6868 | 0.406 | spinal cord |
| SOGA3 | ENSG00000214338 | Q5TF21 | 0.7834 | 0.5599 | spinal cord |
| SORCS3 | ENSG00000156395 | Q9UPU3 | 0.7995 | 0.697 | basal ganglia |
| SPACA3 | ENSG00000141316 | Q8IXA5 | 0.9871 | 0.9361 | pancreas |
| SPATA9 | ENSG00000145757 | Q9BWV2 | 0.9056 | 0.8257 | adipose tissue |
| SPEM2 | ENSG00000184560 | Q0P670 | 0.9688 | 0.9688 | breast |
| SPN | ENSG00000197471 | P16150 | 0.786 | 0.4488 | spleen |
| SPNS3 | ENSG00000182557 | Q6ZMD2 | 0.8849 | 0.7488 | spinal cord |
| SPPL2C | ENSG00000185294 | Q8IUH8 | 0.9759 | 0.9403 | cerebellum |
| SSTR4 | ENSG00000132671 | P31391 | 0.948 | 0.9021 | cerebellum |
| SSTR5 | ENSG00000162009 | P35346 | 0.9367 | 0.8741 | pituitary gland |
| STAB2 | ENSG00000136011 | Q8WWQ8 | 0.9187 | 0.6395 | spleen |
| STEAP1B | ENSG00000105889 | Q6NZ63 | 0.6547 | 0.4009 | spinal cord |
| STOML3 | ENSG00000133115 | Q8TAV4 | 0.9196 | 0.8472 | fallopian tube |
| STRA6 | ENSG00000137868 | Q9BX79 | 0.8656 | 0.6643 | cervix, uterine |
| STX1B | ENSG00000099365 | P61266 | 0.8113 | 0.5745 | cerebellum |
| STYK1 | ENSG00000060140 | Q6J9G0 | 0.6264 | 0.5154 | prostate |
| SUCNR1 | ENSG00000198829 | Q9BXA5 | 0.9052 | 0.7854 | thyroid gland |
| SV2A | ENSG00000159164 | Q7LOJ3 | 0.6219 | 0.4022 | cerebellum |
| SV2B | ENSG00000185518 | Q7L112 | 0.8503 | 0.6858 | cerebellum |
| SV2C | ENSG00000122012 | Q496J9 | 0.9163 | 0.8381 | basal ganglia |
| SVOP | ENSG00000166111 | Q8N4V2 | 0.9699 | 0.95 | cerebellum |
| SVOPL | ENSG00000157703 | Q8N434 | 0.8676 | 0.6357 | salivary gland |
| SYNDIG1 | ENSG00000101463 | Q9H7V2 | 0.6676 | 0.4729 | cerebellum |
| SYNDIG1L | ENSG00000183379 | A6NDD5 | 0.9032 | 0.652 | basal ganglia |
| SYNGR3 | ENSG00000127561 | O43761 | 0.7077 | 0.5612 | hypothalamus |
| SYNGR4 | ENSG00000105467 | O95473 | 0.9533 | 0.7614 | cerebellum |
| SYNPR | ENSG00000163630 | Q8TBG9 | 0.8446 | 0.7911 | basal ganglia |
| SYP | ENSG00000102003 | P08247 | 0.6477 | 0.4304 | cerebral cortex |
| SYT1 | ENSG00000067715 | P21579 | 0.7294 | 0.5434 | cerebral cortex |
| SYT12 | ENSG00000173227 | Q8IV01 | 0.7991 | 0.5778 | cerebellum |
| SYT13 | ENSG00000019505 | Q7L8C5 | 0.737 | 0.6057 | cerebral cortex |
| SYT14 | ENSG00000143469 | Q8NB59 | 0.8711 | 0.7897 | pituitary gland |
| SYT2 | ENSG00000143858 | Q8N910 | 0.9138 | 0.7227 | cerebellum |
| SYT3 | ENSG00000213023 | Q9BQG1 | 0.8065 | 0.689 | cerebellum |
| SYT5 | ENSG00000129990 | O00445 | 0.7731 | 0.6886 | cerebral cortex |
| SYT6 | ENSG00000134207 | Q5T7P8 | 0.8891 | 0.7647 | hypothalamus |
| SYT8 | ENSG00000149043 | Q8NBV8 | 0.8319 | 0.7243 | urinary bladder |
| SYT9 | ENSG00000170743 | Q86SS6 | 0.8437 | 0.6675 | cerebellum |
| TAAR1 | ENSG00000146399 | Q96RJ0 | 1 | 1 | fallopian tube |
| TACR1 | ENSG00000115353 | P25103 | 0.6837 | 0.4932 | adipose tissue |
| TACR2 | ENSG00000075073 | P21452 | 0.7779 | 0.5533 | colon |
| TACR3 | ENSG00000169836 | P29371 | 0.9453 | 0.8839 | urinary bladder |
| TACSTD2 | ENSG00000184292 | P09758 | 0.6687 | 0.523 | esophagus |
| TAS1R1 | ENSG00000173662 | Q7RTX1 | 0.7989 | 0.6518 | spleen |
| TAS1R2 | ENSG00000179002 | Q8TE23 | 1 | 1 | skin |
| TAS1R3 | ENSG00000169962 | Q7RTX0 | 0.7485 | 0.4049 | spleen |
| TAS2R1 | ENSG00000169777 | Q9NYW7 | 0.9688 | 0.9688 | basal ganglia |
| TAS2R10 | ENSG00000121318 | Q9NYWO | 0.6199 | 0.4368 | adrenal gland |
| TAS2R13 | ENSG00000212128 | Q9NYV9 | 1 | 1 | cerebellum |
| TAS2R3 | ENSG00000127362 | Q9NYW6 | 0.8865 | 0.7373 | cerebellum |
| TAS2R30 | ENSG00000256188 | P59541 | 0.9688 | 0.9688 | cerebellum |
| TAS2R38 | ENSG00000257138 | P59533 | 1 | 1 | small intestine |
| TAS2R46 | ENSG00000226761 | P59540 | 0.902 | 0.8341 | cerebellum |
| TAS2R50 | ENSG00000212126 | P59544 | 0.9837 | 0.9785 | cerebellum |
| TECRL | ENSG00000205678 | Q5HYJ1 | 0.9769 | 0.9584 | heart muscle |
| TEDDM1 | ENSG00000203730 | Q5T9Z0 | 0.9183 | 0.8644 | cerebellum |
| TENM2 | ENSG00000145934 | Q9NT68 | 0.9495 | 0.8425 | heart muscle |
| TENM4 | ENSG00000149256 | Q6N022 | 0.7156 | 0.436 | ovary |
| TEX38 | ENSG00000186118 | Q6PEX7 | 0.8148 | 0.4081 | pituitary gland |
| TFR2 | ENSG00000106327 | Q9UP52 | 0.9643 | 0.8723 | liver |
| TGFBR3L | ENSG00000260001 | H3BV60 | 0.9326 | 0.6601 | pituitary gland |
| THSD7B | ENSG00000144229 | Q9C014 | 0.7062 | 0.5439 | adipose tissue |
| TIGIT | ENSG00000181847 | Q495A1 | 0.8507 | 0.5544 | spleen |
| TIMD4 | ENSG00000145850 | Q96H15 | 0.8826 | 0.6597 | spleen |
| TLCD2 | ENSG00000185561 | A6NGC4 | 0.5898 | 0.4102 | adrenal gland |
| TLR10 | ENSG00000174123 | Q9BXR5 | 0.8935 | 0.6191 | spleen |
| TM4SF19 | ENSG00000145107 | Q96DZ7 | 0.8477 | 0.7261 | adipose tissue |
| TM4SF20 | ENSG00000168955 | Q53R12 | 0.9923 | 0.9855 | small intestine |
| TM4SF4 | ENSG00000169903 | P48230 | 0.9259 | 0.8575 | liver |
| TM4SF5 | ENSG00000142484 | O14894 | 0.9322 | 0.9018 | liver |
| TMC1 | ENSG00000165091 | Q8TDI8 | 0.9347 | 0.8943 | urinary bladder |
| TMC2 | ENSG00000149488 | Q8TDI7 | 0.9195 | 0.7355 | cerebellum |
| TMC3 | ENSG00000188869 | Q7Z5M5 | 0.898 | 0.7965 | vagina |
| TMC5 | ENSG00000103534 | Q6UXY8 | 0.7832 | 0.6727 | small intestine |
| TMC7 | ENSG00000170537 | Q7Z402 | 0.6838 | 0.4708 | spinal cord |
| TMCO5A | ENSG00000166069 | Q8N6Q1 | 1 | 1 | stomach |
| TMEFF1 | ENSG00000241697 | Q81YR6 | 0.8178 | 0.7346 | spinal cord |
| TMEFF2 | ENSG00000144339 | Q9UIK5 | 0.7204 | 0.6359 | hypothalamus |
| TMEM114 | ENSG00000232258 | B3SHH9 | 0.978 | 0.9323 | hypothalamus |
| TMEM132B | ENSG00000139364 | Q14DG7 | 0.7703 | 0.5622 | cerebral cortex |
| TMEM132C | ENSG00000181234 | Q8N3T6 | 0.6529 | 0.4026 | cervix, uterine |
| TMEM132D | ENSG00000151952 | Q14C87 | 0.9078 | 0.825 | cerebral cortex |
| TMEM132E | ENSG00000181291 | Q6IEE7 | 0.8656 | 0.6522 | cerebellum |
| TMEM145 | ENSG00000167619 | Q8NBT3 | 0.8388 | 0.6224 | cerebellum |
| TMEM150B | ENSG00000180061 | A6NC51 | 0.8425 | 0.6092 | small intestine |
| TMEM151A | ENSG00000179292 | Q8N4L1 | 0.7856 | 0.6987 | spinal cord |
| TMEM151B | ENSG00000178233 | Q8IW70 | 0.8271 | 0.654 | cerebellum |
| TMEM154 | ENSG00000170006 | Q6P9G4 | 0.7948 | 0.6227 | esophagus |
| TMEM156 | ENSG00000121895 | Q8N614 | 0.8434 | 0.5216 | spleen |
| TMEM163 | ENSG00000152128 | Q8TC26 | 0.7006 | 0.4622 | cerebellum |
| TMEM169 | ENSG00000163449 | Q96HH4 | 0.7776 | 0.5315 | cerebellum |
| TMEM171 | ENSG00000157111 | Q8WVE6 | 0.7866 | 0.652 | colon |
| TMEM178B | ENSG00000261115 | H3BS89 | 0.6796 | 0.4807 | cerebral cortex |
| TMEM179 | ENSG00000258986 | Q6ZVK1 | 0.7476 | 0.6587 | pituitary gland |
| TMEM184A | ENSG00000164855 | Q6ZMB5 | 0.6816 | 0.5286 | esophagus |
| TMEM190 | ENSG00000160472 | Q8WZ59 | 0.8984 | 0.7845 | fallopian tube |
| TMEM196 | ENSG00000173452 | Q5HYL7 | 0.861 | 0.7818 | hypothalamus |
| TMEM200A | ENSG00000164484 | Q86VY9 | 0.6863 | 0.435 | endometrium |
| TMEM200C | ENSG00000206432 | A6NKL6 | 0.6991 | 0.4245 | salivary gland |
| TMEM207 | ENSG00000198398 | Q6UWV9 | 1 | 1 | kidney |
| TMEM210 | ENSG00000185863 | A6NLX4 | 0.9786 | 0.9659 | salivary gland |
| TMEM211 | ENSG00000206069 | Q6ICI0 | 0.9603 | 0.9136 | salivary gland |
| TMEM212 | ENSG00000186329 | A6NML5 | 0.9798 | 0.9663 | fallopian tube |
| TMEM213 | ENSG00000214128 | A2RRL7 | 0.9546 | 0.9152 | kidney |
| TMEM215 | ENSG00000188133 | Q68D42 | 0.9076 | 0.8553 | cervix, uterine |
| TMEM217 | ENSG00000172738 | Q8N7C4 | 0.9101 | 0.7129 | adrenal gland |
| TMEM229A | ENSG00000234224 | B2RXF0 | 0.7703 | 0.6957 | spinal cord |
| TMEM232 | ENSG00000186952 | C9JQ17 | 0.7741 | 0.4475 | fallopian tube |
| TMEM233 | ENSG00000224982 | B4DJY2 | 0.9113 | 0.6351 | skeletal muscle |
| TMEM235 | ENSG00000204278 | A6NFC5 | 0.8266 | 0.8022 | spinal cord |
| TMEM239 | ENSG00000198326 | Q8WWW34 | 0.9062 | 0.9063 | breast |
| TMEM244 | ENSG00000203756 | Q5VVB8 | 0.9701 | 0.9212 | pituitary gland |
| TMEM252 | ENSG00000181778 | Q8N6L7 | 0.7819 | 0.6137 | kidney |
| TMEM253 | ENSG00000232070 | POC7T8 | 0.8315 | 0.4339 | small intestine |
| TMEM26 | ENSG00000196932 | Q6ZUK4 | 0.8377 | 0.6028 | spleen |
| TMEM266 | ENSG00000169758 | Q2M3C6 | 0.9307 | 0.7627 | cerebellum |
| TMEM270 | ENSG00000175877 | Q6UE05 | 0.9006 | 0.8384 | fallopian tube |
| TMEM30B | ENSG00000182107 | Q3MIR4 | 0.6797 | 0.5177 | thyroid gland |
| TMEM40 | ENSG00000088726 | Q8WWA1 | 0.8446 | 0.7586 | esophagus |
| TMEM45B | ENSG00000151715 | Q96B21 | 0.6524 | 0.506 | small intestine |
| TMEM52B | ENSG00000165685 | Q4KMG9 | 0.9756 | 0.8869 | kidney |
| TMEM61 | ENSG00000143001 | Q8NOU2 | 0.7623 | 0.4747 | pituitary gland |
| TMEM63C | ENSG00000165548 | Q9P1W3 | 0.7216 | 0.4869 | cerebellum |
| TMEM72 | ENSG00000187783 | AOPK05 | 0.9636 | 0.9052 | kidney |
| TMEM82 | ENSG00000162460 | AOPJX8 | 0.932 | 0.9166 | liver |
| TMEM88B | ENSG00000205116 | A6NKF7 | 0.8445 | 0.764 | spinal cord |
| TMEM92 | ENSG00000167105 | Q6UXU6 | 0.8144 | 0.6211 | small intestine |
| TMIE | ENSG00000181585 | Q8NEW7 | 0.6817 | 0.4376 | pituitary gland |
| TMIGD1 | ENSG00000182271 | Q6UXZ0 | 0.9739 | 0.9688 | small intestine |
| TMIGD2 | ENSG00000167664 | Q96BF3 | 0.8122 | 0.5306 | spleen |
| TMPRSS11B | ENSG00000185873 | Q86T26 | 0.9249 | 0.9074 | esophagus |
| TMPRSS11D | ENSG00000153802 | O60235 | 0.9193 | 0.915 | vagina |
| TMPRSS11E | ENSG00000087128 | Q9UL52 | 0.8835 | 0.8607 | esophagus |
| TMPRSS11F | ENSG00000198092 | Q6ZWK6 | 0.924 | 0.9039 | esophagus |
| TMPRSS13 | ENSG00000137747 | Q9BYE2 | 0.8273 | 0.6626 | skin |
| TMPRSS15 | ENSG00000154646 | P98073 | 0.996 | 0.9928 | small intestine |
| TMPRSS2 | ENSG00000184012 | O15393 | 0.6925 | 0.5755 | prostate |
| TMPRSS4 | ENSG00000137648 | Q9NRS4 | 0.7954 | 0.7045 | esophagus |
| TMPRSS5 | ENSG00000166682 | Q9H3S3 | 0.6262 | 0.4203 | spinal cord |
| TMPRSS6 | ENSG00000187045 | Q8IU80 | 0.9345 | 0.788 | liver |
| TMPRSS7 | ENSG00000176040 | Q7RTY8 | 1 | 1 | pituitary gland |
| TMPRSS9 | ENSG00000178297 | Q7Z410 | 0.8902 | 0.624 | spleen |
| TNF | ENSG00000232810 | P01375 | 0.8333 | 0.5721 | spleen |
| TNFRSF11A | ENSG00000141655 | Q9Y6Q6 | 0.6687 | 0.4204 | salivary gland |
| TNFRSF13B | ENSG00000240505 | O14836 | 0.912 | 0.7522 | spleen |
| TNFRSF13C | ENSG00000159958 | Q96RJ3 | 0.8653 | 0.5196 | spleen |
| TNFRSF17 | ENSG00000048462 | Q02223 | 0.8645 | 0.7154 | spleen |
| TNFRSF18 | ENSG00000186891 | Q9Y5U5 | 0.6867 | 0.4385 | cervix, uterine |
| TNFRSF8 | ENSG00000120949 | P28908 | 0.816 | 0.5995 | adipose tissue |
| TNFRSF9 | ENSG00000049249 | Q07011 | 0.944 | 0.8963 | spleen |
| TNFSF11 | ENSG00000120659 | O14788 | 0.9505 | 0.7957 | small intestine |
| TNFSF14 | ENSG00000125735 | 043557 | 0.7283 | 0.4245 | liver |
| TNFSF15 | ENSG00000181634 | O95150 | 0.7614 | 0.6706 | salivary gland |
| TNFSF18 | ENSG00000120337 | Q9UNG2 | 1 | 1 | esophagus |
| TNFSF8 | ENSG00000106952 | P32971 | 0.8072 | 0.4803 | spleen |
| TNFSF9 | ENSG00000125657 | P41273 | 0.6988 | 0.4278 | cervix, uterine |
| TNMD | ENSG00000000005 | Q9H2S6 | 0.9036 | 0.7414 | adipose tissue |
| TPBGL | ENSG00000261594 | PODKB5 | 0.8006 | 0.5435 | basal ganglia |
| TPO | ENSG00000115705 | P07202 | 0.8903 | 0.56 | thyroid gland |
| TRABD2A | ENSG00000186854 | Q86V40 | 0.8179 | 0.5502 | ovary |
| TRAT1 | ENSG00000163519 | Q6PIZ9 | 0.8555 | 0.6536 | spleen |
| TRDN | ENSG00000186439 | Q13061 | 0.8566 | 0.6494 | skeletal muscle |
| TREM1 | ENSG00000124731 | Q9NP99 | 0.8308 | 0.4849 | lung |
| TREML1 | ENSG00000161911 | Q86YW5 | 0.8246 | 0.5072 | spleen |
| TREML2 | ENSG00000112195 | Q5T2D2 | 0.9619 | 0.902 | spleen |
| TRHDE | ENSG00000072657 | Q9UKU6 | 0.64 | 0.4173 | cerebellum |
| TRHR | ENSG00000174417 | P34981 | 1 | 1 | pituitary gland |
| TRPA1 | ENSG00000104321 | O75762 | 0.9337 | 0.9021 | urinary bladder |
| TRPC3 | ENSG00000138741 | Q13507 | 0.8383 | 0.6124 | pituitary gland |
| TRPC4 | ENSG00000133107 | Q9UBN4 | 0.7854 | 0.4917 | endometrium |
| TRPC5 | ENSG00000072315 | Q9UL62 | 0.9233 | 0.8898 | cerebral cortex |
| TRPC6 | ENSG00000137672 | Q9Y210 | 0.7402 | 0.4443 | lung |
| TRPC7 | ENSG00000069018 | Q9HCX4 | 0.9742 | 0.962 | pituitary gland |
| TRPM1 | ENSG00000134160 | Q7Z4N2 | 0.9571 | 0.8828 | skin |
| TRPM2 | ENSG00000142185 | O94759 | 0.6896 | 0.4051 | cerebellum |
| TRPM3 | ENSG00000083067 | Q9HCF6 | 0.7819 | 0.5818 | cerebellum |
| TRPM5 | ENSG00000070985 | Q9NZQ8 | 0.9048 | 0.8356 | small intestine |
| TRPM6 | ENSG00000119121 | Q9BX84 | 0.8399 | 0.5254 | colon |
| TRPM8 | ENSG00000144481 | Q7Z2W7 | 0.9742 | 0.9533 | prostate |
| TRPV3 | ENSG00000167723 | Q8NET8 | 0.9569 | 0.8734 | skin |
| TRPV4 | ENSG00000111199 | Q9HBA0 | 0.7071 | 0.5006 | salivary gland |
| TSHR | ENSG00000165409 | P16473 | 0.9723 | 0.7459 | thyroid gland |
| TSPAN16 | ENSG00000130167 | Q9UKR8 | 0.913 | 0.8885 | amygdala |
| TSPAN19 | ENSG00000231738 | POC672 | 0.8803 | 0.7411 | lung |
| TSPAN32 | ENSG00000064201 | Q96QS1 | 0.8633 | 0.6918 | heart muscle |
| TSPO2 | ENSG00000112212 | Q5TGUO | 0.9499 | 0.8765 | spleen |
| TTYH1 | ENSG00000167614 | Q9H313 | 0.628 | 0.4757 | basal ganglia |
| UGT2A3 | ENSG00000135220 | Q6UWM9 | 0.9119 | 0.8918 | small intestine |
| UGT3A1 | ENSG00000145626 | Q6NUS8 | 0.9535 | 0.9401 | kidney |
| UGT3A2 | ENSG00000168671 | Q3SY77 | 0.9703 | 0.9127 | skin |
| UGT8 | ENSG00000174607 | Q16880 | 0.7692 | 0.5469 | spinal cord |
| UMODL1 | ENSG00000177398 | Q5DIDO | 0.9944 | 0.9905 | fallopian tube |
| UNC5A | ENSG00000113763 | Q6ZN44 | 0.8644 | 0.7438 | cerebellum |
| UNC5D | ENSG00000156687 | Q6UXZ4 | 0.8161 | 0.719 | pituitary gland |
| UNC79 | ENSG00000133958 | Q9P2D8 | 0.8482 | 0.751 | cerebellum |
| UNC80 | ENSG00000144406 | Q8N2C7 | 0.8425 | 0.7327 | cerebellum |
| UNC93A | ENSG00000112494 | Q86WB7 | 0.9474 | 0.8875 | skin |
| UPK1A | ENSG00000105668 | O00322 | 0.8966 | 0.7984 | urinary bladder |
| UPK1B | ENSG00000114638 | O75841 | 0.911 | 0.7294 | urinary bladder |
| UPK2 | ENSG00000110375 | O00526 | 0.9681 | 0.8269 | urinary bladder |
| UPK3B | ENSG00000243566 | Q9BT76 | 0.8058 | 0.6621 | adipose tissue |
| USH2A | ENSG00000042781 | O75445 | 0.9853 | 0.97 | liver |
| UTS2R | ENSG00000181408 | Q9UKP6 | 0.941 | 0.8312 | thyroid gland |
| VSIG1 | ENSG00000101842 | Q86XK7 | 0.9562 | 0.8105 | stomach |
| VSIG10L | ENSG00000186806 | Q86VR7 | 0.8816 | 0.7676 | esophagus |
| VSIG2 | ENSG00000019102 | Q96IQ7 | 0.7017 | 0.4572 | stomach |
| VSIG8 | ENSG00000243284 | PODPA2 | 0.8588 | 0.6674 | skin |
| VSTM1 | ENSG00000189068 | Q6UX27 | 0.9317 | 0.8411 | pituitary gland |
| VSTM2B | ENSG00000187135 | A6NLU5 | 0.7787 | 0.7502 | cerebellum |
| VSTM5 | ENSG00000214376 | A8MXK1 | 0.7864 | 0.5312 | hypothalamus |
| VTCN1 | ENSG00000134258 | Q7Z7D3 | 0.8401 | 0.7564 | breast |
| WSCD2 | ENSG00000075035 | Q2TBF2 | 0.8128 | 0.5399 | cerebellum |
| XCR1 | ENSG00000173578 | P46094 | 0.933 | 0.8323 | skin |
| XG | ENSG00000124343 | P55808 | 0.8147 | 0.6858 | skin |
| XKR3 | ENSG00000172967 | Q5GH77 | 1 | 1 | spleen |
| XKR4 | ENSG00000206579 | Q5GH76 | 0.7925 | 0.6118 | colon |
| XKR7 | ENSG00000260903 | Q5GH72 | 0.954 | 0.8965 | cerebellum |
| XKR9 | ENSG00000221947 | Q5GH70 | 0.7779 | 0.4034 | small intestine |
| XKRX | ENSG00000182489 | Q6PP77 | 0.8727 | 0.6825 | skin |
| ZACN | ENSG00000186919 | Q401N2 | 0.9783 | 0.9744 | colon |
| ZDHHC17 | ENSG00000186908 | Q8IUH5 | 0.8693 | 0.773 | cervix, uterine |
| ZP1 | ENSG00000149506 | P60852 | 0.9836 | 0.9712 | pituitary gland |
| ZP2 | ENSG00000103310 | Q05996 | 0.994 | 0.9796 | cerebellum |
| ZP4 | ENSG00000116996 | Q12836 | 1 | 1 | ovary |
| ZPLD1 | ENSG00000170044 | Q8TCW7 | 0.9773 | 0.9605 | kidney |

### Small Molecules

A macromolecule of the invention (for example, an ANDbody) is linked to a small molecule. The macromolecule and the small molecule may be linked by a cleavable linker. Alternatively, the macromolecule and the small molecule may be linked by a non-cleavable linker. Any useful linker may be employed for this purpose.

One or more (for example, one, two, three, four, five, or more) small molecules may be linked to the macromolecule. If multiple small molecules are linked to a macromolecule, the small molecules may be the same. Alternatively, one or more of the small molecules linked to the macromolecule may be different.

A small molecule to be linked to the macromolecule may be any desired small molecule. For example, the small molecule may be a therapeutic agent of interest that is to be localized or concentrated at a particular site by the macromolecule. In one example, the small molecule may be a therapeutic agent that acts together with or complements the effector target binding site domain. Alternatively, the small molecule may modulate the effector target binding site domain. In another example, the small molecule may modulate the address target binding site domain.

Exemplary classes of small molecules which may be linked to a macromolecule according to the invention include those in Table 3.

TABLE 3
Exemplary Small Molecule Classes
Class

1,3 Beta Glucan Synthase (EC 2.4.1.34) Inhibitor
11 Beta Hydroxysteroid Dehydrogenase (Corticosteroid 11 Beta Dehydrogenase or Corticosteroid 11
Reductase or HSD11 or EC 1.1.1.146) Inhibitor
16S Ribosomal RNA (16S rRNA) Inhibitor
20s Proteasome Inhibitor
23S Ribosomal RNA (23S rRNA) Inhibitor
26s Proteasome Inhibitor
3 Hydroxy 3 Methylglutaryl Coenzyme A Reductase (HMG CoA Reductase or Hydroxymethylglutaryl
CoA Reductase or HMGCR or EC 1.1.1.34) Inhibitor
3 Oxo 5 Alpha Steroid 4 Dehydrogenase (Steroid 5 Alpha Reductase or 5 Alpha Reductase or EC
1.3.1.22) Inhibitor
3 Oxo 5 Alpha Steroid 4 Dehydrogenase 1 (Steroid 5 Alpha Reductase 1 or SR Type 1 or SRD5A1 or
EC 1.3.1.22) Inhibitor
3 Oxo 5 Alpha Steroid 4 Dehydrogenase 2 (5 Alpha SR2 or Steroid 5 Alpha Reductase 2 or Type II 5
Alpha Reductase or SR Type 2 or SRD5A2 or EC 1.3.1.22) Inhibitor
30S Ribosomal Subunit (30S RNA) Inhibitor
4 Aminobutyrate Aminotransferase Mitochondrial ((S) 3 Amino 2 Methylpropionate Transaminase or
GABA Aminotransferase or Gamma Amino N Butyrate Transaminase or L AIBAT or ABAT or EC
2.6.1.22 or EC 2.6.1.19) Inhibitor
4 Hydroxyphenylpyruvate Dioxygenase (4 Hydroxyphenylpyruvic Acid Oxidase or 4HPPD or HPD or
EC 1.13.11.27) Inhibitor
50S Ribosomal Subunit (50S RNA) Inhibitor
5-Hydroxytryptamine Receptor 1 (5 HT1 Receptor or HTR1) Agonist
5-Hydroxytryptamine Receptor 1A (5 HT1A or G 21 or Serotonin Receptor 1A or HTR1A) Agonist
5-Hydroxytryptamine Receptor 1B (5 HT1B or S12 or Serotonin 1D Beta Receptor or Serotonin
Receptor 1B or HTR1B) Agonist
5-Hydroxytryptamine Receptor 1D (5 HT1D or Serotonin 1D Alpha Receptor or Serotonin Receptor
1D or HTR1D) Antagonist
5-Hydroxytryptamine Receptor 1F (5 HT1F or Serotonin Receptor 1F or HTR1F) Agonist
5-Hydroxytryptamine Receptor 2 (5 HT2 Receptor or HTR2) Antagonist
5-Hydroxytryptamine Receptor 2A (5 HT2A or Serotonin Receptor 2A or HTR2A) Antagonist
5-Hydroxytryptamine Receptor 2C (5 HT2C or 5 Hydroxytryptamine Receptor 1C or Serotonin
Receptor 2C or HTR2C) Antagonist
5-Hydroxytryptamine Receptor 3 (5HT3 or 5 HT3 Receptor or HTR3) Antagonist
5-Hydroxytryptamine Receptor 4 (5 HT4 or Serotonin Receptor 4 or HTR4) Agonist
5-Hydroxytryptamine Receptor 7 (5 HT7 or 5 HTX or Serotonin Receptor 7 or HTR7) Antagonist
70S Ribosome Inhibitor
Acetylcholinesterase (Acetylcholine Hydrolase or Yt Blood Group or Apoptosis Related
Acetylcholinesterase or ACHE or EC 3.1.1.7) Activator
Acetylcholinesterase (Acetylcholine Hydrolase or Yt Blood Group or Apoptosis Related
Acetylcholinesterase or ACHE or EC 3.1.1.7) Inhibitor
Adenosine Deaminase (Adenosine Aminohydrolase or ADA or EC 3.5.4.4) Inhibitor
Adenosine Monophosphate Activated Protein Kinase ([Hydroxymethylglutaryl CoA Reductase
(NADPH)] Kinase or AMPK or EC 2.7.11.31) Activator
Adenosine Receptor (ADORA) Antagonist
Adenosine Receptor A2a (ADORA2A) Antagonist
Adenosine Receptor A2b (ADORA2B) Antagonist
Adrenocorticotropic Hormone Receptor (Adrenocorticotropin Receptor or Melanocortin Receptor 2 or
ACTHR or MC2R) Agonist
ALK Tyrosine Kinase Receptor (Anaplastic Lymphoma Kinase or CD246 or ALK or EC 2.7.10.1)
Inhibitor
Alpha 1 Adrenergic Receptor (ADRA1) Agonist
Alpha 1 Adrenergic Receptor (ADRA1) Antagonist
Alpha 1B Adrenergic Receptor (Alpha 1B Adrenoreceptor or ADRA1B) Antagonist
Alpha 2 Adrenergic Receptor (ADRA2) Agonist
Alpha 2 Adrenergic Receptor (ADRA2) Antagonist
Alpha 2A Adrenergic Receptor (Alpha 2 Adrenergic Receptor Subtype C10 or Alpha 2A
Adrenoreceptor or ADRA2A) Agonist
Alpha 2C Adrenergic Receptor (Alpha 2 Adrenergic Receptor Subtype C4 or Alpha 2C
Adrenoreceptor or ADRA2C) Antagonist
Alpha Adrenergic Receptor (ADRA) Agonist
Alpha Amylase 2B (1,4-Alpha D-Glucan Glucanohydrolase 2B or Carcinoid Alpha Amylase or AMY2B
or EC 3.2.1.1) Inhibitor
Alpha Galactosidase A (Alpha D-Galactosidase A or Alpha D Galactoside Galactohydrolase or
Melibiase or Agalsidase or GLA or EC 3.2.1.22) Activator
Amine Oxidase [Flavin Containing] A (Monoamine Oxidase Type A or MAOA or EC 1.4.3.4) Inhibitor
Androgen Receptor (Dihydrotestosterone Receptor or Nuclear Receptor Subfamily 3 Group C
Member 4 or DHTR or NR3C4 or AR) Agonist
Androgen Receptor (Dihydrotestosterone Receptor or Nuclear Receptor Subfamily 3 Group C
Member 4 or DHTR or NR3C4 or AR) Antagonist
Angiopoietin 1 Receptor (Endothelial Tyrosine Kinase or Tunica Interna Endothelial Cell Kinase or
Tyrosine Kinase With Ig And EGF Homology Domains 2 or Tyrosine Protein Kinase Receptor TEK or
Tyrosine Protein Kinase Receptor TIE 2 or p140 TEK or CD202b or TIE2 or TEK or EC 2.7.10.1)
Inhibitor
Angiotensin Converting Enzyme (Dipeptidyl Carboxypeptidase I or Kininase II or CD143 or ACE or EC
3.4.15.1 or EC 3.2.1.) Inhibitor
Arachidonate 5 Lipoxygenase (5 Lipoxygenase or Leukotriene A4 Synthase or ALOX5 or EC
1.13.11.34) Inhibitor
Aromatase (Estrogen Synthase or Cytochrome P 450AROM or Cytochrome P450 19A1 or CYP19A1
or EC 1.14.14.14) Inhibitor
Aryl Hydrocarbon Receptor (Class E Basic Helix Loop Helix Protein 76 or bHLHe76 or AHR) Agonist
ATP Binding Cassette Sub Family C Member 8 (Sulfonylurea Receptor 1 or ABCC8) Inhibitor
ATP Citrate Synthase (ATP Citrate (Pro S) Lyase or Citrate Cleavage Enzyme or ACLY or EC 2.3.3.8)
Inhibitor
ATP Sensitive Inward Rectifier Potassium Channel 1 (Potassium Channel Inwardly Rectifying
Subfamily J Member 1 or Inward Rectifier Potassium Channel Kir1.1 or KCNJ1) Activator
B Cell Lymphoma 2 (Bcl 2) Inhibitor
Bacterial Cell Membrane Disruptor
C55 Isoprenyl Pyrophosphate Inhibitor
Glucocorticoid Receptor (GR or Nuclear Receptor Subfamily 3 Group C Member 1 or NR3C1) Agonist
Bacterial Cell Wall Disruptor
Bcr-Abl Tyrosine Kinase (EC 2.7.10.2) Inhibitor
BDNF/NT 3 Growth Factors Receptor (GP145 TrkB or Neurotrophic Tyrosine Kinase Receptor Type 2
or TrkB Tyrosine Kinase or Tropomyosin Related Kinase B or NTRK2 or EC 2.7.10.1) Inhibitor
Beta 1 Adrenergic Receptor (Beta 1 Adrenoreceptor or ADRB1) Agonist
Beta 1 Adrenergic Receptor (Beta 1 Adrenoreceptor or ADRB1) Agonist
Beta 2 Adrenergic Receptor (Beta 2 Adrenoreceptor or ADRB2) Agonist
Beta 3 Adrenergic Receptor (Beta 3 Adrenoreceptor or ADRB3) Agonist
Beta Adrenergic Receptor (ADRB) Agonist
Beta Hematin (Hemozoin) Inhibitor
Beta Lactamase (EC 3.5.2.6) Inhibitor
Bile Acid Receptor (Farnesoid X Activated Receptor or Farnesol Receptor HRR 1 or Nuclear Receptor
Subfamily 1 Group H Member 4 or Retinoid X Receptor Interacting Protein 14 or FXR or NR1H4)
Agonist
Bile Acid Sequestrant
C5a Anaphylatoxin Chemotactic Receptor 1 (CD88 or C5AR1) Antagonist
Calcineurin (Protein Serine/Threonine Phosphatase 3 or Protein Phosphatase 3 or EC 3.1.3.16)
Inhibitor
Calcitonin Gene Related Peptide Type 1 Receptor (Calcitonin Receptor Like Receptor or CALCRL)
Antagonist
Calcium Activated Potassium Channel Blocker
Calcium Channel Blocker
Calcium Chelator
Calcium Sensing Receptor Agonist
Cannabinoid Receptor 1 (CB1 or CANN6 or CNR1) Agonist
Cannabinoid Receptor 2 (CB2 or CX5 or CNR2) Agonist
Carbamoyl Phosphate Synthase [Ammonia] Mitochondrial (Carbamoyl Phosphate Synthetase I or
CPS1 or EC 6.3.4.16) Activator
Carbonic Anhydrase (Carbonate Dehydratase or Carbonic Acid Anhydrase or Carboxyanhydrase or
CA or EC 4.2.1.1) Inhibitor
Carbonic Anhydrase 2 (Carbonate Dehydratase II or Carbonic Anhydrase C or CAC or CA2 or EC
4.2.1.1) Inhibitor
Carbonic Anhydrase 4 (Carbonate Dehydratase IV or CA4 or EC 4.2.1.1) Inhibitor; Gamma-
Aminobutyric Acid Type A Receptor Subunit (GABA(A) Receptor or GABR) Agonist
Catechol O Methyltransferase (Epididymis Secretory Sperm Binding Protein Li 98n or COMT or EC
2.1.1.6) Inhibitor
C-C Chemokine Receptor Type 5 (CHEMR13 or HIV 1 Fusion Coreceptor or CD195 or CCR5)
Antagonist
Cell Membrane Disruptor
Ceramide Glucosyltransferase (Glucosylceramide Synthase or GLCT 1 or UDP Glucose:N
Acylsphingosine D Glucosyltransferase or UDP Glucose Ceramide Glucosyltransferase or UGCG or
EC 2.4.1.80) Inhibitor
CGMP Specific 3′,5′ Cyclic Phosphodiesterase (cGMP Binding cGMP Specific Phosphodiesterase or
PDE5 or PDE5A or EC 3.1.4.35) Inhibitor
Chloride Channel Protein 2 (CLCN2) Activator
Cholinergic Receptor Muscarinic (Muscarinic Acetylcholine Receptor or CHRM) Antagonist
Cholinergic Receptor Muscarinic (Muscarinic Acetylcholine Receptor or CHRM) Agonist
Cholinergic Receptor Nicotinic Subunit (Nicotinic Acetylcholine Receptor or CHRN) Agonist
Cholinergic Receptor Nicotinic Subunit (Nicotinic Acetylcholine Receptor or CHRN) Antagonist
Cholinesterase (Butyrylcholinesterase or Acylcholine Acylhydrolase or Choline Esterase II or
Pseudocholinesterase or BCHE or EC 3.1.1.8) Inhibitor
Coagulation Factor X (Stuart Prower Factor or Stuart Factor or F10 or EC 3.4.21.6) Inhibitor
Copper Chelator
Cyclic Nucleotide Gated Channel (CNG) Blocker
Cyclin Dependent Kinase 4 (Cell Division Protein Kinase 4 or PSK J3 or CDK4 or EC 2.7.11.22)
Inhibitor
Cyclin Dependent Kinase 6 (Cell Division Protein Kinase 6 or Serine/Threonine Protein Kinase
PLSTIRE or CDK6 or EC 2.7.11.22) Inhibitor
Cysteinyl Leukotriene Receptor 1 (Cysteinyl Leukotriene D4 Receptor or G Protein Coupled Receptor
HG55 or HMTMF81 or CYSLTR1) Antagonist
Cysteinyl Leukotriene Receptor 2 (G Protein Coupled Receptor GPCR21 or G Protein Coupled
Receptor HG57 or HPN321 or CYSLTR2) Antagonist
Cystic Fibrosis Transmembrane Conductance Regulator (ATP Binding Cassette Sub Family C
Member 7 or Channel Conductance Controlling ATPase Camp Dependent Chloride Channel or
ABCC7 or CFTR or EC 5.6.1.6) Activator
Cystine Depletor
Cytidine Deaminase (Cytidine Aminohydrolase or CDA or EC 3.5.4.5) Inhibitor
Cytochrome bc1 Complex (Complex III) Inhibitor
Cytochrome c Oxidase (Cytochrome a3 or Indophenolase or Ferrocytochrome c Oxidase or EC
1.9.3.1) Inhibitor
Cytochrome P450 11B2 Mitochondrial (Aldosterone Synthase or Cytochrome P450Aldo or
Cytochrome P450C18 or Steroid 18 Hydroxylase or CYP11B2 or EC 1.14.15.4 or EC 1.14.15.5)
Inhibitor
Cytochrome P450 3A4 (1,8 Cineole 2 Exo Monooxygenase or Albendazole Monooxygenase or
Albendazole Sulfoxidase or Cholesterol 25 Hydroxylase or Cytochrome P450 3A3 or Nifedipine
Oxidase or Quinine 3 Monooxygenase or Taurochenodeoxycholate 6 Alpha Hydroxylase or CYP3A4
or EC 1.14.14. or EC 1.14.14.56 or EC 1.14.14.73 or EC 1.14.14.55) Inhibitor
Cytochrome P450 3A5 (Cytochrome P450 HLp2 or Cytochrome P450 PCN3 or CYP3A5 or EC
1.14.14.1) Inhibitor; HIV 1 Integrase (EC 2.7.7.) Inhibitor
Cytotoxic To Cells Expressing Glutamate Carboxypeptidase 2 (Folate Hydrolase 1 or Prostate
Specific Membrane Antigen or PSMA or Pteroylpoly Gamma Glutamate Carboxypeptidase or Cell
Growth Inhibiting Gene 27 Protein or FOLH1 or EC 3.4.17.21)
D1A Dopamine Receptor (Dopamine D1 Receptor or DRD1) Agonist
D1B Dopamine Receptor (D5 Dopamine Receptor or D1 Beta Dopamine Receptor or Dopamine D5
Receptor or DRD5) Agonist
D2 Dopamine Receptor (Dopamine D2 Receptor or DRD2) Agonist
D3 Dopamine Receptor (Dopamine D3 Receptor or DRD3) Agonist
D4 Dopamine Receptor (D2C Dopamine Receptor or Dopamine D4 Receptor or DRD4) Agonist
Delta Type Opioid Receptor (DOR1 or OPRD or OPRD1) Antagonist
Diacylglycerol O Acyltransferase (Diglyceride Acyltransferase or DGAT or EC 2.3.1.20) Inhibitor
Dihydrofolate Reductase (DHFR or EC 1.5.1.3) Inhibitor
Dihydroorotate Dehydrogenase (Quinone) Mitochondrial (Dihydroorotate Oxidase or DHODH or EC
1.3.5.2) Inhibitor
Dihydropteroate Synthase (EC 2.5.1.15) Inhibitor
Dihydropyrimidinase Related Protein 2 (Collapsin Response Mediator Protein 2 or CRMP2 or N2A3 or
Unc 33 Like Phosphoprotein 2 or DPYSL2) Inhibitor
Dipeptidyl Peptidase 1 (Cathepsin C or Cathepsin J or Dipeptidyl Transferase or DPPI or CTSC or EC
3.4.14.1) Inhibitor
Dipeptidyl Peptidase 4 (ADABP or Adenosine Deaminase Complexing Protein 2 or T Cell Activation
Antigen CD26 or TP103 or CD26 or DPP4 or EC 3.4.14.5) Inhibitor
DNA (Cytosine 5) Methyltransferase 1 (CXXC Type Zinc Finger Protein 9 or DNA Methyltransferase
Hsal or MCMT or DNMT1 or EC 2.1.1.37) Inhibitor
DNA Directed RNA Polymerase (POLR2 or EC 2.7.7.6) Inhibitor
DNA Directed RNA Polymerase Subunit Beta (RNA Polymerase Subunit Beta or Transcriptase
Subunit Beta or rpoB or EC 2.7.7.6) Inhibitor
DNA Gyrase (EC 5.99.1.3) Inhibitor
DNA Helicase (EC 3.6.4.12) Inhibitor
DNA Inhibitor
DNA Ligase (EC 6.5.1.) Inhibitor
DNA Polymerase (EC 2.7.7.7) Inhibitor
DNA Polymerase Alpha (POLA or EC 2.7.7.7) Inhibitor
DNA Primase (EC 2.7.7.6) Inhibitor
DNA Synthesis Inhibitor
DNA Topoisomerase I (TOP1 or EC 5.99.1.2) Inhibitor
DNA Topoisomerase II (EC 5.99.1.3) Inhibitor
DNA Topoisomerase IV (EC 5.99.1.3) Inhibitor
Dopamine Receptor (DRD) Agonist
Dopamine Receptor (DRD) Antagonist
Dual Specificity Mitogen Activated Protein Kinase Kinase 1 (ERK Activator Kinase 1 or MAPK/ERK
Kinase 1 or MAP2K1 or EC 2.7.12.2) Inhibitor
Dual Specificity Mitogen Activated Protein Kinase Kinase 2 (ERK Activator Kinase 2 or MAPK/ERK
Kinase 2 or MAP2K2 or EC 2.7.12.2) Inhibitor
Endothelial PAS Domain Containing Protein 1 (Basic Helix Loop Helix PAS Protein MOP2 or Class E
Basic Helix Loop Helix Protein 73 or HIF 1 Alpha Like Factor or Hypoxia Inducible Factor 2 Alpha or
PAS Domain Containing Protein 2 or EPAS1) Inhibitor
Endothelin 1 Receptor (Endothelin A Receptor or EDNRA) Antagonist
Endothelin B Receptor (Endothelin Receptor Non Selective Type or EDNRB) Antagonist
Enoyl [Acyl Carrier Protein] Reductase [NADH] Fabl (NADH Dependent Enoyl ACP Reductase or
Enoyl ACP Reductase or EC 1.3.1.9) Inhibitor
Epidermal Growth Factor Receptor (Proto Oncogene c ErbB 1 or Receptor Tyrosine Protein Kinase
erbB 1 or HER1 or ERBB1 or EGFR or EC 2.7.10.1) Inhibitor
Equilibrative Nucleoside Transporter 1 (Equilibrative Nitrobenzylmercaptopurine Riboside Sensitive
Nucleoside Transporter or Nucleoside Transporter Es Type or Solute Carrier Family 29 Member 1 or
SLC29A1) Inhibitor
Ergosterol Inhibitor
Estrogen Receptor (ER Alpha or Estradiol Receptor or Nuclear Receptor Subfamily 3 Group A
Member 1 or NR3A1 or ESR1) Agonist
Estrogen Receptor (ER Alpha or Estradiol Receptor or Nuclear Receptor Subfamily 3 Group A
Member 1 or NR3A1 or ESR1) Antagonist
Estrogen Receptor (ESR) Agonist
Estrogen Receptor (ESR) Antagonist
Estrogen Receptor (ESR) Modulator
Estrogen Receptor Beta (ER Beta or Nuclear Receptor Subfamily 3 Group A Member 2 or NR3A2 or
ESR2) Agonist
Estrogen Receptor Beta (ER Beta or Nuclear Receptor Subfamily 3 Group A Member 2 or NR3A2 or
ESR2) Antagonist
Exo Alpha Sialidase (Neuraminidase or Acetylneuraminidase or EC 3.2.1.18) Inhibitor
Exportin 1 (Chromosome Region Maintenance 1 Protein Homolog or XPO1) Inhibitor
Farnesyl Pyrophosphate Synthase (Farnesyl Diphosphate Synthase or (2E,6E) Farnesyl Diphosphate
Synthase or Dimethylallyltranstransferase or Geranyltranstransferase or FDPS or EC 2.5.1.10 or EC
2.5.1.1) Inhibitor
Fibroblast Growth Factor Receptor 1 (Basic Fibroblast Growth Factor Receptor 1 or Fms Like
Tyrosine Kinase 2 or N Sam or Proto Oncogene c Fgr or CD331 or FLT2 or FGFR1 or EC 2.7.10.1)
Inhibitor
Fibroblast Growth Factor Receptor 2 (Keratinocyte Growth Factor Receptor or K Sam or KGFR or
CD332 or FGFR2 or EC 2.7.10.1) Inhibitor
Fibroblast Growth Factor Receptor 3 (Tyrosine Kinase JTK4 or Hydroxyaryl Protein Kinase or CD333
or FGFR3 or EC 2.7.10.1) Inhibitor
Fibroblast Growth Factor Receptor 4 (CD334 or FGFR4 or EC 2.7.10.1) Inhibitor
Free Radical Scavenger
G Protein Coupled Receptor 55 (GPR55) Antagonist
Gamma-Aminobutyric Acid Receptor Subunit Alpha 1 (GABA(A) Receptor Subunit Alpha 1 or
GABRA1) Agonist
Gamma-Aminobutyric Acid Receptor Subunit Alpha 3 (GABA(A) Receptor Subunit Alpha 3 or
GABRA3) Agonist
Gamma-Aminobutyric Acid Receptor Subunit Gamma 2 (GABA(A) Receptor Subunit Gamma 2 or
GABRG2) Agonist
Gamma-Aminobutyric Acid Type A Receptor Subunit (GABA(A) Receptor or GABR) Agonist
Gamma-Aminobutyric Acid Type A Receptor Subunit (GABA(A) Receptor or GABR) Antagonist
Gamma-Aminobutyric Acid Type A Receptor Subunit Alpha (GABA(A) Receptor Subunit Alpha or
GABRA) Agonist
Gamma-Aminobutyric Acid Type B Receptor Subunit (Gamma Aminobutyric Acid (GABA) B Receptor
or GABBR) Agonist
Gastric Triacylglycerol Lipase (Gastric Lipase or LIPF or EC 3.1.1.3) Inhibitor
Geranylgeranyl Pyrophosphate Synthase (Geranylgeranyl Diphosphate Synthase or
Dimethylallyltranstransferase or Farnesyl Diphosphate Synthase or Farnesyltranstransferase or
Geranyltranstransferase or GGPS1 or EC 2.5.1.29 or EC 2.5.1.1 or EC 2.5.1.10) Inhibitor
Glucagon Receptor (GL R or GCGR) Antagonist
Glucocorticoid Receptor (GR or Nuclear Receptor Subfamily 3 Group C Member 1 or NR3C1) Agonist
Glucocorticoid Receptor (GR or Nuclear Receptor Subfamily 3 Group C Member 1 or NR3C1)
Antagonist
Glutamate lonotropic Receptor (GRI) Antagonist
Glutamate lonotropic Receptor AMPA Type Subunit (AMPA Receptor or GRIA) Antagonist
Glutamate lonotropic Receptor Kainate Type Subunit (Kainate Receptor or GRIK) Antagonist
Glutamate lonotropic Receptor NMDA Type Subunit (NMDAR or GRIN) Antagonist
Glutamate Metabotropic Receptor (GRM) Agonist
Glutamine Depletor
Gonadotropin Releasing Hormone Receptor (GNRHR) Agonist
Gonadotropin Releasing Hormone Receptor (GNRHR) Antagonist
GTPase KRas (KRas 2 or Ki Ras or c K Ras or KRAS or EC 3.6.5.2) Inhibitor
Guanylate Cyclase (Guanylyl Cyclase or GTP Diphosphate-Lyase (Cyclizing) or GC or EC 4.6.1.2)
Activator
H+ Transporting Two Sector ATPase (F1F0 ATP Synthase or ATP Synthase or Mitochondrial ATPase
or Bacterial Ca2+/Mg2+ ATPase or EC 7.1.2.2) Inhibitor
Hepacivirin (NS3/4A Protease or NS3 Serine Proteinase or EC 3.4.21.98) Inhibitor
Hepatitis B Virus DNA Polymerase (Hepatitis B Virus Reverse Transcriptase or Hepatitis B Virus
Ribonuclease H or EC 2.7.7.7 or EC 2.7.7.49 or EC 3.1.26.4) Inhibitor
Hepatocyte Growth Factor Receptor (Proto Oncogene c Met or Tyrosine Protein Kinase Met or
HGF/SF Receptor or Scatter Factor Receptor or MET or EC 2.7.10.1) Inhibitor
High Affinity Nerve Growth Factor Receptor (Neurotrophic Tyrosine Kinase Receptor Type 1 or TRK1
Transforming Tyrosine Kinase Protein or Tropomyosin Related Kinase A or Tyrosine Kinase Receptor
or gp140trk or p140 TrkA or NTRK1 or EC 2.7.10.1) Inhibitor
Histamine H1 Receptor (HRH1) Antagonist
Histamine H2 Receptor (Gastric Receptor I or HRH2) Antagonist
Histamine H3 Receptor (G Protein Coupled Receptor 97 or GPCR97 or HRH3) Antagonist
Histone Deacetylase (HDAC or EC 3.5.1.98) Inhibitor
Histone Deacetylase 1 (HDAC1 or EC 3.5.1.98) Inhibitor
Histone Deacetylase 2 (Transcriptional Regulator Homolog RPD3 or YY1 Associated Factor 1 or
HDAC2 or EC 3.5.1.98) Inhibitor
Histone Deacetylase 3 (SMAP45 or RPD3 2 or HDAC3 or EC 3.5.1.98) Inhibitor
Histone Deacetylase 6 (Protein Phosphatase 1 Regulatory Subunit 90 or HDAC6 or EC 3.5.1.98)
Inhibitor
Histone Lysine N Methyltransferase EZH2 (ENX 1 or Enhancer Of Zeste Homolog 2 or Lysine N
Methyltransferase 6 or EZH2 or EC 2.1.1.43) Inhibitor
HIV 1 Integrase (EC 2.7.7.) Inhibitor
HIV 1 Retropepsin (HIV Aspartyl Protease or HIV Proteinase or Retroproteinase or Gag Protease or
HIV Aspartyl Protease or EC 3.4.23.16) Inhibitor
HIV 2 Retropepsin (HIV 2 Protease or EC 3.4.23.47) Inhibitor
HIV Integrase (EC 2.7.7.) Inhibitor
Human Cytomegalovirus Viral Terminase Inhibitor
Hypoxanthine Guanine Phosphoribosyltransferase (HPRT1 or EC 2.4.2.8) Inhibitor
Ileal Sodium/Bile Acid Cotransporter (Apical Sodium Dependent Bile Acid Transporter or ASBT or
Sodium/Taurocholate Cotransporting Polypeptide Ileal or Solute Carrier Family 10 Member 2 or
SLC10A2) Inhibitor
Influenza M2 Proton Channel Blocker
Inosine Monophosphate Dehydrogenase (Inosinic Acid Dehydrogenase or IMP Oxidoreductase or
IMPDH or EC 1.1.1.205) Inhibitor
Integrin Alpha 2b (GPalpha IIb or Platelet Membrane Glycoprotein IIb or CD41 or ITGA2B) Antagonist
Integrin Alpha L (CD11 Antigen Like Family Member A or Leukocyte Adhesion Glycoprotein LFA 1
Alpha Chain or CD11a or ITGAL) Antagonist
Interferon Alpha/Beta Receptor 1 (Cytokine Receptor Class II Member 1 or Cytokine Receptor Family
2 Member 1 or Type I Interferon Receptor 1 or IFNAR1) Agonist
Interferon Alpha/Beta Receptor 2 (Interferon Alpha Binding Protein or Type I Interferon Receptor 2 or
IFNAR2) Agonist
Interleukin 1 Receptor Associated Kinase 1 (IRAK1 or EC 2.7.11.1) Inhibitor
Iron Chelator
Iron Replacement
Isocitrate Dehydrogenase [NADP] Cytoplasmic (Oxalosuccinate Decarboxylase or Cytosolic NADP
Isocitrate Dehydrogenase or IDP or NADP(+) Specific ICDH or IDH1 or EC 1.1.1.42) Inhibitor
Isocitrate Dehydrogenase [NADP] Mitochondrial (NADP(+)-Specific ICDH or Oxalosuccinate
Decarboxylase or ICD M or IDP or IDH2 or EC 1.1.1.42) Inhibitor
Isoleucine tRNA Ligase Cytoplasmic (Isoleucyl tRNA Synthetase or IARS or EC 6.1.1.5) Inhibitor
Kallikrein (KLK or EC 3.4.21.) Inhibitor
Kappa Type Opioid Receptor (KOR1 or OPRK or OPRK1) Agonist
Kappa Type Opioid Receptor (KOR1 or OPRK or OPRK1) Antagonist
Lanosterol 14 Alpha Demethylase (Sterol 14 Alpha Demethylase or Cytochrome P450 14DM or
Cytochrome P450LI or Cytochrome P450 51A1 or CYPLI or CYP51A1 or EC 1.14.14.154) Inhibitor
Lead Chelator
Leucine tRNA Ligase Cytoplasmic (Leucyl tRNA Synthetase or LeuRS or LARS or EC 6.1.1.4)
Inhibitor
Leukotriene B4 (LTB4) Inhibitor
Leukotriene D4 (LTD4) Inhibitor
Leukotriene Inhibitor
Lipoxygenase (LOX or EC 1.13.11.) Inhibitor
Macrophage Colony Stimulating Factor 1 Receptor (CSF 1 Receptor or Proto Oncogene c Fms or
CD115 or CSF1R or EC 2.7.10.1) Inhibitor
Major Histocompatibility Complex (MHC) Inhibitor
Maltase Glucoamylase (Alpha-1 4-Glucosidase or MGAM or EC 3.2.1.20) Inhibitor
Mast/Stem Cell Growth Factor Receptor Kit (Proto Oncogene c Kit or Tyrosine Protein Kinase Kit or v
Kit Hardy Zuckerman 4 Feline Sarcoma Viral Oncogene Homolog or Piebald Trait Protein or p145 c
Kit or CD117 or KIT or EC 2.7.10.1) Inhibitor
Melatonin Receptor Type 1A (MT1 or MTNR1A) Agonist
Melatonin Receptor Type 1B (MT2 or MTNR1B) Agonist
Mercury Chelator
Microsomal Triglyceride Transfer Protein Large Subunit (MTTP) Inhibitor
Mineralocorticoid Receptor (Nuclear Receptor Subfamily 3 Group C Member 2 or Mineralocorticoid
Receptor Delta or Aldosterone Receptor or MR or NR3C2) Agonist
Mineralocorticoid Receptor (Nuclear Receptor Subfamily 3 Group C Member 2 or Mineralocorticoid
Receptor Delta or Aldosterone Receptor or MR or NR3C2) Antagonist
Monoamine Oxidase (Adrenalin Oxidase or Epinephrine Oxidase or MAO or EC 1.4.3.4) Inhibitor
Mu Type Opioid Receptor (MOR1 or Mu Opiate Receptor or Mu Opioid Receptor or OPRM1)
Antagonist
Mu Type Opioid Receptor (MOR1 or Mu Opiate Receptor or Mu Opioid Receptor or OPRM1) Agonist
Muscarinic Acetylcholine Receptor M1 (CHRM1) Agonist
Muscarinic Acetylcholine Receptor M1 (CHRM1) Antagonist
Muscarinic Acetylcholine Receptor M3 (CHRM3) Agonist
Muscarinic Acetylcholine Receptor M3 (CHRM3) Antagonist
Muscarinic Acetylcholine Receptor M4 (CHRM4) Antagonist
Myosin Inhibitor
Na+/K+ Exchanging ATPase (Sodium Potassium ATPase or Sodium Potassium Pump or EC
7.2.2.13) Inhibitor
NAD+ ADP Ribosyltransferase (Poly Adenosine Diphosphate Ribose Polymerase or PARP or EC
2.4.2.30) Inhibitor
Neprilysin (Neutral Endopeptidase 24.11 or Atriopeptidase or Common Acute Lymphocytic Leukemia
Antigen or Skin Fibroblast Elastase or Enkephalinase or CD10 or MME or EC 3.4.24.11) Inhibitor
Niemann Pick C1 Like Protein 1 (NPC1L1) Inhibitor
Non Receptor Tyrosine Protein Kinase TYK2 (TYK2 or EC 2.7.10.2) Inhibitor
Nonstructural Protein 5A (NS5A) Inhibitor
NS5B (Nonstructural Protein 5B Polymerase or EC 2.7.7.48) Inhibitor
NT 3 Growth Factor Receptor (TrkC Tyrosine Kinase or GP145 TrkC or Neurotrophic Tyrosine Kinase
Receptor Type 3 or NTRK3 or EC 2.7.10.1) Inhibitor
Nuclear Factor Erythroid 2 Related Factor 2 (HEBP1 or Nuclear Factor Erythroid Derived 2 Like 2 or
NFE2L2 or NRF2) Activator
Orexin Receptor Type 1 (Hypocretin Receptor Type 1 or HCRTR1) Antagonist
Orexin Receptor Type 2 (Hypocretin Receptor Type 2 or HCRTR2) Antagonist
P2Y Purinoceptor 12 (ADP Glucose Receptor or P2Y12 Platelet ADP Receptor or SP1999 or
P2T(AC) or P2Y(AC) or P2Y(cyc) or P2Y12 or P2RY12) Antagonist
p37 Envelope Protein (Envelope Protein Vaccinia Virus) Inhibitor
Pancreatic Alpha Amylase (1,4 Alpha D Glucan Glucanohydrolase or AMY2A or EC 3.2.1.1) Inhibitor
Pancreatic Triacylglycerol Lipase (Pancreatic Lipase or Triacylglycerol Acylhydrolase or PNLIP or EC
3.1.1.3) Inhibitor
Penicillin Binding Protein (PBP) Inhibitor
Penicillin Binding Protein (PBP) Inhibitor; Potassium Transporting ATPase Alpha Chain 1 (Gastric
H(+)/K(+) ATPase Subunit Alpha or Proton Pump or ATP4A or EC 7.2.2.19) Inhibitor
Penicillin Binding Protein 1A (PBP1A) Inhibitor
Penicillin Binding Protein 1B (PBP1B) Inhibitor
Penicillin Binding Protein 1C (PBP1C) Inhibitor
Penicillin Binding Protein 2a (PBP2a) Inhibitor
Penicillin Binding Protein 3 (PBP3) Inhibitor
Peptidoglycan (Murein) Inhibitor
Peptidyl Prolyl Cis Trans Isomerase FKBP1A (12 kDa FK506 Binding Protein or Calstabin 1 or FK506
Binding Protein 1A or Immunophilin FKBP12 or Rotamase or FKBP12 or FKBP1A or EC 5.2.1.8)
Inhibitor
Peroxisome Proliferator Activated Receptor Alpha (Nuclear Receptor Subfamily 1 Group C Member 1
or NR1C1 or PPARA) Agonist
Peroxisome Proliferator Activated Receptor Gamma (Nuclear Receptor Subfamily 1 Group C Member
3 or NR1C3 or PPARG) Agonist
Phenylalanine 4 Hydroxylase (Phe 4 Monooxygenase or PAH or EC 1.14.16.1) Activator
Phosphate Binder
Phosphatidylinositol 4,5 Bisphosphate 3 Kinase Catalytic Subunit Alpha Isoform (PI3K-Alpha or
Phosphatidylinositol 4,5 Bisphosphate 3 Kinase 110 kDa Catalytic Subunit Alpha or Phosphoinositide
3 Kinase Catalytic Alpha Polypeptide or Serine/Threonine Protein Kinase PIK3CA or PIK3CA or EC
2.7.1.137 or EC 2.7.1.153) Inhibitor
Phosphodiesterase 3 (PDE3 or EC 3.1.4.17) Inhibitor
Phosphodiesterase 4 (PDE4 or EC 3.1.4.53) Inhibitor
Plasminogen Activator (EC 3.4.21.) Inhibitor
Platelet Derived Growth Factor Receptor Alpha (Alpha Type Platelet Derived Growth Factor Receptor
or CD140 Antigen Like Family Member A or Platelet Derived Growth Factor Receptor 2 or CD140a or
PDGFRA or EC 2.7.10.1) Inhibitor
Platelet Derived Growth Factor Receptor Beta (Beta Type Platelet Derived Growth Factor Receptor or
CD140 Antigen Like Family Member B or Platelet Derived Growth Factor Receptor 1 or CD140b or
PDGFRB or EC 2.7.10.1) Inhibitor
Poly [ADP Ribose] Polymerase 1 (ADP Ribosyltransferase Diphtheria Toxin Like 1 or NAD(+) ADP
Ribosyltransferase 1 or Poly[ADP Ribose] Synthase 1 or PARP1 or EC 2.4.2.30) Inhibitor
Poly [ADP Ribose] Polymerase 2 (ADP Ribosyltransferase Diphtheria Toxin Like 2 or NAD(+) ADP
Ribosyltransferase 2 or Poly[ADP Ribose] Synthase 2 or PARP2 or EC 2.4.2.30) Inhibitor
Poly [ADP Ribose] Polymerase 3 (ADP Ribosyltransferase Diphtheria Toxin Like 3 or NAD(+) ADP
Ribosyltransferase 3 or Poly[ADP Ribose] Synthase 3 or PARP3 or EC 2.4.2.30) Inhibitor
Polymerase Acidic Protein (RNA Directed RNA Polymerase Subunit P2 or PA or EC 3.1.) Inhibitor
Potassium Channel (KCN) Activator
Potassium Channel (KCN) Blocker
Potassium Channel Voltage Gated (KCNN) Blocker
Potassium Depletor
Potassium Transporting ATPase Alpha Chain 1 (Gastric H(+)/K(+) ATPase Subunit Alpha or Proton
Pump or ATP4A or EC 7.2.2.19) Inhibitor
Potassium Voltage Gated Channel Subfamily H Member 2 (HERG or Voltage Gated Potassium
Channel Subunit Kv11.1 or Eag Homolog or KCNH2) Blocker
Progesterone Receptor (Nuclear Receptor Subfamily 3 Group C Member 3 or NR3C3 or PGR)
Agonist
Progesterone Receptor (Nuclear Receptor Subfamily 3 Group C Member 3 or NR3C3 or PGR)
Antagonist
Prostacyclin Receptor (Prostaglandin I2 Receptor or Prostanoid IP Receptor or PTGIR) Agonist
Prostaglandin E2 Receptor EP1 Subtype (Prostanoid EP1 Receptor or PTGER1) Agonist
Prostaglandin E2 Receptor EP1 Subtype (Prostanoid EP1 Receptor or PTGER1) Antagonist
Prostaglandin E2 Receptor EP2 Subtype (Prostanoid EP2 Receptor or PTGER2) Agonist
Prostaglandin E2 Receptor EP3 Subtype (PGE2 R or Prostanoid EP3 Receptor or PTGER3) Agonist
Prostaglandin F2 Alpha Receptor (Prostanoid FP Receptor or PTGFR) Agonist
Prostaglandin G/H Synthase (Cyclooxygenase or COX or PTGS or EC 1.14.99.1) Inhibitor
Prostaglandin G/H Synthase 1 (Cyclooxygenase 1 or COX1 or Prostaglandin Endoperoxide Synthase
1 or Prostaglandin H2 Synthase 1 or PTGS1 or EC 1.14.99.1) Inhibitor
Prostaglandin G/H Synthase 2 (Cyclooxygenase 2 or COX2 or Prostaglandin Endoperoxide Synthase
2 or PHS II or Prostaglandin H2 Synthase 2 or PTGS2 or EC 1.14.99.1) Inhibitor
Proteasome Inhibitor
Protein Cereblon (CRBN) Activator
Proteinase Activated Receptor 1 (PAR1 or Coagulation Factor II Receptor or Thrombin Receptor or
F2R) Antagonist
Prothrombin (Coagulation Factor II or F2 or EC 3.4.21.5) Inhibitor
Proto Oncogene Tyrosine Protein Kinase Receptor Ret (Cadherin Family Member 12 or Proto
Oncogene c Ret or RET or EC 2.7.10.1) Inhibitor
Proto Oncogene Tyrosine Protein Kinase ROS (Proto Oncogene c Ros 1 or Receptor Tyrosine Kinase
c Ros Oncogene 1 or c Ros Receptor Tyrosine Kinase or ROS1 or EC 2.7.10.1) Inhibitor
Pyruvate Kinase PKLR (Pyruvate Kinase 1 or Red Cell/Liver Pyruvate Kinase or R Type/L Type
Pyruvate Kinase or Pyruvate Kinase Isozymes L/R or PKLR or EC 2.7.1.40) Activator
Pyruvate Synthase (Pyruvate Ferredoxin Oxidoreductase or PFOR or EC 1.2.7.1) Inhibitor
Receptor Type Tyrosine Protein Kinase FLT3 (FMS Like Tyrosine Kinase 3 or FL Cytokine Receptor
or Stem Cell Tyrosine Kinase 1 or Fetal Liver Kinase 2 or CD135 or FLT3 or EC 2.7.10.1) Inhibitor
Receptor Tyrosine Protein Kinase ERBB 2 (Metastatic Lymph Node Gene 19 Protein or Proto
Oncogene Neu or Proto Oncogene C ErbB 2 or Tyrosine Kinase Type Cell Surface Receptor HER2 or
p185erbB2 or HER2 or CD340 or ERBB2 or EC 2.7.10.1) Inhibitor
Receptor Tyrosine Protein Kinase ERBB 4 (Tyrosine Kinase Type Cell Surface Receptor HER4 or
Proto Oncogene Like Protein c ErbB 4 or p180erbB4 or HER4 or ERBB4 or EC 2.7.10.1) Inhibitor
Renin (Angiotensinogenase or REN or EC 3.4.23.15) Inhibitor
Retinoic Acid Receptor (RAR) Agonist
Retinoic Acid Receptor Alpha (RAR Alpha or Nuclear Receptor Subfamily 1 Group B Member 1 or
NR1B1 or RARA) Agonist
Retinoic Acid Receptor Beta (RAR Beta or HBV Activated Protein or Nuclear Receptor Subfamily 1
Group B Member 2 or RAR Epsilon or NR1B2 or RARB) Agonist
Retinoic Acid Receptor Gamma (RAR Gamma or Nuclear Receptor Subfamily 1 Group B Member 3
or NR1B3 or RARG) Agonist
Retinoic Acid Receptor RXR Alpha (Nuclear Receptor Subfamily 2 Group B Member 1 or Retinoid X
Receptor Alpha or NR2B1 or RXRA) Agonist
Retinoic Acid Receptor RXR Beta (Nuclear Receptor Subfamily 2 Group B Member 2 or Retinoid X
Receptor Beta or NR2B2 or RXRB) Agonist
Retinoic Acid Receptor RXR Gamma (Nuclear Receptor Subfamily 2 Group B Member 3 or Retinoid X
Receptor Gamma or NR2B3 or RXRG) Agonist
Retinoid X Receptor (RXR) Agonist
Reverse Transcriptase (EC 2.7.7.49) Inhibitor
Rho Associated Protein Kinase 2 (Rho Kinase 2 or Rho Associated Coiled Coil Containing Protein
Kinase 2 or p164 ROCK 2 or ROCK2 or EC 2.7.11.1) Inhibitor
Rho Kinase (Rho Associated Coiled Coil Forming Protein Kinase or ROCK or EC 2.7.11.1) Inhibitor
Ribonucleoside Diphosphate Reductase (Ribonucleotide Reductase or RRM or EC 1.17.4.1) Inhibitor
RNA Directed RNA Polymerase (EC 2.7.7.48) Inhibitor
RNA Polymerase (EC 2.7.7.6) Inhibitor
RNA Polymerase II (RNAP II or Pol II or EC 2.7.7.6) Inhibitor
RNA Synthesis Inhibitor
Ryanodine Receptor 1 (Skeletal Muscle Calcium Release Channel or Skeletal Muscle Ryanodine
Receptor or Type 1 Ryanodine Receptor or RYR1) Antagonist
Serine Type D Ala D Ala Carboxypeptidase (D Alanyl D Alanine Carboxypeptidase or DD
Transpeptidase or EC 3.4.16.4) Inhibitor
Serine/Threonine Protein Kinase B Raf (p94 or Proto Oncogene B Raf or v Raf Murine Sarcoma Viral
Oncogene Homolog B1 or BRAF or EC 2.7.11.1) Inhibitor
Serine/Threonine Protein Kinase mTOR (FK506 Binding Protein 12 Rapamycin Complex Associated
Protein 1 or FKBP12 Rapamycin Complex Associated Protein or Mammalian Target Of Rapamycin or
Mechanistic Target Of Rapamycin or Rapamycin And FKBP12 Target 1 or Rapamycin Target Protein
1 or MTOR or EC 2.7.11.1) Inhibitor
Serine/Threonine Protein Kinase UL97 (HSRF3 Protein or Ganciclovir Kinase or UL97 or EC 2.7.11.)
Inhibitor
Sigma Non Opioid Intracellular Receptor 1 (Aging Associated Gene 8 Protein or SR31747 Binding
Protein or Sigma 1 Type Opioid Receptor or SIGMAR1) Agonist
Smoothened Homolog (Protein Gx or SMO) Antagonist
Sodium And Chloride Dependent GABA Transporter 1 (GAT1 or Solute Carrier Family 6 Member 1 or
SLC6A1) Inhibitor
Sodium Channel Blocker
Sodium Channel Protein Type 1 Subunit Alpha (Sodium Channel Protein Brain I Subunit Alpha or
Voltage Gated Sodium Channel Subunit Alpha Nav1.1 or SCN1A) Blocker
Sodium Channel Protein Type 10 Subunit Alpha (Peripheral Nerve Sodium Channel 3 or Voltage
Gated Sodium Channel Subunit Alpha Nav1.8 or SCN10A) Blocker
Sodium Channel Protein Type 2 Subunit Alpha (HBSC II or Sodium Channel Protein Brain II Subunit
Alpha or Sodium Channel Protein Type II Subunit Alpha or Voltage Gated Sodium Channel Subunit
Alpha Nav1.2 or SCN2A) Blocker
Sodium Channel Protein Type 3 Subunit Alpha (Voltage Gated Sodium Channel Subtype III or
Voltage Gated Sodium Channel Subunit Alpha Nav1.3 or SCN3A) Blocker
Sodium Channel Protein Type 4 Subunit Alpha (Sodium Channel Protein Skeletal Muscle Subunit
Alpha or Voltage Gated Sodium Channel Subunit Alpha Nav1.4 or SCN4A) Blocker
Sodium Channel Protein Type 5 Subunit Alpha (Sodium Channel Protein Cardiac Muscle Subunit
Alpha or Voltage Gated Sodium Channel Subunit Alpha Nav1.5 or SCN5A) Blocker
Sodium Channel Protein Type 8 Subunit Alpha (Voltage Gated Sodium Channel Subunit Alpha
Nav1.6 or SCN8A) Blocker
Sodium Channel Protein Type 9 Subunit Alpha (Neuroendocrine Sodium Channel or Peripheral
Sodium Channel 1 or Voltage Gated Sodium Channel Subunit Alpha Nav1.7 or SCN9A) Blocker
Sodium Dependent Dopamine Transporter (DA Transporter or DAT or Solute Carrier Family 6
Member 3 or SLC6A3) Inhibitor
Sodium Dependent Noradrenaline Transporter (Norepinephrine Transporter or NET or Solute Carrier
Family 6 Member 2 or SLC6A2) Inhibitor
Sodium Dependent Serotonin Transporter (5HT Transporter or 5HTT or Solute Carrier Family 6
Member 4 or SLC6A4) Inhibitor
Sodium/Glucose Cotransporter 2 (Low Affinity Sodium-Glucose Cotransporter or Solute Carrier
Family 5 Member 2 or SGLT2 or SLC5A2) Inhibitor
Sodium/Hydrogen Exchanger 3 (NHE3 or Solute Carrier Family 9 Member 3 or SLC9A3) Inhibitor
Sodium/Iodide Cotransporter (Sodium-Iodide Symporter or Solute Carrier Family 5 Member 5 or
SLC5A5) Activator
Soluble Guanylate Cyclase (sGC or EC 4.6.1.2) Activator
Solute Carrier Family 12 Member 1 (Bumetanide Sensitive Sodium (Potassium) Chloride
Cotransporter 2 or Kidney Specific Na-K-Cl Symporter or SLC12A1) Inhibitor
Solute Carrier Family 12 Member 2 (Basolateral Na-K-Cl Symporter or Bumetanide Sensitive Sodium
(Potassium) Chloride Cotransporter 1 or SLC12A2 or NKCC1) Inhibitor
Solute Carrier Family 12 Member 3 (Na-Cl Cotransporter or NCC or Na-Cl Symporter or Thiazide
Sensitive Sodium-Chloride
Sphingosine 1-Phosphate Receptor (S1PR) Modulator
Sphingosine 1-Phosphate Receptor 1 (Endothelial Differentiation G Protein Coupled Receptor 1 or
Sphingosine 1 Phosphate Receptor Edg 1 or CD363 or S1PR1) Agonist
Sphingosine 1-Phosphate Receptor 4 (Endothelial Differentiation G Protein Coupled Receptor 6 or
Sphingosine 1 Phosphate Receptor Edg 6 or S1PR4) Agonist
Sphingosine 1-Phosphate Receptor 5 (Endothelial Differentiation G Protein Coupled Receptor 8 or
Sphingosine 1 Phosphate Receptor Edg 8 or S1PR5) Agonist
Squalene Monooxygenase (Squalene Epoxidase or SQLE or EC 1.14.14.17) Inhibitor
Steroid 17 Alpha Hydroxylase/17,20 Lyase (17 Alpha Hydroxyprogesterone Aldolase or Cytochrome
P450 17A1 or Cytochrome P450 C17 or Steroid 17 Alpha Monooxygenase or CYP17 or CYP17A1 or
EC 1.14.14.19 or EC 1.14.14.32) Inhibitor
Substance P Receptor (Tachykinin Receptor 1 or NK 1 Receptor or NK1R or TACR1) Antagonist
Sucrase Isomaltase Intestinal (SI or EC 3.2.1.48 or EC 3.2.1.10) Inhibitor
Surface Protein gp120 Inhibitor
Survival Motor Neuron Protein (Component Of Gems 1 or Gemin 1 or SMN1 or SMN2) Activator
Synaptic Vesicle Glycoprotein 2A (SV2A) Binder
Synaptic Vesicular Amine Transporter (Monoamine Transporter or Solute Carrier Family 18 Member 2
or Vesicular Amine Transporter 2 or VAT2 or SLC18A2) Inhibitor
Tetrahydrobiopterin (THB or BH4) Replacement
Thioredoxin Disulfide Reductase (NADPH Thioredoxin Reductase or EC 1.8.1.9) Inhibitor
Thrombopoietin Receptor (Myeloproliferative Leukemia Protein or Proto Oncogene c Mpl or CD110 or
MPL) Agonist
Thymidine Kinase (TK or EC 2.7.1.21) Activator
Thymidine Phosphorylase (Gliostatin or Platelet Derived Endothelial Cell Growth Factor or TdRPase
or TYMP or EC 2.4.2.4) Inhibitor
Thymidylate Synthase (TYMS or EC 2.1.1.45) Inhibitor
Thyroid Hormone Receptor Agonist
Thyroid Hormone Receptor Alpha (Nuclear Receptor Subfamily 1 Group A Member 1 or V erbA
Related Protein 7 or c erbA 1 or c erbA Alpha or EAR7 or THRA) Agonist
Thyroid Hormone Receptor Beta (Nuclear Receptor Subfamily 1 Group A Member 2 or c erbA 2 or c
erbA Beta or ERBA2 or THRB) Agonist
Thyroid Peroxidase (Thyroperoxidase or Thyroid Microsomal Antigen or TPO or EC 1.11.1.8) Inhibitor
Toll Like Receptor 7 (TLR7) Agonist
Toll Like Receptor 7 (TLR7) Antagonist
Toll Like Receptor 9 (CD289 or TLR9) Antagonist
Transferrin Receptor (TFR) Agonist
Transient Receptor Potential Cation Channel Subfamily V Member 1 (Capsaicin Receptor or Vanilloid
Receptor 1 or TRPV1) Inhibitor
Transient Receptor Potential Cation Channel Subfamily V Member 1 (Capsaicin Receptor or Vanilloid
Receptor 1 or TRPV1) Activator
Transthyretin (ATTR or Prealbumin or TBPA or TTR) Activator
Transthyretin (ATTR or Prealbumin or TBPA or TTR) Inhibitor
Trifunctional Purine Biosynthetic Protein Adenosine 3 (Phosphoribosylglycinamide Formyltransferase
or Phosphoribosylamine Glycine Ligase or Phosphoribosylformylglycinamidine Cyclo Ligase or GART
or EC 2.1.2.2 or EC 6.3.3.1 or EC 6.3.4.13) Inhibitor
Tryptophan 5 Monooxygenase (Tryptophan Hydroxylase or TPH or EC 1.14.16.4) Inhibitor
Tubulin Inhibitor
Type 1 Angiotensin II Receptor (AT1AR or AT1BR or Angiotensin II Type 1 Receptor or AGTR1)
Antagonist
Tyrosinase (Monophenol Monooxygenase or Tumor Rejection Antigen AB or LB24 AB or SK29 AB or
TYR or EC 1.14.18.1) Inhibitor
Tyrosine 3 Monooxygenase (Tyrosine 3 Hydroxylase or TH or EC 1.14.16.2) Inhibitor
Tyrosine Protein Kinase BTK (Bruton Tyrosine Kinase or B Cell Progenitor Kinase or
Agammaglobulinemia Tyrosine Kinase or BTK or EC 2.7.10.2) Inhibitor
Tyrosine Protein Kinase CSK (C Src Kinase or Protein Tyrosine Kinase CYL or CSK or EC 2.7.10.2)
Inhibitor
Tyrosine Protein Kinase JAK1 (Janus Kinase 1 or JAK1 or EC 2.7.10.2) Inhibitor
Tyrosine Protein Kinase JAK2 (Janus Kinase 2 or JAK2 or EC 2.7.10.2) Inhibitor
Tyrosine Protein Kinase JAK3 (Janus Kinase 3 or Leukocyte Janus Kinase or JAK3 or EC 2.7.10.2)
Inhibitor
Tyrosine Protein Kinase Receptor UFO (AXL Oncogene or AXL or EC 2.7.10.1) Inhibitor
Tyrosine Protein Kinase SYK (Spleen Tyrosine Kinase or p72 Syk or SYK or EC 2.7.10.2) Inhibitor
Tyrosine Protein Kinase Tec (TEC or EC 2.7.10.2) Inhibitor
UDP N-Acetylglucosamine 1 Carboxyvinyltransferase (Enoylpyruvate Transferase or UDP N
Acetylglucosamine 1 Carboxyvinyl Transferase or UDP N Acetylglucosamine Enolpyruvyl Trensferase
or murA or EC 2.5.1.7) Inhibitor
Urease (Urea Amidohydrolase or EC 3.5.1.5) Inhibitor
Vascular Endothelial Growth Factor Receptor 1 (Fms Like Tyrosine Kinase 1 or Tyrosine Protein
Kinase Receptor FLT or Tyrosine Protein Kinase FRT or Vascular Permeability Factor Receptor or
VEGFR1 or FLT1 or EC 2.7.10.1) Inhibitor
Vascular Endothelial Growth Factor Receptor 2 (Fetal Liver Kinase 1 or Kinase Insert Domain
Receptor or Protein Tyrosine Kinase Receptor flk 1 or VEGFR2 or CD309 or KDR or EC 2.7.10.1)
Inhibitor
Vascular Endothelial Growth Factor Receptor 3 (Fms Like Tyrosine Kinase 4 or Tyrosine Protein
Kinase Receptor FLT4 or VEGFR3 or FLT4 or EC 2.7.10.1) Inhibitor
Vasopressin V1a Receptor (AVPR V1a or Antidiuretic Hormone Receptor 1a or Vascular/Hepatic
Type Arginine Vasopressin Receptor or AVPR1A) Antagonist
Vasopressin V2 Receptor (AVPR V2 or Antidiuretic Hormone Receptor or Renal Type Arginine
Vasopressin Receptor or ADHR or AVPR2) Antagonist
Viral mRNA
Vitamin D Receptor (Calcitriol Receptor) Agonist
Vitamin D3 Receptor (1,25 Dihydroxyvitamin D3 Receptor or Nuclear Receptor Subfamily 1 Group I
Member 1 or NR1I1 or VDR) Agonist
Voltage Dependent Calcium Channel Activator
Voltage Dependent Calcium Channel Blocker
Voltage Dependent Calcium Channel Subunit Alpha2/Delta1 (CACNA2D1) Blocker
Voltage Dependent Calcium Channel Subunit Alpha2/Delta2 (CACNA2D2) Blocker
Voltage Dependent L Type Calcium Channel Blocker
Voltage Dependent L Type Calcium Channel Subunit Alpha 1S (Voltage Gated Calcium Channel
Subunit Alpha Cav1.1 or CACNA1S) Blocker
Voltage Dependent N Type Calcium Channel Subunit Alpha 1B (Voltage Gated Calcium Channel
Subunit Alpha Cav2.2 or Brain Calcium Channel III or CACNA1B) Blocker
Voltage Dependent T Type Calcium Channel Blocker
Voltage Dependent T Type Calcium Channel Subunit Alpha 1G (Voltage Gated Calcium Channel
Subunit Alpha Cav3.1 or CACNA1G) Blocker
Voltage Gated Sodium Channel (SCN) Blocker
Xanthine Dehydrogenase/Oxidase (Xanthine Dehydrogenase or Xanthine Oxidase or Xanthine
Oxidoreductase or XDH or EC 1.17.1.4 or EC 1.17.3.2) Inhibitor

Particular examples of small molecules that may be linked to a macromolecule of the invention fall, for example, into any of the classes shown in Table 3. In particular, exemplary glucocorticoid receptor agonists include, without limitation, cortisone, dexamethasone, fluticasone, mometasone, fluocinolone, budesonide, butixicort, and betamethasone. Exemplary tyrosine protein kinase BTK inhibitors include, without limitation, acalabrutinib, evobrutinib, fenebrutinib, ibrutinib, orelabrutinib, pirtobrutinib, remibrutinib, rilzabrutinib, tolebrutinib, and zanubrutinib. Exemplary PI3K inhibitors include, without limitation, alpelisib, idelalisib, copanlisib, and duvelisib. Exemplary JAK inhibitors include, without limitation, abrocitinib, baricitinib, delgocitinib, filgotinib, peficitinib, ruxolitinib, tofacitinib, and upadacitinib. Exemplary cathepsin K inhibitors include, without limitation, odanacatib, relacatib, MIV-711, and KGP-207. Exemplary topoisomerase inhibitors include, without limitation, irinotecan, doxorubicin, daunorubicin, doxorubicin, Ellence, etoposide, idarubicin, topotecan, and valrubicin.

In some embodiments, the small molecule is a steroid. In some embodiments, the small molecule is fluocinolone (PubChem ID: 91488) or a salt, ester, or conjugate thereof.

In some embodiments, the small molecule is a Bruton's tyrosine kinase (BTK) inhibitor. In some embodiments, the small molecule is ibrutinib (PubChem ID: 23821094) or a salt, ester, or conjugate thereof.

In some embodiments, the small molecule is a Janus kinase (JAK) inhibitor. In some embodiments, the small molecule is tofacitinib (PubChem ID: 9926791) or a salt, ester, or conjugate thereof.

Small molecules may be conjugated to a macromolecule of the invention using any conjugation technique known in the art. For example, small molecule carboxy, hydroxyl, and amine residues may be joined to amine and sulfhydryl residues on proteins using linkage techniques.

Alternatively, any complementary functional groups on the two components may be used to react with each other to form a covalent bond. Examples of complementary reactive functional groups include, but are not limited to, e.g., maleimide and cysteine, amine and activated carboxylic acid, thiol and maleimide, activated sulfonic acid and amine, isocyanate and amine, azide and alkyne, and alkene and tetrazine. Further, any available linker may be utilized in the invention including heterobifunctional linkers that allow attachment of small molecules through, for example, disulfide bonds and amide bonds.

### ANDbody Structures

In general, an ANDbody can be any macromolecule, such as a polypeptide or protein that contains both an effector target binding site or binding domain, and an address target binding site or binding domain. The binding sites may be present on the same polypeptide chain or different polypeptide chains that are linked together, e.g., through disulfide bonds.

In some embodiments, the binding site for the effector target and the binding site for the address target of the ANDbody each comprise an antibody heavy chain and/or a light chain domain. In some embodiments the ANDbody comprises a first antibody variable domain which has binding specificity for the effector target and a second antibody variable domain that has binding specificity for the address target. In other embodiments the ANDbody comprises a first antigen binding site of an antibody, which first antigen binding site has binding specificity for the effector target, and a second antigen binding site of an antibody, which second antigen binding site has binding specificity for the address target.

In some embodiments, the ANDbody may have the structure of an antibody molecule. The term “antibody” as used herein includes full-length antibodies and antigen binding antibody fragments (e.g., scFvs). In some embodiments, an antibody molecule has specificity for more than one, e.g., 2, 3, 4 antigens, e.g., the antibody molecule comprises a plurality of variable domain sequences, wherein a first variable domain sequence of the plurality has binding specificity for a first epitope (e.g., the effector target) and a second variable domain sequence of the plurality has binding specificity for a second epitope (e.g., the address target)

In some embodiments, the ANDbody is an antibody molecule that has an arm or domain that binds the effector target and an arm or domain that binds the address target. In embodiments, the ANDbody is an antibody molecule that comprises light chains that bind one of the effector target and address target, and heavy chains that bind the other of the effector target and address target.

In some embodiments, the ANDbody has the structure of an scFv, BsIgG, a BsAb fragment, a BiTE, a dual-affinity re-targeting protein (DART), a tandem diabody (TandAb), a diabody, an Fab2, a di-scFv, chemically linked F(ab′)2, an Ig molecule with 2, 3 or 4 different antigen binding sites, a DVI-IgG four-in-one, an ImmTac, an HSAbody, an IgG-IgG, a Cov-X-Body, an scFv1-PEG-scFv₂, an appended IgG, an DVD-IgG, an affibody, an affilin, an affimer, an affitin, an alphabody, an anticalin, an avimer, a DARPin, a Fynomer, a monobody, a nanoCLAMP, a bis-Fab, an Fv, a Fab, a Fab′-SH, a linear antibody, an scFv, an antibody with only a heavy chain (Hurnabody), an ScFab, an IgG antibody fragment, a single-chain variable region antibody, a single-domain heavy chain antibody. a bispecific triplebody, a BiKE, a CrossMAb, a dsDb, an scDb, tandem a dAb/VHH, a triple dAb VHH, a tetravalent dAb/VHH, a Fab-scFv, a Fab-Fv, or a DART-Fc, an adnectin, a Kunitz-type inhibitor, or a receptor decoy.

The affinity of the effector target binding site and address target binding site of an ANDbody for their respective binding partners may differ. In some embodiments the affinity of the first binding site to the therapeutic effector target it binds is weaker than the affinity of the second binding site to the address target. In some embodiments the affinity of the first binding site to the therapeutic effector target it binds is more than 2-fold, 3-fold, 4-fold, 5-fold, 10-fold, 15-fold, 20-fold, 50-fold, 100-fold, 200-fold, 500-fold, 1000-fold weaker than the affinity of the second binding site to the address target.

The terms “binding affinity” and “binding activity” refer to the tendency of a macromolecule, e.g., a polypeptide molecule, to bind or not to bind to a target. For purposes of the present invention, which combines two binding sites, the relative affinities of the two binding sites can be determined by, for example, measuring their respective affinities when each binding site is present on a common scaffold, such as in the form of a single chain antibody. Such a comparison allows a comparison of the affinities of two binding sites while eliminating any interference from other binding sites present on the macromolecule of the present invention.

Binding affinity may be quantified by determining the dissociation constant (Kd) for a polypeptide and its binder. A lower Kd is indicative of a higher affinity for a binding partner. Similarly, the specificity of binding of a polypeptide to its binding partner may be defined in terms of the comparative dissociation constants (Kd) of the polypeptide for its binding partner as compared to the dissociation constant with respect to the polypeptide and another, non-target molecule.

The value of this dissociation constant can be determined by known methods. For example, the Kd may be established using a double-filter nitrocellulose filter binding assay such as that disclosed by Wong & Lohman (Proc. Natl. Acad. Sci. USA 90, 5428-5432, 1993). Other standard assays to evaluate the binding ability of ligands such as antibodies towards targets are known in the art, including for example, ELISAs, Western blots, RIAs, and flow cytometry analysis. The binding kinetics (e.g., binding affinity) of the antibody also can be assessed by standard assays known in the art, such as by Biacore™ system analysis.

As an alternative to Kd, EC₅₀ or IC₅₀ may be used to determine relative affinities. In this context EC₅₀ indicates the concentration at which a polypeptide achieves 50% of its maximum binding to a fixed quantity of binding partner. IC₅₀ indicates the concentration at which a polypeptide inhibits 50% of the maximum binding of a fixed quantity of competitor to a fixed quantity of binding partner. In both cases, a lower level of EC₅₀ or IC₅₀ indicates a higher affinity for a target. The EC₅₀ and IC₅₀ values of an ANDbody binding site for its binding partner can both be determined by well-known methods, for example ELISA.

In some embodiments the Kd of therapeutic effector target binder might be higher than about 1 pM, about 10 pM, about 100 pM, about 1 nM, about 10 nM, about 100 nM, about 500 nM, or about 1 uM (e.g., may be between 1 pM and 10 pM, between 10 pM and 100 pM, between 100 pM and 1 nM, between 1 nM and 10 nM, between 10 nM and 100 nM, between 100 nM and 500 nM, or between 500 nM and 1 uM). In some embodiments the Kd of the address target binder might be less than about 1 uM, about 500 nM, about 100 nM, about 10 nM, about 1 nM, about 100 pM, about 10 pM, or about 1 pM (e.g., may be between 1 uM and 500 nM, between 500 nM and 100 nM, between 100 nM and 10 nM, between 10 nM and 1 nM, between 1 nM and 100 pM, between 100 pM and 10 pM, or between 10 pM and 1 pM). In some embodiments, the Kd for the therapeutic effector target binder may be about 6-fold, about 5-fold, about 4-fold, about 3-fold, or about 2-fold higher than the Kd for the address target binder.

In some embodiments the EC₅₀ of therapeutic effector target binder might be higher than about 1 pM, about 10 pM, about 100 pM, about 1 nM, about 10 nM, about 100 nM, about 500 nM, or about 1 uM (e.g., may be between 1 pM and 10 pM, between 10 pM and 100 pM, between 100 pM and 1 nM, between 1 nM and 10 nM, between 10 nM and 100 nM, between 100 nM and 500 nM, or between 500 nM and 1 uM). In some embodiments the EC₅₀ of the address target binder might be less than about 1 uM, about 500 nM, about 100 nM, about 10 nM, about 1 nM, about 100 pM, about 10 pM, or about 1 pM (e.g., may be between 1 uM and 500 nM, between 500 nM and 100 nM, between 100 nM and 10 nM, between 10 nM and 1 nM, between 1 nM and 100 pM, between 100 pM and 10 pM, or between 10 pM and 1 pM). In some embodiments, the EC₅₀ for the therapeutic effector target binder may be about 6-fold, about 5-fold, about 4-fold, about 3-fold, or about 2-fold higher than the EC₅₀ for the address target binder.

In some embodiments the IC₅₀ of therapeutic effector target binder might be higher than about 1 pM, about 10 pM, about 100 pM, about 1 nM, about 10 nM, about 100 nM, about 500 nM, or about 1 uM (e.g., may be between 1 pM and 10 pM, between 10 pM and 100 pM, between 100 pM and 1 nM, between 1 nM and 10 nM, between 10 nM and 100 nM, between 100 nM and 500 nM, or between 500 nM and 1 uM). In some embodiments the IC50 of the address target binder might be less than about 1 uM, about 500 nM, about 100 nM, about 10 nM, about 1 nM, about 100 pM, about 10 pM, or about 1 pM (e.g., may be between 1 uM and 500 nM, between 500 nM and 100 nM, between 100 nM and 10 nM, between 10 nM and 1 nM, between 1 nM and 100 pM, between 100 pM and 10 pM, or between 10 pM and 1 pM). In some embodiments, the IC₅₀ for the therapeutic effector target binder may be about 6-fold, about 5-fold, about 4-fold, about 3-fold, or about 2-fold higher than the IC₅₀ for the address target binder.

The cellular or tissue density of the effector target and address target bound by an ANDbody may differ. In embodiments, the density of the therapeutic effector target on a cell bound by the effector target binding site of an ANDbody is more than about 2-fold, about 3-fold, about 4-fold, about 5-fold, about 10-fold, about 15-fold, about 20-fold, about 50-fold, about 100-fold, about 200-fold, about 500-fold, about 1000-fold, about 10,000-fold, about 100,000-fold less than the density of the address target on a cell bound by the address target binding site.

In some embodiments, the affinity of the first binding site to the therapeutic effector target it binds is about one-half (½) X Kd less than the affinity of the second binding site to the address target it binds and the density of the therapeutic effector target on a cell bound by the first binding site is about one-half (½) X Kd less than the density of the address target on a cell bound by the second binding site.

In some embodiments, the ANDbody has both the affinity and density parameters as described hereinabove.

In some embodiments the first binding site and second binding site in the ANDbody are directly joined to each other. By directly joined is meant that the first binding site coding sequences abut the second binding site coding sequences and no sequences derived from other sequences (such as linkers) are present. In some embodiments the first binding site and second binding site in the ANDbody are not directly joined to each other.

In addition to a small molecule, an ANDbody, as disclosed herein, can also be linked to an additional moiety or moieties, e.g., an extracellular component, an intracellular component, a soluble factor (e.g., an enzyme, hormone, cytokine, growth factor, toxin, venom, pollutant, etc.), or a transmembrane protein (e.g., a cell surface receptor).

Exemplary effector target and address target sequences for which ANDbodies of the present technology may have affinity are provided in Table 4 and in the sequence listing. In some instances, the sequences comprise full-length protein sequences and/or Fc fusion sequences with or without the signal peptide regions. In some embodiments, ANDbodies of the present technology include binding domains that bind address target or effector target proteins. In embodiments, binding domains of the present ANDbodies may bind protein sequences that include a signal peptide. In other embodiments, binding domains of the present ANDbodies may bind proteins that lack a signal protein. In some embodiments, binding domains of the present ANDbodies may bind full-length proteins. In other embodiments, binding domains of the present ANDbodies may bind protein fusions, such as full-length protein sequences, or peptide fragments thereof, with or without signal peptide regions, fused to other proteins, such as, for example, Fc sequences. In other embodiments, binding domains of the present ANDbodies may bind proteins that comprise less than the full-length protein sequence, such as a peptide fragment of the address target or effector target.

TABLE 4
Exemplary Effector Target and Address Target Sequences

| | Spe- | Acces- | Acces- | | | | SEQ |
|---|---|---|---|---|---|---|---|
| | cies | sion | sion | Posi- | | | ID |
| Name | (M/H) | # | Location | tions | AA sequence | Notes | NO: |
| RAGE | H | Q15109 | Uniprot | full | MAAGTAVGAWVLVLSLWGAVVG | AA 1-22 | SEQ |
| | | | | length | AQNITARIGEPLVLKCKGAPKKPP | is the | ID |
| | | | | | QRLEWKLNTGRTEAWKVLSPQG | signal | NO: |
| | | | | | GGPWDSVARVLPNGSLFLPAVGI | peptide | 1 |
| | | | | | QDEGIFRCQAMNRNGKETKSNY | | |
| | | | | | RVRVYQIPGKPEIVDSASELTAGV | | |
| | | | | | PNKVGTCVSEGSYPAGTLSWHL | | |
| | | | | | DGKPLVPNEKGVSVKEQTRRHP | | |
| | | | | | ETGLFTLQSELMVTPARGGDPRP | | |
| | | | | | TFSCSFSPGLPRHRALRTAPIQPR | | |
| | | | | | VWEPVPLEEVQLVVEPEGGAVAP | | |
| | | | | | GGTVTLTCEVPAQPSPQIHWMKD | | |
| | | | | | GVPLPLPPSPVLILPEIGPQDQGT | | |
| | | | | | YSCVATHSSHGPQESRAVSISIIE | | |
| | | | | | PGEEGPTAGSVGGSGLGTLALAL | | |
| | | | | | GILGGLGTAALLIGVILWQRRQRR | | |
| | | | | | GEERKAPENQEEEEERAELNQS | | |
| | | | | | EEPEAGESSTGGP | | |
| RAGE | M | Q62151 | Uniprot | full | MPAGTAARAWVLVLALWGAVAG | AA 1-22 | SEQ |
| | | | | length | GQNITARIGEPLVLSCKGAPKKPP | is the | ID |
| | | | | | QQLEWKLNTGRTEAWKVLSPQG | signal | NO: |
| | | | | | GPWDSVARILPNGSLLLPATGIVD | peptide | 2 |
| | | | | | EGTFRCRATNRRGKEVKSNYRV | | |
| | | | | | RVYQIPGKPEIVDPASELTASVPN | | |
| | | | | | KVGTCVSEGSYPAGTLSWHLDG | | |
| | | | | | KLLIPDGKETLVKEETRRHPETGL | | |
| | | | | | FTLRSELTVIPTQGGTHPTFSCSF | | |
| | | | | | SLGLPRRRPLNTAPIQLRVREPG | | |
| | | | | | PPEGIQLLVEPEGGIVAPGGTVTL | | |
| | | | | | TCAISAQPPPQVHWIKDGAPLPLA | | |
| | | | | | PSPVLLLPEVGHEDEGTYSCVAT | | |
| | | | | | HPSHGPQESPPVSIRVTETGDEG | | |
| | | | | | PAEGSVGESGLGTLALALGILGGL | | |
| | | | | | GVVALLVGAILWRKRQPRREERK | | |
| | | | | | APESQEDEEERAELNQSEEAEM | | |
| | | | | | PENGAGGP | | |
| RAGE-Fc, | H | Q15109 | uniprot | A23- | aqnitari geplvlkckg apkkppqrle | Sequence | SEQ |
| extra- | | (RAGE- | (RAGE) | A342 | wklntgrtea wkvlspqggg pwdsvarvlp | is to full | ID |
| cellular | | H) | DrugBank | (RAGE) | ngslflpavg iqdegifrcq amnrngketk | RAGE-Fc; | NO: |
| domain | | DB01281 | (Abata- | | snyrvrvyqi pgkpeivdsa seltagvpnk | RAGE is | 3 |
| | | (Abata- | cept) | | vgtcvsegsy pagtlswhld gkplvpnekg | lowercase | |
| | | cept) | | | vsvkeqtrrh petglftlqs elmvtpargg | and from the | |
| | | | | | dprptfscsf spglprhral rtapiqprvw | accession ID | |
| | | | | | epvpleevql vvepeggava pggtvtltce | at left, and | |
| | | | | | vpaqpspqih wmkdgvplpl ppspvlilpe | the modified | |
| | | | | | igpqdqgtys cvathsshgp qesravsisi | human IgG1 | |
| | | | | | iepgeegpta gsvggsglgt la | Fc region is | |
| | | | | | QEPKSSDKTHTSPPSPAPELLGG | in CAPS and | |
| | | | | | SSVFLFPPKPKDTLMISRTPEVTC | is taken | |
| | | | | | VVVDVSHED | from the | |
| | | | | | PEVKFNWYVDGVEVHNAKTKPR | sequence for | |
| | | | | | EEQYNSTYRVVSVLTVLHQDWLN | Abatacept | |
| | | | | | GKEYKCKVSNKALPA | (DrugBank | |
| | | | | | PIEKTISKAKGQPREPQVYTLPPS | Online) | |
| | | | | | RDELTKNQVSLTCLVKGFYPSDIA | | |
| | | | | | VEWESNGQPENN | | |
| | | | | | YKTTPPVLDSDGSFFLYSKLTVDK | | |
| | | | | | SRWQQGNVFSCSVMHEALHNHY | | |
| | | | | | TQKSLSLSPGK | | |
| RAGE-Fc, | M | Q62151 | uniprot | A23- | gqnitarigeplvlsckgapkkppqqlewklnt | lowercase | SEQ |
| extra- | | (RAGE-M) | (RAGE) | A340 | grteawkvlspqggpwdsvarilpngslllpat | is mouse | ID |
| cellular | | DB01281 | DrugBank | (RAGE) | givdegtfrcratnrrgkevksnyrvrvyqipgk | RAGE-FC | NO: |
| domain | | (Abata- | (Abata- | | peivdpaseltasvpnkvgtcvsegsypagtl | extra- | 4 |
| | | cept) | cept) | | swhldgkllipdgketlvkeetrrhpetglftlrsel | cellular | |
| | | | | | tviptqggthptfscsfslglprrrplntapiqlrvre | without | |
| | | | | | pgppegiqllvepeggivapggtvtltcaisaq | signal | |
| | | | | | pppqvhwikdgaplplapspvlllpevghede | peptide | |
| | | | | | gtyscvathpshgpqesppvsirvtetgdegp | and the | |
| | | | | | aegsvgesglgt | modified | |
| | | | | | QEPKSSDKTHTSPPSPAPELLGG | human IgG1 | |
| | | | | | SSVFLFPPKPKDTLMISRTPEVTC | Fc region is | |
| | | | | | VVVDVSHED | in CAPS and | |
| | | | | | PEVKFNWYVDGVEVHNAKTKPR | is taken | |
| | | | | | EEQYNSTYRVVSVLTVLHQDWLN | from the | |
| | | | | | GKEYKCKVSNKALPA | sequence for | |
| | | | | | PIEKTISKAKGQPREPQVYTLPPS | Abatacept | |
| | | | | | RDELTKNQVSLTCLVKGFYPSDIA | (DrugBank | |
| | | | | | VEWESNGQPENN | Online) | |
| | | | | | YKTTPPVLDSDGSFFLYSKLTVDK | | |
| | | | | | SRWQQGNVFSCSVMHEALHNHY | | |
| | | | | | TQKSLSLSPGK | | |
| Notch | H | Q04721 | uniprot | full | MPALRPALLWALLALWLCCAAPA | 1-25 is | SEQ |
| 2 | | | | length | HA | signal | ID |
| | | | | | LQCRDGYEPCVNEGMCVTYHNG | peptide; | NO: |
| | | | | | TGYCKCPEGFLGEYCQHRDPCE | 26-1677 | 5 |
| | | | | | KNRCQNGGTCVAQAMLGKATCR | is extra- | |
| | | | | | CASGFTGEDCQYSTSHPCFVSR | cellular; | |
| | | | | | PCLNGGTCHMLSRDTYECTCQV | 1678-1698 is | |
| | | | | | GFTGKECQWTDACLSHPCANGS | trans- | |
| | | | | | TCTTVANQFSCKCLTGFTGQKCE | membrane; | |
| | | | | | TDVNECDIPGHCQHGGTCLNLPG | 1699-2471 is | |
| | | | | | SYQCQCPQGFTGQYCDSLYVPC | cytoplasmic | |
| | | | | | APSPCVNGGTCRQTGDFTFECN | | |
| | | | | | CLPGFEGSTCERNIDDCPNHRCQ | | |
| | | | | | NGGVCVDGVNTYNCRCPPQWT | | |
| | | | | | GQFCTEDVDECLLQPNACQNGG | | |
| | | | | | TCANRNGGYGCVCVNGWSGDD | | |
| | | | | | CSENIDDCAFASCTPGSTCIDRVA | | |
| | | | | | SFSCMCPEGKAGLLCHLDDACIS | | |
| | | | | | NPCHKGALCDTNPLNGQYICTCP | | |
| | | | | | QGYKGADCTEDVDECAMANSNP | | |
| | | | | | CEHAGKCVNTDGAFHCECLKGY | | |
| | | | | | AGPRCEMDINECHSDPCQNDAT | | |
| | | | | | CLDKIGGFTCLCMPGFKGVHCEL | | |
| | | | | | EINECQSNPCVNNGQCVDKVNR | | |
| | | | | | FQCLCPPGFTGPVCQIDIDDCSST | | |
| | | | | | PCLNGAKCIDHPNGYECQCATGF | | |
| | | | | | TGVLCEENIDNCDPDPCHHGQC | | |
| | | | | | QDGIDSYTCICNPGYMGAICSDQI | | |
| | | | | | DECYSSPCLNDGRCIDLVNGYQC | | |
| | | | | | NCQPGTSGVNCEINFDDCASNPC | | |
| | | | | | IHGICMDGINRYSCVCSPGFTGQ | | |
| | | | | | RCNIDIDECASNPCRKGATCINGV | | |
| | | | | | NGFRCICPEGPHHPSCYSQVNEC | | |
| | | | | | LSNPCIHGNCTGGLSGYKCLCDA | | |
| | | | | | GWVGINCEVDKNECLSNPCQNG | | |
| | | | | | GTCDNLVNGYRCTCKKGFKGYN | | |
| | | | | | CQVNIDECASNPCLNQGTCFDDI | | |
| | | | | | SGYTCHCVLPYTGKNCQTVLAPC | | |
| | | | | | SPNPCENAAVCKESPNFESYTCL | | |
| | | | | | CAPGWQGQRCTIDIDECISKPCM | | |
| | | | | | NHGLCHNTQGSYMCECPPGFSG | | |
| | | | | | MDCEEDIDDCLANPCQNGGSCM | | |
| | | | | | DGVNTFSCLCLPGFTGDKCQTD | | |
| | | | | | MNECLSEPCKNGGTCSDYVNSY | | |
| | | | | | TCKCQAGFDGVHCENNINECTES | | |
| | | | | | SCFNGGTCVDGINSFSCLCPVGF | | |
| | | | | | TGSFCLHEINECSSHPCLNEGTC | | |
| | | | | | VDGLGTYRCSCPLGYTGKNCQTL | | |
| | | | | | VNLCSRSPCKNKGTCVQKKAES | | |
| | | | | | QCLCPSGWAGAYCDVPNVSCDI | | |
| | | | | | AASRRGVLVEHLCQHSGVCINAG | | |
| | | | | | NTHYCQCPLGYTGSYCEEQLDE | | |
| | | | | | CASNPCQHGATCSDFIGGYRCE | | |
| | | | | | CVPGYQGVNCEYEVDECQNQPC | | |
| | | | | | QNGGTCIDLVNHFKCSCPPGTRG | | |
| | | | | | LLCEENIDDCARGPHCLNGGQC | | |
| | | | | | MDRIGGYSCRCLPGFAGERCEG | | |
| | | | | | DINECLSNPCSSEGSLDCIQLTND | | |
| | | | | | YLCVCRSAFTGRHCETFVDVCPQ | | |
| | | | | | MPCLNGGTCAVASNMPDGFICR | | |
| | | | | | CPPGFSGARCQSSCGQVKCRKG | | |
| | | | | | EQCVHTASGPRCFCPSPRDCES | | |
| | | | | | GCASSPCQHGGSCHPQRQPPYY | | |
| | | | | | SCQCAPPFSGSRCELYTAPPSTP | | |
| | | | | | PATCLSQYCADKARDGVCDEAC | | |
| | | | | | NSHACQWDGGDCSLTMENPWA | | |
| | | | | | NCSSPLPCWDYINNQCDELCNTV | | |
| | | | | | ECLFDNFECQGNSKTCKYDKYCA | | |
| | | | | | DHFKDNHCDQGCNSEECGWDG | | |
| | | | | | LDCAADQPENLAEGTLVIVVLMPP | | |
| | | | | | EQLLQDARSFLRALGTLLHTNLRI | | |
| | | | | | KRDSQGELMVYPYYGEKSAAMK | | |
| | | | | | KQRMTRRSLPGEQEQEVAGSKV | | |
| | | | | | FLEIDNRQCVQDSDHCFKNTDAA | | |
| | | | | | AALLASHAIQGTLSYPLVSVVSES | | |
| | | | | | LTPERTQ | | |
| | | | | | LLYLLAVAVVIILFIILLGVIMAKRKR | | |
| | | | | | KHGSLWLPEGFTLRRDASNHKR | | |
| | | | | | REPVGQDAVGLKNLSVQVSEANL | | |
| | | | | | IGTGTSEHWVDDEGPQPKKVKAE | | |
| | | | | | DEALLSEEDDPIDRRPWTQQHLE | | |
| | | | | | AADIRRTPSLALTPPQAEQEVDVL | | |
| | | | | | DVNVRGPDGCTPLMLASLRGGS | | |
| | | | | | SDLSDEDEDAEDSSANIITDLVYQ | | |
| | | | | | GASLQAQTDRTGEMALHLAARYS | | |
| | | | | | RADAAKRLLDAGADANAQDNMG | | |
| | | | | | RCPLHAAVAADAQGVFQILIRNRV | | |
| | | | | | TDLDARMNDGTTPLILAARLAVEG | | |
| | | | | | MVAELINCQADVNAVDDHGKSAL | | |
| | | | | | HWAAAVNNVEATLLLLKNGANRD | | |
| | | | | | MQDNKEETPLFLAAREGSYEAAK | | |
| | | | | | ILLDHFANRDITDHMDRLPRDVAR | | |
| | | | | | DRMHHDIVRLLDEYNVTPSPPGT | | |
| | | | | | VLTSALSPVICGPNRSFLSLKHTP | | |
| | | | | | MGKKSRRPSAKSTMPTSLPNLAK | | |
| | | | | | EAKDAKGSRRKKSLSEKVQLSES | | |
| | | | | | SVTLSPVDSLESPHTYVSDTTSSP | | |
| | | | | | MITSPGILQASPNPMLATAAPPAP | | |
| | | | | | VHAQHALSFSNLHEMQPLAHGAS | | |
| | | | | | TVLPSVSQLLSHHHIVSPGSGSA | | |
| | | | | | GSLSRLHPVPVPADWMNRMEVN | | |
| | | | | | ETQYNEMFGMVLAPAEGTHPGIA | | |
| | | | | | PQSRPPEGKHITTPREPLPPIVTF | | |
| | | | | | QLIPKGSIAQPAGAPQPQSTCPP | | |
| | | | | | AVAGPLPTMYQIPEMARLPSVAF | | |
| | | | | | PTAMMPQQDGQVAQTILPAYHPF | | |
| | | | | | PASVGKYPTPPSQHSYASSNAAE | | |
| | | | | | RTPSHSGHLQGEHPYLTPSPESP | | |
| | | | | | DQWSSSSPHSASDWSDVTTSPT | | |
| | | | | | PGGAGGGQRGPGTHMSEPPHN | | |
| | | | | | NMQVYA | | |
| Notch2 | M | O35516 | uniprot | full | MPALRPAALRALLWLWLCGAGP | 1-25 is | SEQ |
| | | | | length | AHA | signal | ID |
| | | | | | LQCRGGQEPCVNEGTCVTYHNG | peptide; | NO: |
| | | | | | TGFCRCPEGFLGEYCQHRDPCE | 26-1679 | 6 |
| | | | | | KNRCQNGGTCVPQGMLGKATCR | is extra- | |
| | | | | | CAPGFTGEDCQYSTSHPCFVSR | cellular; | |
| | | | | | PCQNGGTCHMLSRDTYECTCQV | 1680-1700 is | |
| | | | | | GFTGKQCQWTDACLSHPCENGS | trans- | |
| | | | | | TCTSVASQFSCKCPAGLTGQKCE | membrane; | |
| | | | | | ADINECDIPGRCQHGGTCLNLPG | 1701-2473 is | |
| | | | | | SYRCQCPQGFTGQHCDSPYVPC | cytoplasmic | |
| | | | | | APSPCVNGGTCRQTGDFTFECN | | |
| | | | | | CLPGFEGSTCERNIDDCPNHKCQ | | |
| | | | | | NGGVCVDGVNTYNCRCPPQWT | | |
| | | | | | GQFCTEDVDECLLQPNACQNGG | | |
| | | | | | TCTNRNGGYGCVCVNGWSGDD | | |
| | | | | | CSENIDDCAYASCTPGSTCIDRVA | | |
| | | | | | SFSCLCPEGKAGLLCHLDDACIS | | |
| | | | | | NPCHKGALCDTNPLNGQYICTCP | | |
| | | | | | QGYKGADCTEDVDECAMANSNP | | |
| | | | | | CEHAGKCVNTDGAFHCECLKGY | | |
| | | | | | AGPRCEMDINECHSDPCQNDAT | | |
| | | | | | CLDKIGGFTCLCMPGFKGVHCEL | | |
| | | | | | EVNECQSNPCVNNGQCVDKVNR | | |
| | | | | | FQCLCPPGFTGPVCQIDIDDCSST | | |
| | | | | | PCLNGAKCIDHPNGYECQCATGF | | |
| | | | | | TGILCDENIDNCDPDPCHHGQCQ | | |
| | | | | | DGIDSYTCICNPGYMGAICSDQID | | |
| | | | | | ECYSSPCLNDGRCIDLVNGYQCN | | |
| | | | | | CQPGTSGLNCEINFDDCASNPCM | | |
| | | | | | HGVCVDGINRYSCVCSPGFTGQ | | |
| | | | | | RCNIDIDECASNPCRKGATCINDV | | |
| | | | | | NGFRCICPEGPHHPSCYSQVNEC | | |
| | | | | | LSNPCIHGNCTGGLSGYKCLCDA | | |
| | | | | | GWVGVNCEVDKNECLSNPCQNG | | |
| | | | | | GTCNNLVNGYRCTCKKGFKGYN | | |
| | | | | | CQVNIDECASNPCLNQGTCFDDV | | |
| | | | | | SGYTCHCMLPYTGKNCQTVLAP | | |
| | | | | | CSPNPCENAAVCKEAPNFESFSC | | |
| | | | | | LCAPGWQGKRCTVDVDECISKP | | |
| | | | | | CMNNGVCHNTQGSYVCECPPGF | | |
| | | | | | SGMDCEEDINDCLANPCQNGGS | | |
| | | | | | CVDHVNTFSCQCHPGFIGDKCQT | | |
| | | | | | DMNECLSEPCKNGGTCSDYVNS | | |
| | | | | | YTCTCPAGFHGVHCENNIDECTE | | |
| | | | | | SSCFNGGTCVDGINSFSCLCPVG | | |
| | | | | | FTGPFCLHDINECSSNPCLNAGT | | |
| | | | | | CVDGLGTYRCICPLGYTGKNCQT | | |
| | | | | | LVNLCSRSPCKNKGTCVQEKARP | | |
| | | | | | HCLCPPGWDGAYCDVLNVSCKA | | |
| | | | | | AALQKGVPVEHLCQHSGICINAG | | |
| | | | | | NTHHCQCPLGYTGSYCEEQLDE | | |
| | | | | | CASNPCQHGATCNDFIGGYRCE | | |
| | | | | | CVPGYQGVNCEYEVDECQNQPC | | |
| | | | | | QNGGTCIDLVNHFKCSCPPGTRG | | |
| | | | | | LLCEENIDECAGGPHCLNGGQCV | | |
| | | | | | DRIGGYTCRCLPGFAGERCEGDI | | |
| | | | | | NECLSNPCSSEGSLDCVQLKNNY | | |
| | | | | | NCICRSAFTGRHCETFLDVCPQK | | |
| | | | | | PCLNGGTCAVASNMPDGFICRCP | | |
| | | | | | PGFSGARCQSSCGQVKCRRGEQ | | |
| | | | | | CIHTDSGPRCFCLNPKDCESGCA | | |
| | | | | | SNPCQHGGTCYPQRQPPHYSCR | | |
| | | | | | CPPSFGGSHCELYTAPTSTPPAT | | |
| | | | | | CQSQYCADKARDGICDEACNSH | | |
| | | | | | ACQWDGGDCSLTMEDPWANCT | | |
| | | | | | STLRCWEYINNQCDEQCNTAECL | | |
| | | | | | FDNFECQRNSKTCKYDKYCADH | | |
| | | | | | FKDNHCDQGCNSEECGWDGLD | | |
| | | | | | CASDQPENLAEGTLIIVVLLPPEQ | | |
| | | | | | LLQDSRSFLRALGTLLHTNLRIKQ | | |
| | | | | | DSQGALMVYPYFGEKSAAMKKQ | | |
| | | | | | KMTRRSLPEEQEQEQEVIGSKIFL | | |
| | | | | | EIDNRQCVQDSDQCFKNTDAAAA | | |
| | | | | | LLASHAIQGTLSYPLVSVFSELES | | |
| | | | | | PRNAQ | | |
| | | | | | LLYLLAVAVVIILFFILLGVIMAKRK | | |
| | | | | | RKHGFLWLPEGFTLRRDSSNHK | | |
| | | | | | RREPVGQDAVGLKNLSVQVSEA | | |
| | | | | | NLIGSGTSEHWVDDEGPQPKKAK | | |
| | | | | | AEDEALLSEDDPIDRRPWTQQHL | | |
| | | | | | EAADIRHTPSLALTPPQAEQEVDV | | |
| | | | | | LDVNVRGPDGCTPLMLASLRGG | | |
| | | | | | SSDLSDEDEDAEDSSANIITDLVY | | |
| | | | | | QGASLQAQTDRTGEMALHLAAR | | |
| | | | | | YSRADAAKRLLDAGADANAQDN | | |
| | | | | | MGRCPLHAAVAADAQGVFQILIR | | |
| | | | | | NRVTDLDARMNDGTTPLILAARLA | | |
| | | | | | VEGMVAELINCQADVNAVDDHGK | | |
| | | | | | SALHWAAAVNNVEATLLLLKNGA | | |
| | | | | | NRDMQDNKEETPLFLAAREGSYE | | |
| | | | | | AAKILLDHFANRDITDHMDRLPRD | | |
| | | | | | VARDRMHHDIVRLLDEYNVTPSP | | |
| | | | | | PGTVLTSALSPVLCGPNRSFLSLK | | |
| | | | | | HTPMGKKARRPNTKSTMPTSLPN | | |
| | | | | | LAKEAKDAKGSRRKKCLNEKVQL | | |
| | | | | | SESSVTLSPVDSLESPHTYVSDA | | |
| | | | | | TSSPMITSPGILQASPTPLLAAAA | | |
| | | | | | PAAPVHTQHALSFSNLHDMQPLA | | |
| | | | | | PGASTVLPSVSQLLSHHHIAPPGS | | |
| | | | | | SSAGSLGRLHPVPVPADWMNRV | | |
| | | | | | EMNETQYSEMFGMVLAPAEGAH | | |
| | | | | | PGIAAPQSRPPEGKHMSTQREPL | | |
| | | | | | PPIVTFQLIPKGSIAQAAGAPQTQ | | |
| | | | | | SSCPPAVAGPLPSMYQIPEMPRL | | |
| | | | | | PSVAFPPTMMPQQEGQVAQTIVP | | |
| | | | | | TYHPFPASVGKYPTPPSQHSYAS | | |
| | | | | | SNAAERTPSHGGHLQGEHPYLTP | | |
| | | | | | SPESPDQWSSSSPHSASDWSDV | | |
| | | | | | TTSPTPGGGGGGQRGPGTHMSE | | |
| | | | | | PPHSNMQVYA | | |
| Notch2- | H | Q04721 | uniprot | A26- | lqcrdgyepcvnegmcvtyhngtgyckcpe | Extracellular | SEQ |
| Fc, | | (Notch2) | (Notch2) | A1677 | gflgeycqhrdpceknrcqnggtcvaqamlg | domain of | ID |
| extra- | | DB01281 | DrugBank | (Notch2) | katcrcasgftgedcqystshpcfvsrpclngg | Notch2 with | NO: |
| cllular | | (Abata- | (Abata- | | tchmlsrdtyectcqvgftgkecqwtdaclshp | abatacept Fc | 7 |
| domain | | cept) | cept) | | cangstcttvanqfsckcltgftgqkcetdvnec | | |
| | | | | | dipghcqhggtclnlpgsyqcqcpqgftgqyc | | |
| | | | | | dslyvpcapspcvnggtcrqtgdftfecnclpg | | |
| | | | | | fegstcerniddcpnhrcqnggvcvdgvntyn | | |
| | | | | | crcppqwtgqfctedvdecllqpnacqnggtc | | |
| | | | | | anrnggygcvcvngwsgddcseniddcafa | | |
| | | | | | sctpgstcidrvasfscmcpegkagllchldda | | |
| | | | | | cisnpchkgalcdtnplngqyictcpqgykga | | |
| | | | | | dctedvdecamansnpcehagkcvntdgaf | | |
| | | | | | hceclkgyagprcemdinechsdpcqndat | | |
| | | | | | cldkiggftclcmpgfkgvhceleinecqsnpc | | |
| | | | | | vnngqcvdkvnrfqclcppgftgpvcqididdc | | |
| | | | | | sstpclngakcidhpngyecqcatgftgvlcee | | |
| | | | | | nidncdpdpchhgqcqdgidsytcicnpgy | | |
| | | | | | mgaicsdqidecysspclndgrcidlvngyqc | | |
| | | | | | ncqpgtsgvnceinfddcasnpcihgicmdgi | | |
| | | | | | nryscvcspgftgqrcnididecasnpcrkgat | | |
| | | | | | cingvngfrcicpegphhpscysqvneclsnp | | |
| | | | | | cihgnctgglsgykclcdagwvgincevdkne | | |
| | | | | | clsnpcqnggtcdnlvngyrctckkgfkgync | | |
| | | | | | qvnidecasnpcinqgtcfddisgytchcvlpy | | |
| | | | | | tgkncqtvlapcspnpcenaavckespnfes | | |
| | | | | | ytclcapgwqgqrctidideciskpcmnhglc | | |
| | | | | | hntqgsymcecppgfsgmdceediddclan | | |
| | | | | | pcqnggscmdgvntfsclclpgftgdkcqtd | | |
| | | | | | mneclsepcknggtcsdyvnsytckcqagfd | | |
| | | | | | gvhcenninectesscfnggtcvdginsfsclc | | |
| | | | | | pvgftgsfclheinecsshpclnegtcvdglgty | | |
| | | | | | rcscplgytgkncqtlvnlcsrspcknkgtcvqk | | |
| | | | | | kaesqclcpsgwagaycdvpnvscdiaasrr | | |
| | | | | | gvlvehlcqhsgvcinagnthycqcplgytgs | | |
| | | | | | yceeqldecasnpcqhgatcsdfiggyrcecv | | |
| | | | | | pgyqgvnceyevdecqnqpcqnggtcidlv | | |
| | | | | | nhfkcscppgtrgllceeniddcargphclngg | | |
| | | | | | qcmdriggyscrclpgfagercegdineclsn | | |
| | | | | | pcssegsldciqltndylcvcrsaftgrhcetfvd | | |
| | | | | | vcpqmpclnggtcavasnmpdgficrcppgf | | |
| | | | | | sgarcqsscgqvkcrkgeqcvhtasgprcfc | | |
| | | | | | psprdcesgcasspcqhggschpqrqppyy | | |
| | | | | | scqcappfsgsrcelytappstppatclsqyca | | |
| | | | | | dkardgvcdeacnshacqwdggdcsltme | | |
| | | | | | npwancssplpcwdyinnqcdelcntveclfd | | |
| | | | | | nfecqgnsktckydkycadhfkdnhcdqgcn | | |
| | | | | | seecgwdgldcaadqpenlaegtlvivvlmp | | |
| | | | | | peqllqdarsflralgtllhtnlrikrdsqgelmvy | | |
| | | | | | pyygeksaamkkqrmtrrslpgeqeqevag | | |
| | | | | | skvfleidnrqcvqdsdhcfkntdaaaallash | | |
| | | | | | aiqgtlsyplvsvvsesltpertqQEPKSSD | | |
| | | | | | KTHTSPPSPAPELLGGSSVFLFPP | | |
| | | | | | KPKDTLMISRTPEVTCVVVDVSH | | |
| | | | | | EDPEVKFNWYVDGVEVHNAKTK | | |
| | | | | | PREEQYNSTYRVVSVLTVLHQD | | |
| | | | | | WLNGKEYKCKVSNKALPAPIEKTI | | |
| | | | | | SKAKGQPREPQVYTLPPSRDELT | | |
| | | | | | KNQVSLTCLVKGFYPSDIAVEWE | | |
| | | | | | SNGQPENNYKTTPPVLDSDGSFF | | |
| | | | | | LYSKLTVDKSRWQQGNVFSCSV | | |
| | | | | | MHEALHNHYTQKSLSLSPGK | | |
| Notch2- | M | O35516 | uniprot | A26- | lqcrggqepcvnegtcvtyhngtgfcrcpegfl | Extracellular | SEQ |
| Fc, | | (Notch2) | (Notch2) | A1679 | geycqhrdpceknrcqnggtcvpqgmlgkat | domain of | ID |
| extra- | | DB01281 | DrugBank | (Notch2) | crcapgftgedcqystshpcfvsrpcqnggtc | Notch2 with | NO: |
| cllular | | (Abata- | (Abata- | | hmlsrdtyectcqvgftgkqcqwtdaclshpc | abatacept Fc | 8 |
| domain | | cept) | cept) | | engstctsvasqfsckcpagltgqkceadine | | |
| | | | | | cdipgrcqhggtclnlpgsyrcqcpqgftgqhc | | |
| | | | | | dspyvpcapspcvnggtcrqtgdftfecnclp | | |
| | | | | | gfegstcerniddcpnhkcqnggvcvdgvnt | | |
| | | | | | yncrcppqwtgqfctedvdecllqpnacqng | | |
| | | | | | gtctnrnggygcvcvngwsgddcseniddca | | |
| | | | | | yasctpgstcidrvasfsclcpegkagllchldd | | |
| | | | | | acisnpchkgalcdtnplngqyictcpqgykg | | |
| | | | | | adctedvdecamansnpcehagkcvntdg | | |
| | | | | | afhceclkgyagprcemdinechsdpcqnd | | |
| | | | | | atcldkiggftclcmpgfkgvhcelevnecqsn | | |
| | | | | | pcvnngqcvdkvnrfqclcppgftgpvcqidid | | |
| | | | | | dcsstpclngakcidhpngyecqcatgftgilc | | |
| | | | | | denidncdpdpchhgqcqdgidsytcicnpg | | |
| | | | | | ymgaicsdqidecysspclndgrcidlvngyq | | |
| | | | | | cncqpgtsglnceinfddcasnpcmhgvcvd | | |
| | | | | | ginryscvcspgftgqrcnididecasnpcrkg | | |
| | | | | | atcindvngfrcicpegphhpscysqvnecls | | |
| | | | | | npcihgnctgglsgykclcdagwvgvncevd | | |
| | | | | | kneclsnpcqnggtcnnlvngyrctckkgfkg | | |
| | | | | | yncqvnidecasnpclnqgtcfddvsgytchc | | |
| | | | | | mlpytgkncqtvlapcspnpcenaavckeap | | |
| | | | | | nfesfsclcapgwqgkrctvdvdeciskpcm | | |
| | | | | | nngvchntqgsyvcecppgfsgmdceedin | | |
| | | | | | dclanpcqnggscvdhvntfscqchpgfigdk | | |
| | | | | | cqtdmneclsepcknggtcsdyvnsytctcp | | |
| | | | | | agfhgvhcennidectesscfnggtcvdgins | | |
| | | | | | fsclcpvgftgpfclhdinecssnpclnagtcvd | | |
| | | | | | glgtyrcicplgytgkncqtlvnlcsrspcknkgt | | |
| | | | | | cvqekarphclcppgwdgaycdvlnvscka | | |
| | | | | | aalqkgvpvehlcqhsgicinagnthhcqcpl | | |
| | | | | | gytgsyceeqldecasnpcqhgatcndfigg | | |
| | | | | | yrcecvpgyqgvnceyevdecqnqpcqng | | |
| | | | | | gtcidlvnhfkcscppgtrgllceenidecaggp | | |
| | | | | | hclnggqcvdriggytcrclpgfagercegdin | | |
| | | | | | eclsnpcssegsldcvqlknnyncicrsaftgr | | |
| | | | | | hcetfldvcpqkpclnggtcavasnmpdgfic | | |
| | | | | | rcppgfsgarcqsscgqvkcrrgeqcihtdsg | | |
| | | | | | prcfclnpkdcesgcasnpcqhggtcypqrq | | |
| | | | | | pphyscrcppsfggshcelytaptstppatcq | | |
| | | | | | sqycadkardgicdeacnshacqwdggdcs | | |
| | | | | | ltmedpwanctstlrcweyinnqcdeqcntae | | |
| | | | | | clfdnfecqrnsktckydkycadhfkdnhcdq | | |
| | | | | | gcnseecgwdgldcasdqpenlaegtliivvll | | |
| | | | | | ppeqllqdsrsflralgtllhtnlrikqdsqgalmv | | |
| | | | | | ypyfgeksaamkkqkmtrrslpeeqeqeqe | | |
| | | | | | vigskifleidnrqcvqdsdqcfkntdaaaalla | | |
| | | | | | shaiqgtlsyplvsvfselesprnaqQEPKS | | |
| | | | | | SDKTHTSPPSPAPELLGGSSVFL | | |
| | | | | | FPPKPKDTLMISRTPEVTCVVVDV | | |
| | | | | | SHEDPEVKFNWYVDGVEVHNAK | | |
| | | | | | TKPREEQYNSTYRVVSVLTVLHQ | | |
| | | | | | DWLNGKEYKCKVSNKALPAPIEK | | |
| | | | | | TISKAKGQPREPQVYTLPPSRDE | | |
| | | | | | LTKNQVSLTCLVKGFYPSDIAVE | | |
| | | | | | WESNGQPENNYKTTPPVLDSDG | | |
| | | | | | SFFLYSKLTVDKSRWQQGNVFSC | | |
| | | | | | SVMHEALHNHYTQKSLSLSPGK | | |
| Jagged- | H | Q9Y219 | Uniprot | A27- | arpmgyfelqlsalrnvngellsgaccdgdgrtt | LowerCase | SEQ |
| 2-Fc | | (Jagged2) | (Jagged2) | A1080 | raggcghdecdtyvrvclkeyqakvtptgpcs | is Jagged-2 | ID |
| | | DB01281 | DrugBank | (Jagged2) | yghgatpvlggnsfylppagaagdrararara | extracellular | NO: |
| | | (Abata- | (Abata- | | ggdqdpglvvipfqfawprsftliveawdwdn | domain | 9 |
| | | cept) | cept) | | dttpneelliervshagminpedrwkslhfsgh | sequence | |
| | | | | | vahlelqirvrcdenyysatcnkfcrprndffgh | without | |
| | | | | | ytcdqygnkacmdgwmgkeckeavckqg | the signal | |
| | | | | | cnllhggctvpgecrcsygwqgrfcdecvpyp | peptide | |
| | | | | | gcvhgscvepwqcncetnwggllcdkdlnyc | AA 24-1080. | |
| | | | | | gshhpctnggtcinaepdqyrctcpdgysgrn | Uppercase | |
| | | | | | cekaehactsnpcanggschevpsgfechc | is Fc from | |
| | | | | | psgwsgptcaldidecasnpcaaggtcvdqv | Abatacept | |
| | | | | | dgfecicpeqwvgatcqldanecegkpclnaf | | |
| | | | | | scknliggyycdcipgwkginchinvndcrgq | | |
| | | | | | cqhggtckdlvngyqcvcprgfggrhcelerd | | |
| | | | | | ecasspchsgglcedladgfhchcpqgfsgpl | | |
| | | | | | cevdvdlcepspcrngarcynlegdyycacp | | |
| | | | | | ddfggkncsvprepcpggacrvidgcgsdag | | |
| | | | | | pgmpgtaasgvcgphgrcvsqpggnfscic | | |
| | | | | | dsgftgtycheniddclgqpcrnggtcidevda | | |
| | | | | | frcfcpsgwegelcdtnpndclpdpchsrgrc | | |
| | | | | | ydlvndfycacddgwkgktchsrefqcdaytc | | |
| | | | | | snggtcydsgdtfrcacppgwkgstcavakn | | |
| | | | | | ssclpnpcvnggtcvgsgasfscicrdgwegr | | |
| | | | | | tcthntndcnplpcynggicvdgvnwfrceca | | |
| | | | | | pgfagpdcrinidecqsspcaygatcvdeing | | |
| | | | | | yrcscppgragprcqevigfgrscwsrgtpfph | | |
| | | | | | gsswvedcnscrcldgrrdcskvwcgwkpcl | | |
| | | | | | lagqpealsaqcplgqrclekapgqclrppce | | |
| | | | | | awgecgaeeppstpclprsghldnncarltlhf | | |
| | | | | | nrdhvpqgttvgaicsgirslpatravardrllvll | | |
| | | | | | cdrassgasavevavsfspardlpdssliqga | | |
| | | | | | ahaivaaitqrgnsslllavtevkvetvvtggsst | | |
| | | | | | QEPKSSDKTHTSPPSPAPELLGG | | |
| | | | | | SSVFLFPPKPKDTLMISRTPEVTC | | |
| | | | | | VVVDVSHEDPEVKFNWYVDGVE | | |
| | | | | | VHNAKTKPREEQYNSTYRVVSVL | | |
| | | | | | TVLHQDWLNGKEYKCKVSNKALP | | |
| | | | | | APIEKTISKAKGQPREPQVYTLPP | | |
| | | | | | SRDELTKNQVSLTCLVKGFYPSDI | | |
| | | | | | AVEWESNGQPENNYKTTPPVLD | | |
| | | | | | SDGSFFLYSKLTVDKSRWQQGN | | |
| | | | | | VFSCSVMHEALHNHYTQKSLSLS | | |
| | | | | | PGK | | |
| UMOD | H | P07911 | Uniprot | full | MGQPSLTWMLMVVVASWFITTA | 1-24 | SEQ |
| | | | | length | ATDTSEARWCSECHSNATCTED | signal | ID |
| | | | | | EAVTTCTCQEGFTGDGLTCVDLD | peptide; | NO: |
| | | | | | ECAIPGAHNCSANSSCVNTPGSF | 25-614 | 10 |
| | | | | | SCVCPEGFRLSPGLGCTDVDECA | uromodulin; | |
| | | | | | EPGLSHCHALATCVNVVGSYLCV | note: | |
| | | | | | CPAGYRGDGWHCECSPGSCGP | 25-587 is | |
| | | | | | GLDCVPEGDALVCADPCQAHRTL | secreted | |
| | | | | | DEYWRSTEYGEGYACDTDLRGW | form; | |
| | | | | | YRFVGQGGARMAETCVPVLRCN | 615-640 | |
| | | | | | TAAPMWLNGTHPSSDEGIVSRKA | is | |
| | | | | | CAHWSGHCCLWDASVQVKACA | cleaved | |
| | | | | | GGYYVYNLTAPPECHLAYCTDPS | in mature | |
| | | | | | SVEGTCEECSIDEDCKSNNGRW | form | |
| | | | | | HCQCKQDFNITDISLLEHRLECGA | | |
| | | | | | NDMKVSLGKCQLKSLGFDKVFM | | |
| | | | | | YLSDSRCSGFNDRDNRDWVSVV | | |
| | | | | | TPARDGPCGTVLTRNETHATYSN | | |
| | | | | | TLYLADEIIIRDLNIKINFACSYPLD | | |
| | | | | | MKVSLKTALQPMVSALNIRVGGT | | |
| | | | | | GMFTVRMALFQTPSYTQPYQGS | | |
| | | | | | SVTLSTEAFLYVGTMLDGGDLSR | | |
| | | | | | FALLMTNCYATPSSNATDPLKYFII | | |
| | | | | | QDRCPHTRDSTIQVVENGESSQ | | |
| | | | | | GRFSVQMFRFAGNYDLVYLHCE | | |
| | | | | | VYLCDTMNEKCKPTCSGTRFRS | | |
| | | | | | GSVIDQSRVLNLGPITRKGVQATV | | |
| | | | | | SRAFSSLGLLKVWLPLLLSATLTL | | |
| | | | | | TFQ | | |
| UMOD | M | Q91X17 | Uniprot | full | MGIPLTWMLLVMMVTSWFTLAEA | 1-24 | SEQ |
| | | | | length | SNSTEARRCSECHNNATCTVDG | signal | ID |
| | | | | | VVTTCSCQTGFTGDGLVCEDMD | peptide; | NO: |
| | | | | | ECATPWTHNCSNSSCVNTPGSF | 25-618 | 11 |
| | | | | | KCSCQDGFRLTPELSCTDVDECS | uromodulin; | |
| | | | | | EQGLSNCHALATCVNTEGDYLCV | note: | |
| | | | | | CPEGFTGDGWYCECSPGSCEPG | 25-588 is | |
| | | | | | LDCLPQGPDGKLVCQDPCNTYET | secreted | |
| | | | | | LTEYWRSTEYGVGYSCDAGLHG | form; | |
| | | | | | WYRFTGQGGVRMAETCVPVLRC | 619-642 | |
| | | | | | NTAAPMWLNGSHPSSSEGIVSRT | is | |
| | | | | | ACAHWSDQCCRWSTEIQVKACP | cleaved | |
| | | | | | GGFYIYNLTAPPECNLAYCTDPSS | in mature | |
| | | | | | VEGTCEECRVDEDCISDNGRWR | form | |
| | | | | | CQCKQDSNITDVSQLEYRLECGA | | |
| | | | | | NDIKMSLRKCQLQSLGFMNVFMY | | |
| | | | | | LNDRQCSGFSESDERDWMSIVT | | |
| | | | | | PARNGPCGTVLRRNETHATYSNT | | |
| | | | | | LYLANAIIIRDIIIRMNFECSYPLDM | | |
| | | | | | KVSLKTSLQPMVSALNISLGGTGK | | |
| | | | | | FTVRMALFQSPTYTQPHQGPSV | | |
| | | | | | MLSTEAFLYVGTMLDGGDLSRFV | | |
| | | | | | LLMTNCYATPSSNSTDPVKYFIIQ | | |
| | | | | | DSCPRTEDTTIQVTENGESSQAR | | |
| | | | | | FSVQMFRFAGNYDLVYLHCEVYL | | |
| | | | | | CDSTSEQCKPTCSGTRFRSGNFI | | |
| | | | | | DQTRVLNLGPITRQGVQASVSKA | | |
| | | | | | ASSNLRLLSIWLLLFPSATLIFMVQ | | |
| MEP1B | H | Q16820 | Uniprot | full | MDLWNLSWFLFLDALLVISGLATP | 1-22 | SEQ |
| | | | | length | ENFDVDGGMDQDIFDINEGLGLD | signal | ID |
| | | | | | LFEGDIRLDRAQIRNSIIGEKYRW | peptide; | NO: |
| | | | | | PHTIPYVLEDSLEMNAKGVILNAF | 23-652 | 12 |
| | | | | | ERYRLKTCIDFKPWAGETNYISVF | extracellular; | |
| | | | | | KGSGCWSSVGNRRVGKQELSIG | 653-673 | |
| | | | | | ANCDRIATVQHEFLHALGFWHEQ | transmembrane; | |
| | | | | | SRSDRDDYVRIMWDRILSGREHN | 674-701 | |
| | | | | | FNTYSDDISDSLNVPYDYTSVMH | cytosol | |
| | | | | | YSKTAFQNGTEPTIVTRISDFEDVI | | |
| | | | | | GQRMDFSDSDLLKLNQLYNCSSS | | |
| | | | | | LSFMDSCSFELENVCGMIQSSGD | | |
| | | | | | NADWQRVSQVPRGPESDHSNM | | |
| | | | | | GQCQGSGFFMHFDSSSVNVGAT | | |
| | | | | | AVLESRTLYPKRGFQCLQFYLYN | | |
| | | | | | SGSESDQLNIYIREYSADNVDGNL | | |
| | | | | | TLVEEIKEIPTGSWQLYHVTLKVT | | |
| | | | | | KKFRVVFEGRKGSGASLGGLSID | | |
| | | | | | DINLSETRCPHHIWHIRNFTQFIG | | |
| | | | | | SPNGTLYSPPFYSSKGYAFQIYLN | | |
| | | | | | LAHVTNAGIYFHLISGANDDQLQ | | |
| | | | | | WPCPWQQATMTLLDQNPDIRQR | | |
| | | | | | MSNQRSITTDPFMTTDNGNYFW | | |
| | | | | | DRPSKVGTVALFSNGTQFRRGG | | |
| | | | | | GYGTSAFITHERLKSRDFIKGDDV | | |
| | | | | | YILLTVEDISHLNSTQIQLTPAPSV | | |
| | | | | | QDLCSKTTCKNDGVCTVRDGKA | | |
| | | | | | ECRCQSGEDWWYMGERCEKRG | | |
| | | | | | STRDTIVIAVSSTVAVFALMLIITLV | | |
| | | | | | SVYCTRKKYRERMSSNRPNLTP | | |
| | | | | | QNQHAF | | |
| MEP1B | M | Q61847 | Uniprot | full | MDARHQPWFLVFATFLLVSGLPA | 1-20 | SEQ |
| | | | | length | PEKFVKDIDGGIDQDIFDINQGLG | signal | ID |
| | | | | | LDLFEGDIKLEANGKNSIIGDHKR | peptide; | NO: |
| | | | | | WPHTIPYVLEDSLEMNAKGVILNA | 21-654 | 13 |
| | | | | | FERYRLKTCIDFKPWSGEANYISV | extracellular; | |
| | | | | | FKGSGCWSSVGNIHAGKQELSIG | 655-678 | |
| | | | | | TNCDRIATVQHEFLHALGFWHEQ | transmembrane; | |
| | | | | | SRADRDDYVIIVWDRIQPGKEHN | 679-704 | |
| | | | | | FNIYNDSVSDSLNVPYDYTSVMH | cytosol | |
| | | | | | YSKTAFQNGTESTIVTRISEFEDVI | | |
| | | | | | GQRMDFSDYDLLKLNQLYNCTSS | | |
| | | | | | LSFMDSCDFELENICGMIQSSGD | | |
| | | | | | SADWQRVSQVLSGPESDHSKMG | | |
| | | | | | QCKDSGFFMHFNTSILNEGATAM | | |
| | | | | | LESRLLYPKRGFQCLEFYLYNSG | | |
| | | | | | SGNDQLNIYTREYTTGQQGGVLT | | |
| | | | | | LQRQIKEVPIGSWQLHYVTLQVTK | | |
| | | | | | KFRVVFEGLRGPGTSSGGLSIDDI | | |
| | | | | | NLSETRCPHHIWHIQNFTQILGGQ | | |
| | | | | | DTSVYSPPFYSSKGYAFQIYMDL | | |
| | | | | | RSSTNVGIYFHLISGANDDQLQW | | |
| | | | | | PCPWQQATMTLLDQNPDIRQRM | | |
| | | | | | FNQRSITTDPTMTSDNGSYFWDR | | |
| | | | | | PSKVGVTDVFPNGTQFSRGIGYG | | |
| | | | | | TTVFITRERLKSREFIKGDDIYILLT | | |
| | | | | | VEDISHLNSTSAVPDPVPTLAVHN | | |
| | | | | | ACSEVVCQNGGICVVQDGRAEC | | |
| | | | | | KCPAGEDWWYMGKRCEKRGST | | |
| | | | | | RDTVIIAVSSTVTVFAVMLIITLVSV | | |
| | | | | | YCTRRKYRKKARANTAAMTLENQ | | |
| | | | | | HAF | | |
| IL11RA | H | Q14626 | Uniprot | full | MSSSCSGLSRVLVAVATALVSAS | 1-22 is | SEQ |
| | | | | length | SPCPQAWGPPGVQYGQPGRSV | signal | ID |
| | | | | | KLCCPGVTAGDPVSWFRDGEPK | peptide; | NO: |
| | | | | | LLQGPDSGLGHELVLAQADSTDE | 23-422 is | 14 |
| | | | | | GTYICQTLDGALGGTVTLQLGYP | full IL11Ra; | |
| | | | | | PARPVVSCQAADYENFSCTWSP | 371-391 is | |
| | | | | | SQISGLPTRYLTSYRKKTVLGADS | transmembrane; | |
| | | | | | QRRSPSTGPWPCPQDPLGAARC | 392-422 is | |
| | | | | | VVHGAEFWSQYRINVTEVNPLGA | cytoplasmic) | |
| | | | | | STRLLDVSLQSILRPDPPQGLRVE | | |
| | | | | | SVPGYPRRLRASWTYPASWPCQ | | |
| | | | | | PHFLLKFRLQYRPAQHPAWSTVE | | |
| | | | | | PAGLEEVITDAVAGLPHAVRVSA | | |
| | | | | | RDFLDAGTWSTWSPEAWGTPST | | |
| | | | | | GTIPKEIPAWGQLHTQPEVEPQV | | |
| | | | | | DSPAPPRPSLQPHPRLLDHRDSV | | |
| | | | | | EQVAVLASLGILSFLGLVAGALAL | | |
| | | | | | GLWLRLRRGGKDGSPKPGFLAS | | |
| | | | | | VIPVDRRPGAPNL | | |
| IL11RA | M | Q64385 | Uniprot | full | MSSSCSGLTRVLVAVATALVSSS | 1-23 is | SEQ |
| | | | | length | SPCPQAWGPPGVQYGQPGRPV | signal | ID |
| | | | | | MLCCPGVSAGTPVSWFRDGDSR | peptide; | NO: |
| | | | | | LLQGPDSGLGHRLVLAQVDSPDE | 24-432 is | 15 |
| | | | | | GTYVCQTLDGVSGGMVTLKLGFP | full IL11Ra; | |
| | | | | | PARPEVSCQAVDYENFSCTWSP | 373-393 is | |
| | | | | | GQVSGLPTRYLTSYRKKTLPGAE | transmembrane; | |
| | | | | | SQRESPSTGPWPCPQDPLEASR | 394-432 is | |
| | | | | | CVVHGAEFWSEYRINVTEVNPLG | cytoplasmic) | |
| | | | | | ASTCLLDVRLQSILRPDPPQGLRV | | |
| | | | | | ESVPGYPRRLHASWTYPASWRR | | |
| | | | | | QPHFLLKFRLQYRPAQHPAWSTV | | |
| | | | | | EPIGLEEVITDAVAGLPHAVRVSA | | |
| | | | | | RDFLDAGTWSAWSPEAWGTPST | | |
| | | | | | GPLQDEIPDWSQGHGQQLEAVV | | |
| | | | | | AQEDSPAPARPSLQPDPRPLDHR | | |
| | | | | | DPLEQVAVLASLGIFSCLGLAVGA | | |
| | | | | | LALGLWLRLRRSGKDGPQKPGLL | | |
| | | | | | APMIPVEKLPGIPNLQRTPENFS | | |
| IL11RA, | H | Q14626 | Uniprot | A23-391 | SSPCPQAWGPPGVQYGQPGRS | | SEQ |
| extra- | | | | | VKLCCPGVTAGDPVSWFRDGEP | | ID |
| cellular | | | | | KLLQGPDSGLGHELVLAQADSTD | | NO: |
| domain | | | | | EGTYICQTLDGALGGTVTLQLGY | | 16 |
| | | | | | PPARPVVSCQAADYENFSCTWS | | |
| | | | | | PSQISGLPTRYLTSYRKKTVLGAD | | |
| | | | | | SQRRSPSTGPWPCPQDPLGAAR | | |
| | | | | | CVVHGAEFWSQYRINVTEVNPLG | | |
| | | | | | ASTRLLDVSLQSILRPDPPQGLRV | | |
| | | | | | ESVPGYPRRLRASWTYPASWPC | | |
| | | | | | QPHFLLKFRLQYRPAQHPAWSTV | | |
| | | | | | EPAGLEEVITDAVAGLPHAVRVSA | | |
| | | | | | RDFLDAGTWSTWSPEAWGTPST | | |
| | | | | | GTIPKEIPAWGQLHTQPEVEPQV | | |
| | | | | | DSPAPPRPSLQPHPRLLDHRDSV | | |
| | | | | | EQVAVLA | | |
| IL11RA, | M | Q64385 | Uniprot | A24- | SPCPQAWGPPGVQYGQPGRPV | | SEQ |
| extra- | | | | A393 | MLCCPGVSAGTPVSWFRDGDSR | | ID |
| cellular | | | | | LLQGPDSGLGHRLVLAQVDSPDE | | NO: |
| domain | | | | | GTYVCQTLDGVSGGMVTLKLGFP | | 17 |
| | | | | | PARPEVSCQAVDYENFSCTWSP | | |
| | | | | | GQVSGLPTRYLTSYRKKTLPGAE | | |
| | | | | | SQRESPSTGPWPCPQDPLEASR | | |
| | | | | | CVVHGAEFWSEYRINVTEVNPLG | | |
| | | | | | ASTCLLDVRLQSILRPDPPQGLRV | | |
| | | | | | ESVPGYPRRLHASWTYPASWRR | | |
| | | | | | QPHFLLKFRLQYRPAQHPAWSTV | | |
| | | | | | EPIGLEEVITDAVAGLPHAVRVSA | | |
| | | | | | RDFLDAGTWSAWSPEAWGTPST | | |
| | | | | | GPLQDEIPDWSQGHGQQLEAVV | | |
| | | | | | AQEDSPAPARPSLQPDPRPLDHR | | |
| | | | | | DPLEQVAV | | |
| IL11 | H | P20809 | Uniprot | full | MNCVCRLVLVVLSLWPDTAVAPG | 1-22 is | SEQ |
| | | | | length | PPPGPPRVSPDPRAELDSTVLLT | signal | ID |
| | | | | | RSLLADTRQLAAQLRDKFPADGD | peptide; | NO: |
| | | | | | HNLDSLPTLAMSAGALGALQLPG | 23-199 is | 18 |
| | | | | | VLTRLRADLLSYLRHVQWLRRAG | IL11 | |
| | | | | | GSSLKTLEPELGTLQARLDRLLRR | | |
| | | | | | LQLLMSRLALPQPPPDPPAPPLA | | |
| | | | | | PPSSAWGGIRAAHAILGGLHLTLD | | |
| | | | | | WAVRGLLLLKTRL | | |

### Production of ANDbody Compositions

### Production of ANDbody Polypeptides

Polypeptide components of the macromolecules (ANDbodies) of the invention may be produced by any suitable means. For example, all or part of the ANDbody may be expressed by a host cell comprising one or more nucleotides which encode the ANDbody. Accordingly, in some embodiments, the invention provides one or more nucleic acids encoding any one of the macromolecules provided herein or one or more subunits thereof. The nucleic acids may be, e.g., linear nucleic acids, circular nucleic acids, and/or modified nucleic acids. The invention further provides one or more vectors comprising the one or more nucleic acids encoding any one of the macromolecules provided herein or one or more subunits thereof.

The invention also provides one or more host cells (e.g., mammalian host cells) comprising one or more nucleic acids encoding any one of the macromolecules provided herein or one or more subunits thereof (e.g., comprising vectors comprising said one or more nucleic acids). The invention further provides a method of producing any one of the macromolecules provided herein, the method comprising culturing the one or more host cells in a culture medium. The method may further comprise recovering the macromolecule from the one or more host cells or the culture medium.

Such methods of making a therapeutic polypeptide are routine in the art. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013).

Methods for producing an ANDbody may involve expression in mammalian cells, although recombinant proteins can also be produced using insect cells, yeast, bacteria, or other cells under the control of appropriate promoters. Mammalian expression vectors may comprise nontranscribed elements such as an origin of replication, a suitable promoter and enhancer, and other 5′ or 3 flanking nontranscribed sequences, and 5′ or 3′ nontranslated sequences such as necessary ribosome binding sites, a polyadenylation site, splice donor and acceptor sites, and termination sequences. DNA sequences derived from the SV40 viral genome, for example, SV40 origin, early promoter, enhancer, splice, and polyadenylation sites may be used to provide the other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture an ANDbody described herein. Examples of mammalian expression systems include CHO cells, COS cells, HeLA and BHK cell lines. Processes of host cell culture for production of protein therapeutics are described in, e.g., Zhou and Kantardjieff (Eds.), Mammalian Cell Cultures for Biologics Manufacturing (Advances in Biochemical Engineering/Biotechnology), Springer (2014). Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010). Formulation of protein therapeutics is described in Meyer (Ed.), Therapeutic Protein Drug Products: Practical Approaches to formulation in the Laboratory, Manufacturing, and the Clinic, Woodhead Publishing Series (2012).

Antibody production techniques are known. See, for example, Zhiqiang (Editor), Therapeutic Monoclonal Antibodies: From Bench to Clinic. 1st Edition. Wiley 2009; Greenfield (Ed.) Antibodies: A Laboratory Manual. (Second edition) Cold Spring Harbor Laboratory Press 2013; Ferrara et al. 2012. Using Phage and Yeast Display to Select Hundreds of Monoclonal Antibodies: Application to Antigen 85, a Tuberculosis Biomarker. PLoS ONE 7(11): e49535, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

### Production of ANDbody RNAs

In some embodiments, ANDbodies RNAs may be produced, e.g., for delivery to a subject. Generally, therapeutic mRNAs are made by in vitro transcription. Modification such as incorporation of modified bases, 5′cap analogues, and polyA tails can optimize activity and function. For example, translation and stability of mRNA can be accomplished, by cap and poly A tail modifications. E.g., incorporation of cap analogs such as ARCA (anti-reverse cap analogs) and a poly(A) tail of 100-200 bp into in vitro transcribed (IVT) mRNAs improves expression and stability (Kaczmarek et al. Genome Medicine (2017) 9:60). New types of cap analogs, such as 1,2-dithiodiphosphate-modified caps, can further improve efficiency of translation (Strenkowska et al. Nucleic Acids Res. 2016; 44:9578-90). Codon optimization can also improve efficacy of protein synthesis and limit mRNA destabilization by rare codons (Presnyak et al. Cell. 2015; 160:1111-24. 93; Thess et al. Mol Ther. 2015; 23: 1456-64). Modifying 3′ and 5′ untranslated regions (UTRs), which contain sequences responsible for recruiting RNA-binding proteins (RBPs) and miRNAs, can enhance the level of protein product (Kaczmarek). Further, UTRs can be modified to encode regulatory elements (e.g., K-turn motifs and miRNA binding sites), in order to control RNA expression in a cell-specific manner (Wroblewska et al. Nat Biotechnol. 2015; 33:839-41). RNA base modifications (e.g., pseudouridine incorporated mRNA, e.g., N1-methyl-pseudouridine) contribute to masking mRNA immune-stimulatory activity and increase mRNA translation by enhancing translation initiation (Andries et al. J Control Release. 2015; 217:337-44; Svitkin et al. Nucleic Acids Res. 2017; 45:6023-36). mRNA compositions and methods of their manufacture are known and are disclosed, e.g., in WO2016011306; WO2016014846; WO2016022914; WO2016077123; WO2016164762; WO2016201377; WO2017049275; U.S. Pat. Nos. 9,937,233; 8,710,200; U.S. Ser. No. 10/022,425; U.S. Pat. Nos. 9,878,056; 9,572,897; Jemielity et al. RNA. 2003; 9:1108-22. 90; Mockey et al. Biochem Biophys Res Commun. 2006; 340:1062-8. 91; Strenkowska et al. Nucleic Acids Res. 2016; 44:9578-90. 92; Presnyak et al. Cell. 2015; 160:1111-24. 93; Kaczmarek et al. Genome Medicine (2017) 9:60.

Production of ANDbodies with Altered Affinities

ANDbodies with binding sites with altered affinities can be made using methods known in the art, e.g., an ANDbody can be engineered to have a target binding site that has decreased affinity for the effector target. See, e.g., U.S. Pat. No. 10,654,928. In general, an ANDBody may be modified to alter the affinity of an effector target binding site to its effector target or to alter the affinity of an address target binding site to its address target. The modification can increase or decrease affinity for the binding site's binding partner.

### Assessment of Targets and Addresses

Expression of a therapeutic target can be assessed at either the RNA or protein level using methods known in the art. In embodiments, expression of the therapeutic target is assessed by measuring RNA expression, e.g., using an RNA sequence dataset as a proxy for protein expression levels. RNA datasets include those a genotype-Tissue Expression (GTEx) dataset (see, e.g., https://www.genome.gov/Funded-Programs-Projects/Genotype-Tissue-Expression-Project) or a Human Protein Atlas (HPA) dataset (https://www.proteinatlas.org/).

A non-limiting list of tissues in which expression of the therapeutic target can be assessed includes, e.g., the minor salivary gland, thyroid, lung, breast (mammary tissue), pancreas, adrenal gland, liver, kidney (cortex), kidney (medulla), adipose-viscaral (omentum), small intestine—terminal ileum, fallopian tube, ovary, uterus, skin not sun exposed (suprapubic); cervix-endocervix, cervix-ectocervix, vagina, skin sun exposed (lower leg), cells eneanterior cingulate cortex (BA24), caudate (basal ganglia), putamen (basal ganglia), nucleus acumbens (basal ganglia), hypothalamus, amygdala, hippocampus, cerebellum/cerebellar hemisphere, substantia nigra, pituitary, spinal cord (cervical), artery—aorta, heart—atrial appendage, artery—coronary—heart, left ventricle, esophagus—mucosa, esophagus—muscularis, esophagus—gastroesophageal junction, spleen, stomach, colon—transverse, colon—sigmoid, testis, whole blood, cells—(EBV-transformed lymphocytes, artery—tibial, or nerve—tibial tissues.

Address markers can be assessed using methods well known in the art, e.g., gene expression can be assessed at the mRNA level using Northern blots, cDNA or oligonucleotide microarrays, or sequencing (e.g., RNA-Seq), or at the level of protein expression using protein microarrays, Western blots, flow cytometry, immunohistochemistry, etc. Modifications can be assessed, e.g., using antibodies that are specific for a particular modified form of a protein, e.g., phospho-specific antibodies, or mass spectrometry.

### Uses of ANDbodies

ANDbodies and their pharmaceutical compositions provided herein are suitable for administration to a subject in need thereof, wherein the subject is a human or a non-human animal, for example, suitable for human therapeutic or veterinary use. Accordingly, in some aspects, provided herein is a method comprising administering a therapeutically effective amount of any one of the macromolecules (i.e., ANDbodies) provided herein, a composition comprising the same, or one or more nucleic acids encoding the same to a subject in need thereof.

Veterinary use includes use for treatment of mammals, including commercially relevant mammals, e.g., pet and live-stock animals, such as cattle, pigs, horses, sheep, goats, cats, dogs, mice, and/or rats; and/or birds, including commercially relevant birds such as parrots, poultry, chickens, ducks, geese, hens or roosters and/or turkeys; zoo animals, e.g., a feline; non-mammal animals, e.g., reptiles, fish, amphibians, etc.

The invention is further directed to a subject or subject cell comprising the ANDbody composition described herein. In some embodiments, the subject or subject cell is a plant, insect, bacteria, fungus, vertebrate, mammal (e.g., human), or other organism or cell.

In some embodiments, a subject or a subject cell is contacted with (e.g., delivered to or administered to) the ANDbody composition. In some embodiments, the subject is a mammal, such as a human. The amount of the ANDbody composition, expression product, or both in the subject can be measured at any time after administration.

In some aspects, provided herein is a method of potentiating activation of a signaling pathway in a tissue or cell, the method comprising contacting the tissue or cell with a macromolecule provided herein, wherein neither the first binding site nor the second binding site substantially activates the signaling pathway in the absence of localization by the second binding site; and wherein upon contacting the tissue or cell with the macromolecule, activation of the signaling pathway in the tissue or cell by the first binding site is substantially increased relative to a reference macromolecule lacking the second binding site.

### Pharmaceutical Compositions

### Polypeptide Pharmaceutical Compositions

The ANDbody compositions described herein (e.g., ANDbody polypeptide or RNA compositions) may be administered to a subject in need thereof. The invention includes pharmaceutical compositions that include a macromolecule provided herein (i.e., an ANDbody), e.g., combination in with one or more pharmaceutically acceptable carriers or excipients. In some embodiments, the composition is a unit dosage form suitable for administration to a human subject (e.g., intravenous, oral, or subcutaneous administration).

Formulation of protein therapeutics is routine. See, for example, Ribeiro et al., Insights on the Formulation of Recombinant Proteins. Adv Biochem Eng Biotechnol. 2020; 171:23-54. doi: 10.1007/10_2019_119. PMID: 31844925.

### RNA Pharmaceutical Compositions

Nucleic acids (e.g., RNA) encoding an ANDbody can alternatively or additionally be administered to a subject. Generally, therapeutic mRNAs are made by in vitro transcription. Modification such as incorporation of modified bases, 5′cap analogues, and polyA tails can optimize activity and function. For example, translation and stability of mRNA can be accomplished, by cap and poly A tail modifications. E.g., incorporation of cap analogs such as ARCA (anti-reverse cap analogs) and a poly(A) tail of 100-200 bp into in vitro transcribed (IVT) mRNAs improves expression and stability (Kaczmarek et al. Genome Medicine (2017) 9:60). New types of cap analogs, such as 1,2-dithiodiphosphate-modified caps, can further improve efficiency of translation (Strenkowska et al. Nucleic Acids Res. 2016; 44:9578-90). Codon optimization can also improve efficacy of protein synthesis and limit mRNA destabilization by rare codons (Presnyak et al. Cell. 2015; 160:1111-24. 93; Thess et al. Mol Ther. 2015; 23: 1456-64). Modifying 3′ and 5′ untranslated regions (UTRs), which contain sequences responsible for recruiting RNA-binding proteins (RBPs) and miRNAs, can enhance the level of protein product (Kaczmarek). Further, UTRs can be modified to encode regulatory elements (e.g., K-turn motifs and miRNA binding sites), in order to control RNA expression in a cell-specific manner (Wroblewska et al. Nat Biotechnol. 2015; 33:839-41). RNA base modifications (e.g., pseudouridine incorporated mRNA, e.g., N1-methyl-pseudouridine) contribute to masking mRNA immune-stimulatory activity and increase mRNA translation by enhancing translation initiation (Andries et al. J Control Release. 2015; 217:337-44; Svitkin et al. Nucleic Acids Res. 2017; 45:6023-36). mRNA compositions and methods of their manufacture are known and are disclosed, e.g., in WO2016011306; WO2016014846; WO2016022914; WO2016077123; WO2016164762; WO2016201377; WO2017049275; U.S. Pat. Nos. 9,937,233; 8,710,200; U.S. Ser. No. 10/022,425; U.S. Pat. Nos. 9,878,056; 9,572,897; Jemielity et al. RNA. 2003; 9:1108-22. 90; Mockey et al. Biochem Biophys Res Commun. 2006; 340:1062-8. 91; Strenkowska et al. Nucleic Acids Res. 2016; 44:9578-90. 92; Presnyak et al. Cell. 2015; 160:1111-24. 93; Kaczmarek et al. Genome Medicine (2017) 9:60.

In embodiments, the RNA is a circular RNA. See, for example, WO2019118919, describing the expression of a therapeutic RNA, such as an antibody RNA, from a circular RNA. In some embodiments, the invention includes a circular polyribonucleotide that comprises (a) an internal ribosome entry site (IRES), (b) an expression sequence encoding a ANDbody described herein and lacking a poly-A sequence, and (c) a termination element. A circular RNA encoding an ANDbody described herein may be delivered naked (i.e., without formulation with a carrier) or with a carrier.

### Combination Therapies

In some embodiments, an ANDbody or ANDbody composition provided herein is administered in combination with one or more additional therapeutic agents.

### Carriers

### Lipid Nanoparticles

Formulations of the compositions described herein (e.g., polypeptide or RNA ANDbody compositions) for in vivo delivery with a carrier include lipid nanoparticle (LNP) formulations. See, e.g., U.S. Pat. Nos. 9,764,036; 9,682,139; Kauffman et al. Nano Lett. 2015; 15: 7300-6. 37; Fenton et al. Adv Mater. 2016; 28:2939-43). LNPs, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated herein by reference—e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein includes,

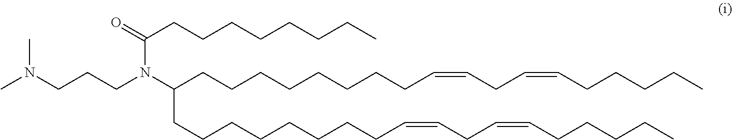 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver an ANDbody RNA composition described herein to the liver and/or hepatocyte cells.

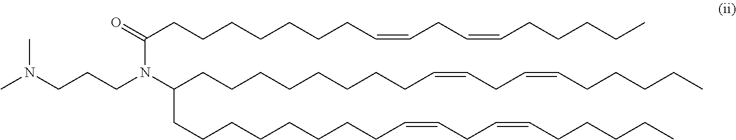 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver an ANDbody RNA composition described herein to the liver and/or hepatocyte cells.

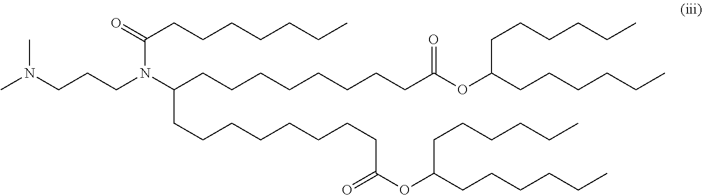 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver an ANDbody RNA composition described herein to the liver and/or hepatocyte cells.

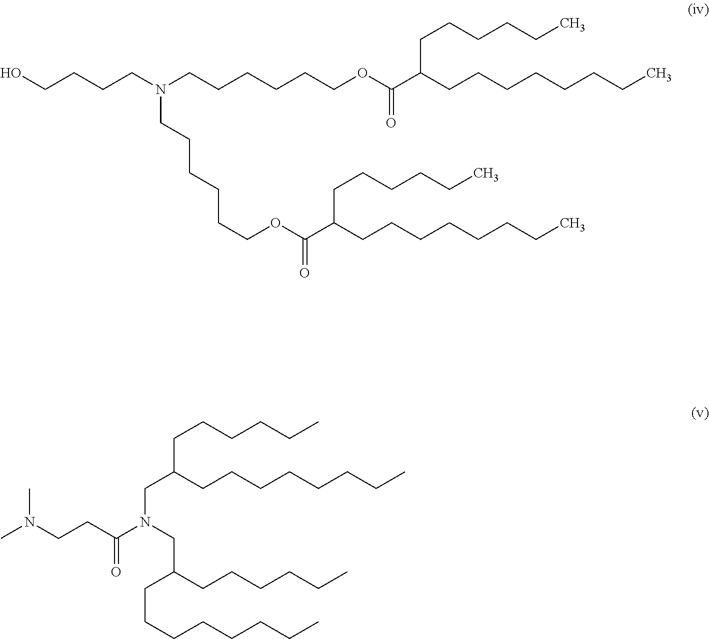

In some embodiments an LNP comprising Formula (v) is used to deliver an ANDbody RNA composition described herein to the liver and/or hepatocyte cells.

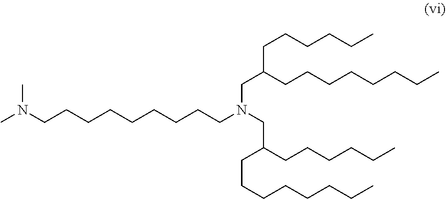 [SMILES: CCCCCCCCC(CCCCCC)CN(CCCCCCCCCN(C)C)CC(CCCCCC)CCCCCCCC]

In some embodiments an LNP comprising Formula (vi) is used to deliver an ANDbody RNA composition described herein to the liver and/or hepatocyte cells.

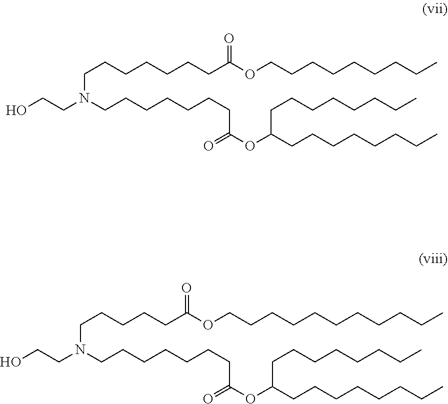 [SMILES: CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

In some embodiments an LNP comprising Formula (viii) is used to deliver an ANDbody RNA composition described herein to the liver and/or hepatocyte cells.

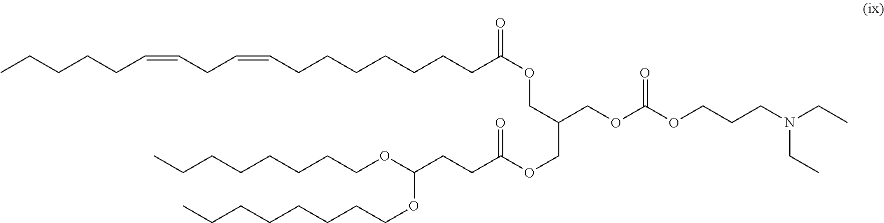 [SMILES: CCCCC/C=C\C/C=C\CCCCCCCC(=O)OCC(COC(=O)CCC(OCCCCCCCC)OCCCCCCCC)COC(=O)OCCCN(CC)CC]

In some embodiments an LNP comprising Formula (ix) is used to deliver an ANDbody RNA composition described herein to the liver and/or hepatocyte cells.

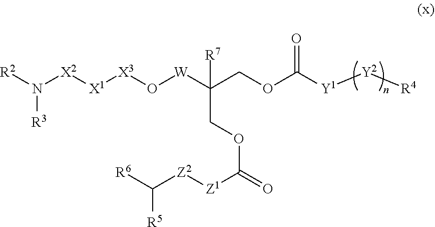

wherein
X1 is O, NR1, or a direct bond, X2 is C2-5 alkylene, X3 is C(═O) or a direct bond, R1 is H or Me, R3 is Ci-3 alkyl, R2 is Ci-3 alkyl, or R2 taken together with the nitrogen atom to which it is attached and 1-3 carbon atoms of X2 form a 4-, 5-, or 6-membered ring, or X1 is NR1, R1 and R2 taken together with the nitrogen atoms to which they are attached form a 5- or 6-membered ring, or R2 taken together with R3 and the nitrogen atom to which they are attached form a 5-, 6-, or 7-membered ring, Y1 is C2-12 alkylene, Y2 is selected from

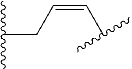 [SMILES: */C=C\C*]

(in either orientation),

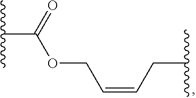 [SMILES: *C/C=C\COC(*)=O]

(in either orientation),

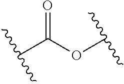 [SMILES: *OC(*)=O]

(in either orientation),
n is 0 to 3, R4 is Ci-15 alkyl, Z1 is Ci-6 alkylene or a direct bond,

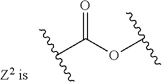 [SMILES: *OC(*)=O]

(in either orientation) or absent, provided that if Z1 is a direct bond, Z2 is absent;
R5 is C5-9 alkyl or C6-10 alkoxy, R6 is C5-9 alkyl or C6-10 alkoxy, W is methylene or a direct bond, and R7 is H or Me, or a salt thereof, provided that if R3 and R2 are C2 alkyls, X1 is O, X2 is linear C3 alkylene, X3 is C(═O), Y1 is linear Ce alkylene, (Y2)n-R4 is

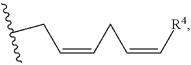

R4 is linear C5 alkyl, Z1 is C2 alkylene, Z2 is absent, W is methylene, and R7 is H, then R5 and R6 are not Cx alkoxy.

In some embodiments an LNP comprising Formula (xii) is used to deliver an ANDbody RNA composition described herein to the liver and/or hepatocyte cells.

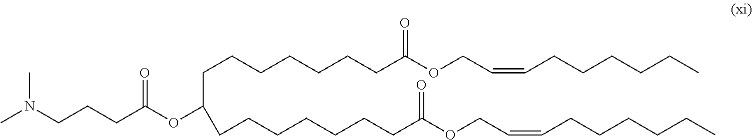 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver an ANDbody RNA composition described herein to the liver and/or hepatocyte cells.

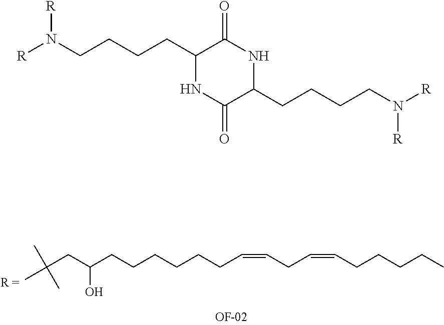

where R=

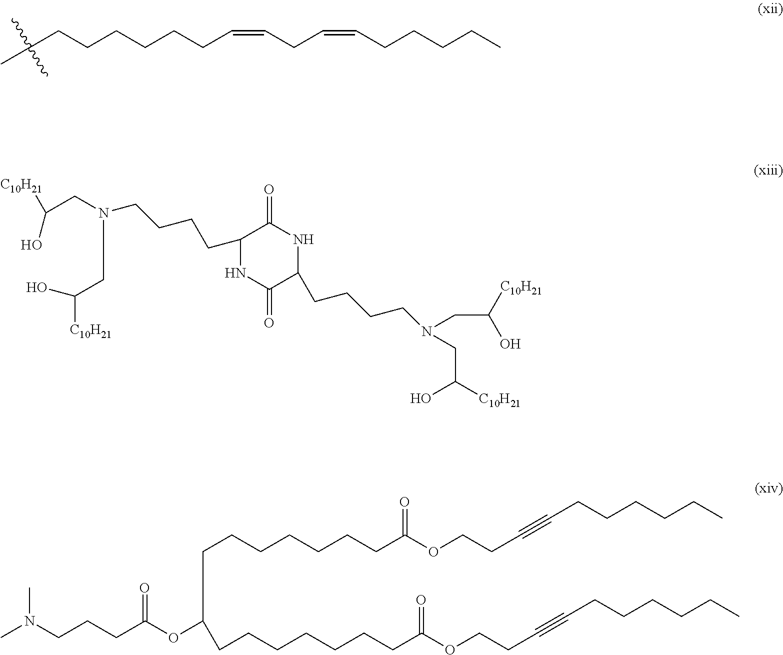

In some embodiments an LNP comprises a compound of Formula (xiii) and a compound of Formula (xiv).

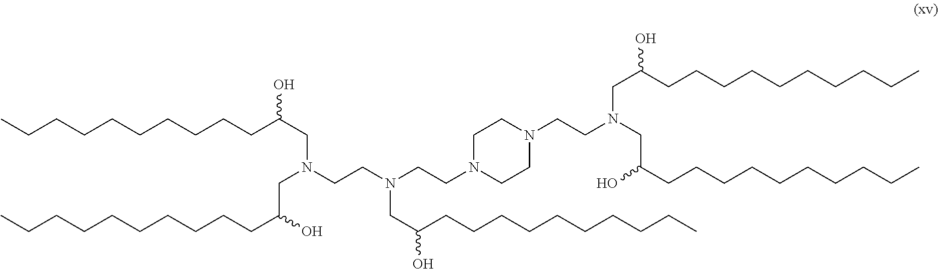 [SMILES: CCCCCCCCCCC(O)CN(CCN1CCN(CCN(CC(O)CCCCCCCCCC)CC(O)CCCCCCCCCC)CC1)CCN(CC(O)CCCCCCCCCC)CC(O)CCCCCCCCCC]

In some embodiments an LNP comprising Formula (xv) is used to deliver an ANDbody RNA composition described herein to the liver and/or hepatocyte cells.

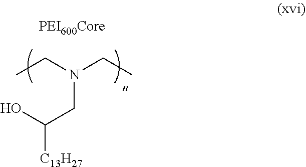

In some embodiments an LNP comprising a formulation of Formula (xvi) is used to deliver an ANDbody RNA composition described herein to the lung endothelial cells.

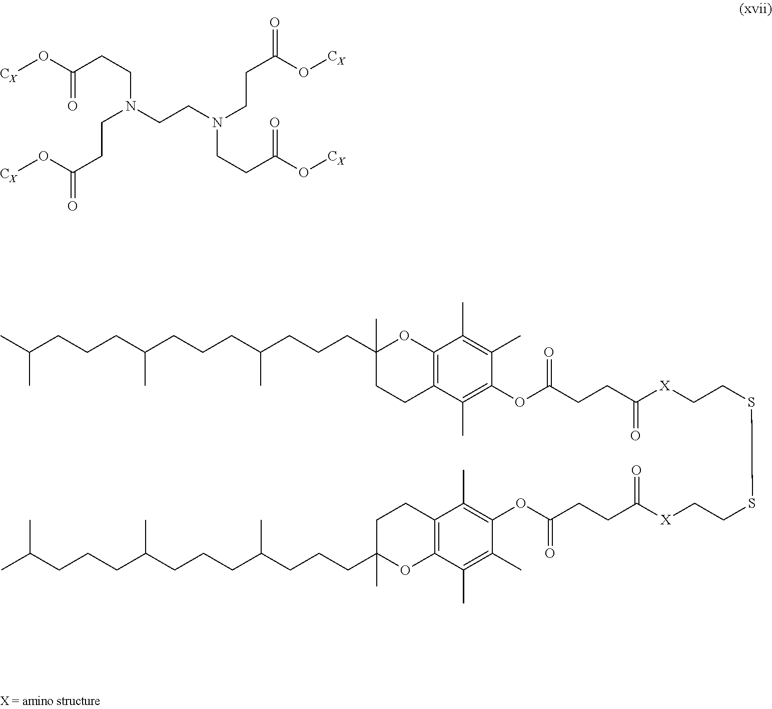

where X=

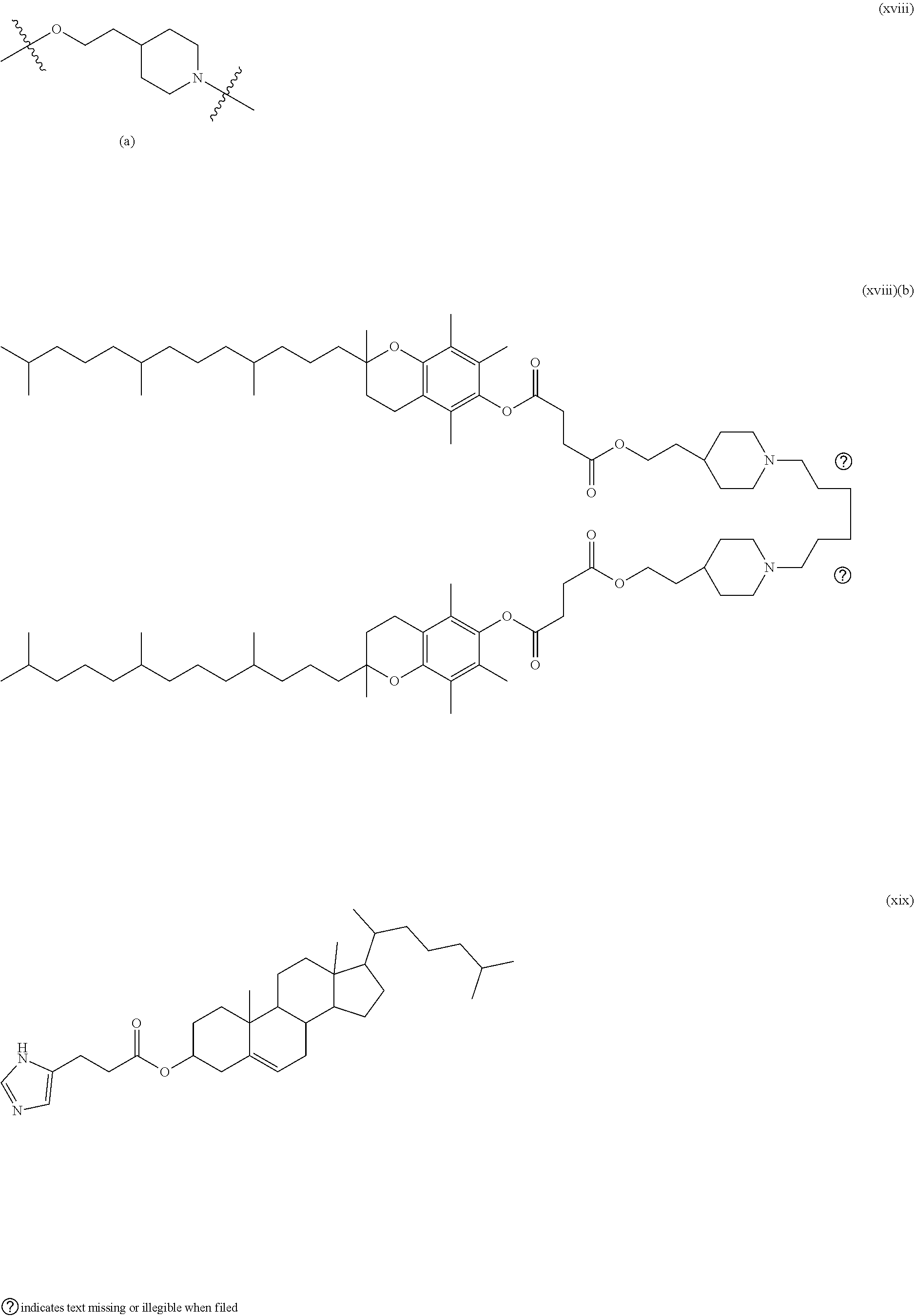 [SMILES: *OCCC1CCN(*)CC1 ; Cc1c(C)c2c(c(C)c1OC(=O)CCC(=O)OCCC1CCN(CCCCCCN3CCC(CCOC(=O)CCC(=O)Oc4c(C)c(C)c5c(c4C)CCC(C)(CCCC(C)CCCC(C)CCCC(C)C)O5)CC3)CC1)CCC(C)(CCCC(C)CCCC(C)CCCC(C)C)O2 ; CC(C)CCCC(C)C1CCC2C3CC=C4CC(OC(=O)CCc5cnc[nH]5)CCC4(C)C3CCC12C]

In some embodiments, a lipid compound used to form lipid nanoparticles for the delivery of compositions described herein, e.g., nucleic acid (e.g., RNA) described herein is made by one of the following reactions:

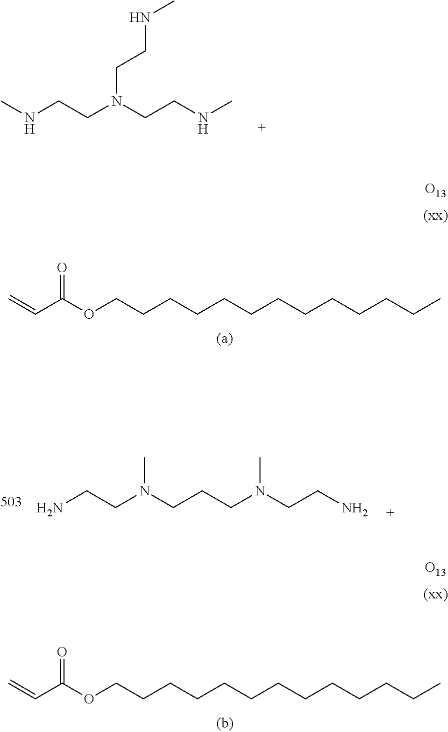

In some embodiments, a composition described herein (e.g., a nucleic acid or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of U.S. Pat. No. 9,867,888 (incorporated by reference herein in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LPO1), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-IH-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyne lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein, encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; 1, 11, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; I-5 or I-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 IZ)-heptatriaconta-6,9,28,3 I-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, I-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle. In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, choiesteryl-(2,-hydroxy)-ethyl ether, choiesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., choiesteryl-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as I-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (I-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

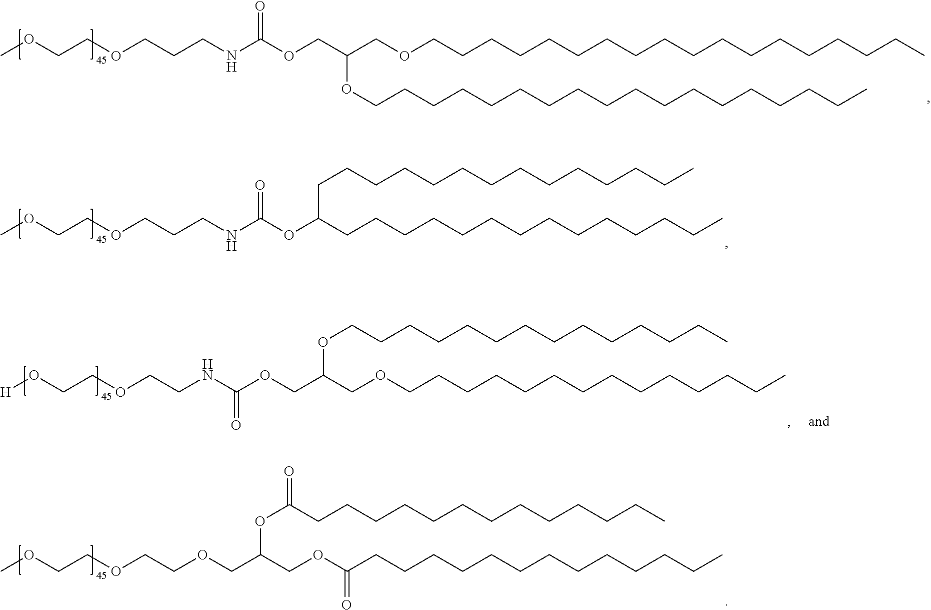

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately, or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

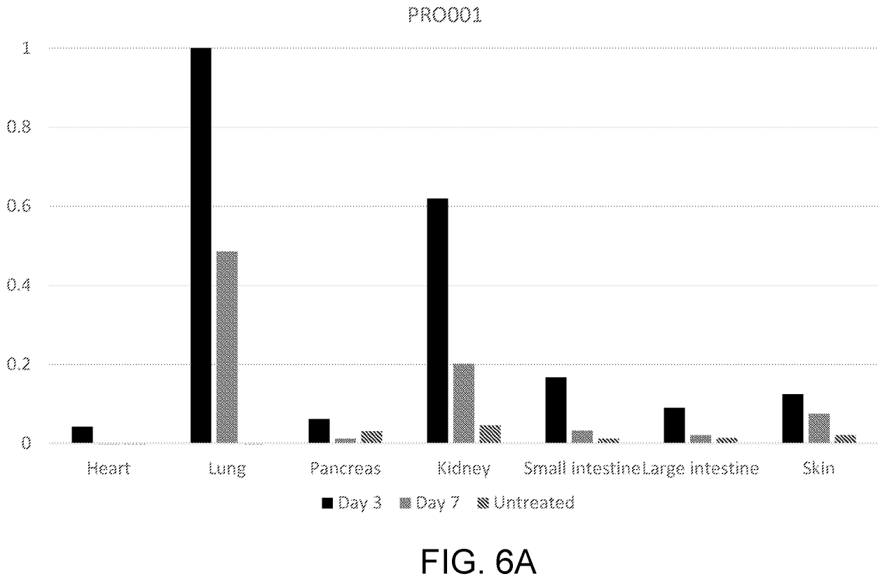

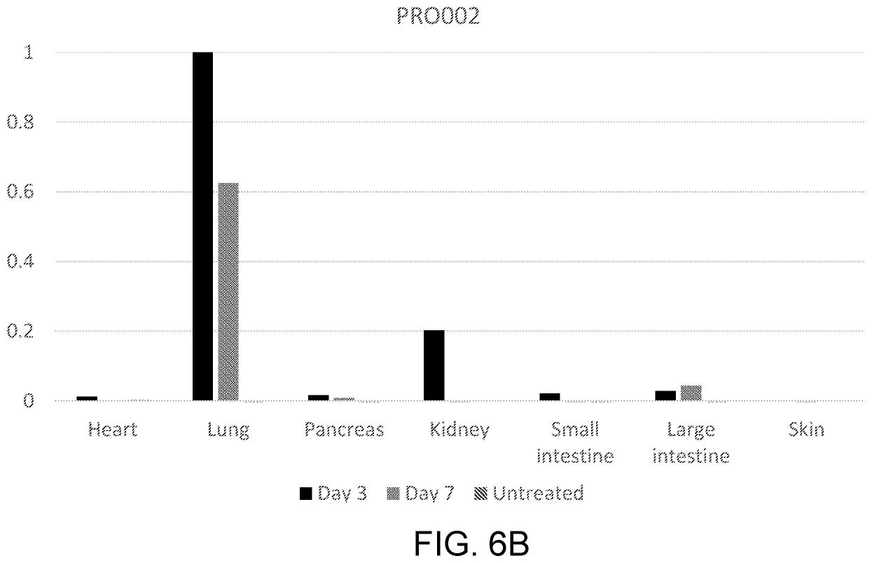

In some embodiments, LNPs are directed to specific tissues by the addition of LNP targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about I mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of an LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of LNPs comprising the RNA compositions described herein may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 1011, 1012, 1013, and 1014 vg/kg.

In some embodiments, the invention includes a lipid nanoparticle (LNP) comprising the ANDbody polypeptide (or RNA encoding the same), nucleic acid molecule, or DNA encoding an ANDbody described herein. In embodiments, the LNP comprises a cationic lipid. In some embodiments, the LNP further comprises one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate. In some embodiments, the cationic lipid of the LNP has a structure according to:

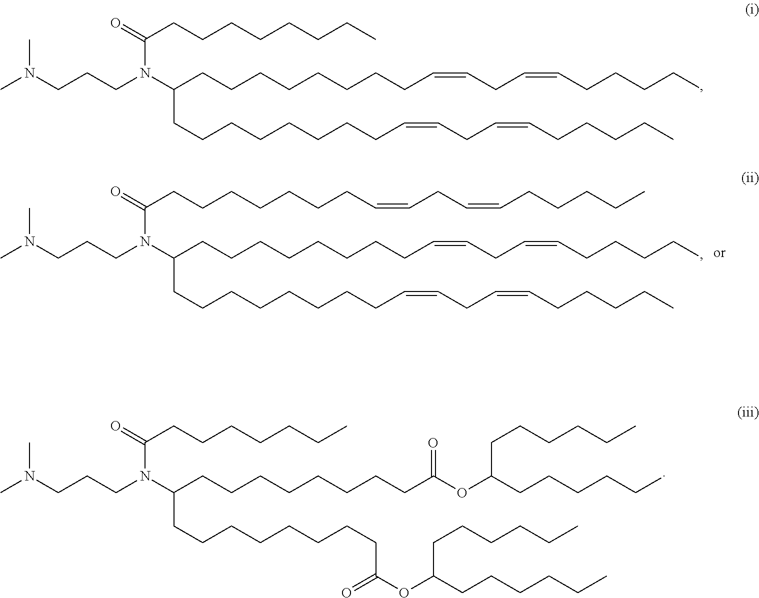

For a review of LNP, see also, e.g., Li et al. 2017, Nanomaterials 7, 122; doi:10.3390/nano7060122.

### Other Carriers

### Viral Vectors

The compositions described herein (e.g., polypeptide or RNA ANDbody compositions), can be delivered by a viral vector (e.g., a viral vector expressing an RNA). A viral vector may be administered to a cell or to a subject (e.g., a human subject or non-human animal). A viral vector may be locally or systemically administered.

Examples of viral vectors include a retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, lentivirus, alpharetrovirus, gammaretrovirus, spumavirus (Coffin, J. M., Retroviridae: The viruses and their replication, Virology (Third Edition) Lippincott-Raven, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030, the teachings of which are incorporated herein by reference.

Anellovirus vectors can also be used for delivering an ANDbody composition described herein. Anellovectors are known in the art and described, e.g., in WO2020123773, WO2020123816, WO2018232017, and WO2020123773. In certain embodiments, an anellovector composition comprises a genomic element that comprises a promoter operably linked to a nucleic acid sequence encoding an ANDbody described herein, the genetic element encapsulated by a proteinaceous exterior comprising an Anellovirus ORF1, e.g., an anellovirus capsid protein.

### Cell and Vesicle-Based Carriers

A composition described herein (e.g., polypeptide or RNA ANDbody compositions), described herein can be administered to a cell in a cell, vesicle or other membrane-based carrier. In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review). Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for an agent (e.g., an inhibitor) described herein, e.g., an antibody or a nucleic acid described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the [agent] or preparation described herein.

Plant nanovesicles and plant messenger packs (PMPs), e.g., as described in WO2011097480, WO2013070324, WO2017004526, or WO2020041784 can also be used as carriers to deliver the compositions described herein.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications and sections thereof cited herein are herein incorporated by reference for the purposes or subject matter referenced herein.

### EXAMPLES

The invention will be further illustrated in the following non-limiting examples.

TABLE OF CONTENTS

| | |
|---|---|
| Example 1 | ANDBODY BINDING MOUSE AND HUMAN RAGE |
| | AND NOTCH2 |
| Example 2 | ANDBODY BINDING MOUSE AND HUMAN UMOD |
| | AND NOTCH2 |
| Example 3 | ANDBODY BINDING MOUSE AND HUMAN MEP1B |
| | AND NOTCH2 |
| Example 4 | ANDBODY BINDING MOUSE AND HUMAN RAGE |
| | AND IL11RA |
| Example 5 | ANDBODY BINDING MOUSE AND HUMAN UMOD |
| | AND IL11RA |
| Example 6 | ANDBODY BINDING MOUSE AND HUMAN MEP1B |
| | AND IL11RA |
| Example 7 | EXEMPLARY ADDRESS-RESTRICTED BINDER |
| | FOR SKIN |
| Example 8 | EXEMPLARY ADDRESS-RESTRICTED BINDER |
| | FOR LUNG |
| Example 9 | EXEMPLARY ADDRESS-RESTRICTED BINDER |
| | FOR KIDNEY |
| Example 10 | EXEMPLARY ADDRESS-RESTRICTED BINDER |
| | FOR INTESTINE |
| Example 11 | TISSUE RESTRICTION OF PREDICTED |
| | ADDRESSES |
| Example 12 | ANDBODY PRODUCTION AND USE |
| Example 13 | ANDBODY PRODUCTION AND USE |
| Example 14 | TNFA-BLOCKING MOLECULES COUPLED TO |
| | DSG1-TARGETING MOIETIES |

### Example 1. ANDbody Binding Mouse and Human Rage and Notch2

### 1.1 Vaccination to Create Anti-RAGE Antibodies

Antibodies against human RAGE extracellular domain, an exemplary address target of the present technology, are created by immunization. The extracellular domain of human RAGE (NCBI protein accession Q15109 positions N24-A344) (huRAGE) fused to the Fc region of human IgG1 (UniProt ID P01857 positions P100-K330) is expressed in HEK293F cells. Briefly, DNA sequences are codon optimized for mammalian expression and ordered in the pcDNA3.4-TOPO expression vector (ThermoFisher Scientific). Proteins are transiently transfected into HEK293 cells and purified using rProtein A Sepharose Fast Flow resin according to manufacturer's instructions (GE Healthcare) similar to prior methods (Rothschilds et al. 2019). 50 μg of the huRAGE-Fc fusion protein is used to immunize female BALB/c mice by i.p. injection in CFA/IFA (Millipore Sigma, catalog #F5881-10ML and F5506-10ML) adjuvant. Subsequently, hybridomas are generated (Listek et al. 2020). Clones are initially screened for IgG reactivity specific for the huRAGE-Fc fusion protein used for immunization in an ELISA format followed by flow cytometry studies using cells stably (CHO) or transiently (HEK293F) transfected with full-length huRAGE. Anti-RAGE hybridoma clones are next evaluated based on murine cross-reactivity. Flow cytometry studies are done using cells stably (CHO) or transiently (HEK293F) transfected with full-length mouse RAGE (mRAGE), and clones are selected that bind to mRAGE. Positive clones expressing anti-RAGE mAbs cross-reactive between human and mouse are then further purified by limited dilution cloning. The hybridomas are grown in DMEM/2% ultra low IgG serum and the mAbs are purified by protein G chromatography according to manufacturer's instructions using (Millipore Sigma, P3296-1 ML).

### 1.2 Selecting for Inert Anti-RAGE Antibodies

Address target binding sites of the present technology are designed to not influence signaling upon binding the address target, such as the exemplary RAGE address target. Accordingly, anti-RAGE hybridoma clones produces as described above are further evaluated based on their inability to block RAGE ligand binding. Human RAGE ligands tested included HMGB1 (full-length, from Creative BioMart catalog #HMGB1-29332TH), Advanced Glycation Endproduct (fused to bovine serum albumin, Millipore Sigma catalog #121800-10MG-M), S100A12 (full-length, from R&D Systems catalog #1052-ER-050), S100A1 (full-length, from R&D Systems catalog #9705-S1-100), S100A4 (R&D Systems catalog #4137-S4-050), S100A10 (full-length, from Creative BioMart catalog #S100A10-157H), S100A11 (R&D Systems catalog #9015-S11-050), S100A13 (R&D Systems catalog #4327-SA-050), S100B (R&D Systems catalog #1820-SB-050), amyloid-p-peptide (DAEFRHDSGYEVHHQKLVFFAEDVGSNKGAIIGLMVGGVVIA from NM_000484.2, Millipore Sigma catalog #AG912-1 MG) and Mac-1 (F17-N1105 from NP_001139280 and Q23-N700 from UniProt P05107, R&D Systems catalog #4047-AM-050). ELISAs are used to quantify ability of ligands to bind in the presence of anti-RAGE antibodies. HuRAGE-Fc is adsorbed to ELISA plates, then after blocking, the plates are incubated with concentrations of 10 fM up to 10 uM of anti-RAGE antibody clones from the hybridomas (one condition per concentration and per clone). After washing the plates, ligands are each biotinylated according to manufacturer's instructions (ThermoFisher catalog #21435), and then incubated on the plates at concentrations ranging from 10 fM up to 10 uM. After washing, SA-linked HRP secondary antibodies are added, followed by TMB substrate and colorimetric readout quantified by absorbance. Ligand binding is compared within a given ligand with versus without anti-RAGE antibodies to isolate anti-RAGE clones that are inert and do not affect binding of one or more ligands.

The inability of anti-RAGE antibodies to inhibit IFNα-induced gene signature is also evaluated, and anti-RAGE hybridoma clones are chosen that do not change cellular signaling based on an IFNα-induced gene signature assay. PBMCs from healthy human donors are stimulated for 4 h with 50% sera from SLE patients. The assay is completed either in the presence of anti-RAGE antibodies or unrelated (negative) isotype control human IgG1 antibodies (Bio X Cell catalog #BE0297) at antibody concentrations ranging from 10 fM up to 10 uM. In addition a huRAGE-Fc fusion molecule is used as a positive control. Total RNA is purified and expression of type I IFN-inducible genes, including DDX58, G1P2, MXI, OAS3, RSAD2, IFITI, IFI35 are measured by real-time qRT-PCR analysis as in prior methods (WO 2008/137552 A2, https://patentimages.storage.googleapis.com/94/26/c8/7b9f27f693c4b6/WO2008137552A2.pdf). The inhibition values of gene expression are normalized to the negative control Ab.

### 1.3 Vaccination to Create Anti-Notch2 Antibodies

Antibodies against the extracellular domain of human Notch2 (huNotch2) fused to the Fc region of human IgG1, an exemplary effector target of the present technology, are created by immunization similar to RAGE as described above. After immunization and hybridoma generation, clones are screened exactly as above but for binding to full length human and mouse Notch2 (instead of RAGE). Positive clones expressing anti-Notch2 mAbs cross-reactive between human and mouse are then further purified by limited dilution cloning. The hybridomas are grown in DMEM/2% ultra low IgG serum and the mAbs are purified by protein G chromatography.

### 1.4 Selecting for Active Anti-Notch2 Antibodies at Wide IC₅₀ Ranges

Effector target binding sites of the present technology, such as Notch2, are designed to not influence signaling upon binding the effector target, unless they are localized to a target tissue by an address target binding site, such as RAGE. Accordingly, the binding affinities of effector target (e.g., Notch2) binding sites are analyzed. Specifically, the IC₅₀s of Notch2 antibodies on the ligand human Jagged-2-Fc fusion protein (Creative BioMart, JAG2-382H) binding to surface Notch2 are evaluated using flow cytometry to choose antibodies at IC₅₀'s ranging from less than 1 nM up to 5 uM. Jagged-2-Fc is labeled with alexa fluor 647 (AF647) according to manufacturer's instructions (ThermoFisher, A20186) and methods previously described (Tzeng et al. 2015).

HEK293F cells are transiently transfected with full-length huNotch2. The cells are incubated with anti-Notch2 antibodies at concentrations increasing from 1 μM up to 50 uM. Subsequently (and without washing the cells), the cells are then incubated for 1 hour at 4 degrees Celsius with a constant concentration of AF647 labeled Jagged-2-Fc, ranging (for different IC₅₀ assays) from 1 μM up to 50 uM. For each IC₅₀ assay, one constant concentration of AF647 Jagged-2-Fc is chosen with varied anti-Notch2. Binding of AF647 Jagged-2-Fc with increasing anti-Notch2 antibody concentrations is quantified on the cells by flow cytometry using a ThermoFisher Attune NxT (B2R3Y3V6).

### 1.5 Expressing and Purifying ANDbodies as Bispecifics

DNA sequences from 10 RAGE antibodies and 10 Notch2 antibodies ranging in IC₅₀ (from <1 nM up to 5 uM) are cloned using In-Fusion HD Cloning (Takara Bio, catalog #638911) into human IgG1 framework with single matching point mutations in the CH3 domain Fc region according to the ‘Controlled Fab-Arm Exchange’ (cFAE) method (Labrijn et al. 2014). After separately expressing antibodies from transient HEK293 expressions and purifying each antibody using protein A affinity resin, parental antibodies (combinations of 1 RAGE antibody with 1 Notch2 antibody) are made into bispecific RAGE/Notch2 ANDbodies according to the cFAE method. Briefly, parental antibodies are mixed under permissive redox conditions to enable recombination of half-molecules. Subsequently, the reductant is removed to allow for reoxidation of interchain disulfide bonds. Lastly, exchange efficiency is quantified using chromatography-based or mass spectrometry-based methods. Around 100 variant ANDbodies of RAGExNotch2 are made.

### 1.6 Affinity of ANDbody Variants

To identify ANDbody™ variants that meet desired effector target and address target binding affinity criteria, SPR-based affinity measurements are carried out on BIAcore model 2000 or T100 (Biacore/GE Healthcare, Piscataway, NJ) at 25° C. using HBS-EP+ buffer (Cytiva catalog #BR100669) with 0.1 mg/ml BSA (Millipore Sigma catalog #A9418) as a running buffer. A Sensor Chip Protein A (Cytiva catalog #29127557) is used to capture mouse RAGE-Fc, human RAGE-Fc, mouse Notch2-Fc, or human Notch2-Fc. ANDbody is injected in a 3-fold dilution series from 60 to 0.74 nM, and dissociation is monitored for 10 min for all proteins. Kinetic analysis is done by simultaneously fitting the association and dissociation phases of the sensorgram using the 1:1 Langmuir binding model in BIAevaluation software (Biacore) as supplied by the manufacturer. Double referencing is applied in each analysis to eliminate background responses from the reference surface and buffer only control.

This assay can quantitatively assess the affinities of every ANDbody variant for RAGE and Notch2. ANDbody variants with higher affinity for RAGE than Notch2 as well as variants with no affinity differences or higher affinity for Notch2 are used in subsequent in vitro and in vivo experiments.

1.7 In Vitro Assays for Notch2 Antagonism on Cells with or without RAGE Expression

To analyze ANDbody characteristics, in vitro, HEK293F cells are transiently transfected with full length huRAGE (R+) or full length huNotch2 (N+), or co-transfected with both (RN+). The many variant RAGExNotch2 ANDbodies are fluorophore-labeled with alexa fluor 647 (AF647) according to manufacturer's instructions (ThermoFisher, A20186) and methods previously described (Tzeng et al. 2015). Subsequently, ANDbodies are incubated with R+, N+, RN+, or combined R+ plus N+ cells at ANDbody concentrations ranging from 10 fM up to 10 uM. The parental anti-Notch2 mono-specific antibodies (one from each variant) are fluorophore labeled with FITC according to manufacturer's instructions (ThermoFisher, 53027). In some conditions, the parental anti-Notch2 FITC labeled antibodies are incubated with the cells pre-bound with AF647 RAGExNotch2 (matching Notch2 variants) at concentrations of 10 fM up to 10 uM to saturate the remaining binding sites for Notch2. The binding EC₅₀s of both the AF647 labeled RAGExNotch2 ANDbody variants and the parental FITC labeled parental anti-Notch2 antibodies are quantified using flow cytometry. When the FITC Notch2 antibody is added after the AF647 RAGExNotch2, the FITC signal from those cells is subtracted from the FITC signal from FITC Notch2 alone on cells (then normalized to the Notch2 alone signal) to quantify the % Notch2 bound by RAGExNotch2. These numbers at different concentrations of the RAGExNotch2 ANDbody are used to create EC₅₀ curves. The assay is also run replacing the AF647-labeled ANDbody with AF647-labeled anti-Notch2 antibody.

A difference in observed EC₅₀s on N+ cells vs RN+ cells reveals an enhanced Notch2 blockade when RAGE is present and identifies cells expressing the RAGExNotch2 ANDbodies.

### 1.8 Biodistribution (In Vivo) for ANDbody and Parental Antibodies

To analyze ANDbody distribution, in vivo, the biodistribution of the RAGExNotch2 ANDbody as well as each of the parental antibodies (anti-Notch2 or anti-RAGE used for the cFAE of the ANDbody) is quantified in female Balb/c and C57BL/6 mice.

To quantify the cellular biodistribution, the proteins (ANDbody and antibodies) are first individually labeled with AF647 according to manufacturer's instructions (ThermoFisher, A20186) and methods previously described (Tzeng et al. 2015). Then, each labeled antibody is injected individually at doses of 10 ug, 100 ug, and 500 ug IV (tail vein). Saline (PBS) is also injected as a control at equal volume.

For cellular biodistribution, at time points of 12 hours, 1 day, 2 days, 3 days, 7 days, and 14 days after injection, mice are euthanized using CO2 and tissues including heart, lung, spleen, blood, kidney, liver, and intestines are processed into single cell suspensions according to methods previously described (Tzeng et al. 2015). Briefly, blood is collected by cardiac puncture into EDTA-treated tubes (BD catalog #365974), and other tissues are harvested, weighed, mechanically dissociated between frosted glass slides, and rendered into single-cell suspensions by filtration through 70-μm mesh screens (Millipore Sigma, catalog #CLS431751-50EA). Splenocytes, whole blood, and lung are treated with ammonium-chloride-potassium (ACK) lysing buffer (Thermofisher Scientific, catalog #A1049201). Heart is digested with collagenase and processed into single cell suspension according to previous methods (Covarrubias et al. 2019). Flow cytometry is performed on immune cells using markers for CD8 T cells (CD3e+ CD8+), CD4 T cells (CD3e+ CD4+ Foxp3−), regulatory T cells (CD4+ CD25+ FOXP3+), monocytes/macrophages (CD3e− CD11b+ CD11c−/lo NK1.1− Ly6G− SSClo), dendritic cells (CD3e− CD11chi), NK cells (NK1.1+ CD3e−), and NKT cells (NK1.1+ CD3e+) as previously described (Tzeng et al. 2015). Lung cells including epithelial (CD326+ CD31−CD45−), endothelial (CD326−CD31+CD45−), and hematopoietic lineages (CD326−CD31−CD45+) are also analyzed as previously defined (Singer et al. 2016). Antibodies are purchased from Biolegend, and flow cytometry is run on a ThermoFisher Attune NxT (B2R3Y3V6). Presence of the labeled ANDbody or other labeled antibody on the cell surface is defined by fluorescence of AF647 (and this fluorophore was avoided in the flow panels).

To quantify the tissue biodistribution, the proteins (ANDbody and antibodies) are first individually labeled with NHS-5/6-FAM (Thermofisher Scientific, catalog #46409) as per the manufacturer's instructions.

For tissue biodistribution, at time points of 12 hours, 1 day, 2 days, 3 days, 7 days, and 14 days after injection, mice are euthanized using CO2 and tissues including lung, spleen, blood, kidney, liver, and intestines are harvested, weighed, and imaged on an IVIS Spectrum imaging system (Caliper Life Sciences; excitation, 500 nm; emission, 540 nm). Images are analyzed using the Living Image software.

### 1.9 In Vivo Bioactivity Quantifying Gene Expression Changes

To analyze ANDbody activity, in vivo, bioactivity is quantified using female Balb/c and C57BL/6 mice. To quantify bioactivity across tissues, the RAGExNotch2 ANDbody or each of the respective parental antibodies (anti-Notch2 or anti-RAGE used for the cFAE of the ANDbody) is injected at doses of 10 ug, 100 ug, and 500 ug IV (tail vein). Saline (PBS) is also injected as a control at equal volume.

At time points of 12 hours, 1 day, 2 days, 3 days, 7 days, and 14 days after injection, tissues including lung, spleen, blood, kidney, liver, heart, and intestines are processed into single cell suspensions according to methods previously described (Tzeng et al. 2015). Briefly, blood is collected by cardiac puncture into EDTA-treated tubes (BD catalog #365974), and other tissues are harvested, weighed, mechanically dissociated between frosted glass slides, and rendered into single-cell suspensions by filtration through 70-μm mesh screens (Millipore Sigma, catalog #CLS431751-50EA). Splenocytes and whole blood are treated with ammonium-chloride-potassium (ACK) lysing buffer (Thermofisher Scientific, catalog #A1049201).

qRT-PCR is performed using methods previously described (Nandagopal et al. 2018). RNA is prepared using the RNeasy kit (QIAGEN). cDNA is prepared from 500 ng RNA using the iScript cDNA synthesis kit (Bio-Rad). 0.5 μL cDNA is used per 10 μL RT-qPCR reaction mix containing 1×iqSYBR Green Supermix (Bio-Rad) and 450 nM total forward and reverse primers. Reactions are performed on a BioRad CFX Real-Time PCR Detection System using a 2-step amplification protocol, with the following thermocycling parameters: 95 C, 3 min followed by 40 cycles of 95 C, 10 s (melting) and 55 C, 30 s (annealing+extension). All reactions are performed in duplicate.

Genes related to Notch2 signaling are mouse Hes1, Hey1, and HeyL, and the reference gene is SdhA. Primers used for amplification are the mouse Hes1 primer set (Forward, 5′-CAACACGACACCGGACAAAC-3′ and Reverse, 5′-AAGAATAAATGAAAGTCTAAGCCAA-3′), mouse Hey1 primer set (Forward, 5′-GCCGAAGTTG CCCGTTATCT-3′ and Reverse, 5′-CGCTGGGATG CGTAGTTGTT-3′), mouse HeyL primer set (Forward, 5′-GAGCTGAC TTCCCACAACCA-3′ and Reverse, 5′-GAGAGG TGCCTTTGCGTAGA-3′), and mouse SdhA primer set (Forward, 5′-AGTGGGCT GTCTTCCTTAAC-3′ and Reverse, 5′-GGATTGCTTCT GTTTGCTTGG-3′) previously described (Nandagopal et al. 2018). All primers are purchased from IDT DNA.

Hes1, Hey1, and HeyL gene expression is measured in the ANDbody treated mice, untreated mice, and anti-Notch2 treated mice, including in the lungs.

### 1.10 In Vivo Bioactivity Using Weights and Histology

Using female Balb/c and C57BL/6 mice, histology is done on organs such as the spleen, kidney, liver, heart, intestines, teeth, and lungs to compare pathology with ANDbody treatment, with treatment with anti-Notch2 alone, or saline (PBS). Starting at 8 weeks of age, 10 ug, 100 ug, and 500 ug of the ANDbody or of the corresponding Notch2 antibody (prior to cFAE) is injected IV (tail vein) 1× or 2× per week. Saline (PBS) is also injected as a control at equal volume 1× or 2× per week. Mice are weighed 2× per week starting prior to the first treatment. After 2 weeks, 4 weeks, and 6 weeks of treatment, mice are euthanized and organs are processed for histology.

Unless otherwise noted, organs are removed into cassettes and then placed directly into 10% neutral-buffered formalin (Sigma-Aldrich) for 12-24 hours prior to embedding in paraffin. Lungs are perfused with 10% neutral-buffered formalin prior to being placed in cassettes for soaking in neutral-buffered formalin. Intestines are thoroughly rinsed before being put in cassettes for soaking in 10% neutral-buffered formalin. Paraffin sections (1-2 μm) are cut and de-waxed prior to histochemical staining. Sections are stained with hematoxylin/eosin (H&E; Merck, Darmstadt, Germany) and scored blindly according to immune infiltrates and tissue morphology.

In addition to lung morphology, weight loss (or not) of ANDbody treated mice over the course of treatment is compared with weight loss (or not) of mice treated with anti-Notch2, and weight loss (or not) of untreated mice.

### Example 2. ANDbody Binding Mouse and Human UMOD and Notch2

### 2.1 Yeast Surface Display to Create Anti-UMOD Antibodies

Yeast surface display (Chao et al. 2006) is used to engineer antibodies to mouse UMOD (Creative BioMart, catalog #UMOD-17835M, untagged), an exemplary address target of the present technology. This is done by using methods described previously (Angelini et al. 2015) and summarized below. The yeast display starts with a synthetic antibody library from the Sidhu laboratory that is based off of natural frameworks, library ‘G’ (Van Deventer et al. 2015). scFvs displayed on the yeast surface are selected for binding to mouse UMOD. Subsequent sorts can be done against the human UMOD antigen (Creative BioMart, catalog #UMOD-001H, untagged), such that binders can be cross-reactive between human and mouse forms. To increase affinity of the scFv binders, affinity maturation is performed using error-prone PCR as described previously (Angelini et al. 2015) and the resulting library is re-sorted for binding to both mouse and human UMOD. Subsequent to engineering, many scFvs that are multi-species cross-reactive are cloned back into human IgG1 antibody format.

### 2.2 Selecting for Inert Anti-UMOD Antibodies

Address target binding sites of the present technology are designed to not influence signaling upon binding the address target, such as the exemplary UMOD address target. Accordingly, anti-UMOD antibodies are further evaluated based on their inability to block UMOD ligand binding, in an assay such as the one described above for RAGE antibodies or an in vivo assay. Inert UMOD antibodies may be identified in such assays whereby kidney architecture is not affected or modified by the screened UMOD antibody.

### 2.3 Vaccination to Create Anti-Notch2 Antibodies

Antibodies cross-reactive to mouse and human Notch2 are created, cloned, and expressed into the human IgG1 framework according to prior methods as described above.

2.4 Selecting for active Notch2 antibodies at wide IC₅₀ ranges

Antibodies against Notch2 will be selected at wide IC₅₀ ranges as described above.

### 2.5 Expressing and Purifying ANDbodies as Bispecifics

DNA sequences from 10 UMOD antibodies and 10 Notch2 antibodies ranging in IC₅₀ (from <1 nM up to 5 uM) are made into about 100 variant ANDbodies as described above.

### 2.6 Affinity of ANDbody Variants for UMOD and Notch2

ANDbody affinities for UMOD and Notch2 are evaluated similarly to above using a BIAcore (as described above). In this case, human and mouse versions of His-tagged UMOD are immobilized on a Sensor Chip NTA (Cytiva catalog #BR100034). Human and mouse notch2-Fc are immobilized as described above.

### 2.7 Biodistribution (In Vivo) for ANDbody and Parental Antibodies

Cellular and tissue biodistribution studies are done using methods described above (as described above). However, the ANDbody used in this case is UMODxNotch2, and the parental antibodies correspond to anti-UMOD and anti-Notch2.

### 2.8 In Vivo Bioactivity Quantifying Gene Expression Changes

The in vivo bioactivity of UMODxNotch2 ANDbodies is quantified using gene expression methods described above.

### 2.9 In Vivo Bioactivity Using Weights and Histology

The in vivo bioactivity of UMODxNotch2 ANDbodies is quantified using weight and histology methods described above.

### Example 3. ANDbody Binding Mouse and Human MEP1B and Notch2

### 3.1 Yeast Surface Display to Create Anti-MEP1B Antibodies

Yeast surface display (Chao et al. 2006) is used to engineer antibodies to mouse MEP1B (Cusabio, CSB-MP730755MO), an exemplary address target of the present technology. Yeast display is performed as described above (2.1) to get cross-reactive mouse/human MEP1B binders (human MEP1B, Cusabio, CSB-MP618098HU). Subsequent to engineering, many scFv's cross-reactive to mouse and human MEP1B are cloned into human IgG1, transiently transfected into HEK293F cells, and purified using protein A resin as described above.

### 3.2 Vaccination to Create Anti-Notch2 Antibodies

Antibodies cross-reactive to mouse and human Notch2 are created, cloned, and expressed into the human IgG1 framework according to prior methods described above.

### 3.3 Selecting for Active Notch2 Antibodies at Wide IC₅₀ Ranges

Antibodies against Notch2 are selected at wide IC₅₀ ranges as described above.

### 3.4 Expressing and Purifying ANDbodies as Bispecifics

DNA sequences from 10 MEP1B antibodies and 10 Notch2 antibodies ranging in IC₅₀ (from <1 nM up to 5 uM) are made into about 100 variant ANDbodies as described above.

### 3.5 Affinity of ANDbody Variants for MEP1B and Notch2

ANDbody affinities for MEP1B and Notch2 are evaluated similarly to above using a BIAcore. In this case, human and mouse versions of His-tagged MEP1B are immobilized on a Sensor Chip NTA (Cytiva catalog #BR100034). Human and mouse notch2-Fc are immobilized as described above.

### 3.6 Biodistribution (In Vivo) for ANDbody and Parental Antibodies

Cellular and tissue biodistribution studies are done using methods described above. However, the ANDbody used in this case is MEP1BxNotch2, and the parental antibodies correspond to anti-MEP1B and anti-Notch2.

### 3.7 In Vivo Bioactivity Quantifying Gene Expression Changes

The in vivo bioactivity of MEP1BxNotch2 ANDbodies is quantified using the gene expression methods described above.

### 3.8 In Vivo Bioactivity Using Weights and Histology

The in vivo bioactivity of MEP1BxNotch2 ANDbodies is quantified using weight and histology methods described above.

### Example 4. ANDbody Binding Mouse and Human Rage and IL11Ra

### 4.1 Vaccination to Create Anti-RAGE Antibodies

Methods are described above to vaccinate in order to create mouse/human cross-reactive anti-RAGE antibodies.

### 4.2 Selecting for Inert Anti-RAGE Antibodies

Methods are described above to select for inert anti-RAGE antibodies.

### 4.3 Yeast Surface Display to Create Anti-IL11Ra Antibodies

Yeast surface display is used similar to above to create antibodies of varying affinities cross-reactive with mouse and human IL11Ra, an exemplary effector target of the present technology. The DNA sequences coding for extracellular domains of mouse IL11Ra (positions 24-372 of UniProt ID Q64385) and human IL11Ra (positions 24-370 of UniProt ID Q14626) are codon optimized for mammalian expression and ordered with a C-terminal His tag in the pcDNA3.4-TOPO expression vector (ThermoFisher Scientific). Proteins are transiently transfected into HEK293F cells and purified using TALON® Metal Affinity Resin according to manufacturer's instructions (Clontech) similar to prior methods (Rothschilds et al. 2019). These soluble recombinant mouse and human IL11Ra are used as the antigens for yeast surface display.

ScFv's cross-reactive to mouse and human IL11Ra are cloned into human IgG1, transiently transfected into HEK293F cells, and purified using protein A resin as described above.

### 4.4 Selecting for Active Anti-IL11Ra Antibodies at Wide IC₅₀ Ranges

The IC₅₀s of IL11Ra antibodies on the ligand human IL11 (R&D Systems, catalog #218-IL-025/CF) binding to surface IL11Ra are evaluated using flow cytometry to choose antibodies at IC₅₀'s ranging from less than 1 nM up to 5 uM, as described above. In this current example, full length human IL11Ra is transiently transfected to the surface of HEK293F cells. The methods from the prior example are used by replacing IL11Ra antibodies in place of Notch2 antibodies, and IL11 in place of Jagged-2-Fc.

### 4.5 Expressing and Purifying ANDbodies as Bispecifics

DNA sequences from 10 RAGE antibodies and 10 IL11Ra antibodies ranging in IC₅₀ (from <1 nM up to 5 uM) are made into about 100 variant ANDbodies as described above.

### 4.6 Affinity of ANDbody Variants for RAGE and IL11Ra

ANDbody affinities for RAGE and IL11Ra are evaluated similarly to above using a BIAcore. In this case, human and mouse versions of His-tagged IL11Ra are immobilized on a Sensor Chip NTA (Cytiva catalog #BR100034), and human and mouse versions of RAGE-Fc are captured on a Sensor Chip Protein A (Cytiva catalog #29127557).

4.7 In Vitro Assays for IL11Ra Antagonism on Cells with or without RAGE Expression

This assay is done as described above, except substituting full length human IL11Ra instead of Notch2, as well as substituting anti-IL11Ra antibodies for anti-Notch antibodies. The corresponding RAGExIL11Ra ANDbodies are also used.

### 4.8 Biodistribution (In Vivo) for ANDbody and Parental Antibodies

Cellular and tissue biodistribution studies are done using methods described above. However, the ANDbody used in this case is RAGExIL11Ra, and the parental antibodies correspond to anti-RAGE and anti-IL11Ra.

### 4.9 In Vivo Bioactivity of RAGExIL11Ra ANDbodies

In response to mouse treatment with murine IL11, there is an increase in collagen content in both the ventricle and the kidney (Schafer et al. 2017). Accordingly, collagen content is measured to quantify amount of IL11Ra bioactivity after ANDbody treatment.

Similar to prior methods (Schafer et al. 2017), 10-week-old male C57BL/6 mice are injected with 2 ug mouse IL11 daily subcutaneously or an identical volume of saline for 21 days. The mouse IL11 is recombinantly made by synthesizing codon-optimized DNA using the sequence for mouse IL11 (UniProt ID P47873) with a C-terminal His tag, doing HEK293F transient transfections, and purifying the His tagged IL11 with TALON resin as described above. Starting 3 days before the first IL11 injection and then 2× per week thereafter, IL11- and saline-treated mice receive therapeutic injections IP constituting 250 μg of ANDbody RAGExIL11Ra, parental anti-IL11Ra alone, or saline (PBS) in equal volume.

At the end of 21 days of IL11 treatment, mice are euthanized and the amounts of total collagen in the lung, spleen, blood, kidney, liver, heart, and intestines are quantified on the basis of colorimetric detection of hydroxyproline using a Quickzyme Total Collagen assay kit (Quickzyme Biosciences) and similar to prior methods (Schafer et al. 2017).

### Example 5. ANDbody Binding Mouse and Human UMOD and IL11Ra

### 5.1 Yeast Surface Display to Create Anti-UMOD Antibodies

Anti-UMOD (e.g., address target) antibodies are selected for as above and cloned into human IgG1.

### 5.2 Selecting for Inert Anti-UMOD Antibodies

Anti-UMOD antibodies are further evaluated based on their inability to block UMOD ligand binding, in an assay such as the ones described above.

### 5.3 Yeast Surface Display to Create Anti-IL11Ra Antibodies

The same IL11Ra antibodies generated above in yeast surface display will be used here as described above. ScFv's cross-reactive to mouse and human IL11Ra are cloned into human IgG1, transiently transfected into HEK293F cells, and purified using protein A resin as described above.

### 5.4 Selecting for Active IL11Ra Antibodies at Wide IC₅₀ Ranges

The method described above is used to select for IL11Ra antibodies at varied IC₅₀s.

### 5.5 Expressing and Purifying ANDbodies as Bispecifics

DNA sequences from 10 UMOD antibodies and 10 IL11Ra antibodies ranging in IC₅₀ (from <1 nM up to 5 uM) are made into about 100 variant ANDbodies as described above.

### 5.6 Affinity of ANDbody Variants for UMOD and IL11Ra

ANDbody affinities for UMOD and IL11Ra are evaluated similarly to above using a BIAcore. In this case, human and mouse versions of His-tagged UMOD and His-tagged IL11Ra are immobilized on a Sensor Chip NTA (Cytiva catalog #BR100034). It is expected that some ANDbody variants have higher affinity for UMOD than for IL11Ra, although all variants are tested in future assays.

### 5.7 Biodistribution (In Vivo) for ANDbody and Parental Antibodies

Cellular and tissue biodistribution studies are done using methods described above. However, the ANDbody used in this example is UMODxIL11Ra, and the parental antibodies correspond to anti-UMOD and anti-IL11Ra.

### 5.8 In Vivo Bioactivity of UMODxIL11Ra ANDbodies

The in vivo bioactivity of UMODxIL11Ra ANDbodies is quantified using the same methods described above.

### Example 6. ANDbody Binding Mouse and Human MEP1B and IL11Ra

### 6.1 Yeast Surface Display to Create Anti-MEP1B Antibodies

Anti-MEP1B (e.g., address target) antibodies are selected for as above and cloned into human IgG1.

### 6.2 Yeast Surface Display to Create Anti-IL11Ra Antibodies

The same IL11Ra (e.g., effector target) antibodies generated above in yeast surface display will be used here. ScFv's cross-reactive to mouse and human IL11Ra are cloned into human IgG2, transiently transfected into HEK293F cells, and purified using protein A resin as described above.

### 6.3 Selecting for Active Anti-IL11Ra Antibodies at Wide IC₅₀ Ranges

The method described above is used to select for IL11Ra antibodies at varied IC₅₀s.

6.4 Expressing and Purifying ANDbodies as Fusion Proteins into Human IgG2

The 10 highest affinity MEP1B scFv's and 10 IL11Ra antibodies ranging in affinity (from <1 nM up to 5 uM) are made as ANDbodies with human IgG2 Fc regions. To do this, the scFv sequences from MEP1B variants are cloned respectively onto the IgG2 IL11Ra antibody variants. The MEP1B scFvs are separated by a flexible linker (3xGGGGS) from either the N or C terminal of either the light or heavy chains of IL11Ra antibodies (each ANDbody has 2 MEP1B scFvs). Variants are made with 4 total MEP1B scFvs per ANDbody by cloning MEP1B scFvs (always separated by the linker) before the N terminal and after the C terminal of the heavy chain or the light chain, respectively. Other variants with 4 or more MEP1B scFvs per IL11Ra antibody on the MEP1BxIL11Ra ANDbody by mixing and matching the locations of the scFv on the IL11Ra antibodies: at N terminal of both heavy and light chains; at C terminal of both heavy and light chains; at N terminal of heavy chain and C terminal of light chain; at C terminal of heavy chain and N terminal of light chain; and other variants with scFvs in 3 or 4 different locations (resulting in 6 or 8 total scFvs per ANDbody, respectively).

### 6.5 Affinity of ANDbody Variants for MEP1B and IL11Ra

ANDbody affinities for MEP1B and IL11Ra are evaluated similarly to above using a BIAcore. In this case, human and mouse versions of His-tagged MEP1B and His-tagged IL11Ra are immobilized on a Sensor Chip NTA (Cytiva catalog #BR100034).

### 6.6 Biodistribution (In Vivo) for ANDbody and Parental Antibodies

Cellular and tissue biodistribution studies are done using methods described above. However, the ANDbody used in this case is MEP1BxIL11Ra, and the parental antibodies correspond to anti-MEP1B and anti-IL11Ra.

### 6.7 In Vivo Bioactivity of MEP1BxIL11Ra ANDbodies

The in vivo bioactivity of MEP1BxIL11Ra ANDbodies is quantified using the same methods described above.

### Example 7. Exemplary Address-Restricted Binder for Skin

This example demonstrates the restricted expression of an anti-DSG1 antibody (address binder) in skin.

### 7.1 Anti-DSG1 Monoclonal Antibody Expression and Purification

Sequences encoding the variable heavy chain regions (HC: SEQ ID NO: 24 and SEQ ID NO: 26, shown in Table 5) of two anti-desmoglein-1 (anti-DSG1) antibodies named 3-09*5 and 3-07/1e (Yamagami et al., J Immunol., 183(9): 5615-5621, 2009) were fused to a human IgG1 (huIgG1) backbone with the effector null mutations L234A, L235A, and P329G (LALA-PG) and cloned into a PCDNA3.4™ vector (ThermoFisher Scientific). The variable light chain regions (SEQ ID NO: 25 and SEQ ID NO: 27) were fused to a constant kappa light chain (for 3-09*5) (SEQ ID NO: 22) or a constant lambda light chain (for 3-07/1e) (SEQ ID NO: 23) and cloned into PCDNA3.4™.

To express and purify the antibodies, a 1:1 ratio of heavy chain to light chain DNA was transfected into EXPI293F™ cells (ThermoFisher Scientific) using the EXPIFECTAMINE™ 293 Transfection Kit (ThermoFisher Scientific) following manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS. The resulting mAbs were designated as PRO003 (3-09*5 HC) (heavy chain sequence: SEQ ID NO: 28; light chain sequence: SEQ ID NO: 29) and PRO004 (3-07/1e HC) (heavy chain sequence: SEQ ID NO: 30; light chain sequence: SEQ ID NO: 31).

The purified mAbs were analyzed by analytical size exclusion chromatography (SEC) for monodispersity and by SDS-PAGE for purity.

TABLE 5
PRO003 and PRO004 sequences

| | | SEQ ID |
|---|---|---|
| Region | Sequence | number |
| Heavy chain | MGWSCIILFLVATATGVHS | SEQ ID NO: 19 |
| signal peptide | | |
| sequence | | |
| Light chain signal | METDTLLLWVLLLWVPGSTG | SEQ ID NO: 20 |
| peptide sequence | | |
| Constant heavy | ASTKGPSVFPLAPSSKSTSGGTAALGCLVKDYFPEPVTVSW | SEQ ID NO: 21 |
| chain region | NSGALTSGVHTFPAVLQSSGLYSLSSVVTVPSSSLGTQTYIC | |
| | NVNHKPSNTKVDKKVEPKSCDKTHTCPPCPAPEAAGGPSVF | |
| | LFPPKPKDTLMISRTPEVTCVVVDVSHEDPEVKFNWYVDGVE | |
| | VHNAKTKPREEQYNSTYRVVSVLTVLHQDWLNGKEYKCKVS | |
| | NKALGAPIEKTISKAKGQPREPQVYTLPPSRDELTKNQVSLTC | |
| | LVKGFYPSDIAVEWESNGQPENNYKTTPPVLDSDGSFFLYSK | |
| | LTVDKSRWQQGNVFSCSVMHEALHNHYTQKSLSLSPG | |
| Kappa light chain | RTVAAPSVFIFPPSDEQLKSGTASVVCLLNNFYPREAKVQWK | SEQ ID NO: 22 |
| constant region | VDNALQSGNSQESVTEQDSKDSTYSLSSTLTLSKADYEKHK | |
| | VYACEVTHQGLSSPVTKSFNRGEC | |
| Lambda light | GQPKAAPSVTLFPPSSEELQANKATLVCLISDFYPGAVTVAW | SEQ ID NO: 23 |
| chain constant | KADSSPVKAGVETTTPSKQSNNKYAASSYLSLTPEQWKSHR | |
| region | SYSCQVTHEGSTVEKTVAPTECS | |
| 3-09*5 variable | EVQLVESGGGLVQPGRSLRLSCAASGFTFDDYAMHWVRQA | SEQ ID NO: 24 |
| heavy chain | PGKGLEWVSGISWNSGSIDYADSVKGRFTISRDNAKNSLYLQ | |
| sequence | MNSLRVEDTALYYCAKDGSRVFGVGGGFDFWGQGTMVTVS | |
| | S | |
| 3-09*5 variable | ELQMTQSPSSLSASVGDRVTITCQASQDIGNYLNWYQQKPG | SEQ ID NO: 25 |
| light chain | KAPKLLIYDASYLETGVPSRFSGSGSGTDFTFTISSLQPEDIAT | |
| sequence | YYCQQYDNLPFTFGPGTKVDIK | |
| 3-07/1e variable | QVQLVQSGGGLVQPGGSLRVSCAASGFTSNIFWMSWVRQA | SEQ ID NO: 26 |
| heavy chain | PGKGLEWVANIDEDGSEKNYVDSVKGRFTISRDNAKNSLYL | |
| sequence | QMNSLRAEDTAVYYCARESFYYGSGTYFDFWGQGTLVTVS | |
| | S | |
| 3-07/1e variable | ELVVTQPPSVSGAPGQRVTISCTGSSSNIGAGYDVHWYQQL | SEQ ID NO: 27 |
| light chain | PGTAPKLLIYGNKNRPSGVPDRFSGSKSGTSASLAITGLRAE | |
| sequence | DEADYYCQSFDSSLGWVFGGGTQLTVL | |
| PRO003 heavy | MGWSCIILFLVATATGVHSEVQLVESGGGLVQPGRSLRLSCA | SEQ ID NO: 28 |
| chain | ASGFTFDDYAMHWVRQAPGKGLEWVSGISWNSGSIDYADS | |
| | VKGRFTISRDNAKNSLYLQMNSLRVEDTALYYCAKDGSRVF | |
| | GVGGGFDFWGQGTMVTVSSASTKGPSVFPLAPSSKSTSGG | |
| | TAALGCLVKDYFPEPVTVSWNSGALTSGVHTFPAVLQSSGL | |
| | YSLSSVVTVPSSSLGTQTYICNVNHKPSNTKVDKKVEPKSCD | |
| | KTHTCPPCPAPEAAGGPSVFLFPPKPKDTLMISRTPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPRE | |
| | PQVYTLPPSRDELTKNQVSLTCLVKGFYPSDIAVEWESNGQP | |
| | ENNYKTTPPVLDSDGSFFLYSKLTVDKSRWQQGNVFSCSVM | |
| | HEALHNHYTQKSLSLSPG | |
| PRO003 light | METDTLLLWVLLLWVPGSTGELQMTQSPSSLSASVGDRVTIT | SEQ ID NO: 29 |
| chain | CQASQDIGNYLNWYQQKPGKAPKLLIYDASYLETGVPSRFS | |
| | GSGSGTDFTFTISSLQPEDIATYYCQQYDNLPFTFGPGTKVDI | |
| | KRTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |
| PRO004 heavy | MGWSCIILFLVATATGVHSQVQLVQSGGGLVQPGGSLRVSC | SEQ ID NO: 30 |
| chain | AASGFTSNIFWMSWVRQAPGKGLEWVANIDEDGSEKNYVD | |
| | SVKGRFTISRDNAKNSLYLQMNSLRAEDTAVYYCARESFYYG | |
| | SGTYFDFWGQGTLVTVSSASTKGPSVFPLAPSSKSTSGGTA | |
| | ALGCLVKDYFPEPVTVSWNSGALTSGVHTFPAVLQSSGLYSL | |
| | SSVVTVPSSSLGTQTYICNVNHKPSNTKVDKKVEPKSCDKTH | |
| | TCPPCPAPEAAGGPSVFLFPPKPKDTLMISRTPEVTCVVVDV | |
| | SHEDPEVKFNWYVDGVEVHNAKTKPREEQYNSTYRVVSVLT | |
| | VLHQDWLNGKEYKCKVSNKALGAPIEKTISKAKGQPREPQVY | |
| | TLPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| PRO004 light | METDTLLLWVLLLWVPGSTGELVVTQPPSVSGAPGQRVTISC | SEQ ID NO: 31 |
| chain | TGSSSNIGAGYDVHWYQQLPGTAPKLLIYGNKNRPSGVPDR | |
| | FSGSKSGTSASLAITGLRAEDEADYYCQSFDSSLGWVFGGG | |
| | TQLTVLGQPKAAPSVTLFPPSSEELQANKATLVCLISDFYPGA | |
| | VTVAWKADSSPVKAGVETTTPSKQSNNKYAASSYLSLTPEQ | |
| | WKSHRSYSCQVTHEGSTVEKTVAPTECS | |

### 7.2 Anti-DSG1 Antibodies PRO003 and PRO004 Bind to Murine DSG1 Expressed on Cells

Murine DSG1 (NCBI accession NP_034209.2) with a c-Myc epitope tag at the protein C terminus was transiently expressed in RAW 264.7 cells using LIPOFECTAMINE™ 3000 (ThermoFisher Scientific) according to the manufacturer's protocol. Expression of DSG1 was confirmed by fixing and permeabilizing the cells, followed by staining with an anti-c-Myc antibody (Life Technologies A-21281) and analysis by flow cytometry. PRO003 and PRO004 bound specifically to the cells transfected with mouse DSG1, confirming that they have the expected binding specificity and are suitable for studies in mice.

7.3 Anti-DSG1 Antibodies Injected into Mice Accumulate Preferentially in Skin

To demonstrate that binding to a skin address can cause accumulation of an antibody in the skin, the anti-DSG1 antibodies PRO003 and PRO004 were chemically conjugated to the near-infrared (IR) dye IRDYE® 800CW using according to the manufacturer's instructions (LI-COR® 928-38044).

The labeled antibodies were each administered to mice by tail vein injection at a dose level of 3 mg/kg. Each antibody was administered to two groups of 3 mice, which were euthanized at 3 days and 7 days after dosing. Following euthanization, 9 organs were collected (heart, lung, pancreas, kidney, small intestine, large intestine, skin, liver, stomach), and the near-IR fluorescence of each tissue was measured on a IVIS® imager (PERKINELMER®). To image the skin, a patch of skin was shaved and approximately 1 cm² was collected for imaging. Samples from each mouse were arranged in a standard format and total fluorescence intensity was measured. Fluorescence intensity from each organ was quantified and averaged across each tissue by measuring total signal and subtracting the local background. High background signal was observed in the livers from all treated mice, so livers were excluded from the analysis. Without wishing to be bound by theory, it is believed that the liver may take up the fluorescent dye independent of antibody targeting. Similarly, background signal was observed in the stomach from all groups, including mice that were not treated with any antibody. Fluorescence was observed from the food fed to the mice, so the stomach was excluded from analysis.

FIGS. 5A and 5B show fluorescent signal across tissues for PRO003 (FIG. 5A) and PRO004 (FIG. 5B). Distribution of both antibodies is strongly skewed to the skin. These data show that DSG1 antibodies can be used as an address for preferential skin targeting of an ANDbody.

### Example 8. Exemplary Address-Restricted Binder for Lung

This example demonstrates the restricted expression of an anti-RAGE antibody (address binder) in the lung.

### 8.1 Anti-RAGE Monoclonal Antibody Expression and Purification

Sequences encoding the variable heavy chain regions (SEQ ID NO: 32 and SEQ ID NO: 34, shown in Table 6) of two anti-RAGE mAbs named h11E6.8 and XT-M4 (Creative Biolabs) were fused to a huIgG1 backbone with the effector null mutations L234A, L235A, and P329G (LALA-PG) and cloned into a PCDNA3.4™ vector (ThermoFisher Scientific). Sequences encoding the variable light chain regions (SEQ ID NO: 33 and SEQ ID NO: 35) were fused to constant kappa light chains and cloned into PCDNA3.4™.

For expression and purification, a 1:1 ratio of heavy chain to light chain DNA was transfected into EXPI293F™ Cells (ThermoFisher Scientific) using the EXPIFECTAMINE™ 293 Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH8.5. The neutralized eluate was buffer exchanged into PBS. The resulting mAbs were designated PRO001 (h11E6.8) (heavy chain sequence: SEQ ID NO: 36; light chain sequence: SEQ ID NO: 37) and PRO002 (XT-M4) (heavy chain sequence: SEQ ID NO: 38; light chain sequence: SEQ ID NO: 39).

The purified mAbs were analyzed by analytical size exclusion chromatography for monodispersity and by SOS-PAGE for purity. PRO001 and PRO002 expressed highly monodispersed and resolved on SOS-PAGE at the expected molecular weight. Binding studies confirmed binding to the RAGE antigen (not shown).

TABLE 6
PRO001 and PRO002 sequences

| | | SEQ ID |
|---|---|---|
| Region | Sequence | number |
| h11E6.8 variable | EIQLVQSGSELKKPGASVKVSCKASGYTFTNFGMNWVRQAP | SEQ ID NO: 32 |
| heavy chain | GQGLEWMGYINTNTGESIYSEEFKGRFVFSLDTSVSTAYLQI | |
| sequence | CSLKAEDTAVYFCARSRMVTAYGMDYWGQGTTVTVSS | |
| h11E6.8 variable | EIVMTQSPATLSLSPGERATLSCKASQNVGTAVAWYQQKPG | SEQ ID NO: 33 |
| light chain | QSPRLLIFSASNRYTGVPARFSGSGSGTDFTLTISSLQSEDFA | |
| sequence | VYFCQQYSSYPLTFGQGTKLEIK | |
| XT-M4 variable | EVQLVESGGGLVQPGGSLRLSCAASGFTFNNYWMTWVRQA | SEQ ID NO: 34 |
| heavy chain | PGKGLEWVASIDNSGDNTYYPDSVKDRFTISRDNAKNSLYLQ | |
| sequence | MNSLRAEDTAVYYCARGGDITTGFDYWGQGTLVTVSS | |
| XT-M4 variable | DIQMTQSPSSLSASVGDRVTITCRASQDVGIYVNWFQQKPG | SEQ ID NO: 35 |
| light chain | KAPRRLIYRATNLADGVPSRFSGSRSGTDFTLTISSLQPEDFA | |
| sequence | TYYCLEFDEHPLTFGGGTKVEIK | |
| PRO001 heavy | MGWSCIILFLVATATGVHSEIQLVQSGSELKKPGASVKVSCK | SEQ ID NO: 36 |
| chain | ASGYTFTNFGMNWVRQAPGQGLEWMGYINTNTGESIYSEEF | |
| | KGRFVFSLDTSVSTAYLQICSLKAEDTAVYFCARSRMVTAYG | |
| | MDYWGQGTTVTVSSASTKGPSVFPLAPSSKSTSGGTAALGC | |
| | LVKDYFPEPVTVSWNSGALTSGVHTFPAVLQSSGLYSLSSVV | |
| | TVPSSSLGTQTYICNVNHKPSNTKVDKKVEPKSCDKTHTCPP | |
| | CPAPEAAGGPSVFLFPPKPKDTLMISRTPEVTCVVVDVSHED | |
| | PEVKFNWYVDGVEVHNAKTKPREEQYNSTYRVVSVLTVLHQ | |
| | DWLNGKEYKCKVSNKALGAPIEKTISKAKGQPREPQVYTLPP | |
| | SRDELTKNQVSLTCLVKGFYPSDIAVEWESNGQPENNYKTT | |
| | PPVLDSDGSFFLYSKLTVDKSRWQQGNVFSCSVMHEALHNH | |
| | YTQKSLSLSPG | |
| PRO001 light | METDTLLLWVLLLWVPGSTGEIVMTQSPATLSLSPGERATLS | SEQ ID NO: 37 |
| chain | CKASQNVGTAVAWYQQKPGQSPRLLIFSASNRYTGVPARFS | |
| | GSGSGTDFTLTISSLQSEDFAVYFCQQYSSYPLTFGQGTKLEI | |
| | KRTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |
| PRO002 heavy | MGWSCIILFLVATATGVHSEVQLVESGGGLVQPGGSLRLSCA | SEQ ID NO: 38 |
| chain | ASGFTFNNYWMTWVRQAPGKGLEWVASIDNSGDNTYYPDS | |
| | VKDRFTISRDNAKNSLYLQMNSLRAEDTAVYYCARGGDITTG | |
| | FDYWGQGTLVTVSSASTKGPSVFPLAPSSKSTSGGTAALGC | |
| | LVKDYFPEPVTVSWNSGALTSGVHTFPAVLQSSGLYSLSSVV | |
| | TVPSSSLGTQTYICNVNHKPSNTKVDKKVEPKSCDKTHTCPP | |
| | CPAPEAAGGPSVFLFPPKPKDTLMISRTPEVTCVVVDVSHED | |
| | PEVKFNWYVDGVEVHNAKTKPREEQYNSTYRVVSVLTVLHQ | |
| | DWLNGKEYKCKVSNKALGAPIEKTISKAKGQPREPQVYTLPP | |
| | SRDELTKNQVSLTCLVKGFYPSDIAVEWESNGQPENNYKTT | |
| | PPVLDSDGSFFLYSKLTVDKSRWQQGNVFSCSVMHEALHNH | |
| | YTQKSLSLSPG | |
| PRO002 light | METDTLLLWVLLLWVPGSTGDIQMTQSPSSLSASVGDRVTIT | SEQ ID NO: 39 |
| chain | CRASQDVGIYVNWFQQKPGKAPRRLIYRATNLADGVPSRFS | |
| | GSRSGTDFTLTISSLQPEDFATYYCLEFDEHPLTFGGGTKVEI | |
| | KRTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |

To test the binding of the anti-RAGE antibodies by ELISA, recombinant His-tagged murine RAGE protein (ab276858 from Abcam) was coated on NUNC-IMMUNO™ MAXISORP™ ELISA plates at 1 μg/mL concentration overnight. The next day, coated antigen was removed and the wells were blocked with 1% IgG-free bovine serum albumin (BSA) followed by incubation with 11 four-fold serially diluted anti-RAGE antibodies (PRO001 and PRO002) with a starting concentration of 20 nM. Bound antibodies were detected with peroxidase-conjugated anti-human IgG antibodies with tetramethylbenzidine (TMB) and acid stop reagents. Both PRO001 and PRO002 bound murine RAGE antigen at similar affinities by ELISA around 90 μM apparent affinity, suggesting that both antibodies are tight binders.

### 8.2 Anti-RAGE Antibodies PRO001 and PRO002 Bind to Murine RAGE Expressed on Cells

PRO001 and PRO002 were tested for binding to mouse RAGE in cell culture. To confirm binding activity and specificity of the two antibodies, mouse RAGE (NCBI accession NP_031451.2) with a c-Myc epitope tag at the protein C terminus was transiently expressed in EXPI293™ cells (ThermoFisher Scientific) using EXPIFECTAMINE™ (ThermoFisher Scientific) according to the manufacturer's protocol. Expression of RAGE was confirmed by fixing and permeabilizing the cells, followed by staining with anti-c-Myc antibody (Life Technologies A-21281) and analysis by flow cytometry. Both antibodies bound specifically to murine RAGE expressed on cells, confirming that they have the expected binding specificity and are suitable for studies in mice.

8.3 Anti-RAGE Antibodies Injected into Mice Accumulate Preferentially in Lungs

To demonstrate that binding to a lung address can cause accumulation of an antibody in the lungs, the anti-RAGE antibodies PRO001 and PRO002 were chemically conjugated to the near-IR dye as described in Example 7.

The labeled antibodies were each administered to mice by tail vein injection and imaged as described in Example 7. FIGS. 6A and 6B show fluorescent signal measured from tissues across mice treated with the two antibodies, each normalized so that the brightest signal is equal to 1. A group of three untreated mice is included as a negative control for autofluorescence. Distribution of both antibodies is strongly skewed to the lung when compared to the other antibodies tested. These data show that two antibodies binding to RAGE accumulate preferentially in lung, demonstrating they can be used as an address for preferential lung targeting of an ANDbody.

### 8.4 an Anti-RAGE Antibody Accumulates Specifically on Alveolar Cells

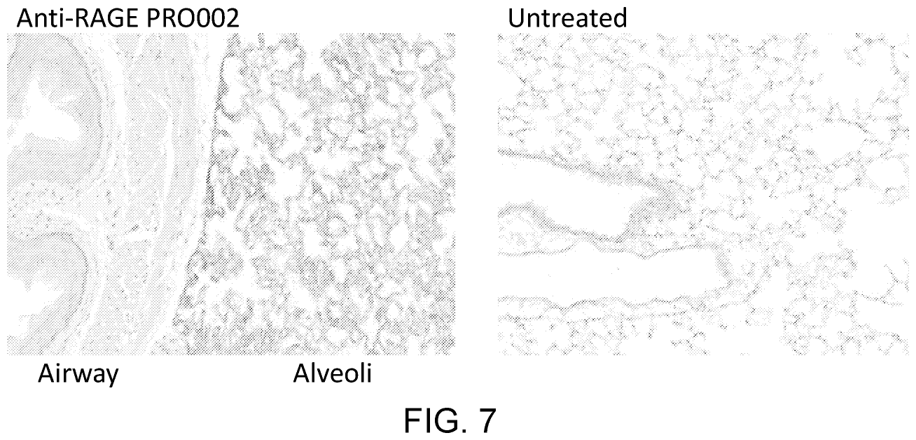

Single-cell expression analysis indicated that RAGE is expressed specifically in type 1 alveolar cells, with lower expression in type 2 alveolar cells. To test the hypothesis that the antibodies can address a specific cell type, three Balb/C mice were treated by tail vein injection with 3 mg/kg of PRO002. Three untreated mice were used as negative control. Three days after dosing, the mice were euthanized, lungs and other tissues were collected, and all tissues were fixed in formalin. Sections from each tissue were analyzed by immunohistochemistry (IHC) using an anti-human secondary antibody conjugated to horseradish peroxidase. FIG. 7 shows representative staining in treated and untreated mice. Strong staining was observed in the alveolar tissue of mice treated with PRO002, but not in adjacent airways or negative control conditions. This result shows that binders to addresses specific to a particular cell type can be used to direct the distribution of antibodies to those cells within a larger tissue.

### Example 9. Exemplary Address-Restricted Binder for Kidney

This example demonstrates the restricted expression of an anti-CDH16 antibody (address binder) in the kidney.

### 9.1 Anti-CDH16 Monoclonal Antibody Expression and Purification

A sequence encoding the variable heavy chain region (SEQ ID NO: 40; shown in Table 7) of the anti-cadherin 16 (anti-CDH16) mAb Ab270263 (Abcam) was fused to a huIgG1 backbone with the effector null mutations L234A, L235A, and P329G (LALA-PG) and cloned into a PCDNA3.4™ vector (ThermoFisher Scientific). A sequence encoding the variable light chain region (SEQ ID NO: 41) was fused to a constant kappa light chain and cloned into PCDNA3.4™.

For expression and purification, a 1:1 ratio of heavy chain to light chain DNA was transfected into EXPI293F™ Cells (ThermoFisher Scientific) using the EXPIFECTAMINE™ Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS. The resulting mAb was designated as PRO056 (heavy chain sequence: SEQ ID NO: 42; light chain sequence: SEQ ID NO: 43).

The purified mAb was analyzed by analytical size exclusion chromatography for monodispersity and by SDS-PAGE for purity. PRO056 expressed highly monodispered and resolved on SDS-PAGE at the expected molecular weight.

TABLE 7
PRO056 sequences

| | | SEQ ID |
|---|---|---|
| Region | Sequence | number |
| Ab270263 variable | QVHLKESGPGLVAPSQSLSITCTVSGFSLTSYAVHWVRQPP | SEQ ID NO: 40 |
| heavy chain | GKGLEWLGVIWAGGNTNYNSVFMSRLTISKDNSKSQVFLKM | |
| sequence | NSLQTDDTAIYYCARLDDYDERFVYWGQGTLVTVSS | |
| Ab270263 variable | DIVMSQSPSSLAVSVGEKVSMNCKSSQSLLYSSNHKNYLAW | SEQ ID NO: 41 |
| light chain | FQQKPGQSPKLLIYWASTRESGVPDRFTGSGSGTDFTLTISS | |
| sequence | VKAEDLAVYYCQQYYTYTWTFGGGTKLEIK | |
| PRO056 heavy | MGWSCIILFLVATATGVHSQVHLKESGPGLVAPSQSLSITCTV | SEQ ID NO: 42 |
| chain | SGFSLTSYAVHWVRQPPGKGLEWLGVIWAGGNTNYNSVFM | |
| | SRLTISKDNSKSQVFLKMNSLQTDDTAIYYCARLDDYDERFV | |
| | YWGQGTLVTVSSASTKGPSVFPLAPSSKSTSGGTAALGCLV | |
| | KDYFPEPVTVSWNSGALTSGVHTFPAVLQSSGLYSLSSVVTV | |
| | PSSSLGTQTYICNVNHKPSNTKVDKKVEPKSCDKTHTCPPCP | |
| | APEAAGGPSVFLFPPKPKDTLMISRTPEVTCVVVDVSHEDPE | |
| | VKFNWYVDGVEVHNAKTKPREEQYNSTYRVVSVLTVLHQD | |
| | WLNGKEYKCKVSNKALGAPIEKTISKAKGQPREPQVYTLPPS | |
| | RDELTKNQVSLTCLVKGFYPSDIAVEWESNGQPENNYKTTP | |
| | PVLDSDGSFFLYSKLTVDKSRWQQGNVFSCSVMHEALHNHY | |
| | TQKSLSLSPG | |
| PRO056 light | METDTLLLWVLLLWVPGSTGDIVMSQSPSSLAVSVGEKVSM | SEQ ID NO: 43 |
| chain | NCKSSQSLLYSSNHKNYLAWFQQKPGQSPKLLIYWASTRES | |
| | GVPDRFTGSGSGTDFTLTISSVKAEDLAVYYCQQYYTYTWTF | |
| | GGGTKLEIKRTVAAPSVFIFPPSDEQLKSGTASVVCLLNNFYP | |
| | REAKVQWKVDNALQSGNSQESVTEQDSKDSTYSLSSTLTLS | |
| | KADYEKHKVYACEVTHQGLSSPVTKSFNRGEC | |

To test the binding of the anti-CDH16 antibodies by ELISA, recombinant His-tagged murine CDH16 protein, expressed and purified in house, was coated on NUNC-IMMUNO™ MAXISORP™ ELISA plates at 1 μg/mL concentration overnight. The next day, coated antigen was removed and the wells were blocked with 1% IgG-free BSA followed by incubation with 11 three-fold serially diluted anti-CDH16 antibody (PRO056) with a starting concentration of 533 nM. Bound antibody was detected with peroxidase-conjugated anti-human IgG antibodies with TMB and acid stop reagents. PRO056 bound murine CDH16 antigen at an affinity of 200 μM by ELISA.

### 9.2 Anti-CDH16 Antibody Accumulates Preferentially in the Kidney

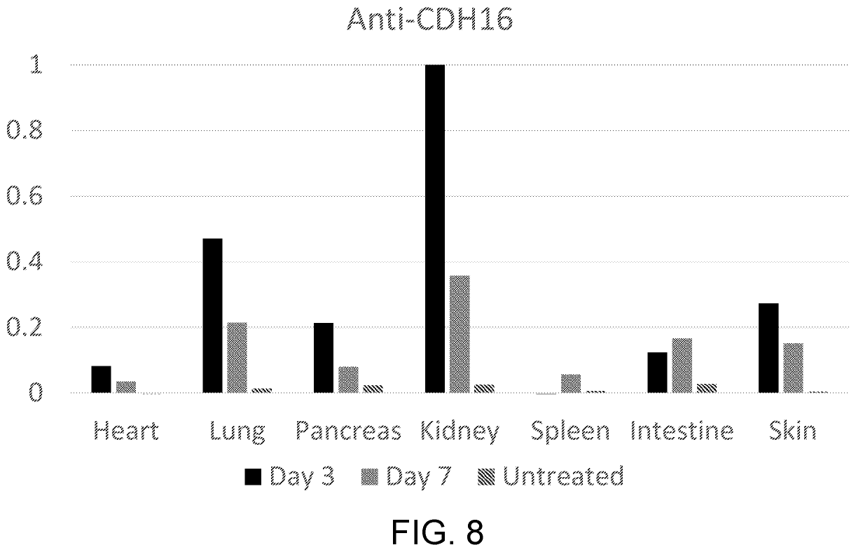

The anti-CDH16 antibody PRO056 was chemically conjugated to near-IR fluorescent dye IRDYE® 800CW, as described in Example 7. The labeled antibody was administered to mice by tail vein injection at a dose level of 3 mg/kg. Two groups of 3 mice were used, which were euthanized at 3 days and 7 days after dosing. Following euthanization, organs were collected, and the near-IR fluorescence of each tissue was measured on a model IVIS® imager (PERKINELMER®), as above. Fluorescence intensity from each organ was quantified and averaged across each tissue by measuring total signal and subtracting the local background. FIG. 8 shows fluorescent signal measured from the tissues across mice treated with PRO056. Each is normalized so that the brightest signal is equal to 1. A group of three untreated mice is included as a negative control for autofluorescence.

The distribution is strongly skewed to the kidney when compared to the antibodies provided herein that target addresses in the skin, lung, or kidney. These data show that an antibody binding to CDH16 accumulates preferentially in kidney, demonstrating they can be used as an address bidding domain for preferential kidney targeting of an ANDbody.

### Example 10. Exemplary Address-Restricted Binder for Intestine

This example demonstrates the restricted expression of an anti-CDH17 antibody (address binder) in the intestine.

### 10.1 Anti-CDH17 Monoclonal Antibody Expression and Purification

A sequence encoding the variable heavy chain region (SEQ ID NO: 44; shown in Table 8) of the anti-cadherin 17 (anti-CDH17) mAb MAB8524 (R&D Systems) was fused to a huIgG1 backbone with the effector null mutations L234A, L235A, P329G (LALA-PG) and cloned into a PCDNA3.4™ vector (ThermoFisher Scientific). A sequence encoding the variable light chain region (SEQ ID NO: 45) was fused to a constant kappa light chain and cloned into PCDNA3.4™.

For expression and purification, a 1:1 ratio of heavy chain to light chain DNA was transfected into EXPI293F™ cells (ThermoFisher Scientific) using the EXPIFECTAMINE™ 293 Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS. The resulting mAb was designated as PRO061 (heavy chain sequence: SEQ ID NO: 46; light chain sequence: SEQ ID NO: 47).

The purified mAb was analyzed by analytical size exclusion chromatography for monodispersity and by SDS-PAGE for purity. PRO061 expressed highly monodispered and resolved on SDS-PAGE at the expected molecular weight.

TABLE 8
PRO061 sequences

| | | SEQ ID |
|---|---|---|
| Region | Sequence | number |
| MAB8524 variable | QSLEESGGRLVTPGTPLTLTCTVSGFSLTSYDMNWVRQAPG | SEQ ID NO: 44 |
| heavy chain | KGLEWIGVVRGSGRTYYASWAKGRFTIARTSSTTVDLKMTSL | |
| sequence | TTGDTATYFCARGDANNNYYEFDIWGPGTLVTVSS | |
| MAB8524 variable | QDMTQTPSPVSAAVGGTVTINCQSSQSVYGDAWLSWFQQK | SEQ ID NO: 45 |
| light chain | PGQPPKLLIYSASTLASGVPSRFKGSGSGTQFTLTISDLESDD | |
| sequence | AATYYCAGGYDGINDIRAFGGGTEVVVK | |
| PRO061 heavy | MGWSCIILFLVATATGVHSQSLEESGGRLVTPGTPLTLTCTVS | SEQ ID NO: 46 |
| chain | GFSLTSYDMNWVRQAPGKGLEWIGVVRGSGRTYYASWAKG | |
| | RFTIARTSSTTVDLKMTSLTTGDTATYFCARGDANNNYYEFDI | |
| | WGPGTLVTVSSASTKGPSVFPLAPSSKSTSGGTAALGCLVK | |
| | DYFPEPVTVSWNSGALTSGVHTFPAVLQSSGLYSLSSVVTVP | |
| | SSSLGTQTYICNVNHKPSNTKVDKKVEPKSCDKTHTCPPCPA | |
| | PEAAGGPSVFLFPPKPKDTLMISRTPEVTCVVVDVSHEDPEV | |
| | KFNWYVDGVEVHNAKTKPREEQYNSTYRVVSVLTVLHQDW | |
| | LNGKEYKCKVSNKALGAPIEKTISKAKGQPREPQVYTLPPSR | |
| | DELTKNQVSLTCLVKGFYPSDIAVEWESNGQPENNYKTTPPV | |
| | LDSDGSFFLYSKLTVDKSRWQQGNVFSCSVMHEALHNHYT | |
| | QKSLSLSPG | |
| PRO061 light | METDTLLLWVLLLWVPGSTGQDMTQTPSPVSAAVGGTVTIN | SEQ ID NO: 47 |
| chain | CQSSQSVYGDAWLSWFQQKPGQPPKLLIYSASTLASGVPSR | |
| | FKGSGSGTQFTLTISDLESDDAATYYCAGGYDGINDIRAFGG | |
| | GTEVVVKRTVAAPSVFIFPPSDEQLKSGTASVVCLLNNFYPR | |
| | EAKVQWKVDNALQSGNSQESVTEQDSKDSTYSLSSTLTLSK | |
| | ADYEKHKVYACEVTHQGLSSPVTKSFNRGEC | |

To confirm binding activity and specificity, mouse CDH17 (NCBI accession NP_062727.1) with a c-Myc epitope tag at the protein C terminus was transiently expressed in RAW 264.7 cells using LIPOFECTAMINE™ 3000 (ThermoFisher Scientific) according to the manufacturer's protocol. Expression of CDH17 was confirmed by fixing and permeabilizing the cells, followed by staining with an anti-c-Myc antibody (Life Technologies A-21281) and analysis by flow cytometry. PRO061 bound specifically to the cells transfected with mouse CDH17, confirming that it has the expected binding specificity and is suitable for studies in mice.

10.2 Anti-CDH17 Antibody Injected into Mice Accumulates Preferentially in the Intestine

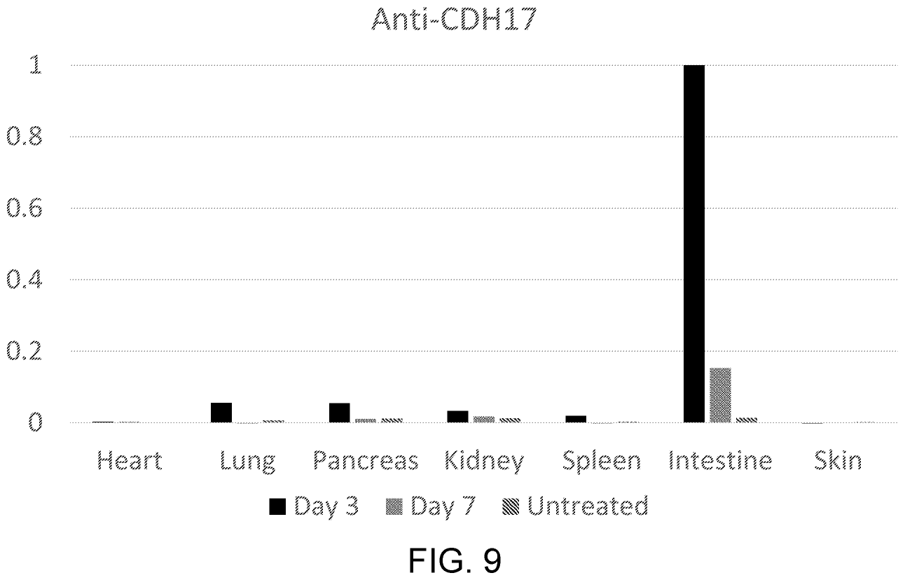

PRO061 was chemically conjugated to near-IR fluorescent dye IRDYE® 800CW, as described in Example 7. The labeled antibody was administered to mice by tail vein injection at a dose level of 3 mg/kg. Two groups of 3 mice were used, which were euthanized at 3 days and 7 days after dosing. Following euthanization, organs were collected, and the near-IR fluorescence of each tissue was measured on a model IVIS® imager (PERKINELMER®), as above. Fluorescence intensity from each organ was quantified and averaged across each tissue by measuring total signal and subtracting the local background. FIG. 9 shows fluorescent signal measured from the tissues across mice treated with PRO061. Each is normalized so that the brightest signal is equal to 1. A group of three untreated mice is included as a negative control for autofluorescence. The distribution is strongly skewed to the intestine, showing that an antibody binding to CDH17 accumulates preferentially in intestine and can be used as an address targeting domain for preferential intestine targeting of an ANDbody.

### Example 11. Tissue Restriction of Predicted Addresses

Immunohistochemistry (IHC) on fresh frozen (FF) healthy mouse tissue microarray (TMA) sections mounted onto glass slides was used for assaying whether predicted organ-specific or preferentially expressed addresses were actually of highest abundance in the predetermined organs, and for determining which monoclonal antibody clones (mAbs) bound with greatest preference to the desired organ.

Glass slides coated with FF TMA were generated by first assembling a fresh frozen tissue microarray block. To enable TMA block formation, individual organs from freshly sacrificed C57BL/6 mice were embedded in optimal cutting (OCT) medium in separate cryomolds and frozen. Cylindrical cores of tissue were then taken from each block and placed into a block to create the final FE TMA. Layers of the TMA were then cut off using a cryostat, mounted onto positively charged microscopy glass slides, and stored at −80° C. until stained.

Addresses were validated by direct binding of FE TMA-coated slides with either polyclonal antibodies or mAbs raised against the address in question. These address-specific antibodies were detected with horseradish peroxidase (HRP)-conjugated antibodies specific for the IgG of the host in which the primary address specific antibody was raised. Location and intensity of binding was determined by the addition of the HRP substrate 3,3′Diaminobenzidine (DAB), which yields a brown color at the site of primary antibody binding, proportional to the abundance of deposited antibody. Nuclei were counterstained with hematoxylin to yield a blue color. For each of the respective addresses, a range of different mAb clones was assayed for tissue specificity, and their patterns of tissue binding were assessed by performing IHC on the same FF TMAs described above.

Table 9 summarizes observed binding of the antibodies tested. All the antibodies tested reacted primarily with the expected target tissue, with varying degrees of weaker reactivity on other tissues. Without wishing to be bound by theory, much of the off-tissue reactivity may reflect non-specific binding by the antibody. For example, four antibodies tested for binding to RAGE each show binding to the lung, but 3 show variable low-level binding to other tissues.

TABLE 9
Candidate addresses evaluated by IHC on mouse tissue microarrays

| | | | | | Primary Ab | |
|---|---|---|---|---|---|---|
| | | | | | reactivity | |
| Ab | Predicted | | | | matches | |
| # | Organ | Address | Clone/Cat # | Source | prediction | Weaker reactivity |
| 1 | Lung | RAGE | NBP2- | Novus Bio | Yes | Spleen, pancreas, |
| | | | 67095 | | | intestine |
| 2 | Lung | RAGE | PR002 | in house | Yes | None detected |
| 3 | Lung | RAGE | MAB11795 | R&D Systems | Yes | Spleen, intestine, |
| | | | | | | heart, kidney, brain |
| 4 | Lung | RAGE | PR001 | in house | Yes | Skin, Pancreas |
| | | | | | | (low) |
| 5 | Kidney | CDH16 | Ab270263 | Abcam | Yes | None detected |
| 6 | Colon | CDH17 | MAB8524 | Novus Bio | Yes | Small intestine, |
| | | | | | | spleen |
| 7 | Pancreas | GP2 | D278-3 | MBL Int. | Yes | None detected |
| | | | | Corp. | | |
| 8 | Liver | ASGR1 | MAB27552 | R&D Systems | Yes | None detected |
| 9 | Small | HEPACAM2 | LS-B888 | LS Bio | Yes | None detected |
| | Intestine | | | | | |
| 10 | Kidney | Nephrin | Ab227806 | Abcam | Yes | None detected |
| 11 | Kidney | Aquaporin 2 | PA5- | ThermoFisher | Yes | Colon (low) |
| | | | 78808 | | | |
| 12 | Negative | n/a | PR0022 | in house | Yes | None detected |
| | Control | | | | | |

### Example 12. ANDbody Production and Use

This example demonstrates the production of an exemplary ANDbody that blocks Notch2 and binds RAGE as an address.

### 12.1 Anti-Notch2 Monoclonal Antibody Expression and Purification

Sequences encoding the variable heavy chain regions (SEQ ID NOs: 48, 50, 52 and 54; shown in Table 10) of four anti-Notch2 mAbs (Wu et al., Nature, 464: 1052-1057, 2010) were each individually fused to a huIgG1 backbone with the effector null mutations L234A, L235A, and P329G (LALA-PG) and cloned into a PCDNA3.4™ vector (ThermoFisher Scientific). Sequences encoding the corresponding variable light chain regions (SEQ ID NOs: 49, 51, 53, and 55) were fused to constant kappa light chains and cloned into PCDNA3.4™.

For expression and purification, a 1:1 ratio of heavy chain to light chain DNA was transfected into EXPI293F™ cells (ThermoFisher Scientific) using the EXPIFECTAMINE™ 293 Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS. The resulting mAbs were designated as PRO034 (heavy chain sequence: SEQ ID NO: 56; light chain sequence: SEQ ID NO: 57), PRO035 (heavy chain sequence: SEQ ID NO: 58; light chain sequence: SEQ ID NO: 59), PRO036 (heavy chain sequence: SEQ ID NO: 60; light chain sequence: SEQ ID NO: 61), and PRO037 (heavy chain sequence: SEQ ID NO: 62; light chain sequence: SEQ ID NO: 63).

TABLE 10
PRO034, PRO035, PRO036, and PRO037 sequences

| | | SEQ ID |
|---|---|---|
| Region | Sequence | number |
| anti-Notch2 mAb 1 | EVQLVESGGGLVQPGGSLRLSCAASGYSFTSYGMSWVRQA | SEQ ID NO: 48 |
| variable heavy | PGKGLEWVSYIYPYSGATYYADSVKGRFTISADTSKNTAYLQ | |
| chain sequence | MNSLRAEDTAVYYCARHSGYYRISSAMDVWGQGTLVTVSS | |
| anti-Notch2 mAb 1 | DIQMTQSPSSLSASVGDRVTITCRASQSISSYLAWYQQKPGK | SEQ ID NO: 49 |
| variable light chain | APKLLIYGASSRASGVPSRFSGSGSGTDFTLTISSLQPEDFAT | |
| sequence | YYCQQYYSSPLTFGQGTKVEIK | |
| anti-Notch2 mAb 2 | EVQLVESGGGLVQPGGSLRLSCAASGYTFSSYGMSWVRQA | SEQ ID NO: 50 |
| variable heavy | PGKGLEWVSYIYPYSGATYYADSVKGRFTISADTSKNTAYLQ | |
| chain sequence | MNSLRAEDTAVYYCARHSGYYRISSAMDVWGQGTLVTVSS | |
| anti-Notch2 mAb 2 | DIQMTQSPSSLSASVGDRVTITCRASQSNRRFLAWYQQKPG | SEQ ID NO: 51 |
| variable light chain | KAPKLLIYGASSRASGVPSRFSGSGSGTDFTLTISSLQPEDFA | |
| sequence | TYYCQQYYISPLTFGQGTKVEIK | |
| anti-Notch2 mAb 3 | EVQLVESGGGLVQPGGSLRLSCAASGYTFSSYGMSWVRQA | SEQ ID NO: 52 |
| variable heavy | PGKGLEWVSYIYPYSGATYYADSVKGRFTISADTSKNTAYLQ | |
| chain sequence | MNSLRAEDTAVYYCARHSGYYRISSAMDVWGQGTLVTVSS | |
| anti-Notch2 mAb 3 | DIQMTQSPSSLSASVGDRVTITCRASQSVRSFLAWYQQKPG | SEQ ID NO: 53 |
| variable light chain | KAPKLLIYRASIRASGVPSRFSGSGSGTDFTLTISSLQPEDFA | |
| sequence | TYYCQQYYISPWTFGQGTKVEIK | |
| anti-Notch2 mAb 4 | EVQLVESGGGLVQPGGSLRLSCAASGYTFSSYGMSWVRQA | SEQ ID NO: 54 |
| variable heavy | PGKGLEWVSYIYPYSGATYYADSVKGRFTISADTSKNTAYLQ | |
| chain sequence | MNSLRAEDTAVYYCARHSGYYRISSAMDVWGQGTLVTVSS | |
| anti-Notch2 mAb 4 | DIQMTQSPSSLSASVGDRVTITCRASQSNRRFLAWYQQKPG | SEQ ID NO: 55 |
| variable light chain | KAPKLLIYGASSRASGVPSRFSGSGSGTDFTLTISSLQPEDFA | |
| sequence | TYYCQQYYISPLTFGQGTKVEIK | |
| PRO034 heavy | MGWSCIILFLVATATGVHSEVQLVESGGGLVQPGGSLRLSCA | SEQ ID NO: 56 |
| chain | ASGYSFTSYGMSWVRQAPGKGLEWVSYIYPYSGATYYADS | |
| | VKGRFTISADTSKNTAYLQMNSLRAEDTAVYYCARHSGYYRI | |
| | SSAMDVWGQGTLVTVSSASTKGPSVFPLAPSSKSTSGGTAA | |
| | LGCLVKDYFPEPVTVSWNSGALTSGVHTFPAVLQSSGLYSLS | |
| | SVVTVPSSSLGTQTYICNVNHKPSNTKVDKKVEPKSCDKTHT | |
| | CPPCPAPEAAGGPSVFLFPPKPKDTLMISRTPEVTCVVVDVS | |
| | HEDPEVKFNWYVDGVEVHNAKTKPREEQYNSTYRVVSVLTV | |
| | LHQDWLNGKEYKCKVSNKALGAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| PRO034 light | METDTLLLWVLLLWVPGSTGDIQMTQSPSSLSASVGDRVTIT | SEQ ID NO: 57 |
| chain | CRASQSISSYLAWYQQKPGKAPKLLIYGASSRASGVPSRFSG | |
| | SGSGTDFTLTISSLQPEDFATYYCQQYYSSPLTFGQGTKVEIK | |
| | RTVAAPSVFIFPPSDEQLKSGTASVVCLLNNFYPREAKVQWK | |
| | VDNALQSGNSQESVTEQDSKDSTYSLSSTLTLSKADYEKHK | |
| | VYACEVTHQGLSSPVTKSFNRGEC | |
| PRO035 heavy | MGWSCIILFLVATATGVHSEVQLVESGGGLVQPGGSLRLSCA | SEQ ID NO: 58 |
| chain | ASGYTFSSYGMSWVRQAPGKGLEWVSYIYPYSGATYYADS | |
| | VKGRFTISADTSKNTAYLQMNSLRAEDTAVYYCARHSGYYRI | |
| | SSAMDVWGQGTLVTVSSASTKGPSVFPLAPSSKSTSGGTAA | |
| | LGCLVKDYFPEPVTVSWNSGALTSGVHTFPAVLQSSGLYSLS | |
| | SVVTVPSSSLGTQTYICNVNHKPSNTKVDKKVEPKSCDKTHT | |
| | CPPCPAPEAAGGPSVFLFPPKPKDTLMISRTPEVTCVVVDVS | |
| | HEDPEVKFNWYVDGVEVHNAKTKPREEQYNSTYRVVSVLTV | |
| | LHQDWLNGKEYKCKVSNKALGAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| PRO035 light | METDTLLLWVLLLWVPGSTGDIQMTQSPSSLSASVGDRVTIT | SEQ ID NO: 59 |
| chain | CRASQSNRRFLAWYQQKPGKAPKLLIYGASSRASGVPSRFS | |
| | GSGSGTDFTLTISSLQPEDFATYYCQQYYISPLTFGQGTKVEI | |
| | KRTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |
| PRO036 heavy | MGWSCIILFLVATATGVHSEVQLVESGGGLVQPGGSLRLSCA | SEQ ID NO: 60 |
| chain | ASGYTFSSYGMSWVRQAPGKGLEWVSYIYPYSGATYYADS | |
| | VKGRFTISADTSKNTAYLQMNSLRAEDTAVYYCARHSGYYRI | |
| | SSAMDVWGQGTLVTVSSASTKGPSVFPLAPSSKSTSGGTAA | |
| | LGCLVKDYFPEPVTVSWNSGALTSGVHTFPAVLQSSGLYSLS | |
| | SVVTVPSSSLGTQTYICNVNHKPSNTKVDKKVEPKSCDKTHT | |
| | CPPCPAPEAAGGPSVFLFPPKPKDTLMISRTPEVTCVVVDVS | |
| | HEDPEVKFNWYVDGVEVHNAKTKPREEQYNSTYRVVSVLTV | |
| | LHQDWLNGKEYKCKVSNKALGAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| PRO036 light | METDTLLLWVLLLWVPGSTGDIQMTQSPSSLSASVGDRVTIT | SEQ ID NO: 61 |
| chain | CRASQSVRSFLAWYQQKPGKAPKLLIYRASIRASGVPSRFSG | |
| | SGSGTDFTLTISSLQPEDFATYYCQQYYISPWTFGQGTKVEIK | |
| | RTVAAPSVFIFPPSDEQLKSGTASVVCLLNNFYPREAKVQWK | |
| | VDNALQSGNSQESVTEQDSKDSTYSLSSTLTLSKADYEKHK | |
| | VYACEVTHQGLSSPVTKSFNRGEC | |
| PRO037 heavy | MGWSCIILFLVATATGVHSEVQLVESGGGLVQPGGSLRLSCA | SEQ ID NO: 62 |
| chain | ASGYTFSSYGMSWVRQAPGKGLEWVSYIYPYSGATYYADS | |
| | VKGRFTISADTSKNTAYLQMNSLRAEDTAVYYCARHSGYYRI | |
| | SSAMDVWGQGTLVTVSSASTKGPSVFPLAPSSKSTSGGTAA | |
| | LGCLVKDYFPEPVTVSWNSGALTSGVHTFPAVLQSSGLYSLS | |
| | SVVTVPSSSLGTQTYICNVNHKPSNTKVDKKVEPKSCDKTHT | |
| | CPPCPAPEAAGGPSVFLFPPKPKDTLMISRTPEVTCVVVDVS | |
| | HEDPEVKFNWYVDGVEVHNAKTKPREEQYNSTYRVVSVLTV | |
| | LHQDWLNGKEYKCKVSNKALGAPIEKTISKAKGQPREPQVYT | |
| | LPPSRDELTKNQVSLTCLVKGFYPSDIAVEWESNGQPENNY | |
| | KTTPPVLDSDGSFFLYSKLTVDKSRWQQGNVFSCSVMHEAL | |
| | HNHYTQKSLSLSPG | |
| PRO037 light | METDTLLLWVLLLWVPGSTGDIQMTQSPSSLSASVGDRVTIT | SEQ ID NO: 63 |
| chain | CRASQSNRRFLAWYQQKPGKAPKLLIYGASSRASGVPSRFS | |
| | GSGSGTDFTLTISSLQPEDFATYYCQQYYISPLTFGQGTKVEI | |
| | KRTVAAPSVFIFPPSDEQLKSGTASVVCLLNNFYPREAKVQW | |
| | KVDNALQSGNSQESVTEQDSKDSTYSLSSTLTLSKADYEKH | |
| | KVYACEVTHQGLSSPVTKSFNRGEC | |

12. 2 Anti Notch2 binding and affinity testing

To test the binding of the anti-Notch2 antibodies by ELISA, recombinant His-tagged human and murine Notch2 NRR domains, expressed and purified in house, were coated on NUNC-IMMUNO™ MAXISORP™ ELISA plates at 1 μg/mL concentration overnight. The next day, coated antigen was removed and the wells were blocked with 1% IgG-free BSA followed by incubation with 11 three-fold serially diluted anti-Notch2 antibodies (PRO034, PRO035, PRO036 and PRO037) with a starting concentration of 200 nM for PRO035 and PRO037 and 666 nM for PRO034 and PRO036. Bound antibody was detected with peroxidase-conjugated anti-human IgG antibodies with TMB and acid stop reagents. Each of the antibodies bound human and murine NRR domain of Notch2 with affinities between 47 μM and 140 nM.

To test the binding affinity of the anti-Notch2 antibodies by biolayer interferometry (BLI), each of PRO034, PRO035, PRO036 and PRO037 were immobilized on anti-human IgG Fc biosensors and dipped into the recombinant His-tagged murine and human Notch2 NRR protein at various concentrations from 1000 nM to 31 nM to measure the rate of association with the antigen. The dissociation rate was then measured by dipping the biosensors into buffer. The binding affinity was calculated as a ratio of the dissociation rate to the association rate. The intrinsic affinities determined by BLI were all in the nM range, suggesting that avidity improves affinity in an ELISA format.

### 12. 3 Design and Production of Notch2/RAGE ANDbodies

ANDbodies containing a first fragment antigen-binding (Fab) arm of a anti-Notch2 antibody described above (PRO034, PRO035, and PRO036) and a second Fab arm of the anti-RAGE antibody PRO002 (Example 8) were generated using a controlled Fab arm exchange (cFAE) reaction (Labrijn et al., Proc Natl Acad Sci U.S.A., 110(13): 5145-5150, 2013). Site-directed mutagenesis was performed to introduce a F405L amino acid substitution mutation in the Fc fragment of the anti-RAGE antibody (PRO002) and a K409R amino acid substitution mutation in the Fc fragment of each of the anti-Notch2 antibodies (PRO034, PRO035, and PRO036). These antibodies were expressed and purified as described previously for the parental antibodies. The individual monoclonal antibodies were then mixed in an equimolar ratio in a controlled reduction and reoxidation reaction that drives recombination of the bispecific antibody guided by the matching point mutations (F405L-K409R). The formation of the ANDbody was analyzed by analytical chromatography and SDS-PAGE. The resulting ANDbodies were designated as PRO051, PRO052 and PRO053 (comprising PRO034, PRO035, and PRO036, respectively).

SDS-PAGE and analytical size exclusion chromatography showed that the major product formed after cFAE reaction had a molecular weight of a typical IgG1 (150 kDa), suggesting complete reoxidation. Analytical hydrophobic interaction chromatography indicated the formation of a new product, the desired heterodimeric antibody.

### 12.4 Notch2/RAGE ANDbodies are Shown to Bind Simultaneously to Notch2 and RAGE by BLI

To test simultaneous dual antigen binding of the Notch2/RAGE ANDbodies by BLI, PRO051, PRO052 and PRO053, along with monovalent parental antibody controls were immobilized on anti-human IgG Fc biosensors and dipped into the recombinant His-tagged murine RAGE protein at 150 nM, followed by a second association step into wells containing recombinant murine Notch2 NRR at 150 nM to measure dual antigen binding. The dissociation rate was then measured by dipping the biosensors into buffer.

The sensorgrams showed that the ANDbodies PRO051, PRO052 and PRO053 were able to bind both RAGE and Notch2 antigens simultaneously, but the monovalent parental antibodies bound only one of RAGE and Notch2 NRR. This supports the conclusion that the ANDbodies were the correct composition and were functional in binding both antigens simultaneously.

### 12.5 Notch2/RAGE ANDbody are Shown to Bind Preferentially to Human Lung Tissue by Immunohistochemistry

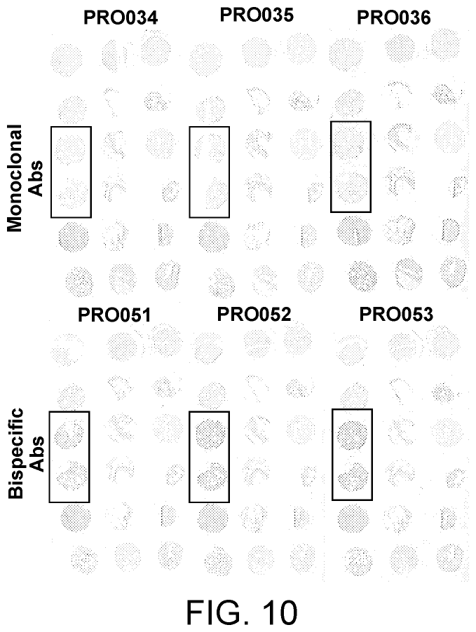

Immunohistochemistry (IHC) on fresh frozen healthy mouse tissue microarray (FF TMA) sections mounted onto glass slides was used to evaluate tissue binding by ANDbodies containing the Notch2 inhibitory antibodies described above. TMAs were constructed and stained as described in Example 11. FIG. 10 shows staining of mouse TMAs by the three anti-Notch2 antibodies PRO034, PRO035, and PRO036 alongside staining by the Notch2/RAGE ANDbodies PRO051, PRO052, and PRO053. In each case, there is a pronounced enhancement of binding to lung tissue in the RAGE-targeted ANDbody. These data show that combining a receptor-targeting binder with an addressing binder in antibody format can impart the tissue specificity of the addressing arm to the ANDbody.

### 12.6 A Notch2/RAGE ANDbody Distributes Preferentially to the Lungs Compared to a Matched Non-Targeted Anti-Notch2 Antibody

To evaluate how an ANDbody targeting Notch2 and RAGE behaves in vivo, mice were treated by IV dosing with the PRO051, PRO052, and PRO053 antibodies at 3 mg/kg. All groups contained 3 mice. At 3, 7, 14, and 21 days after dosing, tissues were collected from each mouse. The accumulation of each antibody in the lungs was measured by homogenizing a fixed amount of lung tissue, normalizing each sample to a fixed amount of extracted protein, and then detecting the human antibody by sandwich ELISA.

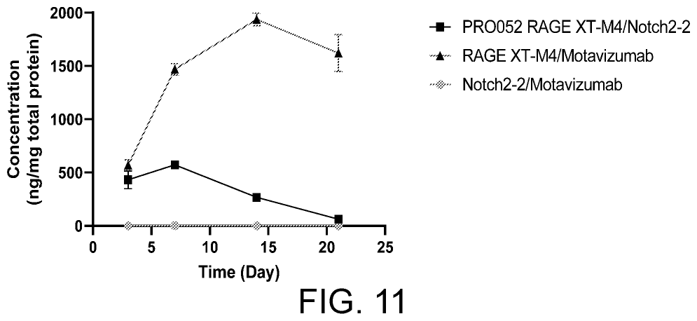

FIG. 11 shows accumulation in the lungs of PRO052 compared with a matched antibody that binds to RAGE and the control target respiratory syncytial virus (RSV) glycoprotein F (RAGE XT-M4/Motavizumab) and one that binds to Notch2 and RSV glycoprotein F (Notch2-2/Motavizumab). The antibody targeting RSV glycoprotein F and Notch2 was not detectable in the lungs at any time point. In contrast, the Notch2/RAGE ANDbody was clearly detectable in the lung for at least two weeks. Overall accumulation of PRO052 was lower than that of the bispecific that binds to RAGE and RSV glycoprotein F, indicating that the overall specificity of PRO052 is intermediate between the two arms independently. These results show that an addressing arm in an ANDbody can significantly redirect the binding specificity of a target-binding arm.

### Example 13. ANDbody Production and Use

This example describes the production of an exemplary ANDbody that (i) comprises a ligand effector that targets the IL-10 pathway and (ii) binds DSG1 as an address.

### 13.1 Description, Design and Production of IL-10/DSG1 ANDbodies

To test formats of IL-10/anti-DSG1 ANDbodies, three parameters were explored: valency of IL-10 (1 or 2 moieties of IL-10), valency of the anti-DSG1 arm (1 or 2 Fab arms) and two versions of IL-10 (dimeric or monomeric IL-10). Formats that represent different combinations of IL-10 molecules, IL-10 valency, and antibody valency were assessed. Wild-type (WT) IL-10 (accession number P22301), a monomeric engineered IL-10 sequence (Josephson et al., J Biol Chem., 275(18): 13552-7, 2000), and a dimeric engineered IL-10 sequence (Minshawi et al., Front Immunol., 11: 1794, 2020) were fused to PRO003 sequences (Example 7) at the C-terminus of heavy chain for the bivalent formats. For monovalent formats, monomeric and dimeric IL-10 were fused at the N-terminus of the Fc and co-expressed with PRO003. The monovalent formats are asymmetric, and mutations in the Fc domain (chain A: S364K/K409S; chain B: K370S/F405K (WO 2017/106462 A1)) are used to enforce asymmetric pairing.

To express and purify the antibodies, a 1:1 ratio of heavy chain to light chain DNA or a 1:1:1 ratio of heavy chain to light chain to IL-10-Fc (listed below) was transfected into EXPI293F™ cells (ThermoFisher Scientific) using the EXPIFECTAMINE™ 293 Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS.

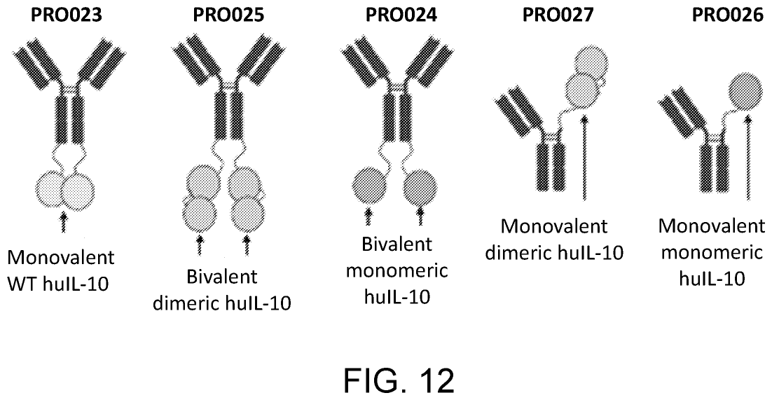

The resulting mAbs were designated as PRO023, PRO024, PRO025, PRO026 and PRO027 (FIG. 12).

PRO023 comprises (a) a heavy chain sequence (SEQ ID NO: 67) comprising the heavy chain sequence of PRO003 (SEQ ID NO: 28) and the wild-type human IL-10 sequence (SEQ ID NO: 64) and (b) the light chain sequence of PRO003 (SEQ ID NO: 29).

PRO024 comprises (a) a heavy chain sequence (SEQ ID NO: 68) comprising the heavy chain sequence of PRO003 (SEQ ID NO: 28) and the monomeric human IL-10 sequence (SEQ ID NO:64) and (b) the light chain sequence of PRO003 (SEQ ID NO: 29).

PRO025 comprises (a) a heavy chain sequence (SEQ ID NO: 69) comprising the heavy chain sequence of PRO003 (SEQ ID NO: 28) and the dimeric human IL-10 sequence (SEQ ID NO:66) and (b) the light chain sequence of PRO003 (SEQ ID NO: 29).

PRO026 comprises (a) the heavy chain sequence of PRO003, further comprising mutations in the Fc domain to enforce asymmetric pairing (SEQ ID NO: 70), (b) the light chain sequence of PRO003 (SEQ ID NO: 29), and (c) an IL-10-Fc fusion protein (SEQ ID NO: 72) comprising an Fc region including mutations to enforce asymmetric pairing (SEQ ID NO: 71) and a monomeric human IL-10 sequence (SEQ ID NO:64).

PRO027 comprises (a) the heavy chain sequence of PRO003, further comprising mutations in the Fc domain to enforce asymmetric pairing (SEQ ID NO: 70), (b) the light chain sequence of PRO003 (SEQ ID NO: 29), and (c) an IL-10-Fc fusion protein (SEQ ID NO: 73) comprising an Fc region including mutations to enforce asymmetric pairing (SEQ ID NO: 71) and the dimeric human IL-10 sequence (SEQ ID NO:66).

The purified ANDbodies were analyzed by analytical size exclusion chromatography for monodispersity and by SDS-PAGE for purity. PRO024 and PRO026 had the highest yields and monodispersity after single-step purification. PRO023 and PRO027 had moderate yields and were ˜70% monodispersed. PRO025 had lower yields with ˜89% monodispersity.

TABLE 11
PRO023, PRO024, PRO025, PRO026, PRO058, and PRO027 sequences

| | | SEQ ID |
|---|---|---|
| Region | Sequence | number |
| Wild-type (WT) IL- | SPGQGTQSENSCTHFPGNLPNMLRDLRDAFSRVKTFFQMK | SEQ ID NO: 64 |
| 10 (accession | DQLDNLLLKESLLEDFKGYLGCQALSEMIQFYLEEVMPQAEN | |
| number P22301) | QDPDIKAHVNSLGENLKTLRLRLRRCHRFLPCENKSKAVEQV | |
| | KNAFNKLQEKGIYKAMSEFDIFINYIEAYMTMKIRN | |
| Monomeric | SPGQGTQSENSCTHFPGNLPNMLRDLRDAFSRVKTFFQMK | SEQ ID NO: 65 |
| engineered IL-10 | DQLDNLLLKESLLEDFKGYLGCQALSEMIQFYLEEVMPQAEN | |
| sequence | QDPDIKAHVNSLGENLKTLRLRLRRCHRFLPCENGGGSGGK | |
| (Josephson et al., | SKAVEQVKNAFNKLQEKGIYKAMSEFDIFINYIEAYMTMKIRN | |
| J Biol Chem., | | |
| 275(18):13552-7, | | |
| 2000) | | |
| Dimeric | SPGQGTQSENSCTHFPGNLPNMLRDLRDAFSRVKTFFQMK | SEQ ID NO: 66 |
| engineered IL-10 | DQLDNLLLKESLLEDFKGYLGCQALSEMIQFYLEEVMPQAEN | |
| sequence | QDPDIKAHVNSLGENLKTLRLRLRRCHRFLPCENKSKAVEQV | |
| (Minshawi et al., | KNAFNKLQEKGIYKAMSEFDIFINYIEAYMTMKIRNGGGSGGG | |
| Front Immunol., | SPGQGTQSENSCTHFPGNLPNMLRDLRDAFSRVKTFFQMK | |
| 11:1794, 2020) | DQLDNLLLKESLLEDFKGYLGCQALSEMIQFYLEEVMPQAEN | |
| | QDPDIKAHVNSLGENLKTLRLRLRRCHRFLPCENKSKAVEQV | |
| | KNAFNKLQEKGIYKAMSEFDIFINYIEAYMTMKIRN | |
| PRO023 heavy | MGWSCIILFLVATATGVHSEVQLVESGGGLVQPGRSLRLSCA | SEQ ID NO: 67 |
| chain | ASGFTFDDYAMHWVRQAPGKGLEWVSGISWNSGSIDYADS | |
| | VKGRFTISRDNAKNSLYLQMNSLRVEDTALYYCAKDGSRVF | |
| | GVGGGFDFWGQGTMVTVSSASTKGPSVFPLAPSSKSTSGG | |
| | TAALGCLVKDYFPEPVTVSWNSGALTSGVHTFPAVLQSSGL | |
| | YSLSSVVTVPSSSLGTQTYICNVNHKPSNTKVDKKVEPKSCD | |
| | KTHTCPPCPAPEAAGGPSVFLFPPKPKDTLMISRTPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPRE | |
| | PQVYTLPPSRDELTKNQVSLTCLVKGFYPSDIAVEWESNGQP | |
| | ENNYKTTPPVLDSDGSFFLYSKLTVDKSRWQQGNVFSCSVM | |
| | HEALHNHYTQKSLSLSPGGGGGSGGGGSGGGGSSPGQGT | |
| | QSENSCTHFPGNLPNMLRDLRDAFSRVKTFFQMKDQLDNLL | |
| | LKESLLEDFKGYLGCQALSEMIQFYLEEVMPQAENQDPDIKA | |
| | HVNSLGENLKTLRLRLRRCHRFLPCENKSKAVEQVKNAFNKL | |
| | QEKGIYKAMSEFDIFINYIEAYMTMKIRN | |
| PRO024 heavy | MGWSCIILFLVATATGVHSEVQLVESGGGLVQPGRSLRLSCA | SEQ ID NO: 68 |
| chain | ASGFTFDDYAMHWVRQAPGKGLEWVSGISWNSGSIDYADS | |
| | VKGRFTISRDNAKNSLYLQMNSLRVEDTALYYCAKDGSRVF | |
| | GVGGGFDFWGQGTMVTVSSASTKGPSVFPLAPSSKSTSGG | |
| | TAALGCLVKDYFPEPVTVSWNSGALTSGVHTFPAVLQSSGL | |
| | YSLSSVVTVPSSSLGTQTYICNVNHKPSNTKVDKKVEPKSCD | |
| | KTHTCPPCPAPEAAGGPSVFLFPPKPKDTLMISRTPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPRE | |
| | PQVYTLPPSRDELTKNQVSLTCLVKGFYPSDIAVEWESNGQP | |
| | ENNYKTTPPVLDSDGSFFLYSKLTVDKSRWQQGNVFSCSVM | |
| | HEALHNHYTQKSLSLSPGGGGGSGGGGSGGGGSSPGQGT | |
| | QSENSCTHFPGNLPNMLRDLRDAFSRVKTFFQMKDQLDNLL | |
| | LKESLLEDFKGYLGCQALSEMIQFYLEEVMPQAENQDPDIKA | |
| | HVNSLGENLKTLRLRLRRCHRFLPCENGGGSGGKSKAVEQV | |
| | KNAFNKLQEKGIYKAMSEFDIFINYIEAYMTMKIRN | |
| PRO025 heavy | MGWSCIILFLVATATGVHSEVQLVESGGGLVQPGRSLRLSCA | SEQ ID NO: 69 |
| chain | ASGFTFDDYAMHWVRQAPGKGLEWVSGISWNSGSIDYADS | |
| | VKGRFTISRDNAKNSLYLQMNSLRVEDTALYYCAKDGSRVF | |
| | GVGGGFDFWGQGTMVTVSSASTKGPSVFPLAPSSKSTSGG | |
| | TAALGCLVKDYFPEPVTVSWNSGALTSGVHTFPAVLQSSGL | |
| | YSLSSVVTVPSSSLGTQTYICNVNHKPSNTKVDKKVEPKSCD | |
| | KTHTCPPCPAPEAAGGPSVFLFPPKPKDTLMISRTPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPRE | |
| | PQVYTLPPSRDELTKNQVSLTCLVKGFYPSDIAVEWESNGQP | |
| | ENNYKTTPPVLDSDGSFFLYSKLTVDKSRWQQGNVFSCSVM | |
| | HEALHNHYTQKSLSLSPGGGGGSGGGGSGGGGSSPGQGT | |
| | QSENSCTHFPGNLPNMLRDLRDAFSRVKTFFQMKDQLDNLL | |
| | LKESLLEDFKGYLGCQALSEMIQFYLEEVMPQAENQDPDIKA | |
| | HVNSLGENLKTLRLRLRRCHRFLPCENKSKAVEQVKNAFNKL | |
| | QEKGIYKAMSEFDIFINYIEAYMTMKIRNGGGSGGGSPGQGT | |
| | QSENSCTHFPGNLPNMLRDLRDAFSRVKTFFQMKDQLDNLL | |
| | LKESLLEDFKGYLGCQALSEMIQFYLEEVMPQAENQDPDIKA | |
| | HVNSLGENLKTLRLRLRRCHRFLPCENKSKAVEQVKNAFNKL | |
| | QEKGIYKAMSEFDIFINYIEAYMTMKIRN | |
| PRO026, | MGWSCIILFLVATATGVHSEVQLVESGGGLVQPGRSLRLSCA | SEQ ID NO: 70 |
| PRO058, and | ASGFTFDDYAMHWVRQAPGKGLEWVSGISWNSGSIDYADS | |
| PRO027 heavy | VKGRFTISRDNAKNSLYLQMNSLRVEDTALYYCAKDGSRVF | |
| chain | GVGGGFDFWGQGTMVTVSSASTKGPSVFPLAPSSKSTSGG | |
| | TAALGCLVKDYFPEPVTVSWNSGALTSGVHTFPAVLQSSGL | |
| | YSLSSVVTVPSSSLGTQTYICNVNHKPSNTKVDKKVEPKSCD | |
| | KTHTCPPCPAPEAAGGPSVFLFPPKPKDTLMISRTPEVTCVV | |
| | VDVSHEDPEVKFNWYVDGVEVHNAKTKPREEQYNSTYRVV | |
| | SVLTVLHQDWLNGKEYKCKVSNKALGAPIEKTISKAKGQPRE | |
| | PQVYTLPPSRDELTKNQVKLTCLVKGFYPSDIAVEWESNGQP | |
| | ENNYKTTPPVLDSDGSFFLYSSLTVDKSRWQQGNVFSCSVM | |
| | HEALHNHYTQKSLSLSPG | |
| Fc domain of IL-10 | DKTHTCPPCPAPEAAGGPSVFLFPPKPKDTLMISRTPEVTCV | SEQ ID NO: 71 |
| Fc fusion | VVDVSHEDPEVKFNWYVDGVEVHNAKTKPREEQYNSTYRV | |
| | VSVLTVLHQDWLNGKEYKCKVSNKALGAPIEKTISKAKGQPR | |
| | EPQVYTLPPSRDELTKNQVSLTCLVSGFYPSDIAVEWESNGQ | |
| | PENNYKTTPPVLDSDGSFKLYSKLTVDKSRWQQGNVFSCSV | |
| | MHEALHNHYTQKSLSLSPG | |
| PRO026 IL-10-Fc | SPGQGTQSENSCTHFPGNLPNMLRDLRDAFSRVKTFFQMK | SEQ ID NO: 72 |
| | DQLDNLLLKESLLEDFKGYLGCQALSEMIQFYLEEVMPQAEN | |
| | QDPDIKAHVNSLGENLKTLRLRLRRCHRFLPCENGGGSGGK | |
| | SKAVEQVKNAFNKLQEKGIYKAMSEFDIFINYIEAYMTMKIRN | |
| | GGGGSGGGGSDKTHTCPPCPAPEAAGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALGAPI | |
| | EKTISKAKGQPREPQVYTLPPSRDELTKNQVSLTCLVSGFYP | |
| | SDIAVEWESNGQPENNYKTTPPVLDSDGSFKLYSKLTVDKSR | |
| | WQQGNVFSCSVMHEALHNHYTQKSLSLSPG | |
| PRO027 IL-10-Fc | SPGQGTQSENSCTHFPGNLPNMLRDLRDAFSRVKTFFQMK | SEQ ID NO: 73 |
| | DQLDNLLLKESLLEDFKGYLGCQALSEMIQFYLEEVMPQAEN | |
| | QDPDIKAHVNSLGENLKTLRLRLRRCHRFLPCENKSKAVEQV | |
| | KNAFNKLQEKGIYKAMSEFDIFINYIEAYMTMKIRNGGGSGGG | |
| | SPGQGTQSENSCTHFPGNLPNMLRDLRDAFSRVKTFFQMK | |
| | DQLDNLLLKESLLEDFKGYLGCQALSEMIQFYLEEVMPQAEN | |
| | QDPDIKAHVNSLGENLKTLRLRLRRCHRFLPCENKSKAVEQV | |
| | KNAFNKLQEKGIYKAMSEFDIFINYIEAYMTMKIRNGGGGSGG | |
| | GGSDKTHTCPPCPAPEAAGGPSVFLFPPKPKDTLMISRTPEV | |
| | TCVVVDVSHEDPEVKFNWYVDGVEVHNAKTKPREEQYNST | |
| | YRVVSVLTVLHQDWLNGKEYKCKVSNKALGAPIEKTISKAKG | |
| | QPREPQVYTLPPSRDELTKNQVSLTCLVSGFYPSDIAVEWES | |
| | NGQPENNYKTTPPVLDSDGSFKLYSKLTVDKSRWQQGNVFS | |
| | CSVMHEALHNHYTQKSLSLSPG | |
| Fc domain of IL-10 | DKTHTCPPCPAPEAAGGPSVFLFPPKPKDTLMISRTPEVTCV | SEQ ID NO: 74 |
| Fc fusion (with | VVDVSHEDPEVKFNWYVDGVEVHNAKTKPREEQYNSTYRV | |
| substitutions) | VSVLTVLHQDWLNGKEYKCKVSNKALGAPIEKTISKAKGQPR | |
| | EPQVYTLPPSRDELTKNQVSLTCLVSGFYPSDIAVEWESNGQ | |
| | PENNYKTTPPVLDSDGSFKLYSKLTVDKSRWQQGNVFSCSV | |
| | MHEALHNRFTQKSLSLSPG | |
| PRO058 IL-10-Fc | SPGQGTQSENSCTHFPGNLPNMLRDLRDAFSRVKTFFQMK | SEQ ID NO: 75 |
| | DQLDNLLLKESLLEDFKGYLGCQALSEMIQFYLEEVMPQAEN | |
| | QDPDIKAHVNSLGENLKTLRLRLRRCHRFLPCENGGGSGGK | |
| | SKAVEQVKNAFNKLQEKGIYKAMSEFDIFINYIEAYMTMKIRN | |
| | GGGGSGGGGSDKTHTCPPCPAPEAAGGPSVFLFPPKPKDT | |
| | LMISRTPEVTCVVVDVSHEDPEVKFNWYVDGVEVHNAKTKP | |
| | REEQYNSTYRVVSVLTVLHQDWLNGKEYKCKVSNKALGAPI | |
| | EKTISKAKGQPREPQVYTLPPSRDELTKNQVSLTCLVSGFYP | |
| | SDIAVEWESNGQPENNYKTTPPVLDSDGSFKLYSKLTVDKSR | |
| | WQQGNVFSCSVMHEALHNRFTQKSLSLSPG | |

### 13.2 IL-10/DSG1 ANDbodies Bind to Both IL-10 Receptors

To test the binding of the IL-10/anti-DSG1 ANDbodies to IL-10 receptor alpha (IL-10Ra) by ELISA, recombinant His-tagged human IL-10Ra (Creative Biomart) was coated on NUNC-IMMUNO™ MAXISORP™ ELISA plates at 1 μg/mL concentration overnight. The next day, coated antigen was removed and the wells were blocked with 1% IgG-free BSA followed by incubation with 11 three-fold serially diluted anti-IL-10/anti-DSG1 ANDbodies (as provided above) with a starting concentration of 30 μg/mL. Bound antibody was detected with peroxidase-conjugated anti-human IgG antibodies with TMB and acid stop reagents.

Of the molecules tested, only those containing an IL-10 moiety (PRO023, PRO024, PRO025, PRO026, PRO027, and the positive control IL-10 Fc) (Creative Biomart IL10-326H) bound IL-10Ra, demonstrating that the binding was driven by IL-10 and not the negative control anti-DSG1 antibody (PRO003) and that the IL-10 moiety was functional in binding its receptor.

### 13.3 IL-10/DSG1 ANDbodies Activate the IL-10 Signaling Pathway

To show that IL-10 retains its biological activity as a part of the various ANDbodies described above, the ability to each IL-10/DSG1 ANDbody to activate the IL-10 signaling pathway was tested. HEK-BLUE™ IL-10 cells (InvivoGen) were used to evaluate the signaling activity and relative potency of each molecule. These cells express all the components of the IL-10 signaling pathway, including an IL-10-inducible gene encoding secreted embryonic alkaline phosphatase (SEAP). When IL-10 signaling is activated in these cells, they express and secrete SEAP in to the cell culture media. The degree of IL-10 signal is measured by adding QUANTI-BLUE™ solution colorimetric reagent (InvivoGen) to the cell culture media, followed by reading absorbance at 630 nm.

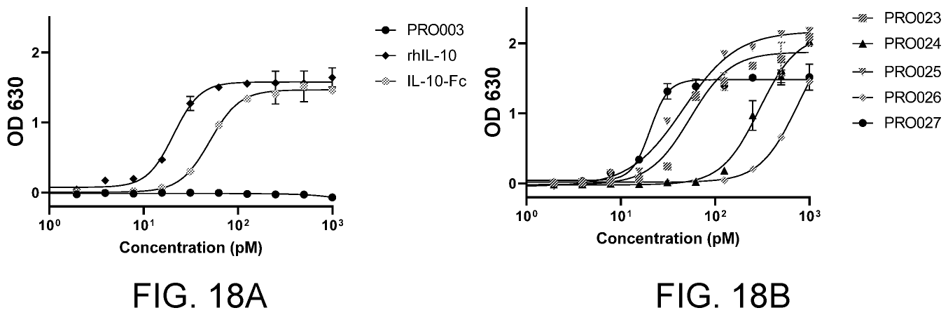

In this experiment, each of PRO023, PRO024, PRO025, PRO026, and PRO027 was titrated from 1 pM to >1 nM with overnight incubation in cell culture. FIGS. 18A and 18B show representative activity data for each IL-10/DSG1 ANDbody, along with positive and negative controls as described in Table 12. Table 12 shows the EC50 for response of HEK-BLUE™ cells to three control molecules ((1) recombinant human IL-10 (BioLegend #573204) (rhIL-10), (2) recombinant human IL-10 fused to a human Fc domain (hIL-10 Fc fusion), and (3) the parental anti-DSG1 antibody (PRO003)) and to each IL-10/DSG1 ANDbody described above. These data confirm that all five IL-10/DSG1 ANDbodies retain the signaling activity of human IL-10. They show a range of potency, indicating that the relative biological potency of ANDbodies can be adjusted by variations in the format of the molecule, structure of the biologically active moiety, and valency of the active moiety.

TABLE 12
Potency of IL-10/DSG1 ANDbodies in the HEK-
BLUE ™ IL-10 signaling assay

| | | Address | IL-10 | EC50 |
|---|---|---|---|---|
| Molecule | Description | valency | valency | (pM) |
| rhIL-10 | Human IL-10 (positive control) | 0 | 2 | 21 |
| hIL-10 Fc | Human IL-10 fused (positive | 0 | 2 | 50 |
| fusion | control) | | | |
| Anti- | Negative control (targeting | 2 | 0 | NA |
| DSG1 | moiety alone) | | | |
| mAb | | | | |
| PRO003 | | | | |
| PRO023 | Native IL-10 fused to mAb C- | 2 | 1 | 54 |
| | termini | | | |
| PRO024 | Monomeric IL-10 fused to mAb C- | 2 | 2 | 293 |
| | termini | | | |
| PRO025 | IL-10 dimer fused to each mAb C- | 2 | 2 | 46 |
| | terminus | | | |
| PRO026 | Monomeric IL-10 replacing one | 1 | 1 | 750 |
| | Fab of the anti-DSG1 mAb | | | |
| PRO027 | IL-10 dimer replacing one Fab | 1 | 1 | 19 |
| | of the anti-DSG1 mAb | | | |

### 13.4 IL-10/DSG1 ANDbodies Suppress the Inflammatory Response in Primary Mouse Macrophages

To show that IL-10/DSG1 ANDbodies are able to inhibit inflammatory immune responses, the effects of IL-10/DSG1 ANDbodies on mouse peripheral blood mononucleocytes (PBMCs) and macrophages treated with lipid polysaccharide (LPS) as an inflammatory stimulus were evaluated. In these experiments, PBMCs were isolated from blood and macrophages were isolated from the spleens of Balb/C mice by negative enrichment on magnetic beads (Miltenyi Biotech #130-110-434). Macrophage activation was assayed by measuring the level of TNFα cytokine present in the media after 3 hours and 5-6 hours of stimulation with LPS.

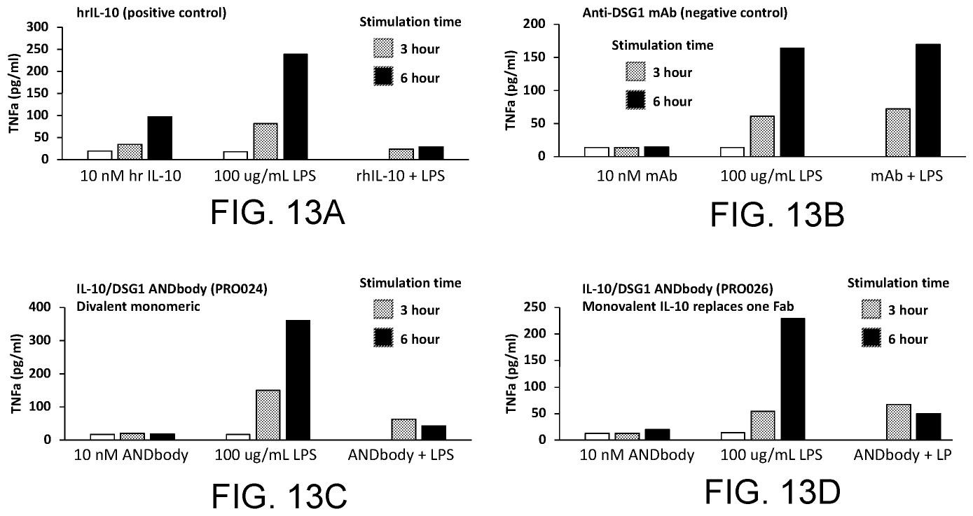

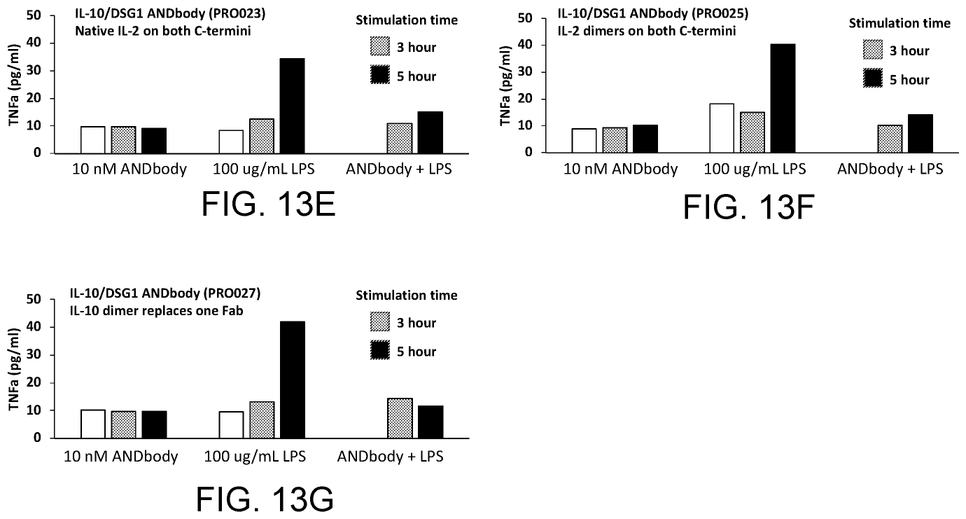

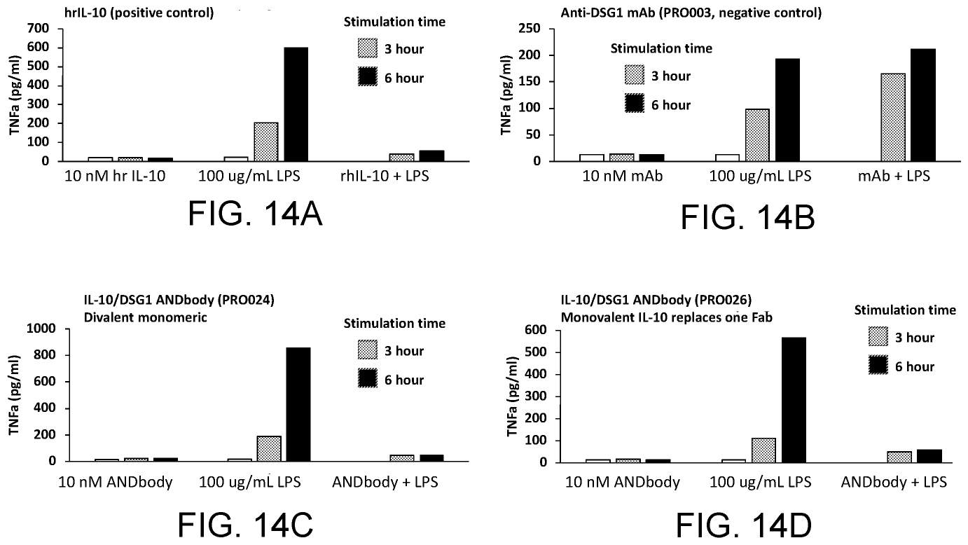

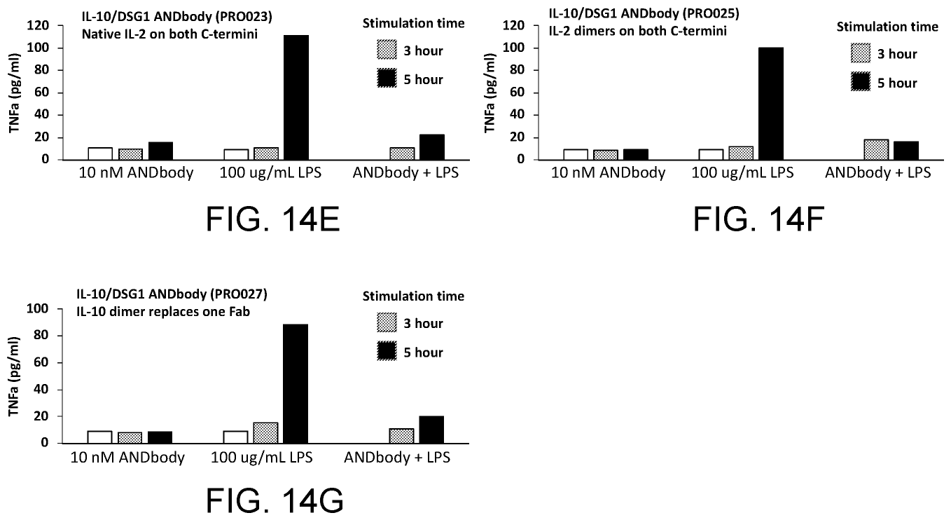

FIGS. 13A-13G show the level of tumor necrosis factor alpha (TNFα) in PBMC cell culture after pre-stimulation with the indicated IL-10/DSG1 ANDbodies or control molecules followed by treatment with LPS for the indicated lengths of time. FIGS. 14A-14G show the level of TNFα in primary macrophage cell culture after pre-stimulation with the indicated IL-10/DSG1 ANDbodies or control molecules followed by treatment with LPS for the indicated lengths of time. Because the panels represent experiments run across multiple days, data are not comparable between panels. These data show that all five IL-10/DSG1 ANDbodies are able to suppress inflammatory stimuli in primary macrophages.

### 13.5 Engagement of an Address Target Enhances the Activity/Potency of the Effector Function of an ANDbody

The combination of addressing (e.g., using an address targeting domain) with a biologically active molecule has the potential to enhance a biological activity in a variety of ways. One exemplary enhancement is by increasing the potency of the effector moiety on specific cells where the address target is also present.

To test whether signaling potency of the effector targeting domain can be enhanced by the presence of an address targeting domain, human DSG1 was expressed on the HEK-BLUE™ IL-10 cells using stable expression with a lentivirus (the stable expressing cells are denoted HEKBLUE™ IL-10/DSG1). The DSG1 gene (NP_034209.2) was cloned into a suitable lentiviral plasmid backbone, packaged into viral particles using the VIRAPOWER™ Lentiviral Packing Mix (ThermoFisher Scientific) and transduced according to the manufacturer's instructions. Expression of DSG1 was confirmed by qPCR.

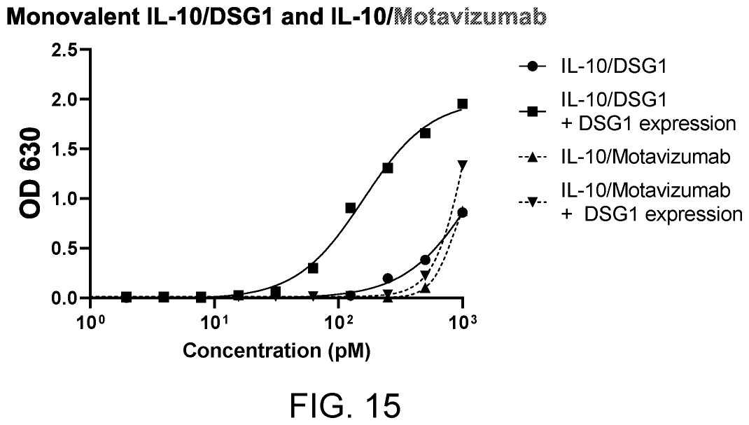

The potency of recombinant human IL-10, an ANDbody in which monomeric IL-10 replaces one Fab of the anti-DSG1 mAb (PRO058, functionally equivalent to PRO026), and a matched control in which the antibody sequence contains Motavizumab as a negative control were evaluated. Activity was tested on HEK-BLUE™ IL-10 cells and on HEK-BLUE™ IL-10 cells stably expressing DSG1. FIG. 15 shows the representative signaling response of each molecule in the parental HEK-BLUE™ IL-10 cells and the HEK-BLUE™ IL-10/DSG1 cells. The two cell lines responded similarly to recombinant IL-10, confirming that DSG1 expression did not have a large impact on their sensitivity to IL-10. The DSG1/IL-10 ANDbody showed approximately a 15-fold increase in potency when DSG1 was expressed on the target cells. No effect was seen on the potency of a matched IL-10/Motavizumab protein, confirming that the effect is mediated by binding to DSG1. This shows that the ANDbody designs provided herein are capable of address-mediated enhancement in their biological potency, allowing the use of less potent target binder moieties in ANDbodies to further decrease unwanted off-target effects.

### 13.6 IL-10/DSG1 ANDbodies Retain the Pharmacokinetic and Tissue Distribution Properties of the Parental Anti-DSG1 Antibody

In some ANDbodies the targeting moiety is intended to impart the tissue or cellular targeting of a parental mAb or other targeting molecule onto a biologically active moiety that would otherwise have undesirable pharmacokinetics or tissue distribution.

The IL-10/DSG1 ANDbodies are intended to direct IL-10 activity to the skin. In Example 7, it was shown that this anti-DSG1 antibody distributes preferentially into the skin of mice. In contrast, IL-10 is reported to clear from circulation in humans with a half-life of approximately 2 hours (Radwanski et al, Pharm Res. 1998 December; 15(12):1895-901. We therefore evaluated whether IL-10/DSG1 ANDbodies retain the skin targeting capability of the parental antibody.

BALB/c mice were dosed by tail vein injection with 3 mg/kg of PRO003, PRO024, or PRO058. PRO058 is functionally equivalent to PRO026, with a substitution in the Fc domain to improve purification of the recombinant protein. PRO058 comprises (a) the heavy chain sequence of PRO0026 (SEQ ID NO: 70), (b) the light chain sequence of PRO003 (SEQ ID NO: 29), and (c) an IL-10-Fc fusion protein (SEQ ID NO: 75) comprising an Fc region (SEQ ID NO: 74) and a monomeric human IL-10 sequence (SEQ ID NO:64).

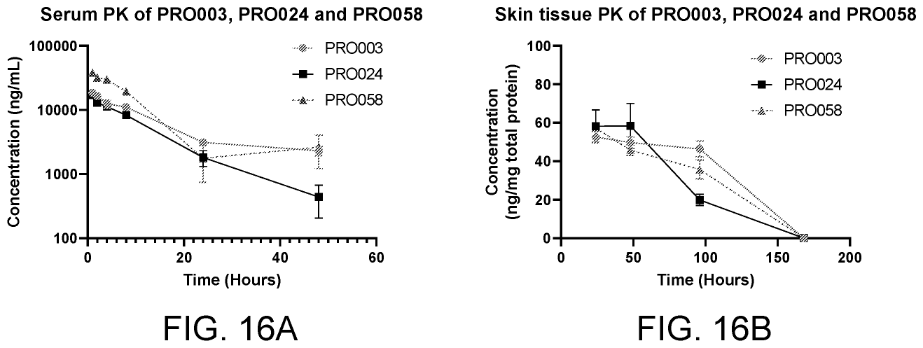

Serum samples were collected at time points from 1 hour to 48 hours. Tissue samples were collected at 1, 2, 4 and 7 days after dosing. The amount of anti-DSG1 or ANDbody in each serum or tissue sample was measured by ELISA. FIGS. 16A and 16B show that the IL-10/anti-DSG1 ANDbodies PRO024 and PRO058 have PK properties in skin and serum that are similar to the parental anti-DSG1 antibody PRO003. These data demonstrate that antibody-cytokine fusions comprising a recombinant IL-10 can retain the pharmacokinetic properties of the parental antibody.

### Example 14. TNFα-Blocking Molecules Coupled to DSG1-Targeting Moieties

This example describes the production of exemplary ANDbodies that block TNFα and bind DSG1 as an address.

### 14.1 Anti TNFα Monoclonal Antibody Expression and Purification

Anti TNFα antibodies having VH and VL sequences from a commercial antibody fused to a huIgG1 backbone with effector null mutations L234A, L235A, P329G (LALA-PG) were produced and their binding and affinity to TNFα was characterized as described above. The resulting mAbs were designated as PRO076 and PRO078.

### 14.2 Anti TNFα—DSG1 ANDbody Design, Expression and Purification

ANDbodies were designed by combining PRO004 (Example 7) with a previously reported dominant-negative TNFα (Steed et al. Science. 2003 Sep. 26; 301(5641) or clinically validated anti-TNFα antibodies listed below with the aim of locally downmodulating TNFα in the extracellular milieu of the inflamed skin. The TNFα-blocking anti-DSG1 ANDbody designs explored various formats and valencies, including cytokine/antibody and TNF receptor 2 (TNFR2)/antibody fusions.

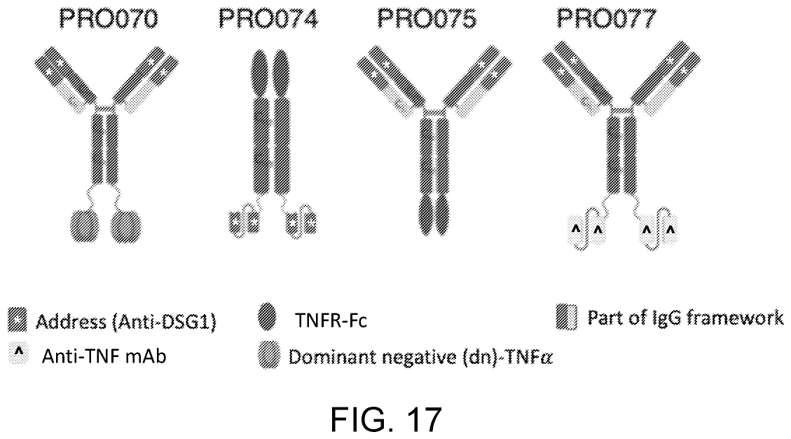

To express and purify the antibodies, a 1:1 ratio of heavy chain to light chain DNA was transfected into EXP1293F™ Cells (ThermoFisher Scientific) using the EXPIFECTAMINE™ 293 Transfection Kit (ThermoFisher Scientific) following the manufacturer's recommendations. Transiently expressed antibodies were purified from conditioned media 5 days post-transfection by filtering out the transfected cells. Conditioned media was incubated with protein A agarose beads for 1 hour. The bound beads were washed with Phosphate Buffered Saline (PBS) pH 7.4 followed by elution of the bound antibody with 0.1M Glycine pH 2.5 and neutralized with 1/10 volume of Tris pH 8.5. The neutralized eluate was buffer exchanged into PBS. The resulting mAbs were designated as PRO070, PRO074, PRO075 and PRO077 (FIG. 17).

The purified ANDbodies were analyzed by analytical size exclusion chromatography for monodispersity and by SDS-PAGE for purity. Standard additional purification steps were performed to remove aggregation. PRO077 had the highest final yield, followed by PRO074 and PRO075, while PRO070 had the lowest final yield. The polished ANDbodies were pure and of the correct composition as seen by SDS-PAGE.

### 14.3 Anti TNFα—DSG1 ANDbody Binding and Affinity Testing

ELISA binding assays demonstrated that all constructs were active in binding human and murine TNFα with varying affinities. PRO074 and PRO075 had similar affinities to both human and murine TNFα, which was within two/three-fold difference to the parent antibody. PRO077 had a 5-fold and a 12-fold decrease in binding affinity to human and murine TNFα, respectively, when compared to the parent antibody. This reduction in affinity is probably due to the change of format from Fab to single-chain variable fragment (scFv).

### 14.4 Anti TNFα—DSG1 ANDbody In Vitro Activity Assays

The ability of each anti-TNFα/anti-DSG1 ANDbody to inhibit TNFα signaling was evaluated using HEK-BLUE™ TNFα cells (InvivoGen). These cells are engineered to express secreted embryonic alkaline phosphatase (SEAP) in response to signaling to TNFα signaling. TNFα was measured according to the manufacturer's instructions. To evaluate inhibitory activity, the concentration of TNFα was fixed at 225 μm (approximately the EC₈₀ of a recombinant human TNFα in this assay), and cells were pre-incubated with concentrations of TNFα blocking molecules from 10 nM to approximately 10 μM. Table 13 shows the IC₅₀ of each anti-TNFα/DSG1 ANDbody alongside matched parent antibodies as positive controls. These data show that the ANDbodies retain TNFα blocking activity comparable to the original anti-TNFα parent antibodies.

TABLE 13
IC₅₀ of anti-TNFα/anti-DSG1 ANDbodies
compared to matched controls

| Protein | IC₅₀ (nM, replicate 1) | IC₅₀ (nM, replicate 2) |
|---|---|---|
| Adalimumab (PRO076) | 0.05 | 0.03 |
| Etanercept (PRO078) | 0.06 | 0.07 |
| PRO070 | No inhibition | No inhibition |
| PRO074 | 0.3 | 0.2 |
| PRO075 | 0.2 | 0.1 |
| PRO077 | 0.04 | 0.04 |

### VII. OTHER EMBODIMENTS

Some embodiments of the technology described herein can be defined according to any of the following numbered embodiments:
1. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site, wherein the macromolecule is linked to a small molecule. 2. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and wherein localization of the macromolecule to a non-target tissue or cell is substantially reduced relative to localization of a reference macromolecule lacking the second binding site, wherein the macromolecule is linked to a small molecule.
3. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site; and wherein localization of the macromolecule to a target tissue or cell is substantially increased relative to localization of a reference macromolecule lacking the second binding site, wherein the macromolecule is linked to a small molecule.
4. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and wherein at least 25% of the macromolecule administered to a subject is detected at the target tissue or cell at a time point between 1 and 7 days following administration, wherein the macromolecule is linked to a small molecule.
5. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and wherein the affinity of the first binding site for the effector target is lower than the affinity of the second binding site for the address target, wherein the macromolecule is linked to a small molecule.
6. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and wherein the avidity of the first binding site for the effector target is lower than the avidity of the second binding site for the address target, wherein the macromolecule is linked to a small molecule.
7. A macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and wherein the potency of the first binding site at the target tissue or cell is substantially increased relative to a reference macromolecule lacking the second binding site, wherein the macromolecule is linked to a small molecule.
8. The macromolecule of any one of embodiments 1-7, wherein the first binding site has a low affinity for the effector target.
9. The macromolecule of any one of embodiments 1-7, wherein the first binding site has a low avidity for the effector target.
10. The macromolecule of any one of embodiments 1-4 and 6-9, wherein the affinity of the first binding site for the effector target is lower than the affinity of the second binding site for the address target.
11. The macromolecule of any one of embodiments 1-10, wherein the avidity of the first binding site for the effector target is lower than the avidity of the second binding site for the address target.
12. The macromolecule of any one of embodiments 1-11, wherein:
(a) the Kd of the first binding site for the effector target is higher than the Kd of the second binding site for the address target;
(b) the EC₅₀ of the first binding site for the effector target is higher than the EC₅₀ of the second binding site for the address target; or
(c) the IC₅₀ of the first binding site for the effector target is higher than the IC₅₀ of the second binding site for the address target.
13. The macromolecule of any one of embodiments 1-12, wherein the first binding site has an affinity to the effector target of at least about 2 times, at least about 5 times, or at least about 10 times less than the affinity of the second binding site to the address target.
14. The macromolecule of any one of embodiments 1-13, wherein the affinity of the second binding site to the address target has a Kd of greater than about 1 nM, greater than about 2 nM, or greater than about 50 nm.
15. The macromolecule of any one of embodiments 1-14, wherein the effector target is a protein, lipid, or sugar.
16. The macromolecule of any one of embodiments 1-15, wherein the effector target is a cell membrane-associated target.
17. The macromolecule of embodiment 15 or 16, wherein the effector target is a protein.
18. The macromolecule of embodiment 17, wherein the effector target is a secreted protein.
19. The macromolecule of embodiment 17 or 18, wherein the effector target is encoded by a gene selected from the group consisting of the genes recited in Table 1.
20. The macromolecule of any one of embodiments 1-19, wherein the macromolecule agonizes the effector target.
21. The macromolecule of any one of embodiments 1-19, wherein the macromolecule antagonizes the effector target.
22. The macromolecule of any one of embodiments 1-21, wherein the address target is a protein, lipid, or sugar.
23. The macromolecule of embodiment 22, wherein the address target is a protein.
24. The macromolecule of any one of embodiments 17-23, wherein expression of the effector target or the address target is expression of an RNA sequence encoding the effector target or the address target.
25. The macromolecule of embodiment 24, wherein the expression level of the effector target or the address target is assessed by using a RNA sequence dataset.
26. The macromolecule of embodiment 25, wherein the RNA sequence dataset is a Genotype-Tissue Expression (GTEx) dataset or a Human Protein Atlas (HPA) dataset.
27. The macromolecule of embodiment 23, wherein expression of the effector target or the address target is protein expression.
28. The macromolecule of any one of embodiments 1-27, wherein the effector target is systemically expressed in the subject.
29. The macromolecule of any one of embodiments 1-27, wherein the effector target is regionally expressed in the subject.
30. The macromolecule of any one of embodiments 1-27, wherein the effector target is locally expressed in the subject.
31. The macromolecule of any one of embodiments 1-30, wherein the address target is regionally expressed in the subject.
32. The macromolecule of any one of embodiments 1-30, wherein the address target is locally expressed in the subject.
33. The macromolecule of any one of embodiments 1-30, wherein the expression of the address target is restricted to a cell type in the subject.
34. The macromolecule of any one of embodiments 1-33, wherein the address target is a soluble protein or an extracellular matrix (ECM)-associated protein and is not present in detectable amounts on the cell surface.
35. The macromolecule of embodiment 34, wherein the address target is expressed in the ECM and is not present in detectable amounts elsewhere in the subject.
36. The macromolecule of any one of embodiments 1-35, wherein the address target is expressed only by a cell in the subject when in a specific cell state.
37. The macromolecule of any one of embodiments 1-36, wherein the address target is expressed only by a cell in the subject when in a disease state.
38. The macromolecule of any one of embodiments 1-37, wherein the address target is not expressed in a tissue in which binding of the second binding site to the effector target is deleterious to the subject.
39. The macromolecule of any one of embodiments 1-38, wherein the binding site for the address target does not bind in detectable amounts to the binding site of a natural ligand of the address target.
40. The macromolecule of any one of embodiments 1-39, wherein expression of the effector target or address target includes expression in one or more of minor salivary gland, thyroid, lung, breast, mammary tissue, pancreas, adrenal gland, liver, kidney, kidney cortex, kidney medulla, adipose-visceral tissue, omentum, small intestine, terminal ileum, fallopian tube, ovary, uterus, skin, skin not sun exposed, suprapubic skin, cervix, endocervix, ectocervix, vagina, skin sun exposed, lower leg skin, eneanterior cingulate cortex, Brodmann area 24 (BA24), basal ganglia, caudate nucleus, putamen, nucleus acumbens, hypothalamus, amygdala, hippocampus, cerebellum, cerebellar hemisphere, substantia nigra, pituitary gland, spinal cord, cervical spinal cord, artery, aorta, heart, atrial appendage, coronary artery, left ventricle, esophagus, esophagus mucosa, esophagus muscularis, gastroesophageal junction, spleen, stomach, colon, transverse colon, sigmoid colon, testis, whole blood cells, EBV-transformed lymphocytes, artery-tibial, or nerve-tibial tissues.
41. The macromolecule of embodiment 40, wherein expression of the effector target or address target includes expression in skin tissue, lung tissue, kidney tissue, or intestine tissue.
42. The macromolecule of embodiment 41, wherein expression of the address target is substantially higher in skin tissue, lung tissue, kidney tissue, or intestine tissue than in any other tissue.
43. The macromolecule of any one of embodiments 1-42, wherein the effector target and/or the address target is expressed on a structural tissue in the subject.
44. The macromolecule of any one of embodiments 1-43, wherein the effector target and address target are on the same cell.
45. The macromolecule of any one of embodiments 1-43, wherein the effector target and address target are on different cells.
46. The macromolecule of embodiment 45, wherein the effector target and address target are on different cells of the same cell type.
47. The macromolecule of embodiment 45, wherein the effector target and address target are on different cells of different cell types.
48. The macromolecule of embodiment 45, wherein the effector target and address target are on different cells in the same tissue.
49. The macromolecule of any one of embodiments 45, 47, and 48, wherein:
(a) the effector target is on a circulating cell and the address target is on a tissue-restricted cell; or
(b) the effector target is on a tissue-restricted cell and the address target is on a circulating cell.
50. The macromolecule of any one of embodiments 45-49, wherein the effector target and address target are on different cells located within 100 nm of each other in the subject.
51. The macromolecule of any one of embodiments 45-49, wherein either the effector target or the address target is present on a cell surface.
52. The macromolecule of any one of embodiments 1-51, wherein the macromolecule is a DNA polynucleotide.
53. The macromolecule of any one of embodiments 1-51, wherein the macromolecule comprises an RNA or RNA-polypeptide conjugate.
54. The macromolecule of any one of embodiments 1-51 and 53, wherein the macromolecule comprises a polypeptide.
55. The macromolecule of any one of embodiments 1-51, wherein the macromolecule is a polypeptide.
56. The macromolecule of embodiment 54 or 55, wherein the polypeptide is an antibody or antigen-binding fragment thereof.
57. The macromolecule of embodiment 56, wherein the first binding site and the second binding site each comprise a VH and/or a VL.
58. The macromolecule of embodiment 57, wherein the macromolecule is an antibody comprising a first binding site that is specific for the effector target in the subject and a second binding site that is specific for the address target.
59. The macromolecule of embodiment 57 or 58, wherein the macromolecule is an asymmetric antibody or a symmetric antibody.
60. The macromolecule of any one of embodiments 56-59, wherein the antibody or antigen-binding fragment thereof comprises an scFv, BsIgG, a BsAb fragment, a BiTE, a dual-affinity re-targeting protein (DART), a tandem diabody (TandAb), a diabody, an Fab2, a di-scFv, chemically linked F(ab′)2, an Ig molecule with 2, 3 or 4 different antigen binding sites, a DVI-IgG four-in-one, an ImmTac, an HSAbody, an IgG-IgG, a Cov-X-Body, an scFv1-PEG-scFv₂, an appended IgG, an DVD-IgG, an affibody, an affilin, an affimer, an affitin, an alphabody, an anticalin, an avimer, a DARPin, a Fynomer, a monobody, a nanoCLAMP, a bis-Fab, an Fv, a Fab, a Fab′-SH, a linear antibody, an scFv, an antibody with only a heavy chain (Humabody), an ScFab, an IgG antibody fragment, a single-chain variable region antibody, a single-domain heavy chain antibody. a bispecific triplebody, a BiKE, a CrossMAb, a dsDb, an scDb, tandem a dAb/VHH, a triple dAb VHH, a tetravalent dAb/VHH, a Fab-scFv, a Fab-Fv, or a DART-Fc, an adnectin, a Kunitz-type inhibitor, or a receptor decoy.
61. The macromolecule of embodiment 54, wherein the polypeptide is a ligand of the effector target or a ligand of the address target.
62. The macromolecule of embodiment 61, wherein the ligand is a natural ligand, a modified ligand, or a synthetic ligand.
63. The macromolecule of embodiment 61 or 62, wherein the effector target or address target is a receptor and the polypeptide is a ligand thereof.
64. The macromolecule of any one of embodiments 61-63, wherein the first binding site comprises an antibody or antigen-binding fragment thereof and the second binding site comprises a ligand of the address target.
65. The macromolecule of any one of embodiments 61-63, wherein the first binding site comprises a ligand of the effector target and the second binding site comprises an antibody or antigen-binding fragment thereof.
66. The macromolecule of any one of embodiments 1-51 and 54-65, wherein the amino acid sequences of the first and second binding sites are at least about 10% identical, at least about 20% identical, at least about 30% identical, at least about 40% identical, at least about 50% identical, at least about 60% identical, or at least about 70% identical.
67. The macromolecule of any one of embodiments 1-66, wherein the address target has a Gini coefficient higher than about 0.4, about 0.5, about 0.57, about 0.65, about 0.7, about 0.85, about 0.90, or about 0.95.
68. The macromolecule of any one of embodiments 1-67, wherein the address target has a Tau coefficient higher than about 0.67, about 0.75, about 0.8, about 0.85, about 0.90, or about 0.95.
69. The macromolecule of any one of embodiments 1-68, wherein the effector target has a Gini coefficient lower than about 0.25, about 0.20, or about 0.15.
70. The macromolecule of any one of embodiments 1-69, wherein the effector target has a Tau coefficient lower than about 0.25, about 0.20, or about 0.15.
71. The macromolecule of any one of embodiments 1-70, further comprising a third binding site.
72. The macromolecule of embodiment 71, wherein the third binding site is the same as the first binding site.
73. The macromolecule of embodiment 71, wherein the third binding site is the same as the second binding site.
74. The macromolecule of any one of embodiments 1-73, wherein the first binding site and second binding site are directly joined to each other in the macromolecule.
75. The macromolecule of any one of embodiments 1-73, wherein the first binding site and the second binding site in the macromolecule are joined by a stable domain.
76. The macromolecule of any one of embodiments 1-75, wherein the effector target is Notch2 and the address target is RAGE.
77. The macromolecule of embodiment 76, wherein RAGE signaling is not influenced by the second site binding the RAGE address target.
78. The macromolecule of any one of embodiments 1-75, wherein the effector target is Notch2 and the address target is uromodulin (UMOD).
79. The macromolecule of embodiment 78, wherein UMOD signaling is not influenced by the second site binding the UMOD address target.
80. The macromolecule of any one of embodiments 1-75, wherein the effector target is Notch2 and the address target is meprin A subunit beta (MEP1B).
81. The macromolecule of embodiment 80, wherein MEP1B signaling is not influenced by the second site binding the MEP1B address target.
82. The macromolecule of any one of embodiments 1-75, wherein the effector target is I-11Ra and the address target is RAGE.
83. The macromolecule of embodiment 82, wherein RAGE signaling is not influenced by the second site binding the RAGE address target.
84. The macromolecule of any one of embodiments 1-75, wherein the effector target is IL 11Ra and the address target is UMOD.
85. The macromolecule of embodiment 84, wherein UMOD signaling is not influenced by the second site binding the UMOD address target.
86. The macromolecule of any one of embodiments 1-85, wherein the subject is a human.
87. A method of delivering a moiety to a target tissue or cell in a subject, comprising administering to the subject a macromolecule of any one of embodiments 1-86, wherein the target tissue comprises the address target.
88. The method of embodiment 87, wherein the moiety is a molecule.
89. The method of embodiment 87 or 88, wherein the moiety is not a toxin.
90. The method of embodiment 87, wherein the moiety is a cell.
91. The method of embodiment 90, wherein the moiety is not a T cell or an NK cell.
92. The method of any one of embodiments 87-91, wherein the target tissue is not a tumor.
93. A method of modulating an effector target in a target tissue, comprising administering to the tissue a macromolecule of any one of embodiments 1-86, wherein the target tissue comprises the address target and the effector target.
94. A method of biasing a binding agent away from binding an effector target when the effector target is found in the heart or lungs, comprising administering the macromolecule of any one of embodiments 1-86, wherein the address target is not substantially expressed in the heart or lungs.
95. A method of modulating a target tissue in a subject, comprising administering to the subject a macromolecule of any one of embodiments 1-86, wherein the target tissue comprises the address target and the effector target.
96. A method of treating a subject having a disease or condition associated with an effector target, comprising administering to the subject a macromolecule of any one of embodiments 1-86, wherein the first binding site of the macromolecule binds the effector target.
97. A macromolecule comprising a first binding site and a second binding site, wherein:
(a) the first binding site is specific for an effector target in a subject, and
(b) the second binding site is specific for an address target expressed in a target tissue or cell in the subject;
wherein the second binding site localizes the first binding site to the address target such that the first binding site influences effector target signaling in the target tissue or cell,
wherein the first binding site does not substantially influence effector target signaling in the absence of localization by the second binding site, and
wherein the second binding site does not bind to the binding site of the natural ligand of the address target, wherein the macromolecule is linked to a small molecule.
98. A macromolecule comprising a first binding site and a second binding site, wherein:
(a) the first binding site is specific for an effector target in a subject, and
(b) the second binding site is specific for an address target expressed in a target tissue or cell in the subject;
wherein the second binding site localizes the first binding site to the address target such that the first binding site influences effector target signaling in the target tissue or cell,
wherein the first binding site does not substantially influence effector target signaling in the absence of localization by the second binding site, and
wherein the first binding site and second binding site are directly joined to each other in the macromolecule, wherein the macromolecule is linked to a small molecule.
99. A macromolecule comprising a first binding site and a second binding site, wherein:
(a) the first binding site is specific for an effector target in a subject, and
(b) the second binding site is specific for an address target expressed in a target tissue or cell in the subject;
wherein the second binding site localizes the first binding site to the address target such that the first binding site influences effector target signaling in the target tissue or cell,
wherein the first binding site does not substantially influence effector target signaling in the absence of localization by the second binding site, and
wherein the first binding site and second binding are joined to each other by a stable domain, wherein the macromolecule is linked to a small molecule.
100. A macromolecule comprising a first binding site and a second binding site, wherein:
(a) the first binding site is specific for an effector target in a subject, and
(b) the second binding site is specific for an address target expressed in a target tissue or cell in the subject;
wherein the second binding site localizes the first binding site to the address target such that the first binding site influences effector target signaling in the target tissue or cell,
wherein the first binding site does not substantially influence effector target signaling in the absence of localization by the second binding site, and
wherein the effector target and/or the address target is expressed on a structural tissue in a host, wherein the macromolecule is linked to a small molecule.
101. A pharmaceutical composition comprising the macromolecule of any one of embodiments 1-86.
102. A pharmaceutical composition comprising a macromolecule and one or more pharmaceutically acceptable excipients,
wherein the macromolecule comprises a first binding site and a second binding site, wherein:
(a) the first binding site is specific for an effector target in a subject, and
(b) the second binding site is specific for an address target expressed in a target tissue or cell in the subject;
wherein the second binding site localizes the first binding site to the address target such that the first binding site influences effector target signaling in the target tissue or cell, and
wherein the first binding site does not substantially influence effector target signaling in the absence of localization by the second binding site, wherein the macromolecule is linked to a small molecule.
103. The pharmaceutical composition of embodiment 101 or 102, wherein the pharmaceutical composition is an RNA pharmaceutical composition.
104. The pharmaceutical composition of any one of embodiments 101-103, further comprising a carrier.
105. The pharmaceutical composition of embodiment 104, wherein the carrier is a lipid nanoparticle.
106. The pharmaceutical composition of embodiment 104, wherein the carrier is a viral vector.
107. The pharmaceutical composition of embodiment 104, wherein the carrier is a membrane-based carrier.
108. The pharmaceutical composition of embodiment 107, wherein the membrane-based carrier is a cell.
109. The pharmaceutical composition of embodiment 107, wherein the membrane-based carrier is a vesicle.
110. A method for modulating activity of an effector target in the skin of a subject, the method comprising administering to the subject a macromolecule comprising a first binding site and a second binding site, wherein:
(a) the first binding site is specific for an effector target in the subject, and
(b) the second binding site is specific for desmoglein-1 (DSG-1), wherein the macromolecule is linked to a small molecule.
111. A method for modulating activity of an effector target in the lung of a subject, the method comprising administering to the subject a macromolecule comprising a first binding site and a second binding site, wherein:
(a) the first binding site is specific for an effector target in the subject, and
(b) the second binding site is specific for RAGE, wherein the macromolecule is linked to a small molecule.
112. A method for modulating activity of an effector target in the kidney of a subject, the method comprising administering to the subject a macromolecule comprising a first binding site and a second binding site, wherein:
(a) the first binding site is specific for an effector target in the subject, and
(b) the second binding site is specific for cadherin 16 (CDH16), wherein the macromolecule is linked to a small molecule.
113. A method for modulating activity of an effector target in the intestine of a subject, the method comprising administering to the subject a macromolecule comprising a first binding site and a second binding site, wherein:
(a) the first binding site is specific for an effector target in the subject, and
(b) the second binding site is specific for cadherin 17 (CDH17), wherein the macromolecule is linked to a small molecule.
114. A method of localizing a macromolecule at a target tissue or cell of a subject, the method comprising administering to the subject a macromolecule comprising a first binding site and a second binding site, wherein:
(a) the first binding site is specific for an effector target in the subject, and
(b) the second binding site is specific for an address target expressed in the target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site; and
allowing the macromolecule to localize at the target tissue or cell of the subject, wherein the macromolecule is linked to a small molecule.
115. A method of concentrating a macromolecule in a target tissue or cell in a subject, the method comprising administering to the subject a macromolecule comprising a first binding site and a second binding site, wherein:
(a) the first binding site is specific for an effector target in a subject, and
(b) the second binding site is specific for an address target expressed in a target tissue or cell in the subject; wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site;
and allowing the macromolecule to concentrate at the target tissue or cell of the subject, wherein at least 25% of the macromolecule detectable in the subject is detected at the target tissue or cell at a time point between 1 and 7 days following administration of the macromolecule to the subject, wherein the macromolecule is linked to a small molecule.
116. The method of embodiment 114 or 115, wherein the potency of the first binding site at the target tissue or cell is substantially increased relative to a reference macromolecule lacking the second binding site.
117. The method of embodiment 114 or 115, wherein effector target signaling by the macromolecule in a non-target tissue or cell of the subject is substantially decreased relative to a reference macromolecule lacking the second binding site.
118. The method of embodiments 110-117, wherein the macromolecule is a macromolecule of any one of embodiments 1-86.
119. The macromolecule, method, or pharmaceutical composition of any one of claims 1-118, wherein the macromolecule and the small molecule are connected by a linker.
120. The macromolecule, method, or pharmaceutical composition of claim 119, wherein the linker is a cleavable linker.
121. The macromolecule, method, or pharmaceutical composition of claim 119, wherein the linker is a non-cleavable linker.
122. The macromolecule, method, or pharmaceutical composition of any one of claims 1-121, wherein the first binding site, the second binding site, or both is a polypeptide.
123. The macromolecule, method, or pharmaceutical composition of any one of claims 1-122, wherein a single small molecule is linked to the macromolecule.
124. The macromolecule, method, of pharmaceutical composition of any one of claims 1-122, wherein multiple small molecules are linked to the macromolecule.
125. The macromolecule, method, or pharmaceutical composition of claim 124, wherein each of the small molecules is the same.
126. The macromolecule, method, or pharmaceutical composition of claim 124, wherein at least two of the small molecules are different from each other.
127. The macromolecule, method, or pharmaceutical composition of any one of embodiments 1-126, wherein the target cell is an immune cell and the second binding site is specific for CD20.
128. The macromolecule, method, or pharmaceutical composition of any one of embodiments 1-126, wherein the target cell is an immune cell and the second binding site is specific for CD33.
129. The macromolecule, method, or pharmaceutical composition of any one of embodiments 1-128, wherein the small molecule is fluocinolone.
130. The macromolecule, method, or pharmaceutical composition of any one of embodiments 1-128, wherein the small molecule is ibrutinib.
131. The macromolecule, method, or pharmaceutical composition of any one of embodiments 1-128, wherein the small molecule is tofacitinib.
132. The macromolecule, method, or pharmaceutical composition of any one of embodiments 1-128, wherein, relative to a control macromolecule not linked to the small molecule, the macromolecule linked to the small molecule exhibits increased internalization.
133. The macromolecule, method, or pharmaceutical composition of any one of embodiments 1-128, wherein the macromolecule does not substantially induce cell death.
134. The macromolecule, method, or pharmaceutical composition of any one of embodiments 1-128, wherein the macromolecule modulates immune cell function, e.g., of B-cells or myeloid cells.

Although the foregoing invention has been described in some detail by way of illustration and example for purposes of clarity of understanding, the descriptions and examples should not be construed as limiting the scope of the invention.

What is claimed is:

1. A method of localizing a macromolecule at a target tissue or cell of a subject, the method comprising administering to the subject a macromolecule comprising a first binding site and a second binding site, wherein:
(a) the first binding site is specific for an effector target in the subject, and
(b) the second binding site is specific for an address target expressed in the target tissue or cell in the subject;
wherein:
(i) the second binding site localizes the first binding site to the address target such that the first binding site influences effector target signaling in the target tissue or cell;
(ii) the second binding site does not substantially influence signaling upon binding the address target; and
(iii) the first binding site does not substantially influence effector target signaling in the absence of localization by the second binding site; and
allowing the macromolecule to localize at the target tissue or cell of the subject, wherein the macromolecule is linked to a small molecule.

2. The method of claim 1, wherein the macromolecule and the small molecule are connected by a linker.

3. The method of claim 2, wherein the linker is a cleavable linker.

4. The method of claim 2, wherein the linker is a non-cleavable linker.

5. The method of claim 1, wherein at least 25% of the macromolecule detectable in the subject is detected at the target tissue or cell at a time point between 1 and 7 days following administration of the macromolecule to the subject.

6. The method of claim 1, wherein the potency of the first binding site at the target tissue or cell is substantially increased relative to a reference macromolecule lacking the second binding site.

7. The method of claim 6, wherein the first binding site has a low affinity for the effector target.

8. The method of claim 6, wherein the first binding site has a low avidity for the effector target.

9. The method of claim 1, wherein the affinity of the first binding site for the effector target is lower than the affinity of the second binding site for the address target.

10. The method of claim 1, wherein the avidity of the first binding site for the effector target is lower than the avidity of the second binding site for the address target.

11. The method of claim 1, wherein effector target signaling by the macromolecule in a non-target tissue or cell of the subject is substantially decreased relative to a reference macromolecule lacking the second binding site.

12. The method of claim 1, wherein the address target is regionally expressed in the subject

13. The method of claim 1, wherein the address target is locally expressed in the subject.

14. The method of claim 1, wherein the expression of the address target is restricted to a cell type in the subject.

15. The method of claim 1, wherein the address target is expressed only by a cell in the subject when in a specific cell state.

16. The method of claim 1, wherein the address target is expressed only by a cell in the subject in a disease state.

17. The method of claim 1, wherein the first binding site or the second binding site comprises a polypeptide.

18. The method of claim 17, wherein the polypeptide is an antibody or antigen-binding fragment thereof.

19. The method of claim 18, wherein the macromolecule is an antibody comprising a first binding site that is specific for the effector target in the subject and a second binding site that is specific for the address target.

20. The method of claim 17, wherein the polypeptide is a ligand of the effector target or a ligand of the address target.

21. The method of claim 20, wherein:
(a) the first binding site comprises an antibody or antigen-binding fragment thereof and the second binding site comprises a ligand of the address target; or
(b) the first binding site comprises a ligand of the effector target and the second binding site comprises an antibody or antigen-binding fragment thereof.

22. The method of claim 1, wherein the target tissue is skin and the second binding site is specific for desmoglein-1 (DSG-1).

23. The method of claim 1, wherein the target tissue is lung tissue and the second binding site is specific for RAGE.

24. The method of claim 1, wherein the target tissue is kidney tissue and the second binding site is specific for cadherin 16 (CDH16).

25. The method of claim 1, wherein the target tissue is intestine tissue and the second binding site is specific for cadherin 17 (CDH17).

26. The method of claim 1, wherein the target cell is an immune cell and the second binding site is specific for CD20.

27. The method of claim 1, wherein the target cell is an immune cell and the second binding site is specific for CD33.
